(19) United States
(12) Patent Application Publication
(10) Pub. No.: US 2010/0286044 A1
(43) Pub. Date: Nov. 11, 2010

(54) DETECTION OF TISSUE ORIGIN OF CANCER

(75) Inventors: Thomas Litman, Vaerlose (DK); Søren Møller, Holte (DK); Søren Morgenthaler Echwald, Humlebaek (DK); Nana Jacobsen, Gentofte (DK); Christian Glue, Frederiksberg (DK)

Correspondence Address: CLARK & ELBING LLP, 101 FEDERAL STREET, BOSTON, MA, 02110, US

(73) Assignee: Exiqon A/S, Vedbaek (DK)

(21) Appl. No.: 11/795,104

(22) PCT Filed: Dec. 29, 2006
(86) PCT No.: PCT/DK2006/000748

§ 371 Date: Feb. 26, 2010

(30) Foreign Application Priority Data
Dec. 29, 2005 (DK) PCT/DK2005/000838
Jul. 6, 2006 (DK) PA 2006 00930

(51) Int. Cl. A61K 38/22; C12Q 1/68; A61P 35/00; A61B 19/00

(52) U.S. Cl. 514/9.7; 435/6; 128/898

(57) ABSTRACT

Disclosed is a method for determining the cellular or tissue origin of tumor cells. The method entails determining the presence of at least one micro RNA (miRNA) in a sample derived from tumor tissue and based on the said determination establishing a miRNA expression profile and comparing this with pre-established miRNA expression profiles from cells, tissues or tumors. The determination uses short oligonucleotide probes comprising modified affinity-enhancing nucleobases.

The present invention relates to ribonucleic acids and oligonucleotide probes useful for detection and analysis of microRNAs and their target mRNAs, as well as small interfering RNAs (siRNAs). The invention furthermore relates to oligonucleotide probes for detection and analysis of other non-coding RNAs, as well as mRNAs, mRNA splice variants, allelic variants of single transcripts, mutations, deletions, or duplications of particular exons in transcripts, e.g. alterations associated with human disease, such as cancer.

### BACKGROUND OF THE INVENTION

The present invention relates to the detection and analysis of target nucleotide sequences in RNA samples derived from tumours, and more specifically to the methods employing the design and use of oligonucleotide probes that are useful for detecting and analysing target miRNA sequences in order to detect the origin of tumours.

MicroRNAs

The expanding inventory of international sequence databases and the concomitant sequencing of more than 200 genomes representing all three domains of life—bacteria, archea and eukaryota—have been the primary drivers in the process of deconstructing living organisms into comprehensive molecular catalogs of genes, transcripts and proteins. The importance of the genetic variation within a single species has become apparent, extending beyond the completion of genetic blueprints of several important genomes, culminating in the publication of the working draft of the human genome sequence in 2001 (Lander, Linton, Birren et al., 2001 Nature 409: 860-921; Venter, Adams, Myers et al., 2001 Science 291: 1304-1351; Sachidanandam, Weissman, Schmidt et al., 2001 Nature 409: 928-933). On the other hand, the increasing number of detailed, large-scale molecular analyses of transcription originating from the human and mouse genomes along with the recent identification of several types of non-protein-coding RNAs, such as small nucleolar RNAs, siRNAs, microRNAs and antisense RNAs, indicate that the transcriptomes of higher eukaryotes are much more complex than originally anticipated (Wong et al. 2001, Genome Research 11: 1975-1977; Kampa et al. 2004, Genome Research 14: 331-342).

As a result of the Central Dogma: ‘DNA makes RNA, and RNA makes protein’, RNAs have been considered as simple molecules that just translate the genetic information into protein. Recently, it has been estimated that although most of the genome is transcribed, almost 97% of the genome does not encode proteins in higher eukaryotes, but putative, non-coding RNAs (Wong et al. 2001, Genome Research 11: 1975-1977). The non-coding RNAs (ncRNAs) appear to be particularly well suited for regulatory roles that require highly specific nucleic acid recognition. Therefore, the view of RNA is rapidly changing from the merely informational molecule to comprise a wide variety of structural, informational and catalytic molecules in the cell.

Recently, a large number of small non-coding RNA genes have been identified and designated as microRNAs (miRNAs) (for review, see Ke et al. 2003, Curr. Opin. Chem. Biol. 7:516-523). The first miRNAs to be discovered were the lin-4 and let-7 that are heterochronic switching genes essential for the normal temporal control of diverse developmental events (Lee et al. 1993, Cell 75:843-854; Reinhart et al. 2000, Nature 403: 901-906) in the roundworm C. elegans. miRNAs have been evolutionarily conserved over a wide range of species and exhibit diversity in expression profiles, suggesting that they occupy a wide variety of regulatory functions and exert significant effects on cell growth and development (Ke et al. 2003, Curr. Opin. Chem. Biol. 7:516-523). Recent work has shown that miRNAs can regulate gene expression at many levels, representing a novel gene regulatory mechanism and supporting the idea that RNA is capable of performing similar regulatory roles as proteins. Understanding this RNA-based regulation will help us to understand the complexity of the genome in higher eukaryotes as well as understand the complex gene regulatory networks.

miRNAs are 18-25 nucleotide (nt) RNAs that are processed from longer endogenous hairpin transcripts (Ambros et al. 2003, RNA 9: 277-279). To date more than 1420 microRNAs have been identified in humans, worms, fruit flies and plants according to the miRNA registry database release 5.1 in December 2004, hosted by Sanger Institute, UK, and many miRNAs that correspond to putative genes have also been identified. Some miRNAs have multiple loci in the genome (Reinhart et al. 2002, Genes Dev. 16: 1616-1626) and occasionally, several miRNA genes are arranged in tandem clusters (Lagos-Quintana et al. 2001, Science 294: 853-858). The fact that many of the miRNAs reported to date have been isolated just once suggests that many new miRNAs will be discovered in the future. A recent in-depth transcriptional analysis of the human chromosomes 21 and 22 found that 49% of the observed transcription was outside of any known annotation, and furthermore, that these novel transcripts were both coding and non-coding RNAs (Kampa et al. 2004, Genome Research 14: 331-342). Another recent paper describes the use of phylogenetic shadowing profiles to predict 976 novel candidate miRNA genes in the human genome (Berezikov et al. 2005, Cell 120: 21-24) from whole-genome human/mouse and human/rat alignments. Most of the candidate miRNA genes were found to be conserved in other vertebrates, including dog, cow, chicken, opossum and zebrafish. Thus, the identified miRNAs to date represent most likely the tip of the iceberg, and the number of miRNAs might turn out to be very large.

The combined characteristics of microRNAs characterized to date (Ke et al. 2003, Curr. Opin. Chem. Biol. 7:516-523; Lee et al. 1993, Cell 75:843-854; Reinhart et al. 2000, Nature 403: 901-906) can be summarized as:

1. miRNAs are single-stranded RNAs of about 18-25 nt that regulate the expression of complementary messenger RNAs

2. They are cleaved from a longer endogenous double-stranded hairpin precursor by the enzyme Dicer.

3. miRNAs match precisely the genomic regions that can potentially encode precursor miRNAs in the form of double-stranded hairpins.

4. miRNAs and their predicted precursor secondary structures may be phylogenetically conserved.

Several lines of evidence suggest that the enzymes Dicer and Argonaute are crucial participants in miRNA biosynthesis, maturation and function (Grishok et al. 2001, Cell 106: 23-24). Mutations in genes required for miRNA biosynthesis lead to genetic developmental defects, which are, at least in part, derived from the role of generating miRNAs. The current view is that miRNAs are cleaved by Dicer from the hairpin precursor in the form of duplex, initially with 2 or 3 nt overhangs in the 3′ ends, and are termed pre-miRNAs. Cofactors join the pre-miRNP (microRNA RiboNucleoProtein-complexes) and unwind the pre-miRNAs into single-stranded miRNAs, and pre-miRNP is then transformed to miRNP. miRNAs can recognize regulatory targets while part of the miRNP complex. There are several similarities between miRNP and the RNA-induced silencing complex, RISC, including similar sizes and both containing RNA helicase and the PPD proteins. It has therefore been proposed that miRNP and RISC are the same RNP with multiple functions (Ke et al. 2003, Curr. Opin. Chem. Biol. 7:516-523). Different effectors direct miRNAs into diverse pathways. The structure of pre-miRNAs is consistent with the observation that 22 nt RNA duplexes with 2 or 3 nt overhangs at the 3′ ends are beneficial for reconstitution of the protein complex and might be required for high affinity binding of the short RNA duplex to the protein components (for review, see Ke et al. 2003, Curr. Opin. Chem. Biol. 7:516-523).

Growing evidence suggests that miRNAs play crucial roles in eukaryotic gene regulation. The first miRNAs genes to be discovered, lin-4 and let-7, base-pair incompletely to repeated elements in the 3′ untranslated regions (UTRs) of other heterochronic genes, and regulate the translation directly and negatively by antisense RNA-RNA interaction (Lee et al. 1993, Cell 75:843-854; Reinhart et al. 2000, Nature 403: 901-906). Other miRNAs are thought to interact with target mRNAs by limited complementary and suppressed translation as well (Lagos-Quintana et al. 2001, Science 294: 853-858; Lee and Ambros 2001, Science 294: 858-862). Many studies have shown, however, that given a perfect complementarity between miRNAs and their target RNA, could lead to target RNA degradation rather than inhibit translation (Hutvagner and Zamore 2002, Science 297: 2056-2060), suggesting that the degree of complementarity determines their functions. By identifying sequences with near complementarity, several targets have been predicted, most of which appear to be potential transcriptional factors that are crucial in cell growth and development. The high percentage of predicted miRNA targets acting as developmental regulators and the conservation of target sites suggest that miRNAs are involved in a wide range of organism development and behaviour and cell fate decisions (for review, see Ke et al. 2003, Curr. Opin. Chem. Biol. 7:516-523). For example, John et al. 2004 (PLoS Biology 2: e363) used known mammalian miRNAs to scan the 3′ untranslated regions (UTRs) from human, mouse and rat genomes for potential miRNA target sites using a scanning algorithm based on sequence complementarity between the mature miRNA and the target site, binding energy of the miRNA:mRNA duplex and evolutionary conservation. They identified a total of 2307 target mRNAs conserved across the mammals with more than one target site at 90% conservation of target site sequence and 660 target genes at 100% conservation level. Scanning of the two fish genomes; Danio rerio (zebrafish) and Fugu rubripes (Fugu) identified 1000 target genes with two or more conserved miRNA sites between the two fish species (John et al. 2004 PLoS Biology 2: e363). Among the predicted targets, particularly interesting groups included mRNA encoding transcription factors, components of the miRNA machinery, other proteins involved in the translational regulation as well as components of the ubiquitin machinery. In a recent paper, Lewis et al. (Lewis et al. 2005, Cell 120: 15-20) predicted regulatory mRNA targets of vertebrate microRNAs by identifying conserved complementarity to the so-called seed (comprising nucleotides 2 to 7) sequence of the miRNAs. In a comparative four-genome analysis of all the 3′ UTRs, ca. 5300 human genes were implicated as miRNA targets, which represented ca 30% of the gene set used in the analysis. In another recent publication, Lim et al. (Lim et al. 2005, Nature 433: 769-773) showed that transfection of HeLa cells with miR-124, a brain-specific microRNA, caused the expression profile of the HeLa cells to shift towards that of brain, as revealed by genome-wide expression profiling of the HeLa mRNA pool. By comparison, delivery of miR-1 to the HeLa cells shifted the mRNA profile toward muscle, the tissue where miR-1 is preferentially expressed. Lim et al. (Lim et al. 2005, Nature 433: 769-773) subsequently showed that the 3′ un-translated regions of the downregulated mRNAs had a significant propensity to pair to the seed sequence of the 5′ end of the two miRNAs, thus implying that metazoan miRNAs can reduce the levels of many of their target mRNAs. Wang et al. 2004 (Genome Biology 5:R65) have developed and applied a computational algorithm to predict 95 Arabidopsis thaliana miRNAs, which included 12 known ones and 83 new miRNAs. The 83 new miRNAs were found to be conserved with more than 90% sequence identity between the Arabidopsis and rice genomes. Using the Smith-Waterman nucleotide-alignment algorithm to predict mRNA targets for the 83 new miRNAs and by focusing on target sites that were conserved in both Arabidopsis and rice, Wang et al. 2004 (Genome Biology 5:R65) predicted 371 mRNA targets with an average of 4.8 targets per miRNA. A large proportion of these mRNA targets encoded proteins with transcription regulatory activity. Brennecke et al. 2005 (Brennecke et al. 2005 PLoS Biology 3: e85) have systematically evaluated the minimal requirements for functional miRNA:mRNA target duplexes in vivo and have grouped the target sites into two categories. The so-called 5′ dominant sites have sufficient complementarity to the 5′-end on the miRNA, so that little or no pairing with the 3′-end of the miRNA is needed. The second class comprises the so-called 3′ compensatory sites, which have insufficient 5′-end pairing and require strong 3′-end duplex formation in order to be functional. In addition to presenting experimental examples from both types of miRNA:target pairing in vivo, Brennecke et al. 2005 (Brennecke et al. 2005 PLoS Biology 3: e85) provide evidence that a given miRNA has in average ca. 100 mRNA target sites, further supporting the notion that miRNAs can regulate the expression of a large fraction of the protein-coding genes in multicellular eukaryotes.

MicroRNAs and Human Disease

Analysis of the genomic location of miRNAs indicates that they play important roles in human development and disease. Several human diseases have already been pinpointed in which miRNAs or their processing machinery might be implicated. One of them is spinal muscular atrophy (SMA), a paediatric neurodegenerative disease caused by reduced protein levels or loss-of-function mutations of the survival of motor neurons (SMN) gene (Paushkin et al. 2002, Curr. Opin. Cell Biol. 14: 305-312). Two proteins (Gemin3 and Gemin4) that are part of the SMN complex are also components of miRNPs, whereas it remains to be seen whether miRNA biogenesis or function is dysregulated in SMA and what effect this has on pathogenesis. Another neurological disease linked to mi/siRNAs is fragile X mental retardation (FXMR) caused by absence or mutations of the fragile X mental retardation protein (FMRP) (Nelson et al. 2003, TIBS 28: 534-540), and there are additional clues that miRNAs might play a role in other neurological diseases. Yet another interesting finding is that the miR-224 gene locus lies within the minimal candidate region of two different neurological diseases: early-onset Parkinsonism and X-linked mental retardation (Dostie et al. 2003, RNA: 9: 180-186). Links between cancer and miRNAs have also been recently described. The most frequent single genetic abnormality in chronic lymphocytic leukaemia (CLL) is a deletion localized to chromosome 13q14 (50% of the cases). A recent study determined that two different miRNA (miR15 and miR16) genes are clustered and located within the intron of LEU2, which lies within the deleted minimal region of the B-cell chronic lymphocytic leukaemia (B-CLL) tumour suppressor locus, and both genes are deleted or down-regulated in the majority of CLL cases (Calin et al. 2002, Proc. Natl. Acad. Sci. U.S.A. 99: 15524-15529). Calin et al. 2004 (Calin et al. 2004, Proc. Natl. Acad. Sci. U.S.A. 101: 2999-3004) have further investigated the possible involvement of microRNAs in human cancers on a genome-wide basis, by mapping 186 miRNA genes and compared their location to the location of previous reported non-random genetic alterations. Interestingly, they showed that microRNA genes are frequently located at fragile sites, as well as in minimal regions of loss of heterozygosity, minimal regions of amplification (minimal amplicons), or common breakpoint regions. Overall, 98 of 186 (52.5%) of the microRNA genes in their study were in cancer-associated genomic regions or in fragile sites. Moreover, by Northern blotting, Calin et al. 2004 (Calin et al. 2004, Proc. Natl. Acad. Sci. U.S.A. 101: 2999-3004) showed that several miRNAs located in deleted regions had low levels of expression in cancer samples. These data provide the first catalog of miRNA genes that may have roles in cancer and indicate that the full complement of human miRNAs may be extensively involved in different cancers.

In a recent study, Eis et al. (Eis et al. 2005, Proc. Natl. Acad. Sci. U.S.A. 102: 3627-3632) showed that the human miR-155 is processed from sequences present in BIC RNA, which is a spliced and polyadenylated non-protein-coding RNA that accumulates in lymphoma cells. The precursor of miR-155 is most likely a transient spliced or unspliced nuclear BIC transcript rather than accumulated BIC RNA, which is primarily cytoplasmic. Eis et al. (Eis et al. 2005, Proc. Natl. Acad. Sci. U.S.A. 102: 3627-3632) also observed that clinical isolates of several types of B cell lymphomas, including diffuse large B cell lymphoma (DLBCL), have 10- to 30-fold higher copy numbers of miR-155 than do normal circulating B cells. Significantly higher levels of miR-155 were present in DLBCLs with an activated B cell phenotype than with the germinal center phenotype. Because patients with activated B cell-type DLBCL have a poorer clinical prognosis, Eis et al. (Eis et al. 2005, Proc. Natl. Acad. Sci. U.S.A. 102: 3627-3632) propose that quantification of this microRNA would be diagnostically useful.

In another recent paper, Poy et al. (Poy et al. 2004, Nature 432: 226-230) identified a novel, evolutionarily conserved and pancreatic islet-specific miRNA (miR-375), and showed that overexpression of miR-375 suppressed glucose-induced insulin secretion, and conversely, inhibition of endogenous miR-375 function enhanced insulin secretion. The mechanism by which secretion is modified by miR-375 is independent of changes in glucose metabolism or intracellular Ca²⁺-signalling but correlated with a direct effect on insulin exocytosis. In the study, Myotrophin was validated as a target of miR-375. inhibition of Myotrophin by small interfering (si)RNA mimicked the effects of miR-375 on glucose-stimulated insulin secretion and exocytosis. Poy et al. (Poy et al. 2004, Nature 432: 226-230) thus conclude that miR-375 is a regulator of insulin secretion and could constitute a novel pharmacological target for the treatment of diabetes.

Yet another recent publication by Johnson et al. (Johnson et al. 2005, Cell 120: 635-647) showed that the let-7 miRNA family negatively regulates RAS in two different C. elegans tissues and two different human cell lines. Another interesting finding was that let-7 is expressed in normal adult lung tissue but is poorly expressed in lung cancer cell lines and lung cancer tissue. Furthermore, the expression of let-7 inversely correlates with expression of RAS protein in lung cancer tissues, suggesting a possible causal relationship. Overexpression of let-7 inhibited growth of a lung cancer cell line in vitro, suggesting a causal relationship between let-7 and cell growth in these cells. The combined results of Johnson et al. (Johnson et al. 2005, Cell 120: 635-647) that let-7 expression is reduced in lung tumors, that several let-7 genes map to genomic regions that are often deleted in lung cancer patients, that overexpression of let-7 can inhibit lung tumor cell line growth, that the expression of the RAS oncogene is regulated by let-7, and that RAS is significantly overexpressed in lung tumor samples strongly implicate let-7 as a tumor suppressor in lung tissue and also suggests a possible mechanism.

In conclusion, it has been anticipated that connections between miRNAs and human diseases will only strengthen in parallel with the knowledge of miRNAs and the gene networks that they control. Moreover, the understanding of the regulation of RNA-mediated gene expression is leading to the development of novel therapeutic approaches that will be likely to revolutionize the practice of medicine (Nelson et al. 2003, TIBS 28: 534-540).

Detection and Analysis of microRNAs

The current view that miRNAs may represent a newly discovered, hidden layer of gene regulation has resulted in high interest among researchers around the world in the discovery of miRNAs, their targets and mechanism of action. Detection and analysis of these small RNAs is, however not trivial. Thus, the discovery of more than 1400 miRNAs to date has required taking advantage of their special features. First, the research groups have used the small size of the miRNAs as a primary criterion for isolation and detection. Consequently, standard cDNA libraries would lack miRNAs, primarily because RNAs that small are normally excluded by sixe selection in the cDNA library construction procedure. Total RNA from fly embryos, worms or HeLa cells have been size fractionated so that only molecules 25 nucleotides or smaller would be captured (Moss 2002, Curr. Biology 12: R138-R140). Synthetic oligomers have then been ligated directly to the RNA pools using T4 RNA ligase. Then the sequences have been reverse-transcribed, amplified by PCR, cloned and sequenced (Moss 2002, Curr. Biology 12: R138-R140). The genome databases have subsequently been queried with the sequences, confirming the origin of the miRNAs from these organisms as well as placing the miRNA genes physically in the context of other genes in the genome. The vast majority of the cloned sequences have been located in intronic regions or between genes, occasionally in clusters, suggesting that the tandemly arranged miRNAs are processed from a single transcript to allow coordinate regulation. Furthermore, the genomic sequences have revealed the fold-back structures of the miRNA precursors (Moss 2002, Curr. Biology 12: R138-R140).

The size and often low level of expression of different miRNAs require the use of sensitive and quantitative analysis tools. Due to their small size of 18-25 nt, the use of conventional quantitative real-time PCR for monitoring expression of mature miRNAs is excluded. Therefore, most miRNA researchers currently use Northern blot analysis combined with polyacrylamide gels to examine expression of both the mature and pre-miRNAs (Reinhart et al. 2000, Nature 403: 901-906; Lagos-Quintana et al. 2001, Science 294: 853-858; Lee and Ambros 2001, Science 294: 862-864). Primer extension has also been used to detect the mature miRNA (Zeng and Cullen 2003, RNA 9: 112-123). The disadvantage of all the gel-based assays (Northern blotting, primer extension, RNase protection assays etc.) as tools for monitoring miRNA expression includes low throughput and poor sensitivity. Consequently, a large amount of total RNA per sample is required for Northern analysis of miRNAs, which is not feasible when the cell or tissue source is limited.

DNA microarrays would appear to be a good alternative to Northern blot analysis to quantify miRNAs in a genome-wide scale, since microarrays have excellent throughput. Krichevsky et al. 2003 used cDNA microarrays to monitor the expression of miRNAs-during neuronal development with 5 to 10 μg aliquot of input total RNA as target, but the mature miRNAs had to be separated from the miRNA precursors using micro concentrators prior to microarray hybridizations (Krichevsky et al. 2003, RNA 9: 1274-1281). Liu et al 2004 (Liu et al. 2004, Proc. Natl. Acad. Sci, U.S.A 101:9740-9744) have developed a microarray for expression profiling of 245 human and mouse miRNAs using 40-mer DNA oligonucleotide capture probes. Thomson et al. 2004 (Thomson et al. 2004, Nature Methods 1: 1-6) describe the development of a custom oligonucleotide microarray platform for expression profiling of 124 mammalian miRNAs conserved in human and mouse using oligonucleotide capture probes complementary to the mature microRNAs. The microarray was used in expression profiling of the 124 miRNAs in question in different adult mouse tissues and embryonic stages. A similar approach was used by Miska et al. 2004 (Genome Biology 2004; 5:R68) for the development of an oligoarray for expression profiling of 138 mammalian miRNAs, including 68 miRNAs from rat and monkey brains. Yet another approach was taken by Barad et al. 2004 (Genome Research 2004; 14: 2486-2494), who developed a 60-mer oligonucleotide microarray platform for known human mature miRNAs and their precursors. The drawback of all DNA-based oligonucleotide arrays regardless of the capture probe length is the requirement of high concentrations of labelled input target RNA for efficient hybridization and signal generation, low sensitivity for rare and low-abundant miRNAs, and the necessity for post-array validation using more sensitive assays such as real-time quantitative PCR, which is not currently feasible. In addition, at least in some array platforms discrimination of highly homologous miRNA differing by just one or two nucleotides could not be achieved, thus presenting problems in data interpretation, although the 60-mer microarray by Barad et al. 2004 (Genome Research 2004; 14: 2486-2494) appears to have adequate specificity.

A PCR approach has also been used to determine the expression levels of mature miRNAs (Grad et al. 2003, Mol. Cell 11: 1253-1263). This method is useful to clone miRNAs, but highly impractical for routine miRNA expression profiling, since it involves gel isolation of small RNAs and ligation to linker oligonucleotides. Allawi et al. (2004, RNA 10: 1153-1161) have developed a method for quantitation of mature miRNAs using a modified invader assay. Although apparently sensitive and specific for the mature miRNA, the drawback of the invader quantitation assay is the number of oligonucleotide probes and individual reaction steps needed for the complete assay, which increases the risk of cross-contamination between different assays and samples, especially when high-throughput analyses are desired. Schmittgen et al. (2004, Nucleic Acids Res. 32: e43) describe an alternative method to Northern blot analysis, in which they use real-time PCR assays to quantify the expression of miRNA precursors. The disadvantage of this method is that it only allows quantification of the precursor miRNAs, which does not necessarily reflect the expression levels of mature miRNAs. In order to fully characterize the expression of large numbers of miRNAs, it is necessary to quantify the mature miRNAs, such as those expressed in human disease, where alterations in miRNA biogenesis produce levels of mature miRNAs that are very different from those of the precursor miRNA. For example, the precursors of 26 miRNAs were equally expressed in non-cancerous and cancerous colorectal tissues from patients, whereas the expression of mature human miR143 and miR145 was greatly reduced in cancer tissues compared with non-cancer tissues, suggesting altered processing for specific miRNAs in human disease (Michael et al. 2003, Mol. Cancer Res. 1: 882-891). On the other hand, recent findings in maize with miR166 and miR165 in Arabidopsis thaliana, indicate that microRNAs act as signals to specify leaf polarity in plants and may even form movable signals that emanate from a signalling centre below the incipient leaf (Juarez et al. 2004, Nature 428: 84-88; Kidner and Martienssen 2004, Nature 428: 81-84).

Most of the miRNA expression studies in animals and plants have utilized Northern blot analysis, tissue-specific small RNA cloning and expression profiling by microarrays or real-time PCR of the miRNA hairpin precursors, as described above. However, these techniques lack the resolution for addressing the spatial and temporal expression patterns of mature miRNAs. Due to the small size of mature miRNAs, detection of them by standard RNA in situ hybridization has proven difficult to adapt in both plants and vertebrates, even though in situ hybridization has recently been reported in A. thaliana and maize using RNA probes corresponding to the stem-loop precursor miRNAs (Chen et al. 2004, Science 203: 2022-2025; Juarez et al. 2004, Nature 428: 84-88). Brennecke et al. 2003 (Cell 113: 25-36) and Mansfield et al. 2004 (Nature Genetics 36: 1079-83) report on an alternative method in which reporter transgenes, so-called sensors, are designed and generated to detect the presence of a given miRNA in an embryo. Each sensor contains a constitutively expressed reporter gene (e.g. lacZ or green fluorescent protein) harbouring miRNA target sites in its 3′-UTR. Thus, in cells that lack the miRNA in question, the transgene RNA is stable allowing detection of the reporter, whereas cells expressing the miRNA, the sensor mRNA is targeted for degradation by the RNAi pathway. Although sensitive, this approach is time-consuming since it requires generation of the expression constructs and transgenes. Furthermore, the sensor-based technique detects the spatiotemporal miRNA expression patterns via an indirect method as opposed to direct in situ hybridization of the mature miRNAs.

The large number of miRNAs along with their small size makes it difficult to create loss-of-function mutants for functional genomics analyses. Another potential problem is that many miRNA genes are present in several copies per genome occurring in different loci, which makes it even more difficult to obtain mutant phenotypes. Boutla et al. 2003 (Nucleic Acids Research 31: 4973-4980) describe the use of DNA antisense oligonucleotides complementary to 11 different miRNAs in Drosophila as well as their use to inactivate the miRNAs by injecting the DNA oligonucleotides into fly emryos. Of the 11 DNA antisense oligonucleotides, only 4 constructs showed severe interference with normal development, while the remaining 7 oligonucleotides didn't show any phenotypes presumably due to their inability to inhibit the miRNA in question. Thus, the success rate for using DNA antisense oligonucleotides to inhibit miRNA function would most likely be too low to allow functional analyses of miRNAs on a larger, genomic scale. An alternative approach to this has been reported by Hutvagner et al. 2004 (PLoS Biology 2: 1-11), in which 2′-O-methyl antisense oligonucleotides could be used as potent and irreversible inhibitors of siRNA and miRNA function in vitro and in vivo in Drosophila and C. elegans, thereby inducing a loss-of-function phenotype. A drawback of this method is the need of high 2′-O-methyl oligonucleotide concentrations (100 micromolar) in transfection and injection experiments, which may be toxic to the animal.

In conclusion, the biggest challenge in detection, quantitation and functional analysis of the mature miRNAs as well as siRNAs using currently available methods is their small size of the of 18-25 nt and often low level of expression. The present invention provides the design and development of novel oligonucleotide compositions and probe sequences for accurate, highly sensitive and specific detection and functional analysis of miRNAs, their target mRNAs and siRNA transcripts.

Cancer Diagnosis and Identification of Tumor Origin

Cancer classification relies on the subjective interpretation of both clinical and histopathological information by eye with the aim of classifying tumors in generally accepted categories based on the tissue of origin of the tumor. However, clinical information can be incomplete or misleading. In addition, there is a wide spectrum in cancer morphology and many tumors are atypical or lack morphologic features that are useful for differential diagnosis. These difficulties may result in diagnostic confusion, with the need for mandatory second opinions in all surgical pathology cases (Tomaszewski and LiVolsi 1999, Cancer 86: 2198-2200).

Molecular diagnostics offer the promise of precise, objective, and systematic human cancer classification, but these tests are not widely applied because characteristic molecular markers for most solid tumors have yet to be identified. In the recent years microarray-based tumor gene expression profiling has been used for cancer diagnosis. However, studies are still limited and have utilized different array platforms making it difficult to compare the different datasets (Golub et al. 1999, Science 286: 531-537; Alizadeh et al. 2000, Nature 403: 503-511; Bittner et al. 2000, Nature 406: 536-540). In addition, comprehensive gene expression databases have to be developed, and there are no established analytical methods yet capable of solving complex, multiclass, gene expression-based classification problems.

Another problem for cancer diagnostics is the identification of tumor origin for metastatic carcinomas. For example, in the United States, 51,000 patients (4% of all new cancer cases) present annually with metastases arising from occult primary carcinomas of unknown origin (ACS Cancer Facts & Figures 2001: American Cancer Society). Adenocarcinomas represent the most common metastatic tumors of unknown primary site. Although these patients often present at a late stage, the outcome can be positively affected by accurate diagnoses followed by appropriate therapeutic regimens specific to different types of adenocarcinoma (Hilien 2000, Postgrad. Med. J. 76: 690-693). The lack of unique microscopic appearance of the different types of adenocarcinomas challenges morphological diagnosis of adenocarcinomas of unknown origin. The application of tumor-specific serum markers in identifying cancer type could be feasible, but such markers are not available at present (Milovic et al. 2002, Med. Sci. Monit. 8: MT25-MT30). Microarray expression profiling has recently been used to successfully classify tumors according to their site of origin (Ramaswamy et al. 2001, Proc. Natl. Acad. Sci. U.S.A. 98: 15149-15154), but the lack of a standard for array data collection and analysis make them difficult to use in a clinical setting. SAGE (serial analysis of gene expression), on the other hand, measures absolute expression levels through a tag counting approach, allowing data to be obtained and compared from different samples. The drawback of this method is, however, its low throughput, making it inappropriate for routine clinical applications. Quantitative real-time PCR is a reliable method for assessing gene expression levels from relatively small amounts of tissue (Bustin 2002, J. Mol. Endocrinol. 29: 23-39). Although this approach has recently been successfully applied to the molecular classification of breast tumors into prognostic subgroups based on the analysis of 2,400 genes (Iwao et al. 2002, Hum. Mol. Genet. 11: 199-206), the measurement of such a large number of randomly selected genes by PCR is clinically impractical.

US 2006/0094035 discloses a method for tissue typing of cancer cells in a sample by utilising the expression levels of >50 transcribed genes and comparing the expression levels of these genes with their expression levels in known tumour/tissue/cell samples. US 2006/0094035 does not mention the possibility of typing tumours based on their expression levels of a variety of miRNAs.

US 2006/0265138 relates to methods of profiling tumours and characterisation of the tissue types associated with the tumours. A gene expression profile is obtained from the tissue sample, the genes ranked in order of their relative expression levels and the tissue type is identified by comparing the gene ranking obtained with a database of relative gene expression level rankings of different tissue types. This gives a means to identify primary tumours and to determine the identity of a tumour of unknown primary. The application does not mention the possibility of typing tumours of unknown origin according to their expression of miRNA species.

Since the discovery of the first miRNA gene lin-4, in 1993, microRNAs have emerged as important non-coding RNAs, involved in a wide variety of regulatory functions during cell growth, development and differentiation. Furthermore, an expanding inventory of microRNA studies has shown that many miRNAs are mutated or down-regulated in human cancers, implying that miRNAs can act as tumor supressors or even oncogenes. Thus, detection and quantitation of all the microRNAs with a role in human disease, including cancers, would be highly useful as biomarkers for diagnostic purposes or as novel pharmacological targets for treatment. The biggest challenge, on the other hand, in detection and quantitation of the mature miRNAs using currently available methods is the small size of 18-25 nt and sometimes low level of expression.

The present invention solves the abovementioned problems by providing the design and development of novel oligonucleotide compositions and probe sequences for accurate, highly sensitive and specific detection and quantitation of microRNAs and other non-coding RNAs, useful as biomarkers for diagnostic purposes of human disease as well as for antisense-based intervention, which is targeted against tumorigenic miRNAs and other non-coding RNAs. The invention furthermore provides novel oligonucleotide compositions and probe sequences for sensitive and specific detection and quantitation of microRNAs, useful as biomarkers for the identification of the primary site of metastatic tumors of unknown origin.

### SUMMARY OF THE INVENTION

The challenges of establishing genome function and understanding the layers of information hidden in the complex transcriptomes of higher eukaryotes call for novel, improved technologies for detection and analysis of non-coding RNA and protein-coding RNA molecules in complex nucleic acid samples. Thus, it is highly desirable to be able to detect and analyse the expression of mature microRNAs of eukaryotes using methods based on specific and sensitive oligonucleotide detection probes in order to determine the origin of metastatic tumours, where the primary tumour cannot be readily identified.

The present invention solves the current problems faced by conventional approaches used in detection and analysis of mature miRNAs and utilises this in the identification of tumour tissue origin. The invention utilises oligonucleotide probes which comprise a recognition sequence complementary to the RNA target sequence, which said recognition sequence is substituted with high-affinity nucleotide analogues, e.g. LNA, to increase the sensitivity and specificity of conventional oligonucleotides, such as DNA oligonucleotides, for hybridization to short target sequences, e.g. mature miRNAs and stem-loop precursor miRNAs.

In the broadest aspect, the present invention relates to a method for specifically identifying, in a mammal (such as a human being), the primary site of a metastatic tumour of unknown origin, said method comprising

a) contacting a sample derived from tumour cells of said metastatic tumour with at least one detection probe, which is a member from a collection of detection probes wherein each member of said collection comprises a recognition sequence consisting of nucleobases and affinity enhancing nucleobase analogues, and wherein the recognition sequences exhibit a combination of high melting temperatures and low self-complementarity scores, said melting temperatures being the melting temperature of the duplex between the recognition sequence and its complementary RNA sequence, said collection of detection probes being capable of specifically identifying target RNA sequences in all miRNAs of said mammal and said sample being contacted with said at least one member under conditions that facilitate hybridization between said member and its complementary RNA sequence, and

b) subsequently detecting hybridization between said at least one detection probe and its complementary RNA.

The present invention is based on the disclosure the present assignee's own WO 2006/069584 which discloses the majority of the necessary means and methods necessary in order to practice the current invention.

The present invention thus utilises the method disclosed in WO 2006/069584 of designing the detection probe sequences by selecting optimal substitution patterns for the high-affinity analogues, e.g. LNAs for the detection probes. This method involves (a) substituting the detection probe sequence with the high affinity analogue LNA in chimeric LNA-DNA oligonucleotides using regular spacing between the LNA substitutions, e.g. at every second nucleotide position, every third nucleotide position, or every fourth nucleotide position, in order to promote the A-type duplex geometry between the substituted detection probe and its complementary RNA target; with the said LNA monomer substitutions spiked in all the possible phases in the probe sequence with an unsubstituted monomer at the 5′-end position and 3′-end position in all the substituted designs; (b) determining the ability of the designed detection probes with different regular substitution patterns to self-anneal; and (c) determining the melting temperature of the substituted probes sequences of the invention, and (d) selecting the probe sequences with the highest melting temperatures and lowest self-complementarity score, i.e. lowest ability to self-anneal are selected.

The invention also utilises the method disclosed in WO 2006/069584 of designing the detection probe sequences by selecting optimal substitution patterns for the LNAs, which said method involves substituting the detection probe sequence with the high affinity analogue LNA in chimeric LNA-DNA oligonucleotides using irregular spacing between the LNA monomers and selecting the probe sequences with the highest melting temperatures and lowest self-complementarity score. In yet another aspect the invention utilises a computer code for the design and selection of the said substituted detection probe sequences.

The present invention also utilises detection probes disclosed in WO 2006/069584, which are derived from a collection of detection probes, wherein each member of said collection comprises a recognition sequence consisting of nucleobases and affinity enhancing nucleobase analogues, and wherein the recognition sequences exhibit a combination of high melting temperatures and low self-complementarity scores, said melting temperatures being the melting temperature of the duplex between the recognition sequence and its complementary DNA or RNA sequence.

The invention also utilises the finding disclosed in WO 2006/069584 that it is possible to expand or build a collection of detection probes defined above, by

A) defining a reference nucleotide sequence consisting of nucleobases, said reference nucleotide sequence being complementary to a target sequence for which the collection does not contain a detection probe,

B) substituting the reference nucleotide sequence's nucleobases with affinity enhancing nucleobase analogues to provide a set of chimeric sequences wherein,

C) determining usefulness of each of the chimeric sequences based on assessment of their ability to self-anneal and their melting temperature, and

D) synthesizing and adding, to the collection, a probe comprising as its recognition sequence the chimeric sequence with the optimum combination of high melting temperature and low self-annealing.

The invention further takes advantage of the fact that one, according to WO 2006/069584, can design an optimized detection probe for a target nucleotide sequence by

1) defining a reference nucleotide sequence consisting of nucleobases, said reference nucleotide sequence being complementary to said target nucleotide sequence,

2) substituting the reference nucleotide sequence's nucleobases with affinity enhancing nucleobase analogues to provide a set of chimeric sequences

3) determining usefulness of each of the chimeric sequences based on assessment of their ability to self-anneal and their melting temperatures, and

4) defining the optimized detection probe as the one in the set having as its recognition sequence the chimeric sequence with the optimum combination of high melting temperature and low self-annealing.

Furthermore, the present invention also relies on a computer system disclosed in WO 2006/069584 for designing an optimized detection probe for a target nucleic acid sequence, said system comprising

a) input means for inputting the target nucleotide,

b) storage means for storing the target nucleotide sequence,

c) optionally executable code which can calculate a reference nucleotide sequence being complementary to said target nucleotide sequence and/or input means for inputting the reference nucleotide sequence,

d) optionally storage means for storing the reference nucleotide sequence,

e) executable code which can generate chimeric sequences from the reference nucleotide sequence or the target nucleic acid sequence, wherein said chimeric sequences comprise the reference nucleotide sequence, wherein has been in-substituted affinity enhancing nucleobase analogues,

f) executable code which can determine the usefulness of such chimeric sequences based on assessment of their ability to self-anneal and their melting temperatures and either rank such chimeric sequences according to their usefulness,

g) storage means for storing at least one chimeric sequence, and

h) output means for presenting the sequence of at least one optimized detection probe.

In another aspect the invention relies on detection probe sequences containing a ligand. Such ligand-containing detection probes of the invention are useful for isolating target RNA molecules from complex mixtures of miRNAs. Ligands comprise biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicar-bazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C1-C20 alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also affinity ligands, i.e. functional groups or biomolecules that have a specific affinity for sites on particular proteins, antibodies, poly- and oligosaccharides, and other biomolecules.

In another aspect the invention features relies on the use of probe sequences, where said sequences have been furthermore modified by Selectively Binding Complementary (SBC) nucleobases, i.e. modified nucleobases that can make stable hydrogen bonds to their complementary nucleobases, but are unable to make stable hydrogen bonds to other SBC nucleobases. Such SBC monomer substitutions are especially useful when highly self-complementary detection probe sequences are employed. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called 2SU) (2-thio-4-oxo-pyrimidine) and 2-thio-thymine (T′, also called 2ST) (2-thio-4-oxo-5-methyl-pyrimidine).

In another aspect the detection probe sequences used in the invention are covalently bonded to a solid support by reaction of a nucleoside phosphoramidite with an activated solid support, and subsequent reaction of a nucleoside phosphoramide with an activated nucleotide or nucleic acid bound to the solid support. In some embodiments, the solid support or the detection probe sequences bound to the solid support are activated by illumination, a photogenerated acid, or electric current. In other embodiments the detection probe sequences contain a spacer, e.g. a randomized nucleotide sequence or a non-base sequence, such as hexaethylene glycol, between the reactive group and the recognition sequence. Such covalently bonded detection probe sequence populations are highly useful for large-scale detection and expression profiling of mature miRNAs and stem-loop precursor miRNAs.

The oligonucleotide compositions and detection probe sequences disclosed in WO 2006/069584 are highly useful and applicable for detection of individual small RNA molecules in complex mixtures composed of hundreds of thousands of different nucleic acids, such as detecting mature miRNAs, their target mRNAs or siRNAs, by Northern blot analysis or for addressing the spatiotemporal expression patterns of miRNAs, siRNAs or other non-coding RNAs as well as mRNAs by in situ hybridization in whole-mount embryos, whole-mount animals or plants or tissue sections of plants or animals, such as human, mouse, rat, zebrafish, Caenorhabditis elegans, Drosophila melanogaster, Arabidopsis thaliana, rice and maize. These oligonucleotide compositions and detection probe sequences of invention are furthermore highly useful and applicable for large-scale and genome-wide expression profiling of mature miRNAs, siRNAs or other non-coding RNAs in animals and plants by oligonucleotide microarrays. These oligonucleotide compositions and detection probe sequences are furthermore highly useful in functional analysis of miRNAs, siRNAs or other non-coding RNAs in vitro and in vivo in plants or animals, such as human, mouse, rat, zebrafish, Caenorhabditis elegans, Drosophila melanogaster, Arabidopsis thaliana, rice and maize, by inhibiting their mode of action, e.g. the binding of mature miRNAs to their cognate target mRNAs. The oligonucleotide compositions and detection probe sequences disclosed in WO 2006/069584 are also applicable to detecting, testing, diagnosing or quantifying miRNAs, siRNAs, other non-coding RNAs, RNA-edited transcripts or alternative mRNA splice variants implicated in or connected to human disease in complex human nucleic acid samples, e.g. from cancer patients. These oligonucleotide compositions and probe sequences are especially applicable for accurate, highly sensitive and specific detection and quantitation of microRNAs and other non-coding RNAs, which are useful as biomarkers for diagnostic purposes of human diseases, such as cancers, as well as for antisense-based intervention, targeted against tumorigenic miRNAs and other non-coding RNAs.

Finally the oligonucleotide compositions and probe sequences disclosed in WO 2006/069584 are furthermore applicable for sensitive and specific detection and quantitation of microRNAs, which can be used as biomarkers for the identification of the primary site of metastatic tumors of unknown origin.

### BRIEF DESCRIPTION OF THE DRAWINGS

FIG. 1: The structures of DNA, LNA and RNA nucleosides.

FIG. 2: The structures of LNA 2,6-diaminopurine and LNA 2-thiothymidine nucleosides.

FIG. 3. The specificity of microRNA detection by in situ hybridization with LNA-substituted probes.

The LNA probes containing one 1 MM) or two (2 MM) mismatches were designed for the three different miRNAs miR-206, miR-124a and miR-122a (see Table 3 below). The hybridizations were performed on embryos at 72 hours post fertilization at the same temperature as the perfect match probe (0 MM).

FIG. 4: Examples of miRNA whole-mount in situ expression patterns in zebrafish detected by LNA-substituted probes.

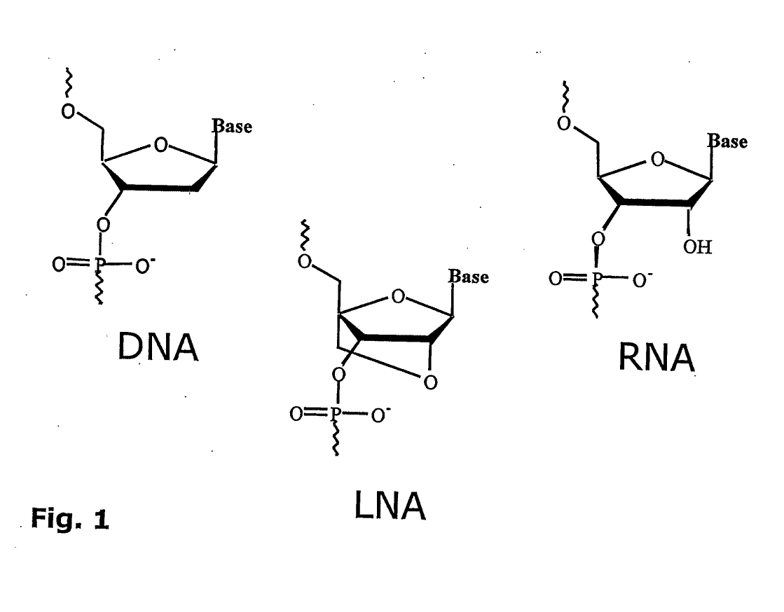

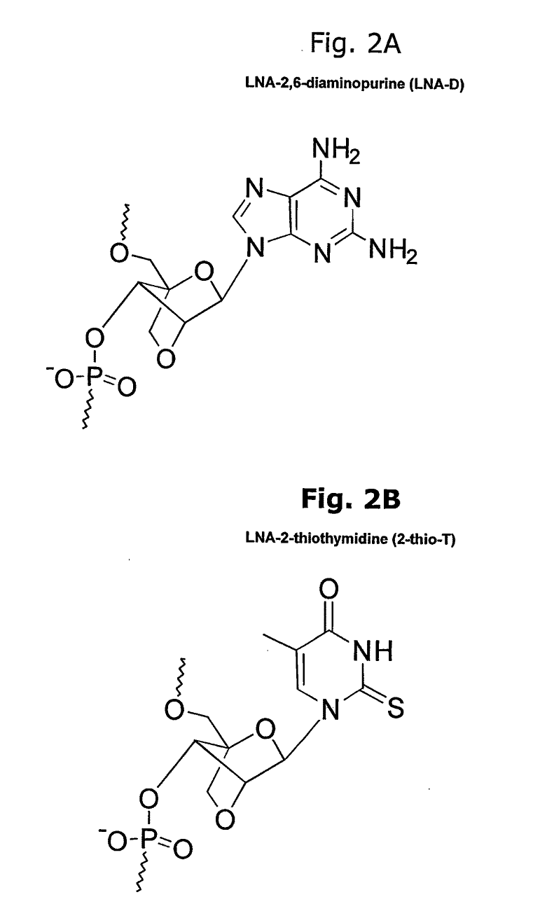

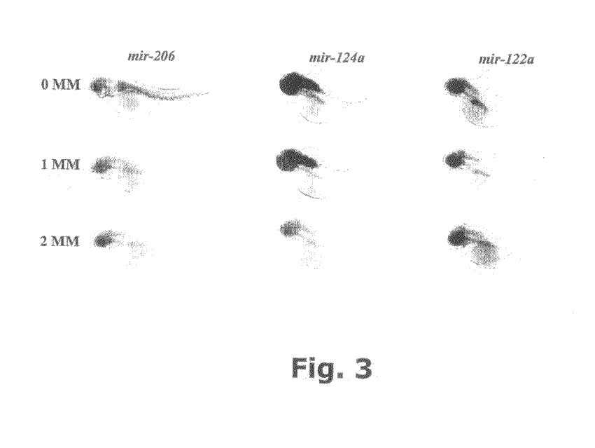

Representatives for miRNAs expressed in the organ systems are shown. miRNAs were expressed in: (A) liver of the digestive system, (B) brain, spinal cord and cranial nerves/ganglia of the central and peripheral nervous systems, (C, M) muscles, (D) restricted parts along the head-to-tail axis, (E) pigment cells of the skin, (F, L) pronephros and presumably mucous cells of the excretory system, (G, M) cartilage of the skeletal system, (H) thymus, (I, N) blood vessels of the circulatory system, (J) lateral line system of the sensory organs. Embryos in (K, L, M, N) are higher magnifications of the embryos in (C, D, G, I), respectively. (A-J, N) are lateral views; (K-M) are dorsal views. All embryos are 72 hours post fertilization, except for (H), which is a five-day old larva.

FIG. 5: Detection of let-7a miRNA by in situ hybridization in paraffin-embedded mouse brain sections using 3′ digoxigenin-labeled LNA probe.

Part of the hippocampus can be seen as an arrow-like structure.

FIG. 6: Detection of let-7a miRNA by in situ hybridization in paraffin-embedded mouse brain sections using 3′ digoxigenin-labeled LNA probe.

The Purkinje cells can be seen in the cerebellum.

FIG. 7: Detection of miR-124a, miR-122a and miR-206 with DIG-labeled DNA and LNA probes in 72 h zebrafish embryos.

(a) Dot-blot of DIG labeled DNA and LNA probes. Per probe, 1 pmol was spotted on a positively charged nylon membrane. All probes show approximately equal incorporation of the DIG-label.

(b) Only LNA probes give clear staining. LNA probes were hybridized at 59 ° C. (miR-122a and miR-124a) and 54° C. (miR-206). DNA probes were hybridized at 45° C.

FIG. 8: Determination of the optimal hybridization temperature and time for in situ hybridization on 72 h zebrafish embryos using LNA probes.

(a) LNA probes for miR-122a and miR-206 were hybridized at different temperatures. The optimal hybridization temperature lies around 21 ° C. below the calculated Tm of the probe. While specific staining remains at the lower temperatures, background increases significantly. At higher temperatures staining is completely lost.

(b) Hybridization time series with probes for miR-122a and miR-206. An incubation time of 10 min is already sufficient to get a detectable signal, while increasing the hybridization time beyond one hour does not increase the signal significantly. All in situ hybridizations were performed in parallel.

FIG. 9: Assessment of the specificity of LNA probes using perfectly matched and mismatched probes for the detection of miR-124a, miR-122a and miR-206 by in situ hybridization on 72 h zebrafish embryos.

Mismatched probes were hybridized under the same conditions as the perfectly matching probe. In most cases a central single mismatch is sufficient to loose signal. For the very highly expressed miR-124a specific staining was only lost upon introduction of two consecutive central mismatches in the probe.

FIG. 10: In situ detection of miR-124a and miR-206 in 72 h zebrafish embryos using shorter LNA probe versions.

In situ hybridizations were performed with probes of 2, 4, 6, 8, 10, 12 and 14 nt shorter than the original 22 nt probes. Signals of probes that were 14 nt in length still resulted in readily detectable and specific signals. A single central mismatch in the 14 nt probes for miR-124a and miR-206 prevents hybridization. Probes that were 12 nt in length gave slightly reduced staining for both miR-124a and miR-206. Staining was virtually lost when 10 and 8 nt probes were used, although weak staining in the brain could still be observed for the highly expressed miR-124a.

FIG. 11: In situ hybridizations for miRNAs on Xenopus tropicalis and mouse embryos.

(a) Expression of miR-1 is restricted to the muscles in the body and the head in X. tropicalis. miR-124a is expressed throughout the central nervous system.

(b) Expression of 15 miRNAs in 9.5 and 10.5 dpc (days post coitum) mouse embryos: miR-10a and 10b, posterior trunk; miR-196a, tailbud; miR-126, blood vessels; miR-125b, midbrain hindbrain boundary; miR-219, midbrain, hindbrain and spinal cord; miR-124a, central nervous system; miR-9, forebrain and the spinal cord; miR-206, somites; miR-1, heart and somites; miR-182, miR-96 and miR-183, cranial and dorsal root ganglia; miR-17-5p and miR-20 are expressed ubiquitously, like the other members of its genomic cluster.

FIG. 12: Quality of the logarithm-transformed raw intensities from microarray slides assessed using different diagnostic plots (histograms, MA-plots and scatter plots).

The graphs show the intensities before and after global Lowess normalization (on the left and right hand side, respectively). 12A: Graphs from array applied on glandular metastasis (GM); the distribution of the log-transformed raw intensities from the GM sample showed a bimodal distribution, however, after normalization only one peak was observed. 12B: Graphs from array applied on normal jejunum (NJ). 12C: Graphs from array applied on total RNA from colon tissue. 12D: Graphs from array applied on total RNA from lymph node.

FIG. 13: One-way hierarchical clustering of the data from FIG. 12.

Distance: Pearson correlation coefficient. Linkage method: Centroid.

FIG. 14: Principal Components Analysis (PCA) plot, illustrating the differences between the samples from FIG. 12.

FIG. 15: Experimental design for establishing origin of head and neck tumours.

FIG. 16: Heat map diagram showing the result of two-way unsupervised hierarchical clustering of genes and samples.

FIG. 17: Heat map diagram showing the result of two-way unsupervised hierarchical clustering of genes and samples.

FIG. 18: Principal Components Analysis (PCA) plot, illustrating the differences between the samples from FIG. 17.

FIG. 19: Heat map diagram showing the result of two-way unsupervised hierarchical clustering of genes and samples.

FIG. 20: Principal Components Analysis (PCA) plot, illustrating the differences between the samples from FIG. 19.

### DEFINITIONS

For the purposes of the subsequent detailed description of the invention the following definitions are provided for specific terms, which are used in the disclosure of the present invention:

In the present context “ligand” means something, which binds. Ligands comprise biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e, functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as small nucleolar RNAs, siRNAs, microRNAs and antisense RNAs, which comprise important structural and regulatory roles in the cell.

A “multi-probe library” or “library of multi-probes” comprises a plurality of multi-probes, such that the sum of the probes in the library are able to recognise a major proportion of a transcriptome, including the most abundant sequences, such that about 60%, about 70%, about 80%, about 85%, more preferably about 90%, and still more preferably 95%, of the target nucleic acids in the transcriptome, are detected by the probes.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumours, and also to samples of in vitro cell culture constituents (including but not limited to conditioned medium resulting from the growth of cells in cell culture medium, recombinant cells and cell components). The term also embraces extracted samples such as extracted RNA and DNA, including total RNA from a tissue or cell sample.

An “organism” refers to a living entity, including but not limited to, for example, human, mouse, rat, Drosophila, C. elegans, yeast, Arabidopsis thallana, maize, rice, zebra fish, primates, domestic animals, etc.

The terms “Detection probes” or “detection probe” or “detection probe sequence” refer to an oligonucleotide, which oligonucleotide comprises a recognition sequence complementary to a RNA (or DNA) target sequence, which said recognition sequence is substituted with high-affinity nucleotide analogues, e.g. LNA, to increase the sensitivity and specificity of conventional oligonucleotides, such as DNA oligonucleotides, for hybridization to short target sequences, e.g. mature miRNAs, stem-loop precursor miRNAs, pri-miRNAs, siRNAs or other non-coding RNAs as well as miRNA binding sites in their cognate mRNA targets, mRNAs, mRNA splice variants, RNA-edited mRNAs and antisense RNAs.

The terms “miRNA” and “microRNA” refer to 18-25 nt non-coding RNAs derived from endogenous genes. They are processed from longer (ca 75 nt) hairpin-like precursors termed pre-miRNAs. MicroRNAs assemble in complexes termed miRNPs and recognize their targets by antisense complementarity. If the microRNAs match 100% their target, i.e. the complementarity is complete, the target mRNA is cleaved, and the miRNA acts like a siRNA. If the match is incomplete, i.e. the complementarity is partial, then the translation of the target mRNA is blocked.

The terms “Small interfering RNAs” or “siRNAs” refer to 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences

The term “RNA interference” (RNAi) refers to a phenomenon where double-stranded RNA homologous to a target mRNA leads to degradation of the targeted mRNA. More broadly defined as degradation of target mRNAs by homologous siRNAs.

The term “Recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The terms “oligonucleotlde” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature. Because mononucleotides are reacted to make oligonucleotides in a manner such that the 5′-phosphate of one mononucleotide pentose ring is attached to the 3′ oxygen of its neighbour in one direction via a phosphodiester linkage, an end of an oligonucleotlde is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

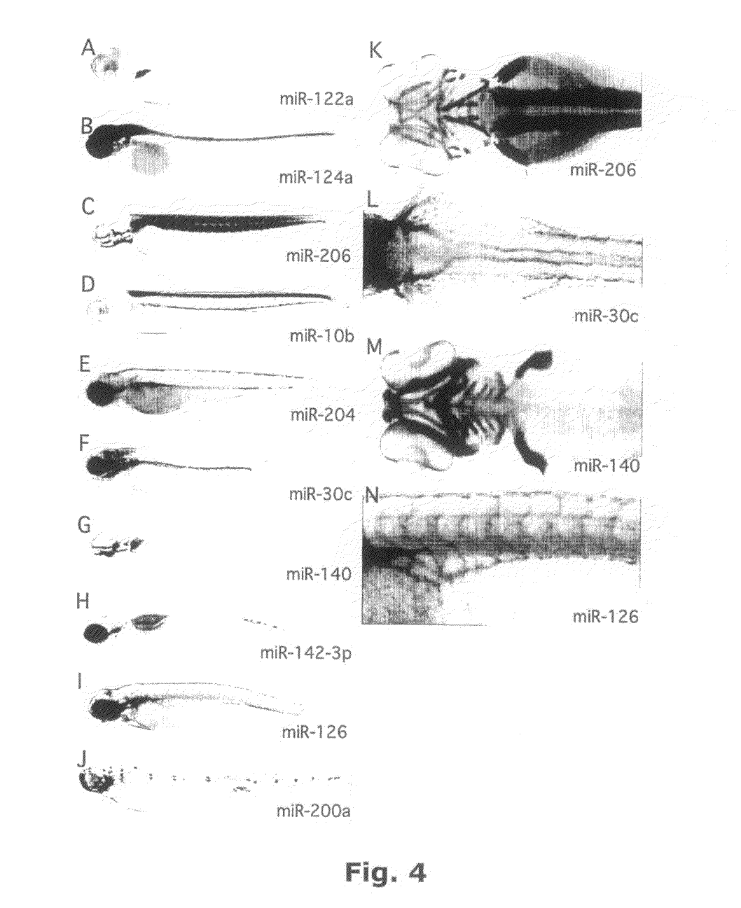

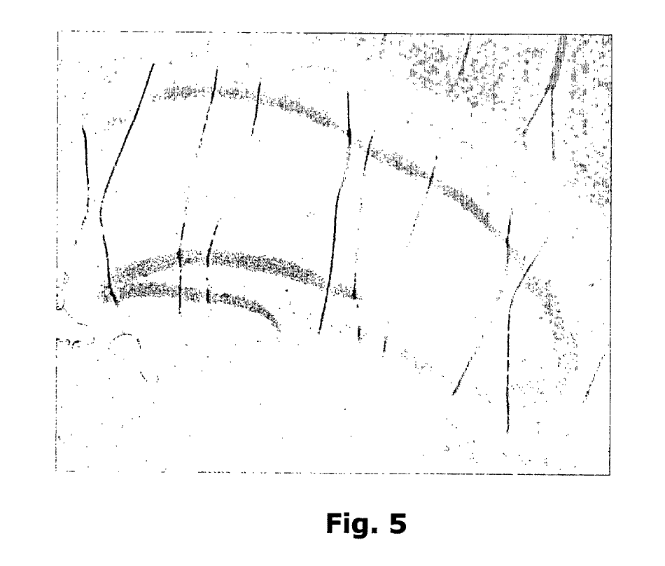

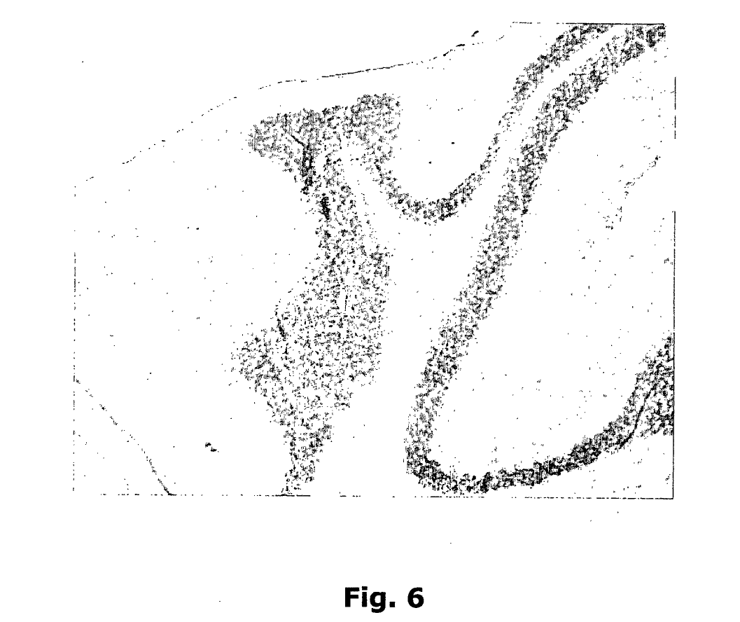

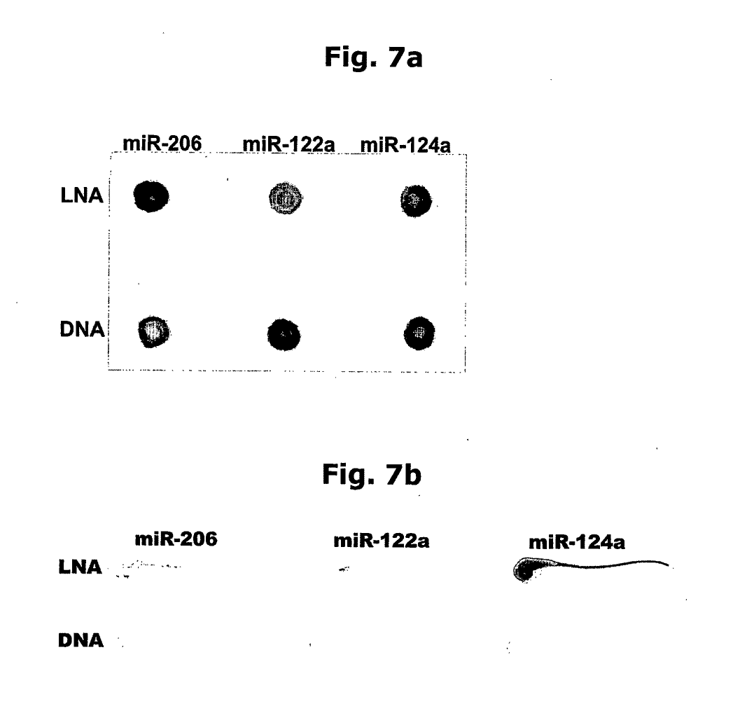

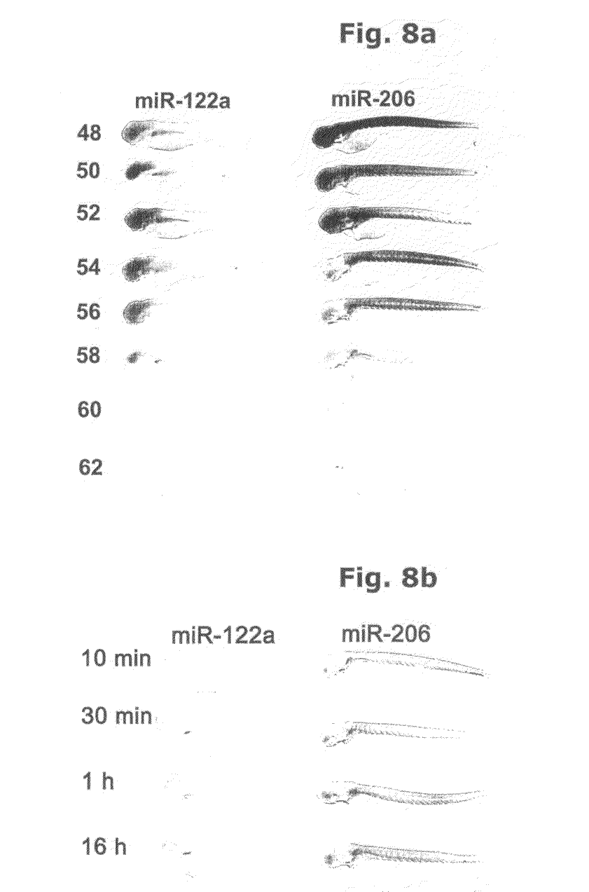

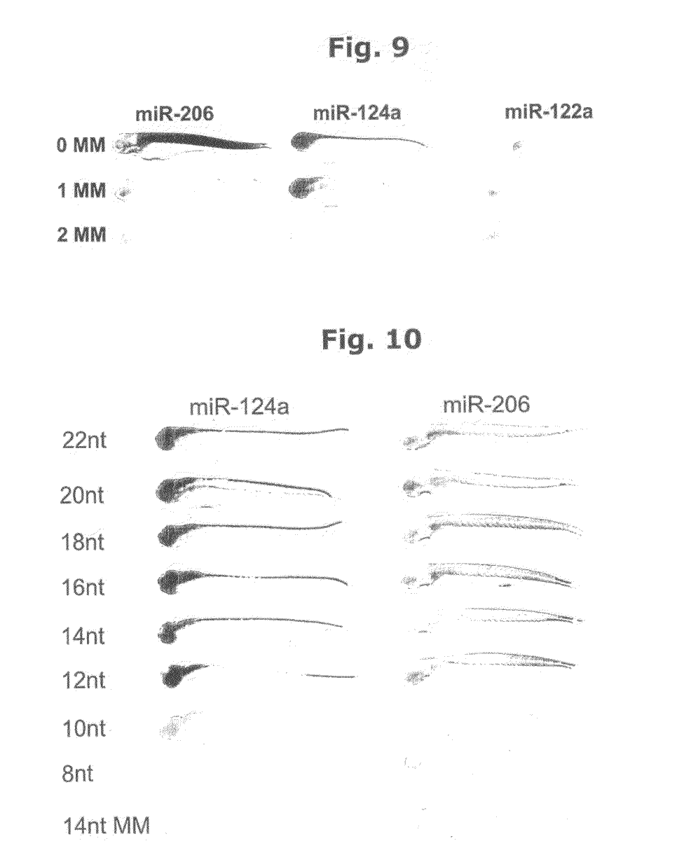

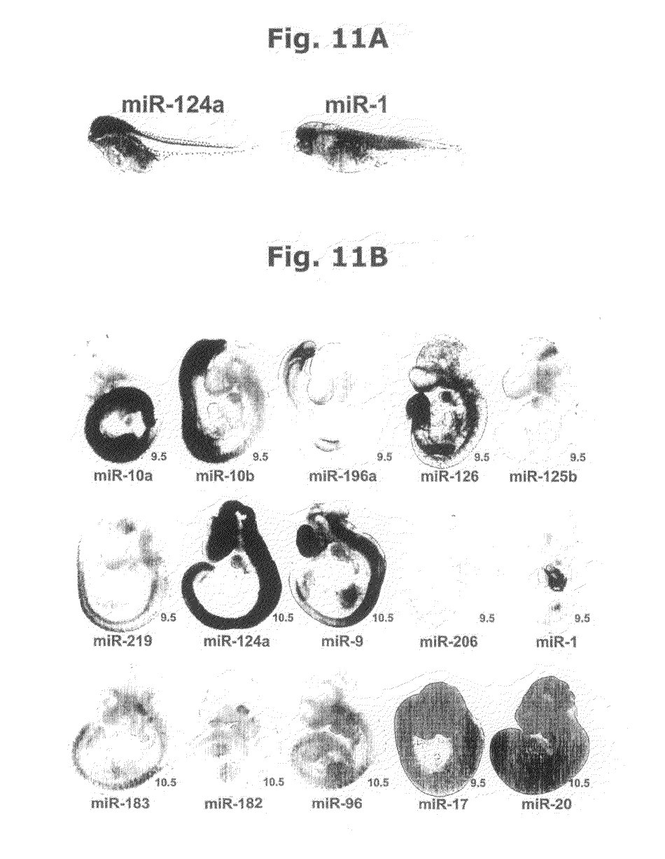

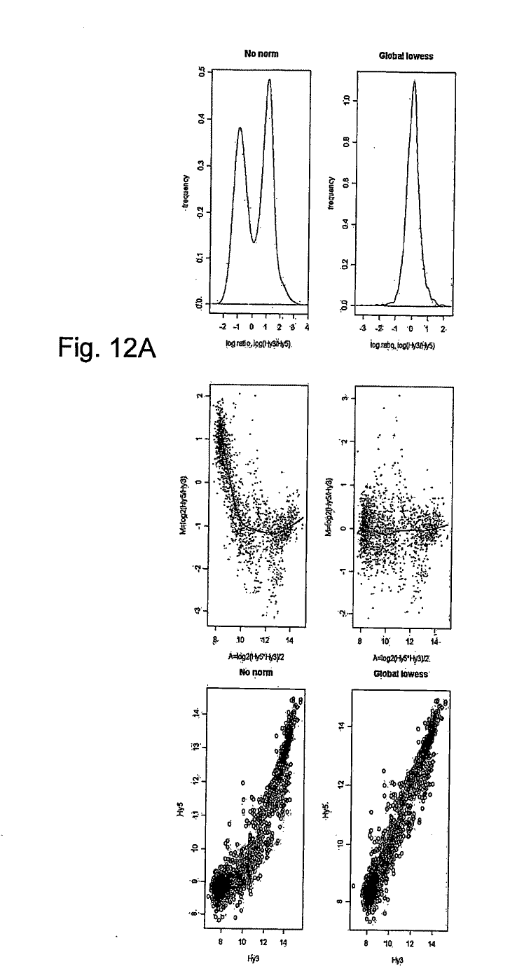

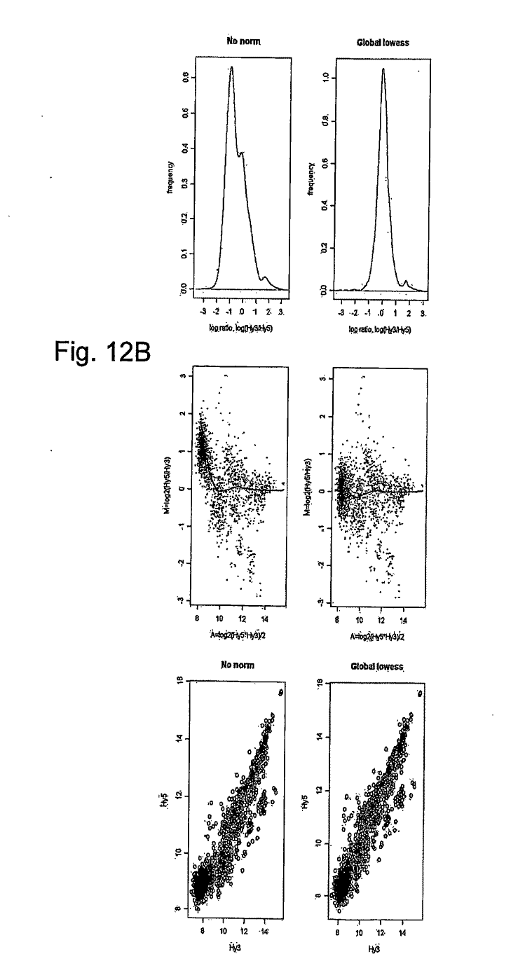

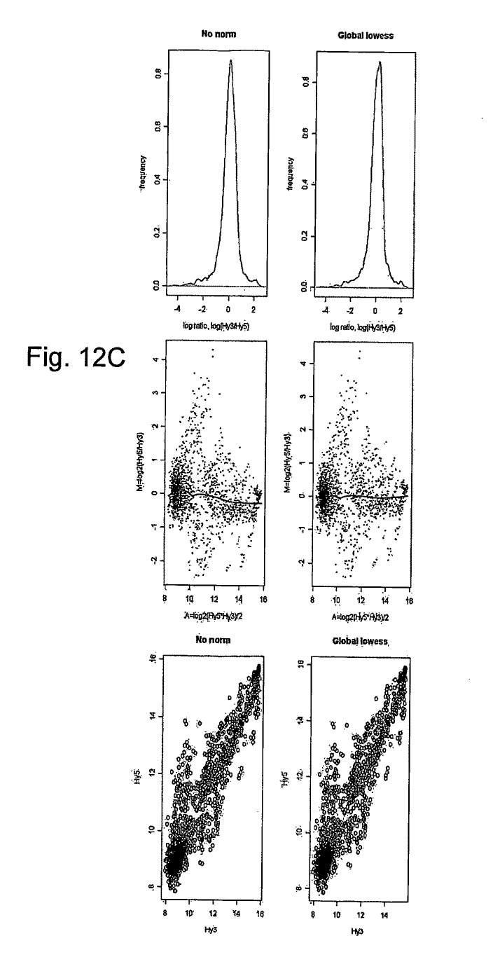

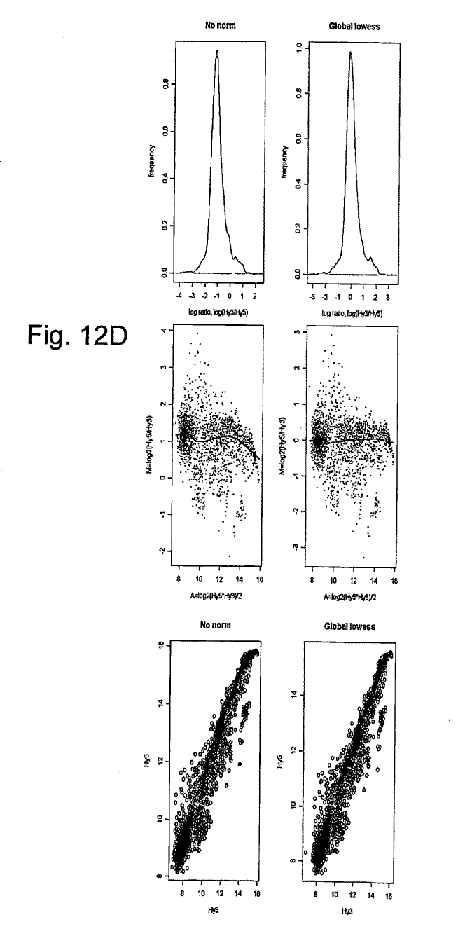

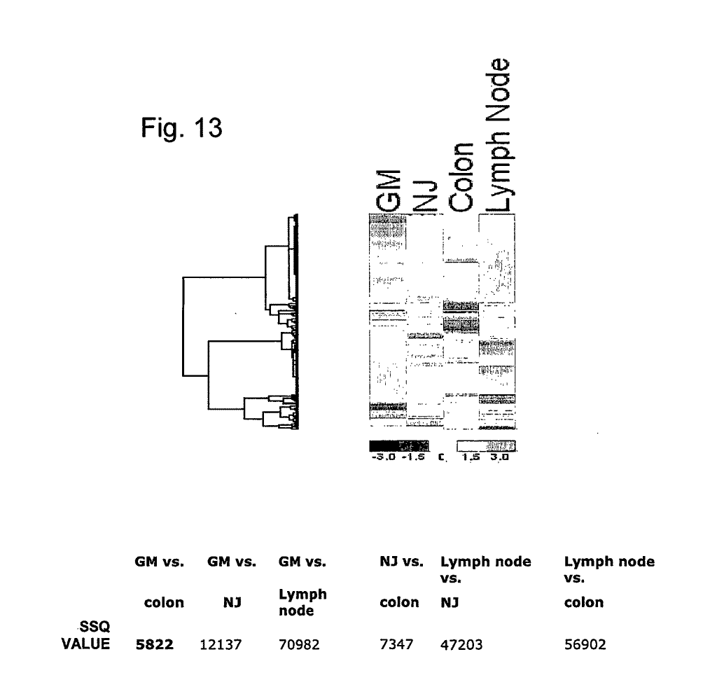

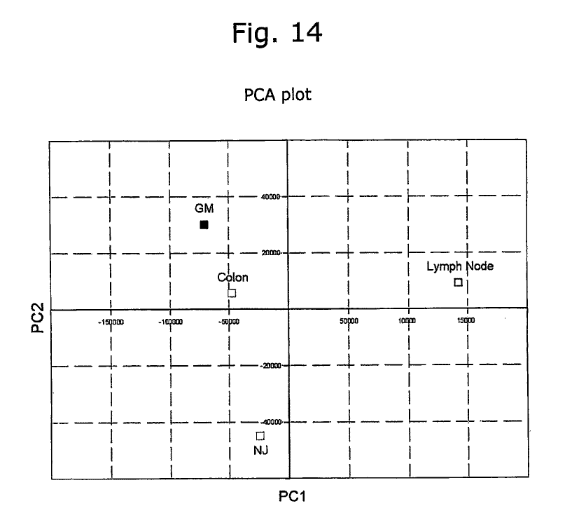

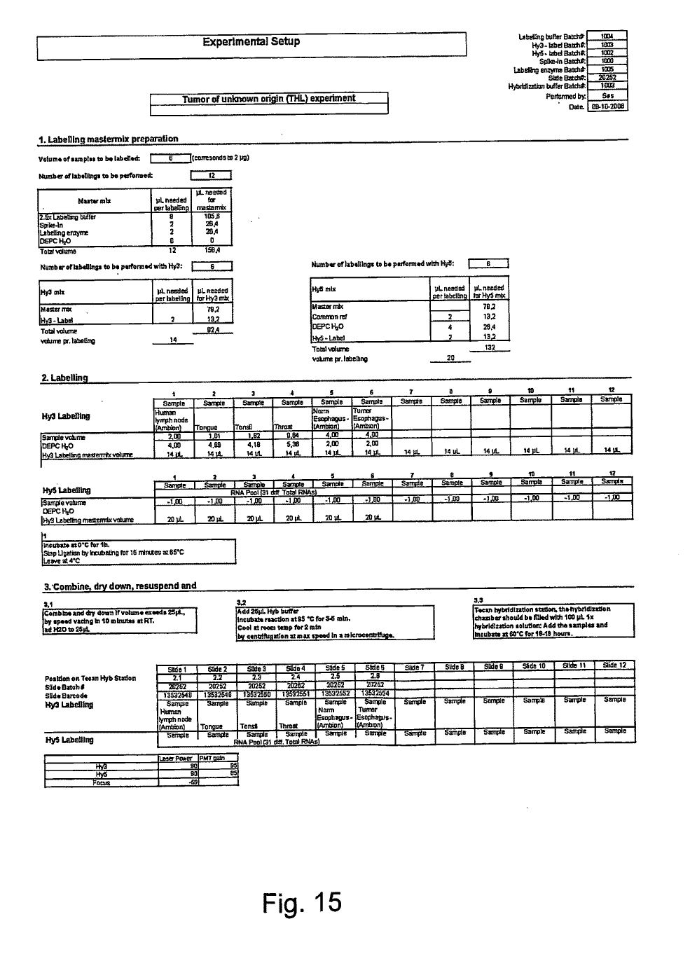

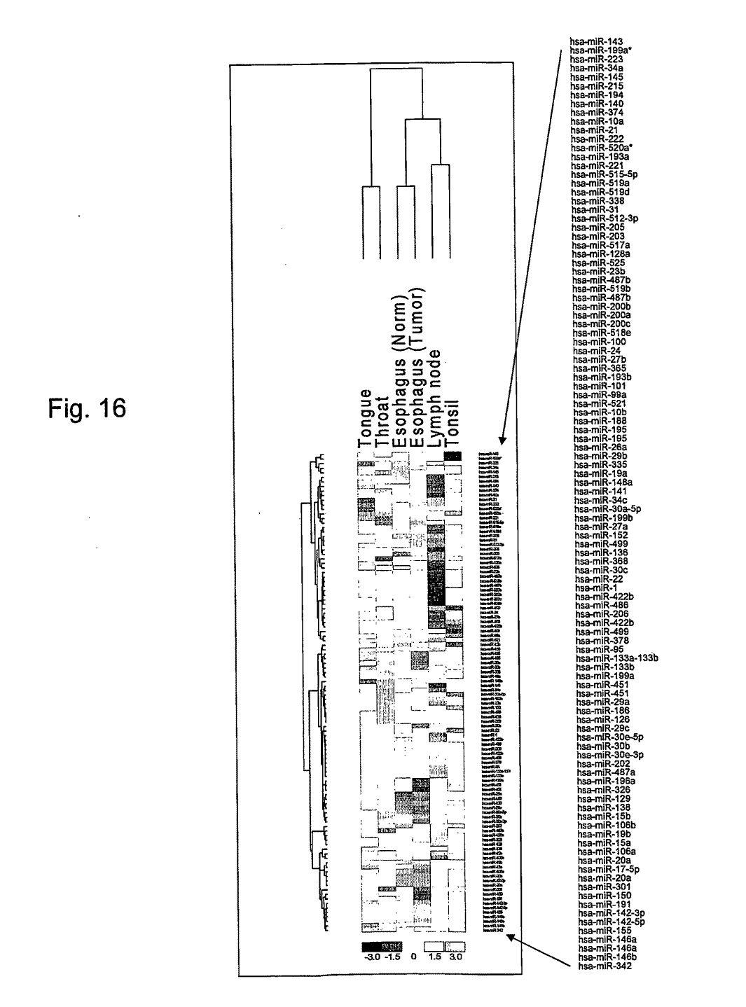

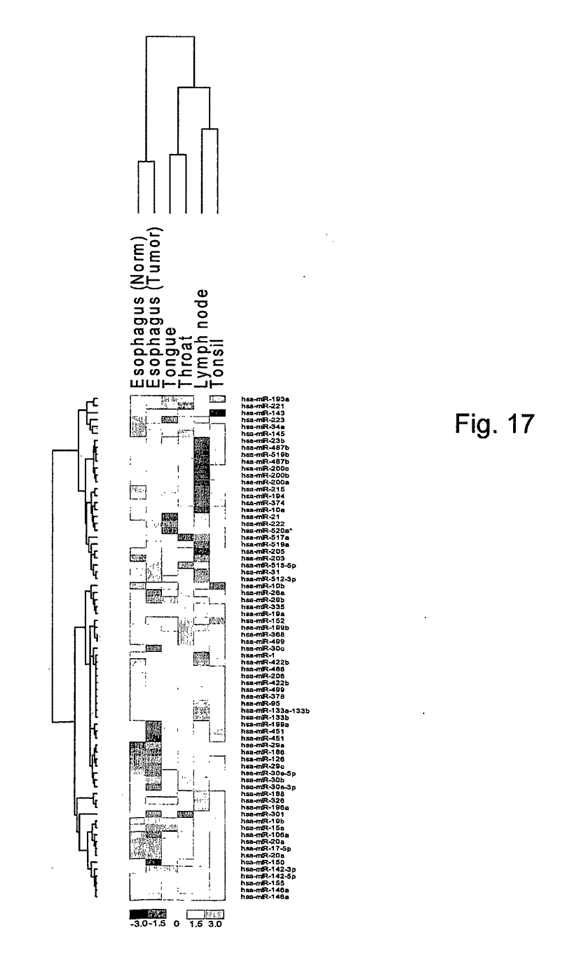

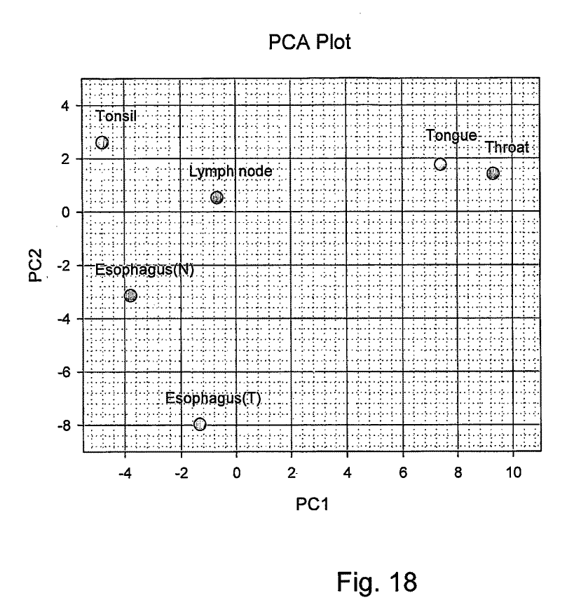

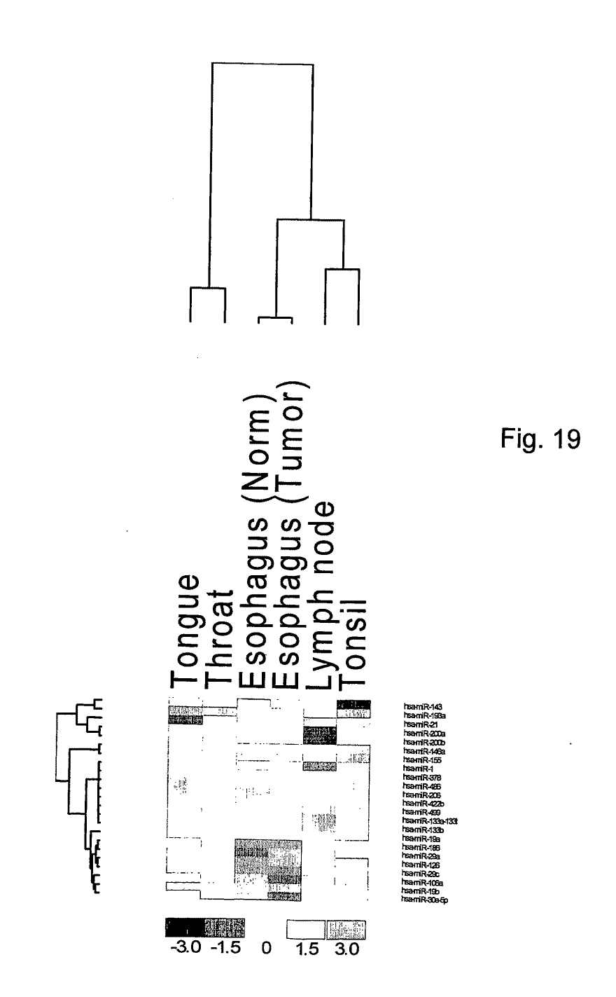

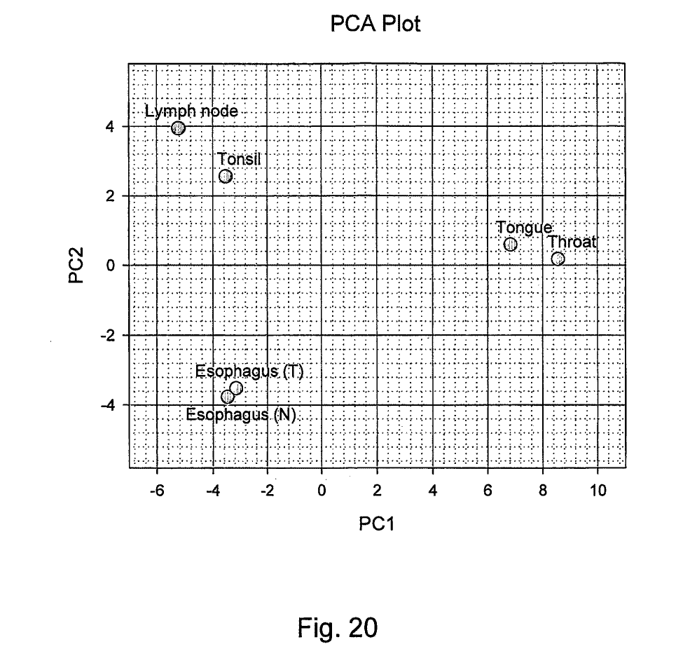

By the term “SBC nucleobases” is meant “Selective Binding Complementary” nucleobases, i.e. modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called ²⁵U) (2-thio-4-oxo-pyrimidine) and 2-thio-thymine (T′, also called ²⁵T) (2-thio-4-oxo-5-methyl-pyrimidine). FIG. 4 in PCT Publication No. WO 2004/024314 illustrates that the pairs A-²⁵T and D-T have 2 or more than 2 hydrogen bonds whereas the D-²⁵T pair forms a single (unstable) hydrogen bond. Likewise the SBC nucleobases pyrrolo-[2,3-d]pyrimidine-2(3H)-one (C′, also called PyrroloPyr) and hypoxanthine (G′, also called I) (6-oxo-purine) are shown in FIG. 4 in PCT Publication No. WO 2004/024314 where the pairs PyrroloPyr-G and C-I have 2 hydrogen bonds each whereas the PyrroloPyr-I pair forms a single hydrogen bond.

“SBC LNA oligomer” refers to a “LNA oligomer” containing at least one LNA monomer where the nucleobase is a “SBC nucleobase”. By “LNA monomer with an SBC nucleobase” is meant a “SBC LNA monomer”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding modified oligonucleotide e.g. the sequences agTtcATg is equal to agTscD²⁵Ug where s is equal to the SBC DNA monomer 2-thio-t or 2-thio-u, D is equal to the SBC LNA monomer LNA-D and ²⁵U is equal to the SBC LNA monomer LNA ²⁵U.

The complement of a nucleic acid sequence as used herein refers to an oligonucleotide which, when aligned with the nucleic add sequence such that the 5′ end of one sequence is paired with the 3′ end of the other, is in “antiparallel association.” Bases not commonly found in natural nucleic acids may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer Drug Design 6: 585-607, 1991, each of which are hereby incorporated by reference in their entirety).

The term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyioxazoie derivatives, pyrenyl, pyrenylmethylglycerol derivatives and the like. Other preferred universal bases include, pyrrole, diazole or triazole derivatives, including those universal bases known in the art.

By “oligonucleotide,” “oligomer,” or “oligo” is meant a successive chain of monomers (e.g., glycosides of heterocyclic bases) connected via internucleoside linkages. The linkage between two successive monomers in the oligo consist of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′-position, whereas the right-hand side is bound to the 5′-position of a preceding monomer.

By “LNA” or “LNA monomer” (e.g., an LNA nucleoside or LNA nucleotide) or an LNA oligomer (e.g., an oligonucleotide or nucleic acid) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R^{4′} and R^{2′} as shown in formula (I) below together designate —CH₂—O— or —CH₂—CH₂—O—.

By “LNA modified oligonucleotide” or “LNA substituted oligonucleotide” is meant a oligonucleotide comprising at least one LNA monomer of formula (I), described infra, having the below described illustrative examples of modifications:

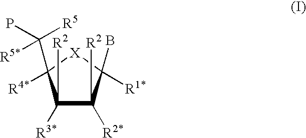

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*).

B is selected from a modified base as discussed above e.g. an optionally substituted carbocyclic aryl such as optionally substituted pyrene or optionally substituted pyrenyimethyiglycerol, or an optionally substituted heteroallcylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴*, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkylamino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ, differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“Oligonucleotide analogue” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone in PNA, nucleobases are attached to the uncharged polyamide backbone yielding a chimeric pseudopeptide-nucleic acid structure, which is homomorphous to nucleic acid forms.

“High affinity nucleotide analogue” or “affinity-enhancing nucleotide analogue” refers to a non-naturally occurring nucleotide analogue that increases the “binding affinity” of an oligonucleotide probe to its complementary recognition sequence when substituted with at least one such high-affinity nucleotide analogue.

As used herein, a probe with an increased “binding affinity” for a recognition sequence compared to a probe which comprises the same sequence but does not comprise a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the recognition sequence in the target sequence in a double stranded molecule.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighbouring monomer in the 5′-terminal direction and the “preceding monomer” relates to the neighbouring monomer in the 3′-terminal direction.

The term “target nucleic acid” or “target ribonucleic acid” refers to any relevant nucleic acid of a single specific sequence, e. g., a biological nucleic acid, e. g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid, typically to an RNA sequence in a miRNA.

The term “stringent conditions”, as used herein, is the “stringency” which occurs within a range from about Tₘ-5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969.

When using the terms “specificity”, “specifically” and “specific”, e.g. when discussing “specific hybridization”, “specific binding” or “specific detection”, is herein meant that such specificity is limited to the relevant type of sample being tested, i.e. a specifically binding probe used in the invention is a probe, which can distinguish one miRNA from other miRNAs in the same type of sample. It is well-known that cross-reactivity in binding between ligands exists across species barriers, but since the present invention relates to typing of tumours in a mammal (typically in a human being), it is in practice only relevant to ensure that a particular probe used in the invention is capable of distinguishing between miRNAs from the species in which the tumour is present. It is therefore not a problem that a particular probe cross-reacts with other nucleic acids (even other miRNAs), if these other nucleic acids will not be able to interfer in the real-life assay of the present invention where the probe is used. So, in brief, in the present context a specifically binding probe is a probe which is complementary to a miRNA from a certain species having a tumour of unknown origin but the probe is not capable of binding to other miRNAs from the same species under stringent hybridization conditions as these are defined in e.g. Sambrook et al (“Molecular cloning: a laboratory manual”). Especially preferred “specific” probes are those which are only complementary to a sequence in one single human miRNA.

### DETAILED DESCRIPTION OF THE INVENTION

Collection of Probes Used in the Invention

As briefly stated above, the probe collections or libraries used in the present invention are so designed that each member of said collection comprises a recognition sequence consisting of nucleobases and affinity enhancing nucleobase analogues, and wherein the recognition sequences exhibit a combination of high melting temperatures and low self-complementarity scores, said melting temperatures being the melting temperature of the duplex between the recognition sequence and its complementary DNA or RNA sequence.

This design provides for probes which are highly specific for their target sequences but which at the same time exhibits a very low risk of self-annealing (as evidenced by a low self-complementarity score)—self-annealing is, due to the presence of affinity enhancing nucleobases (such as LNA monomers) a problem which is more serious than when using conventional deoxyribonucleotide probes.

In one embodiment of the detection method of the present invention the recognition sequences exhibit a melting temperature (or a measure of melting temperature) corresponding to at least 5° C. higher than a melting temperature or a measure of melting temperature of the self-complementarity score under conditions where the probe hybridizes specifically to its complementary target sequence (alternatively, one can quantify the “risk of self-annealing” feature by requiring that the melting temperature of the probe-target duplex must be at least 5° C. higher than the melting temperature of duplexes between the probes or the probes internally). The collection may be so constituted that at least 90% (such as at least 95%) of the recognition sequences exhibit a melting temperature or a measure of melting temperature corresponding to at least 5° C. higher than a melting temperature or a measure of melting temperature of the self-complementarity score under conditions where the probe hybridizes specifically to its complementary target sequence (or that at least the same percentages of probes exhibit a melting temperature of the probe-target duplex of at least 5° C. more than the melting temperature of duplexes between the probes or the probes internally). In a preferred embodiment all of the detection probes include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

However, it is preferred that this temperature difference is higher, such as at least least 10° C., such as at least 15, at least 20, at least 25, at least 30, at least 35, at least 40, at least 45, and at least 50° C. higher than a melting temperature or measure of melting temperature of the self-complementarity score.

In one embodiment a collection of probes according to the present invention comprises at least 10 detection probes, 15 detection probes, such as at least 20, at least 25, at least 50, at least 75, at least 100, at least 200, at least 500, at least 1000, and at least 2000 members.

It if preferred that the collection of probes of the invention is capable of specifically detecting all or substantially all miRNAs in the mammal in question.

In another preferred embodiment, the collection of probes is capable of specifically detecting all such miRNAs.

In one embodiment, the affinity-enhancing nucleobase analogues are regularly spaced between the nucleobases in at least 80% of the members of said collection, such as in at least 90% or at least 95% of said collection (in one embodiment, all members of the collection contains regularly spaced affinity-enhancing nucleobase analogues). One reason for this is that the time needed for adding each nucleobase or analogue during synthesis of the probes of the invention is dependent on whether or not a nucleobase analogue is added. By using the “regular spacing strategy” considerable production benefits are achieved. Specifically for LNA nucleobases, the required coupling times for incorporating LNA amidites during synthesis may exceed that required for incorporating DNA amidites. Hence, in cases involving simultaneous parallel synthesis of multiple oligonucleotides on the same instrument, it is advantageous if the nucleotide analogues such as LNA are spaced evenly in the same pattern as derived from the 3′-end, to allow reduced cumulative coupling times for the sytnthesis. The affinity enhancing nucleobase analogues are conveniently regularly spaced as every 2^{nd}, every 3^{rd}, every 4^{th} or every 5^{th} nucleobase in the recognition sequence, and preferably as every 3^{rd} nucleobase.

In one embodiment of the the collection of probes, all members contain affinity enhancing nucleobase analogues with the same regular spacing in the recognition sequences.

The presence of the affinity enhancing nucleobases in the recognition sequence preferably confers an increase in the binding affinity between a probe and its complementary target nucleotide sequence relative to the binding affinity exhibited by a corresponding probe, which only include nucleobases. Since LNA nucleobases/monomers have this ability, it is preferred that the affinity enhancing nucleobase analogues are LNA nucleobases.

In some embodiments, the 3′ and 5′ nucleobases are not substituted by affinity enhancing nucleobase analogues.

As detailed herein, one huge advantage of the probes of the invention is their short lengths which provides for high target specificity and advantages in detecting small RNAs and detecting nucleic acids in samples not normally suitable for hybridization detection strategies. It is, however, preferred that the probes comprise a recognition sequence is at least a 6-mer, such as at least a 7-mer, at least an 8-mer, at least a 9-mer, at least a 10-mer, at least an 11-mer, at least a 12-mer, at least a 13-mer, at least a 14-mer, at least a 15-mer, at least a 16-mer, at least a 17-mer, at least an 18-mer, at least a 19-mer, at least a 20-mer, at least a 21-mer, at least a 22-mer, at least a 23-mer, and at least a 24-mer. On the other hand, the recognition sequence is preferably at most a 25-mer, such as at most a 24-mer, at most a 23-mer, at most a 22-mer, at most a 21-mer, at most a 20-mer, at most a 19-mer, at most an 18-mer, at most a 17-mer, at most a 16-mer, at most a 15-mer, at most a 14-mer, at most a 13-mer, at most a 12-mer, at most an 11-mer, at most a 10-mer, at most a 9-mer, at most an 8-mer, at most a 7-mer, and at most a 6-mer.

Also for production purposes, it is an advantage that a majority of the probes in a collection are of the same length. In preferred embodiments, the collection of probes of the invention is one wherein at least 80% of the members comprise recognition sequences of the same length, such as at least 90% or at least 95%.

As discussed above, it is advantageous, in order to avoid self-annealing, that at least one of the nucleobases in the recognition sequence is substituted with its corresponding selectively binding complementary (SBC) nucleobase.

Typically, the nucleobases in the sequence are selected from ribonucleotides and deoxyribonucleotides, preferably deoxyribonucleotides. It is preferred that the recognition sequence consists of affinity enhancing nucleobase analogues together with either ribonucleotides or deoxyribonucleotides.

In certain embodiments, each member of a collection is covalently bonded to a solid support. Such a solid support may be selected from a bead, a microarray, a chip, a strip, a chromatographic matrix, a microtiter plate, a fiber or any other convenient solid support generally accepted in the art in order to facilitate the exercise of the methods discussed generally and-specifically

As also detailed herein, each detection probe in a collection of the invention may include a detection moiety and/or a ligand, optionally placed in the recognition sequence but also placed outside the recognition sequence. The detection probe may thus include a photochemically active group, a thermochemically active group, a chelating group, a reporter group, or a ligand that facilitates the direct of indirect detection of the probe or the immobilisation of the oligonucleotide probe onto a solid support.

Probes Used in the Inventive Method

The present invention utilises oligonucleotide compositions and probe sequences in identification and optionally quantification/capture of miRNAs and stem-loop precursor miRNAs where the probe sequences contain a number of nucleoside analogues.

In a preferred embodiment the number of nucleoside analogue corresponds to from 20 to 40% of the oligonucleotide.

In a preferred embodiment the probe sequences are substituted with a nucleoside analogue with regular spacing between the substitutions.

In another preferred embodiment the probe sequences are substituted with a nucleoside analogue with irregular spacing between the substitutions.

In a preferred embodiment the nucleoside analogue is LNA.

In a further preferred embodiment the detection probe sequences comprise a photochemically active group, a thermochemically active group, a chelating group, a reporter group, or a ligand that facilitates the direct of indirect detection of the probe or the immobilisation of the oligonucleotide probe onto a solid support.

In a further preferred embodiment:

(a) the photochemically active group, the thermochemically active group, the chelating group, the reporter group, or the ligand includes a spacer (K), said spacer comprising a chemically cleavable group; or

(b) the photochemically active group, the thermochemically active group, the chelating group, the reporter group, or the ligand is attached via the biradical of at least one of the LNA(s) of the oligonucleotide.

Especially preferred detection probes of the invention are those that include the LNA containing recognition sequences set forth in tables A-K, 1, 3 and 15-I herein.

Furthermore, the teachings in WO 2006/199266, where tables G and G1 sets forth the tissue expression of a number of miRNAs, adds to the demonstration provided herein, namely that the expression pattern of miRNA varies between tissues. It is hence possible to devise probes useful in the present invention and produced according to the teachings in WO 2006/069584 by utilising the sequence information given for the miRNAs disclosed in WO 2006/199266.

In general, all known human miRNA sequences are applicable when preparing e.g. a microarray constituted of probes described herein. The currently known human miRNA sequences are available from http://microrna.sanger.ac.uk/. At present, the known human miRNAs are the following provided in Table T:

TABLE T
sequences of human miRNAs

| Accession | ID | Chromosome | Start | End | Strand |
|---|---|---|---|---|---|
| MI0000342 | hsa-mir-200b | 1 | 1092347 | 1092441 | + |
| MI0000737 | hsa-mir-200a | 1 | 1093106 | 1093195 | + |
| MI0001641 | hsa-mir-429 | 1 | 1094248 | 1094330 | + |
| MI0003556 | hsa-mir-551a | 1 | 3467119 | 3467214 | − |
| MI0000268 | hsa-mir-34a | 1 | 9134314 | 9134423 | − |
| MI0005202 | hsa-mir-801 | 1 | 28847698 | 28847793 | + |
| MI0003557 | hsa-mir-552 | 1 | 34907787 | 34907882 | − |
| MI0000749 | hsa-mir-30e | 1 | 40992614 | 40992705 | + |
| MI0000736 | hsa-mir-30c-1 | 1 | 40995543 | 40995631 | + |
| MI0000103 | hsa-mir-101-1 | 1 | 65296705 | 65296779 | − |
| MI0000483 | hsa-mir-186 | 1 | 71305902 | 71305987 | − |
| MI0000454 | hsa-mir-137 | 1 | 98284214 | 98284315 | − |
| MI0003558 | hsa-mir-553 | 1 | 100519385 | 100519452 | + |
| MI0000239 | hsa-mir-197 | 1 | 109943038 | 109943112 | + |
| MI0003559 | hsa-mir-554 | 1 | 149784896 | 149784991 | + |
| MI0003560 | hsa-mir-92b | 1 | 153431592 | 153431687 | + |
| MI0003561 | hsa-mir-555 | 1 | 153582765 | 153582860 | − |
| MI0000466 | hsa-mir-9-1 | 1 | 154656757 | 154656845 | − |
| MI0005116 | hsa-mir-765 | 1 | 155172547 | 155172660 | − |
| MI0003562 | hsa-mir-556 | 1 | 160578960 | 160579054 | + |
| MI0003563 | hsa-mir-557 | 1 | 166611386 | 166611483 | + |
| MI0000290 | hsa-mir-214 | 1 | 170374561 | 170374670 | − |
| MI0000281 | hsa-mir-199a-2 | 1 | 170380298 | 170380407 | − |
| MI0003123 | hsa-mir-488 | 1 | 175265122 | 175265204 | − |
| MI0000270 | hsa-mir-181b-1 | 1 | 197094625 | 197094734 | − |
| MI0000289 | hsa-mir-181a-1 | 1 | 197094796 | 197094905 | − |
| MI0000810 | hsa-mir-135b | 1 | 203684053 | 203684149 | − |
| MI0000735 | hsa-mir-29c | 1 | 206041820 | 206041907 | − |
| MI0000107 | hsa-mir-29b-2 | 1 | 206042411 | 206042491 | − |
| MI0000285 | hsa-mir-205 | 1 | 207672101 | 207672210 | + |
| MI0000291 | hsa-mir-215 | 1 | 218357818 | 218357927 | − |
| MI0000488 | hsa-mir-194-1 | 1 | 218358122 | 218358206 | − |
| MI0003564 | hsa-mir-558 | 2 | 32610724 | 32610817 | + |
| MI0003565 | hsa-mir-559 | 2 | 47458318 | 47458413 | + |
| MI0000293 | hsa-mir-217 | 2 | 56063606 | 56063715 | − |
| MI0000292 | hsa-mir-216 | 2 | 56069589 | 56069698 | − |
| MI0003566 | hsa-mir-560 | 2 | 132731971 | 132732065 | − |
| MI0000447 | hsa-mir-128a | 2 | 136139437 | 136139518 | + |
| MI0000267 | hsa-mir-10b | 2 | 176723277 | 176723386 | + |
| MI0003567 | hsa-mir-561 | 2 | 188870464 | 188870560 | + |
| MI0000084 | hsa-mir-26b | 2 | 218975613 | 218975689 | + |
| MI0000783 | hsa-mir-375 | 2 | 219574611 | 219574674 | − |
| MI0000463 | hsa-mir-153-1 | 2 | 219867077 | 219867166 | − |
| MI0003568 | hsa-mir-562 | 2 | 232745607 | 232745701 | + |
| MI0000478 | hsa-mir-149 | 2 | 241044091 | 241044179 | + |
| MI0003569 | hsa-mir-563 | 3 | 15890282 | 15890360 | + |
| MI0000727 | hsa-mir-128b | 3 | 35760972 | 35761055 | + |
| MI0000083 | hsa-mir-26a-1 | 3 | 37985899 | 37985975 | + |
| MI0000476 | hsa-mir-138-1 | 3 | 44130708 | 44130806 | + |
| MI0003570 | hsa-mir-564 | 3 | 44878384 | 44878477 | + |
| MI0003571 | hsa-mir-565 | 3 | 45705468 | 45705564 | − |
| MI0001448 | hsa-mir-425 | 3 | 49032585 | 49032671 | − |
| MI0000465 | hsa-mir-191 | 3 | 49033055 | 49033146 | − |
| MI0003572 | hsa-mir-566 | 3 | 50185763 | 50185856 | + |
| MI0000433 | hsa-let-7g | 3 | 52277334 | 52277417 | − |
| MI0000452 | hsa-mir-135a-1 | 3 | 52303275 | 52303364 | − |
| MI0003573 | hsa-mir-567 | 3 | 113314338 | 113314435 | + |
| MI0003574 | hsa-mir-568 | 3 | 115518012 | 115518106 | − |
| MI0000240 | hsa-mir-198 | 3 | 121597205 | 121597266 | − |
| MI0000438 | hsa-mir-15b | 3 | 161605070 | 161605167 | + |
| MI0000115 | hsa-mir-16-2 | 3 | 161605227 | 161605307 | + |
| MI0003575 | hsa-mir-551b | 3 | 169752336 | 169752431 | + |
| MI0003576 | hsa-mir-569 | 3 | 172307147 | 172307242 | − |
| MI0000086 | hsa-mir-28 | 3 | 189889263 | 189889348 | + |
| MI0003577 | hsa-mir-570 | 3 | 196911452 | 196911548 | + |
| MI0003578 | hsa-mir-571 | 4 | 333946 | 334041 | + |
| MI0000097 | hsa-mir-95 | 4 | 8057928 | 8058008 | − |
| MI0003579 | hsa-mir-572 | 4 | 10979549 | 10979643 | + |
| MI0000294 | hsa-mir-218-1 | 4 | 20138996 | 20139105 | + |
| MI0003580 | hsa-mir-573 | 4 | 24130913 | 24131011 | − |
| MI0003581 | hsa-mir-574 | 4 | 38546048 | 38546143 | + |
| MI0003582 | hsa-mir-575 | 4 | 83893514 | 83893607 | − |
| MI0003583 | hsa-mir-576 | 4 | 110629303 | 110629400 | + |
| MI0000775 | hsa-mir-367 | 4 | 113788479 | 113788546 | − |
| MI0000774 | hsa-mir-302d | 4 | 113788609 | 113788676 | − |
| MI0000738 | hsa-mir-302a | 4 | 113788788 | 113788856 | − |
| MI0000773 | hsa-mir-302c | 4 | 113788968 | 113789035 | − |
| MI0000772 | hsa-mir-302b | 4 | 113789090 | 113789162 | − |
| MI0003584 | hsa-mir-577 | 4 | 115797364 | 115797459 | + |
| MI0003585 | hsa-mir-578 | 4 | 166526844 | 166526939 | + |
| MI0003586 | hsa-mir-579 | 5 | 32430241 | 32430338 | − |
| MI0003587 | hsa-mir-580 | 5 | 36183751 | 36183847 | − |
| MI0003588 | hsa-mir-581 | 5 | 53283091 | 53283186 | − |
| MI0001648 | hsa-mir-449 | 5 | 54502117 | 54502207 | − |
| MI0003673 | hsa-mir-449b | 5 | 54502231 | 54502327 | − |
| MI0003589 | hsa-mir-582 | 5 | 59035189 | 59035286 | − |
| MI0000467 | hsa-mir-9-2 | 5 | 87998427 | 87998513 | − |
| MI0003590 | hsa-mir-583 | 5 | 95440598 | 95440672 | + |
| MI0003591 | hsa-mir-584 | 5 | 148422069 | 148422165 | − |
| MI0000459 | hsa-mir-143 | 5 | 148788674 | 148788779 | + |
| MI0000461 | hsa-mir-145 | 5 | 148790402 | 148790489 | + |
| MI0000786 | hsa-mir-378 | 5 | 149092581 | 149092646 | + |
| MI0000477 | hsa-mir-146a | 5 | 159844937 | 159845035 | + |
| MI0000109 | hsa-mir-103-1 | 5 | 167920479 | 167920556 | − |
| MI0000295 | hsa-mir-218-2 | 5 | 168127729 | 168127838 | − |
| MI0003592 | hsa-mir-585 | 5 | 168623183 | 168623276 | − |
| MI0000802 | hsa-mir-340 | 5 | 179374909 | 179375003 | − |
| MI0003593 | hsa-mir-548a-1 | 6 | 18679994 | 18680090 | + |
| MI0000296 | hsa-mir-219-1 | 6 | 33283590 | 33283699 | + |
| MI0003594 | hsa-mir-586 | 6 | 45273389 | 45273485 | − |
| MI0000490 | hsa-mir-206 | 6 | 52117106 | 52117191 | + |
| MI0000822 | hsa-mir-133b | 6 | 52121680 | 52121798 | + |
| MI0000254 | hsa-mir-30c-2 | 6 | 72143384 | 72143455 | − |
| MI0000088 | hsa-mir-30a | 6 | 72169975 | 72170045 | − |
| MI0003595 | hsa-mir-587 | 6 | 107338693 | 107338788 | + |
| MI0003596 | hsa-mir-548b | 6 | 119431911 | 119432007 | − |
| MI0003597 | hsa-mir-588 | 6 | 126847470 | 126847552 | + |
| MI0003598 | hsa-mir-548a-2 | 6 | 135601991 | 135602087 | + |
| MI0000815 | hsa-mir-339 | 7 | 1029095 | 1029188 | − |
| MI0003599 | hsa-mir-589 | 7 | 5501976 | 5502074 | − |
| MI0000253 | hsa-mir-148a | 7 | 25956064 | 25956131 | − |
| MI0001150 | hsa-mir-196b | 7 | 27175624 | 27175707 | − |
| MI0003600 | hsa-mir-550-1 | 7 | 30295935 | 30296031 | + |
| MI0003601 | hsa-mir-550-2 | 7 | 32739118 | 32739214 | + |
| MI0003602 | hsa-mir-590 | 7 | 73243464 | 73243560 | + |
| MI0003674 | hsa-mir-653 | 7 | 92950008 | 92950103 | − |
| MI0003124 | hsa-mir-489 | 7 | 92951184 | 92951267 | − |
| MI0003603 | hsa-mir-591 | 7 | 95686910 | 95687004 | − |
| MI0000082 | hsa-mir-25 | 7 | 99529119 | 99529202 | − |
| MI0000095 | hsa-mir-93 | 7 | 99529327 | 99529406 | − |
| MI0000734 | hsa-mir-106b | 7 | 99529552 | 99529633 | − |
| MI0003604 | hsa-mir-592 | 7 | 126485378 | 126485474 | − |
| MI0003605 | hsa-mir-593 | 7 | 127509149 | 127509248 | + |
| MI0000252 | hsa-mir-129-1 | 7 | 127635161 | 127635232 | + |
| MI0000272 | hsa-mir-182 | 7 | 129197459 | 129197568 | − |
| MI0000098 | hsa-mir-96 | 7 | 129201768 | 129201845 | − |
| MI0000273 | hsa-mir-183 | 7 | 129201981 | 129202090 | − |
| MI0000816 | hsa-mir-335 | 7 | 129923188 | 129923281 | + |
| MI0000087 | hsa-mir-29a | 7 | 130212046 | 130212109 | − |
| MI0000105 | hsa-mir-29b-1 | 7 | 130212758 | 130212838 | − |
| MI0003125 | hsa-mir-490 | 7 | 136238454 | 136238581 | + |
| MI0003606 | hsa-mir-594 | 7 | 138675997 | 138676085 | + |
| MI0003760 | hsa-mir-671 | 7 | 150566440 | 150566557 | + |
| MI0000464 | hsa-mir-153-2 | 7 | 157059789 | 157059875 | − |
| MI0003607 | hsa-mir-595 | 7 | 158018171 | 158018266 | − |
| MI0003608 | hsa-mir-596 | 8 | 1752804 | 1752880 | + |
| MI0003609 | hsa-mir-597 | 8 | 9636592 | 9636688 | + |
| MI0000443 | hsa-mir-124a-1 | 8 | 9798308 | 9798392 | − |
| MI0003610 | hsa-mir-598 | 8 | 10930126 | 10930222 | − |
| MI0000791 | hsa-mir-383 | 8 | 14755318 | 14755390 | − |
| MI0000542 | hsa-mir-320 | 8 | 22158420 | 22158501 | − |
| MI0002470 | hsa-mir-486 | 8 | 41637116 | 41637183 | + |
| MI0000444 | hsa-mir-124a-2 | 8 | 65454260 | 65454368 | + |
| MI0003611 | hsa-mir-599 | 8 | 100618040 | 100618134 | − |
| MI0003612 | hsa-mir-548a-3 | 8 | 105565773 | 105565869 | − |
| MI0003668 | hsa-mir-548d-1 | 8 | 124429455 | 124429551 | − |
| MI0000441 | hsa-mir-30b | 8 | 135881945 | 135882032 | − |
| MI0000255 | hsa-mir-30d | 8 | 135886301 | 135886370 | − |
| MI0000809 | hsa-mir-151 | 8 | 141811845 | 141811934 | − |
| MI0003669 | hsa-mir-661 | 8 | 145091347 | 145091435 | − |
| MI0000739 | hsa-mir-101-2 | 9 | 4840297 | 4840375 | + |
| MI0003126 | hsa-mir-491 | 9 | 20706104 | 20706187 | + |
| MI0000089 | hsa-mir-31 | 9 | 21502114 | 21502184 | − |
| MI0000284 | hsa-mir-204 | 9 | 72614711 | 72614820 | − |
| MI0000263 | hsa-mir-7-1 | 9 | 85774483 | 85774592 | − |
| MI0000060 | hsa-let-7a-1 | 9 | 95978060 | 95978139 | + |
| MI0000067 | hsa-let-7f-1 | 9 | 95978450 | 95978536 | + |
| MI0000065 | hsa-let-7d | 9 | 95980937 | 95981023 | + |
| MI0000439 | hsa-mir-23b | 9 | 96887311 | 96887407 | + |
| MI0000440 | hsa-mir-27b | 9 | 96887548 | 96887644 | + |
| MI0000080 | hsa-mir-24-1 | 9 | 96888124 | 96888191 | + |
| MI0000090 | hsa-mir-32 | 9 | 110848330 | 110848399 | − |
| MI0003513 | hsa-mir-455 | 9 | 116011535 | 116011630 | + |
| MI0000262 | hsa-mir-147 | 9 | 122047078 | 122047149 | − |
| MI0003613 | hsa-mir-600 | 9 | 124913646 | 124913743 | − |
| MI0003614 | hsa-mir-601 | 9 | 125204625 | 125204703 | − |
| MI0000269 | hsa-mir-181a-2 | 9 | 126494542 | 126494651 | + |
| MI0000683 | hsa-mir-181b-2 | 9 | 126495810 | 126495898 | + |
| MI0000282 | hsa-mir-199b | 9 | 130046821 | 130046930 | − |
| MI0000740 | hsa-mir-219-2 | 9 | 130194718 | 130194814 | − |
| MI0000471 | hsa-mir-126 | 9 | 138684875 | 138684959 | + |
| MI0003615 | hsa-mir-602 | 9 | 139852692 | 139852789 | + |
| MI0003127 | hsa-mir-511-1 | 10 | 17927113 | 17927199 | + |
| MI0003128 | hsa-mir-511-2 | 10 | 18174042 | 18174128 | + |
| MI0003616 | hsa-mir-603 | 10 | 24604620 | 24604716 | + |
| MI0003617 | hsa-mir-604 | 10 | 29873939 | 29874032 | − |
| MI0003618 | hsa-mir-605 | 10 | 52729339 | 52729421 | + |
| MI0003619 | hsa-mir-606 | 10 | 76982222 | 76982317 | + |
| MI0000826 | hsa-mir-346 | 10 | 88014424 | 88014509 | − |
| MI0000114 | hsa-mir-107 | 10 | 91342484 | 91342564 | − |
| MI0003620 | hsa-mir-607 | 10 | 98578416 | 98578511 | − |
| MI0003621 | hsa-mir-608 | 10 | 102724732 | 102724831 | + |
| MI0003129 | hsa-mir-146b | 10 | 104186259 | 104186331 | + |
| MI0003622 | hsa-mir-609 | 10 | 105968537 | 105968631 | − |
| MI0003130 | hsa-mir-202 | 10 | 134911006 | 134911115 | − |
| MI0000286 | hsa-mir-210 | 11 | 558089 | 558198 | − |
| MI0002467 | hsa-mir-483 | 11 | 2111940 | 2112015 | − |
| MI0003623 | hsa-mir-610 | 11 | 28034938 | 28035033 | + |
| MI0000473 | hsa-mir-129-2 | 11 | 43559520 | 43559609 | + |
| MI0000448 | hsa-mir-130a | 11 | 57165247 | 57165335 | + |
| MI0003624 | hsa-mir-611 | 11 | 61316543 | 61316609 | − |
| MI0000234 | hsa-mir-192 | 11 | 64415185 | 64415294 | − |
| MI0000732 | hsa-mir-194-2 | 11 | 64415403 | 64415487 | − |
| MI0003625 | hsa-mir-612 | 11 | 64968505 | 64968604 | + |
| MI0000261 | hsa-mir-139 | 11 | 72003755 | 72003822 | − |
| MI0000808 | hsa-mir-326 | 11 | 74723784 | 74723878 | − |
| MI0000742 | hsa-mir-34b | 11 | 110888873 | 110888956 | + |
| MI0000743 | hsa-mir-34c | 11 | 110889374 | 110889450 | + |
| MI0000446 | hsa-mir-125b-1 | 11 | 121475675 | 121475762 | − |
| MI0000061 | hsa-let-7a-2 | 11 | 121522440 | 121522511 | − |
| MI0000102 | hsa-mir-100 | 11 | 121528147 | 121528226 | − |
| MI0000650 | hsa-mir-200c | 12 | 6943123 | 6943190 | + |
| MI0000457 | hsa-mir-141 | 12 | 6943521 | 6943615 | + |
| MI0003626 | hsa-mir-613 | 12 | 12808850 | 12808944 | + |
| MI0003627 | hsa-mir-614 | 12 | 12960030 | 12960119 | + |
| MI0000279 | hsa-mir-196a-2 | 12 | 52671789 | 52671898 | + |
| MI0003628 | hsa-mir-615 | 12 | 52714001 | 52714096 | + |
| MI0000811 | hsa-mir-148b | 12 | 53017267 | 53017365 | + |
| MI0003629 | hsa-mir-616 | 12 | 56199213 | 56199309 | − |
| MI0000750 | hsa-mir-26a-2 | 12 | 56504659 | 56504742 | − |
| MI0000434 | hsa-let-7l | 12 | 61283733 | 61283816 | + |
| MI0003630 | hsa-mir-548c | 12 | 63302556 | 63302652 | + |
| MI0003631 | hsa-mir-617 | 12 | 79750443 | 79750539 | − |
| MI0003632 | hsa-mir-618 | 12 | 79853646 | 79853743 | − |
| MI0003131 | hsa-mir-492 | 12 | 93752305 | 93752420 | + |
| MI0000812 | hsa-mir-331 | 12 | 94226327 | 94226420 | + |
| MI0000453 | hsa-mir-135a-2 | 12 | 96481721 | 96481820 | + |
| MI0003633 | hsa-mir-619 | 12 | 107754813 | 107754911 | − |
| MI0003634 | hsa-mir-620 | 12 | 115070748 | 115070842 | − |
| MI0003635 | hsa-mir-621 | 13 | 40282902 | 40282997 | + |
| MI0000070 | hsa-mir-16-1 | 13 | 49521110 | 49521198 | − |
| MI0000069 | hsa-mir-15a | 13 | 49521256 | 49521338 | − |
| MI0003636 | hsa-mir-622 | 13 | 89681437 | 89681532 | + |
| MI0000071 | hsa-mir-17 | 13 | 90800860 | 90800943 | + |
| MI0000072 | hsa-mir-18a | 13 | 90801006 | 90801076 | + |
| MI0000073 | hsa-mir-19a | 13 | 90801146 | 90801227 | + |
| MI0000076 | hsa-mir-20a | 13 | 90801320 | 90801390 | + |
| MI0000074 | hsa-mir-19b-1 | 13 | 90801447 | 90801533 | + |
| MI0000093 | hsa-mir-92-1 | 13 | 90801569 | 90801646 | + |
| MI0003637 | hsa-mir-623 | 13 | 98806386 | 98806483 | + |
| MI0000251 | hsa-mir-208 | 14 | 22927645 | 22927715 | − |
| MI0003638 | hsa-mir-624 | 14 | 30553603 | 30553699 | − |
| MI0003639 | hsa-mir-625 | 14 | 65007573 | 65007657 | + |
| MI0000805 | hsa-mir-342 | 14 | 99645745 | 99645843 | + |
| MI0000825 | hsa-mir-345 | 14 | 99843949 | 99844046 | + |
| MI0005118 | hsa-mir-770 | 14 | 100388480 | 100388577 | + |
| MI0003132 | hsa-mir-493 | 14 | 100405150 | 100405238 | + |
| MI0000806 | hsa-mir-337 | 14 | 100410583 | 100410675 | + |
| MI0001721 | hsa-mir-431 | 14 | 100417097 | 100417210 | + |
| MI0001723 | hsa-mir-433 | 14 | 100417976 | 100418068 | + |
| MI0000472 | hsa-mir-127 | 14 | 100419069 | 100419165 | + |
| MI0003133 | hsa-mir-432 | 14 | 100420573 | 100420666 | + |
| MI0000475 | hsa-mir-136 | 14 | 100420792 | 100420873 | + |
| MI0000778 | hsa-mir-370 | 14 | 100447229 | 100447303 | + |
| MI0000787 | hsa-mir-379 | 14 | 100558156 | 100558222 | + |
| MI0003675 | hsa-mir-411 | 14 | 100559415 | 100559510 | + |
| MI0000744 | hsa-mir-299 | 14 | 100559884 | 100559946 | + |
| MI0000788 | hsa-mir-380 | 14 | 100561107 | 100561167 | + |
| MI0000807 | hsa-mir-323 | 14 | 100561822 | 100561907 | + |
| MI0003757 | hsa-mir-758 | 14 | 100562110 | 100562197 | + |
| MI0001725 | hsa-mir-329-1 | 14 | 100562875 | 100562954 | + |
| MI0001726 | hsa-mir-329-2 | 14 | 100563190 | 100563273 | + |
| MI0003134 | hsa-mir-494 | 14 | 100565724 | 100565804 | + |
| MI0003135 | hsa-mir-495 | 14 | 100569845 | 100569926 | + |
| MI0000776 | hsa-mir-368 | 14 | 100575780 | 100575845 | + |
| MI0003529 | hsa-mir-376a-2 | 14 | 100576159 | 100576238 | + |
| MI0003676 | hsa-mir-654 | 14 | 100576309 | 100576389 | + |
| MI0002466 | hsa-mir-376b | 14 | 100576526 | 100576625 | + |
| MI0000784 | hsa-mir-376a-1 | 14 | 100576872 | 100576939 | + |
| MI0000789 | hsa-mir-381 | 14 | 100582010 | 100582084 | + |
| MI0003530 | hsa-mir-487b | 14 | 100582545 | 100582628 | + |
| MI0003514 | hsa-mir-539 | 14 | 100583411 | 100583488 | + |
| MI0003515 | hsa-mir-544 | 14 | 100584748 | 100584838 | + |
| MI0003677 | hsa-mir-655 | 14 | 100585640 | 100585736 | + |
| MI0002471 | hsa-mir-487a | 14 | 100588536 | 100588615 | + |
| MI0000790 | hsa-mir-382 | 14 | 100590396 | 100590471 | + |
| MI0000474 | hsa-mir-134 | 14 | 100590777 | 100590849 | + |
| MI0003761 | hsa-mir-668 | 14 | 100591348 | 100591413 | + |
| MI0002469 | hsa-mir-485 | 14 | 100591509 | 100591581 | + |
| MI0001727 | hsa-mir-453 | 14 | 100592280 | 100592359 | + |
| MI0000480 | hsa-mir-154 | 14 | 100595845 | 100595928 | + |
| MI0003136 | hsa-mir-496 | 14 | 100596663 | 100596764 | + |
| MI0000785 | hsa-mir-377 | 14 | 100598140 | 100598208 | + |
| MI0001735 | hsa-mir-409 | 14 | 100601390 | 100601468 | + |
| MI0002464 | hsa-mir-412 | 14 | 100601537 | 100601627 | + |
| MI0000777 | hsa-mir-369 | 14 | 100601688 | 100601757 | + |
| MI0002465 | hsa-mir-410 | 14 | 100602002 | 100602081 | + |
| MI0003678 | hsa-mir-656 | 14 | 100602814 | 100602891 | + |
| MI0000283 | hsa-mir-203 | 14 | 103653495 | 103653604 | + |
| MI0000287 | hsa-mir-211 | 15 | 29144527 | 29144636 | − |
| MI0003640 | hsa-mir-626 | 15 | 39771075 | 39771168 | + |
| MI0003641 | hsa-mir-627 | 15 | 40279060 | 40279156 | − |
| MI0003642 | hsa-mir-628 | 15 | 53452430 | 53452524 | − |
| MI0000486 | hsa-mir-190 | 15 | 60903209 | 60903293 | + |
| MI0001444 | hsa-mir-422a | 15 | 61950182 | 61950271 | − |
| MI0003643 | hsa-mir-629 | 15 | 68158765 | 68158861 | − |
| MI0003644 | hsa-mir-630 | 15 | 70666612 | 70666708 | + |
| MI0003645 | hsa-mir-631 | 15 | 73433005 | 73433079 | − |
| MI0000481 | hsa-mir-184 | 15 | 77289185 | 77289268 | + |
| MI0003679 | hsa-mir-549 | 15 | 78921374 | 78921469 | − |
| MI0000264 | hsa-mir-7-2 | 15 | 86956060 | 86956169 | + |
| MI0000468 | hsa-mir-9-3 | 15 | 87712252 | 87712341 | + |
| MI0003670 | hsa-mir-662 | 16 | 760184 | 760278 | + |
| MI0003137 | hsa-mir-193b | 16 | 14305325 | 14305407 | + |
| MI0000767 | hsa-mir-365-1 | 16 | 14310643 | 14310729 | + |
| MI0002468 | hsa-mir-484 | 16 | 15644652 | 15644730 | + |
| MI0000455 | hsa-mir-138-2 | 16 | 55449931 | 55450014 | + |
| MI0000804 | hsa-mir-328 | 16 | 65793725 | 65793799 | − |
| MI0000456 | hsa-mir-140 | 16 | 68524485 | 68524584 | + |
| MI0005117 | hsa-mir-768 | 16 | 70349796 | 70349899 | − |
| MI0000078 | hsa-mir-22 | 17 | 1563947 | 1564031 | − |
| MI0000449 | hsa-mir-132 | 17 | 1899952 | 1900052 | − |
| MI0000288 | hsa-mir-212 | 17 | 1900315 | 1900424 | − |
| MI0000489 | hsa-mir-195 | 17 | 6861658 | 6861744 | − |
| MI0003138 | hsa-mir-497 | 17 | 6861954 | 6862065 | − |
| MI0000813 | hsa-mir-324 | 17 | 7067340 | 7067422 | − |
| MI0003646 | hsa-mir-33b | 17 | 17657875 | 17657970 | − |
| MI0001729 | hsa-mir-451 | 17 | 24212513 | 24212584 | − |
| MI0000460 | hsa-mir-144 | 17 | 24212677 | 24212762 | − |
| MI0001445 | hsa-mir-423 | 17 | 25468223 | 25468316 | + |
| MI0000487 | hsa-mir-193a | 17 | 26911128 | 26911215 | + |
| MI0000769 | hsa-mir-365-2 | 17 | 26926543 | 26926653 | + |
| MI0003647 | hsa-mir-632 | 17 | 27701241 | 27701334 | + |
| MI0000462 | hsa-mir-152 | 17 | 43469526 | 43469612 | − |
| MI0000266 | hsa-mir-10a | 17 | 44012199 | 44012308 | − |
| MI0000238 | hsa-mir-196a-1 | 17 | 44064851 | 44064920 | − |
| MI0000458 | hsa-mir-142 | 17 | 53763592 | 53763678 | − |
| MI0003820 | hsa-mir-454 | 17 | 54569901 | 54570015 | − |
| MI0000745 | hsa-mir-301 | 17 | 54583279 | 54583364 | − |
| MI0000077 | hsa-mir-21 | 17 | 55273409 | 55273480 | + |
| MI0003648 | hsa-mir-633 | 17 | 58375308 | 58375405 | + |
| MI0003649 | hsa-mir-634 | 17 | 62213652 | 62213748 | + |
| MI0003671 | hsa-mir-548d-2 | 17 | 62898067 | 62898163 | − |
| MI0003650 | hsa-mir-635 | 17 | 63932187 | 63932284 | − |
| MI0003651 | hsa-mir-636 | 17 | 72244127 | 72244225 | − |
| MI0003681 | hsa-mir-657 | 17 | 76713671 | 76713768 | − |
| MI0000814 | hsa-mir-338 | 17 | 76714278 | 76714344 | − |
| MI0000450 | hsa-mir-133a-1 | 18 | 17659657 | 17659744 | − |
| MI0000437 | hsa-mir-1-2 | 18 | 17662963 | 17663047 | − |
| MI0000274 | hsa-mir-187 | 18 | 31738779 | 31738887 | − |
| MI0000442 | hsa-mir-122a | 18 | 54269286 | 54269370 | + |
| MI0003652 | hsa-mir-637 | 19 | 3912412 | 3912510 | − |
| MI0000265 | hsa-mir-7-3 | 19 | 4721682 | 4721791 | + |
| MI0003653 | hsa-mir-638 | 19 | 10690080 | 10690179 | + |
| MI0000242 | hsa-mir-199a-1 | 19 | 10789102 | 10789172 | − |
| MI0000081 | hsa-mir-24-2 | 19 | 13808101 | 13808173 | − |
| MI0000085 | hsa-mir-27a | 19 | 13808254 | 13808331 | − |
| MI0000079 | hsa-mir-23a | 19 | 13808401 | 13808473 | − |
| MI0000271 | hsa-mir-181c | 19 | 13846513 | 13846622 | + |
| MI0003139 | hsa-mir-181d | 19 | 13846689 | 13846825 | + |
| MI0003654 | hsa-mir-639 | 19 | 14501355 | 14501452 | + |
| MI0003655 | hsa-mir-640 | 19 | 19406872 | 19406967 | + |
| MI0003656 | hsa-mir-641 | 19 | 45480290 | 45480388 | − |
| MI0000803 | hsa-mir-330 | 19 | 50834092 | 50834185 | − |
| MI0003657 | hsa-mir-642 | 19 | 50870026 | 50870122 | + |
| MI0003834 | hsa-mir-769 | 19 | 51214030 | 51214147 | + |
| MI0000479 | hsa-mir-150 | 19 | 54695854 | 54695937 | − |
| MI0000746 | hsa-mir-99b | 19 | 56887677 | 56887746 | + |
| MI0000066 | hsa-let-7e | 19 | 56887851 | 56887929 | + |
| MI0000469 | hsa-mir-125a | 19 | 56888319 | 56888404 | + |
| MI0003658 | hsa-mir-643 | 19 | 57476862 | 57476958 | + |
| MI0003140 | hsa-mir-512-1 | 19 | 58861745 | 58861828 | + |
| MI0003141 | hsa-mir-512-2 | 19 | 58864223 | 58864320 | + |
| MI0003142 | hsa-mir-498 | 19 | 58869263 | 58869386 | + |
| MI0003143 | hsa-mir-520e | 19 | 58870777 | 58870863 | + |
| MI0003144 | hsa-mir-515-1 | 19 | 58874069 | 58874151 | + |
| MI0003145 | hsa-mir-519e | 19 | 58875006 | 58875089 | + |
| MI0003146 | hsa-mir-520f | 19 | 58877225 | 58877311 | + |
| MI0003147 | hsa-mir-515-2 | 19 | 58880075 | 58880157 | + |
| MI0003148 | hsa-mir-519c | 19 | 58881535 | 58881621 | + |
| MI0003149 | hsa-mir-520a | 19 | 58885947 | 58886031 | + |
| MI0003150 | hsa-mir-526b | 19 | 58889459 | 58889541 | + |
| MI0003151 | hsa-mir-519b | 19 | 58890279 | 58890359 | + |
| MI0003152 | hsa-mir-525 | 19 | 58892599 | 58892683 | + |
| MI0003153 | hsa-mir-523 | 19 | 58893451 | 58893537 | + |
| MI0003154 | hsa-mir-518f | 19 | 58895081 | 58895167 | + |
| MI0003155 | hsa-mir-520b | 19 | 58896293 | 58896353 | + |
| MI0003156 | hsa-mir-518b | 19 | 58897803 | 58897885 | + |
| MI0003157 | hsa-mir-526a-1 | 19 | 58901318 | 58901402 | + |
| MI0003158 | hsa-mir-520c | 19 | 58902519 | 58902605 | + |
| MI0003159 | hsa-mir-518c | 19 | 58903801 | 58903901 | + |
| MI0003160 | hsa-mir-524 | 19 | 58906068 | 58906154 | + |
| MI0003161 | hsa-mir-517a | 19 | 58907334 | 58907420 | + |
| MI0003162 | hsa-mir-519d | 19 | 58908413 | 58908500 | + |
| MI0003163 | hsa-mir-521-2 | 19 | 58911660 | 58911746 | + |
| MI0003164 | hsa-mir-520d | 19 | 58915162 | 58915248 | + |
| MI0003165 | hsa-mir-517b | 19 | 58916142 | 58916208 | + |
| MI0003166 | hsa-mir-520g | 19 | 58917232 | 58917321 | + |
| MI0003167 | hsa-mir-516-3 | 19 | 58920508 | 58920592 | + |
| MI0003168 | hsa-mir-526a-2 | 19 | 58921988 | 58922052 | + |
| MI0003169 | hsa-mir-518e | 19 | 58924904 | 58924991 | + |
| MI0003170 | hsa-mir-518a-1 | 19 | 58926072 | 58926156 | + |
| MI0003171 | hsa-mir-518d | 19 | 58929943 | 58930029 | + |
| MI0003172 | hsa-mir-516-4 | 19 | 58931911 | 58932000 | + |
| MI0003173 | hsa-mir-518a-2 | 19 | 58934399 | 58934485 | + |
| MI0003174 | hsa-mir-517c | 19 | 58936379 | 58936473 | + |
| MI0003175 | hsa-mir-520h | 19 | 58937578 | 58937665 | + |
| MI0003176 | hsa-mir-521-1 | 19 | 58943702 | 58943788 | + |
| MI0003177 | hsa-mir-522 | 19 | 58946277 | 58946363 | + |
| MI0003178 | hsa-mir-519a-1 | 19 | 58947463 | 58947547 | + |
| MI0003179 | hsa-mir-527 | 19 | 58949084 | 58949168 | + |
| MI0003180 | hsa-mir-516-1 | 19 | 58951807 | 58951896 | + |
| MI0003181 | hsa-mir-516-2 | 19 | 58956199 | 58956288 | + |
| MI0003182 | hsa-mir-519a-2 | 19 | 58957410 | 58957496 | + |
| MI0000779 | hsa-mir-371 | 19 | 58982741 | 58982807 | + |
| MI0000780 | hsa-mir-372 | 19 | 58982956 | 58983022 | + |
| MI0000781 | hsa-mir-373 | 19 | 58983771 | 58983839 | + |
| MI0000108 | hsa-mir-103-2 | 20 | 3846141 | 3846218 | + |
| MI0003672 | hsa-mir-663 | 20 | 26136822 | 26136914 | − |
| MI0003659 | hsa-mir-644 | 20 | 32517791 | 32517884 | + |
| MI0003183 | hsa-mir-499 | 20 | 33041840 | 33041961 | + |
| MI0003660 | hsa-mir-645 | 20 | 48635730 | 48635823 | + |
| MI0000747 | hsa-mir-296 | 20 | 56826065 | 56826144 | − |
| MI0003661 | hsa-mir-646 | 20 | 58316927 | 58317020 | + |
| MI0000651 | hsa-mir-1-1 | 20 | 60561958 | 60562028 | + |
| MI0000451 | hsa-mir-133a-2 | 20 | 60572564 | 60572665 | + |
| MI0000445 | hsa-mir-124a-3 | 20 | 61280297 | 61280383 | + |
| MI0003662 | hsa-mir-647 | 20 | 62044428 | 62044523 | − |
| MI0000101 | hsa-mir-99a | 21 | 16833280 | 16833360 | + |
| MI0000064 | hsa-let-7c | 21 | 16834019 | 16834102 | + |
| MI0000470 | hsa-mir-125b-2 | 21 | 16884428 | 16884516 | + |
| MI0000681 | hsa-mir-155 | 21 | 25868163 | 25868227 | + |
| MI0003906 | hsa-mir-802 | 21 | 36014883 | 36014976 | + |
| MI0003663 | hsa-mir-648 | 22 | 16843634 | 16843727 | − |
| MI0000482 | hsa-mir-185 | 22 | 18400662 | 18400743 | + |
| MI0003664 | hsa-mir-649 | 22 | 19718465 | 19718561 | − |
| MI0000748 | hsa-mir-130b | 22 | 20337593 | 20337674 | + |
| MI0003665 | hsa-mir-650 | 22 | 21495270 | 21495365 | + |
| MI0003682 | hsa-mir-658 | 22 | 36570225 | 36570324 | − |
| MI0003683 | hsa-mir-659 | 22 | 36573631 | 36573727 | − |
| MI0000091 | hsa-mir-33 | 22 | 40626894 | 40626962 | + |
| MI0000062 | hsa-let-7a-3 | 22 | 44887293 | 44887366 | + |
| MI0000063 | hsa-let-7b | 22 | 44888230 | 44888312 | + |
| MI0003666 | hsa-mir-651 | X | 8055006 | 8055102 | + |
| MI0000298 | hsa-mir-221 | X | 45490529 | 45490638 | − |
| MI0000299 | hsa-mir-222 | X | 45491365 | 45491474 | − |
| MI0003205 | hsa-mir-532 | X | 49654494 | 49654584 | + |
| MI0000484 | hsa-mir-188 | X | 49654849 | 49654934 | + |
| MI0003184 | hsa-mir-500 | X | 49659779 | 49659862 | + |
| MI0000762 | hsa-mir-362 | X | 49660312 | 49660376 | + |
| MI0003185 | hsa-mir-501 | X | 49661070 | 49661153 | + |
| MI0003684 | hsa-mir-660 | X | 49664589 | 49664685 | + |
| MI0003186 | hsa-mir-502 | X | 49665946 | 49666031 | + |
| MI0000100 | hsa-mir-98 | X | 53599909 | 53600027 | − |
| MI0000068 | hsa-let-7f-2 | X | 53600878 | 53600960 | − |
| MI0000300 | hsa-mir-223 | X | 65155437 | 65155546 | + |
| MI0003685 | hsa-mir-421 | X | 73354937 | 73355021 | − |
| MI0003516 | hsa-mir-545 | X | 73423664 | 73423769 | − |
| MI0000782 | hsa-mir-374 | X | 73423846 | 73423917 | − |
| MI0001145 | hsa-mir-384 | X | 76056092 | 76056179 | − |
| MI0000824 | hsa-mir-325 | X | 76142220 | 76142317 | − |
| MI0000760 | hsa-mir-361 | X | 85045297 | 85045368 | − |
| MI0003667 | hsa-mir-652 | X | 109185213 | 109185310 | + |
| MI0001637 | hsa-mir-448 | X | 113964273 | 113964383 | + |
| MI0003836 | hsa-mir-766 | X | 118664729 | 118664839 | − |
| MI0000297 | hsa-mir-220 | X | 122523627 | 122523736 | − |
| MI0000764 | hsa-mir-363 | X | 133131074 | 133131148 | − |
| MI0000094 | hsa-mir-92-2 | X | 133131234 | 133131308 | − |
| MI0000075 | hsa-mir-19b-2 | X | 133131367 | 133131462 | − |
| MI0001519 | hsa-mir-20b | X | 133131505 | 133131573 | − |
| MI0001518 | hsa-mir-18b | X | 133131737 | 133131807 | − |
| MI0000113 | hsa-mir-106a | X | 133131894 | 133131974 | − |
| MI0001652 | hsa-mir-450-1 | X | 133502037 | 133502127 | − |
| MI0003187 | hsa-mir-450-2 | X | 133502204 | 133502303 | − |
| MI0003686 | hsa-mir-542 | X | 133503037 | 133503133 | − |
| MI0003188 | hsa-mir-503 | X | 133508024 | 133508094 | − |
| MI0001446 | hsa-mir-424 | X | 133508310 | 133508407 | − |
| MI0003189 | hsa-mir-504 | X | 137577538 | 137577620 | − |
| MI0003190 | hsa-mir-505 | X | 138833973 | 138834056 | − |
| MI0003191 | hsa-mir-513-1 | X | 146102673 | 146102801 | − |
| MI0003192 | hsa-mir-513-2 | X | 146115036 | 146115162 | − |
| MI0003193 | hsa-mir-506 | X | 146119930 | 146120053 | − |
| MI0003194 | hsa-mir-507 | X | 146120194 | 146120287 | − |
| MI0003195 | hsa-mir-508 | X | 146126123 | 146126237 | − |
| MI0003196 | hsa-mir-509 | X | 146149742 | 146149835 | − |
| MI0003197 | hsa-mir-510 | X | 146161545 | 146161618 | − |
| MI0003198 | hsa-mir-514-1 | X | 146168457 | 146168554 | − |
| MI0003199 | hsa-mir-514-2 | X | 146171153 | 146171240 | − |
| MI0003200 | hsa-mir-514-3 | X | 146173851 | 146173938 | − |
| MI0000301 | hsa-mir-224 | X | 150877706 | 150877786 | − |
| MI0001733 | hsa-mir-452 | X | 150878756 | 150878840 | − |
| MI0000111 | hsa-mir-105-1 | X | 151311347 | 151311427 | − |
| MI0003763 | hsa-mir-767 | X | 151312549 | 151312657 | − |
| MI0000112 | hsa-mir-105-2 | X | 151313540 | 151313620 | − |

According to the teachings herein, detection probes can be prepared which bind specifically to any one of these human miRNA molecules, i.e. the detection probes are complementary to a sequence in these human miRNAs (e.g. in their mature form) and include modified nucleobases as discussed in detail herein.

A thorough investigation of tissue and tumour expression of these miRNAs (e.g. using microarrays comprising a plurality of such probes) will allow for the preparation of an “expression pattern library” prepared e.g. as shown in Example 16 herein, where a number of head and neck tumours are shown to exhibit differences in miRNA expression patterns. This, in turn, enables a simple comparison of a miRNA expression profile from a metastatic tumour of unknown origin with the existing expression patterns in this expression pattern library and thereby a precise and statistically reliable determination of the true origin of the tested tumour.

A number of specific probes useful in the present invention are the following which have i.a. been used for testing expression levels of their targets in breast cancer:

TABLE U

| Human miRNA | PROBE SEQUENCE 5′-3′ |
|---|---|
| Has-miR-142-3 | tmCcaTaaAgtAggAaamCacTaca |
| hsa-miR-451 | mCtcAgtAatGgtAacGgt |
| hsa-miR-451 | AaamCtcAgtAatGgtAacGg |
| hsa-miR-136 | tccAtcAtcAaaAcaAatGgaGt |
| hsa-miR-193a | GggActTtgTagGccAg |
| hsa-miR-199a | gaAcaGgtAgtmCtgAacActGgg |
| hsa-miR-492 | gaAtcTtgTccmCgcAggt |
| hsa-miR-193b | ggActTtgAggGccAgtt |
| hsa-miR-199a* | aacmCaaTgtGcaGacTacTgta |
| hsa-miR-365 | aTaaGgaTttTtaGggGcaTt |
| hsa-miR-15a | cAcaAacmCatTatGtgmCtgmCta |
| hsa-miR-22 | acaGttmCttmCaamCtgGcaGctt |
| hsa-miR-140 | ctAccAtaGggTaaAacmCact |
| hsa-miR-518c* | aGtgmCttmCccTccAgag |
| hsa-miR-34a | aamCaamCcaGctAagAcamCtgmCca |
| hsa-miR-15b | tgtAaamCcaTgaTgtGctGcta |
| hsa-miR-370 | ccAggTtcmCacmCccAgcAggc |
| hsa-miR-214 | ctGccTgtmCtgTgcmCtgmCtgt |
| hsa-miR-525 | AaaGtgmCatmCccTctGga |
| hsa-miR-373* | acamCccmCaaAatmCgaAgcActTc |
| hsa-miR-148b | acaAagTtcTgtGatGcamCtga |
| hsa-miR-185 | gAacTgcmCttTctmCtcmCa |
| hsa-miR-516-5p | agTgcTtcTtamCctmCcaGa |
| hsa-miR-503 | AacTgtTccmCgcTgcTa |
| hsa-miR-27a | gcGgaActTagmCcamCtgTgaa |
| hsa-miR-223 | GggGtaTttGacAaamCtgAca |
| hsa-miR-222 | gaGacmCcaGtaGccAgaTgtAgct |
| hsa-miR-30a-5p | cTtcmCagTcgAggAtgTttAca |
| hsa-miR-30e-5p | tcmCagTcaAggAtgTttAca |
| hsa-miR-148b | acaAagTtcTgtGatGcamCtga |
| hsa-miR-342 | gacGggTgcGatTtcTgtGtgAga |
| hsa-miR-195 | gmCcaAtaTttmCtgTgcTgcTa |
| hsa-miR-27b | gcAgaActTagmCcamCtgTgaa |
| hsa-miR-326 | ctgGagGaaGggmCccAgaGg |
| hsa-miR-146b | aGccTatGgaAttmCagTtcTca |
| hsa-miR-195 | gmCcaAtaTttmCtgTgcTgcTa |
| hsa-let-7g | amCtgTacAaamCtamCtamCctmCa |
| hsa-miR-221 | gAaamCccAgcAgamCaaTgtAgct |
| hsa-miR-483 | aaGacGggAggAgag |
| hsa-miR-30c | gmCtgAgaGtgTagGatGttTaca |
| hsa-miR-29c | amCcgAttTcaAatGgtGcta |
| hsa-miR-296 | acAggAttGagGggGggmCcct |
| hsa-let-7e | actAtamCaamCctmCctAccTca |
| hsa-let-7f | aamCtaTacAatmCtamCtamCctmCa |
| hsa-miR-199b | gAacAgaTagTctAaamCacTggg |
| hsa-miR-21 | tmCaamCatmCagTctGatAagmCta |
| hsa-miR-202 | ttTtcmCcaTgcmCctAtamCct |
| hsa-miR-129 | gcAagmCccAgamCcgmCaaAaag |
| hsa-miR-513 | aaTgamCacmCtcmCctGtga |
| hsa-miR-494 | aGagGttTccmCgtGtaTg |
| hsa-miR-126 | gcAttAttActmCacGgtAcga |
| hsa-let-7i | amCagmCacAaamCtamCtamCctmCa |
| hsa-miR-23a | gGaaAtcmCctGgcAatGtgAt |
| hsa-miR-498 | gAaaAacGccmCccTgg |
| hsa-miR-24 | cTgtTccTgcTgaActGagmCca |
| hsa-miR-16 | ccaAtaTttAcgTgcTgcTa |
| hsa-miR-320 | tTcgmCccTctmCaamCccAgcTttt |
| hsa-miR-205 | caGacTccGgtGgaAtgAagGa |
| hsa-miR-200c | ccAtcAttAccmCggmCagTatTa |
| hsa-miR-200b | cAtcAttAccAggmCagTatTaga |
| hsa-miR-100 | cacAagTtcGgaTctAcgGgtt |
| hsa-let-7c | aamCcaTacAacmCtamCtamCctmCa |
| hsa-let-7b | aamCcamCacAacmCtamCtamCctmCa |
| hsa-miR-26a | gcmCtaTccTggAttActTgaa |
| hsa-miR-130a | aTgcmCctTttAacAttGcamCtg |
| hsa-miR-26b | aacmCtaTccTgaAttActTgaa |
| hsa-miR-195 | gmCcaAtaTttmCtgTgcTgcTa |
| hsa-miR-10a | cAcaAatTcgGatmCtamCagGgta |
| hsa-miR-326 | ctgGagGaaGggmCccAgaGg |
| hsa-miR-10b | amCaaAttmCggTtcTacAggGta |
| hsa-miR-141 | cmCatmCttTacmCagAcaGtgTta |
| hsa-miR-30b | agcTgaGtgTagGatGttTaca |
| hsa-miR-191 | agcTgcTttTggGatTccGttg |
| hsa-miR-195 | gmCcaAtaTttmCtgTgcTgcTa |
| hsa-let-7g | amCtgTacAaamCtamCtamCctmCa |
| hsa-miR-455 | gAaaAacGccmCccTgg |
| hsa-miR-526b | aAgtGctTccmCtcAagAg |
| hsa-miR-99a | cacAagAtcGgaTctAcgGgtt |
| hsa-miR-515-5p | aGtgmCttTctTttGgaGa |
| hsa-miR-191* | agcTgcTttTggGatTccGttg |
| hsa-miR-217 | atcmCaaTcaGttmCctGatGcaGta |
| hsa-miR-150 | cacTggTacAagGgtTggGaga |
| hsa-miR-29a | aamCcgAttTcaGatGgtGcta |
| hsa-miR-452* | tmCttTgcAgaTgaGacTga |
| hsa-miR-320 | tTcgmCccTctmCaamCccAgcTttt |
| hsa-let-7a | aamCtaTacAacmCtamCtamCctmCa |
| hsa-miR-125b | tcamCaaGttAggGtcTcaGgga |
| hsa-miR-133b | taGctGgtTgaAggGgamCcaa |
| hsa-miR-101 | cTtcAgtTatmCacAgtActg |
| hsa-miR-145 | tmCctGggAaaActGga |
| hsa-miR-9 | cAtamCagmCtaGatAacmCaaAga |
| hsa-miR-122a | camCcaTtgTcamCacTccA |
| hsa-miR-128b | GaaAgaGacmCggTtcActG |
| hsa-miR-149 | AgtGaaGacAcgGagmC |
| hsa-miR-125a | acAggTtaAagGgtmCtcAg |
| hsa-miR-143 | AgcTacAgtGctTcaTctmCa |
| hsa-miR-136 | cmCatmCatmCaaAacAaaTggAg |

LNA nucleotides are depicted by capital letters, DNA nucleotides by lowercase letters, mC denotes LNA methyl-cytosine.

Methods for Defining and Preparing Probes and Probe Collections

The invention utilises a method for expanding or building a collection defined above, a method to design single probes, a computer system for designing an optimized detection probe for a target nucleic acid sequence, and a storage means embedding executable code for designing the optimized detection probes—all disclosures relating to these practical tools for carrying out the present invention are described in detail in WO 2006/069584 and all disclosures therein apply mutatis mutandis to the teachings of the present invention.

Methods/Uses of Probes and Probe Collections

The main aspect of the invention relates to identification of a target miRNA derived sequence in a sample from a metastatic tumour of unknown origin, by contacting said sample with a member of a collection of probes or a probe defined herein under conditions that facilitate hybridization between said member/probe and said target nucleotide sequence and subsequently detecting the duplex formed between the probe and the target sequence—this, in turn allows for identification of the tissue origin of the expressed miRNA found in the sample, thus e.g. allowing for rational therapy targeting the metastatic tumour.

Typically, the miRNA which is identified and optionally quantified is a mature miRNA. A very surprising finding of the present invention is that it is possible to effect specific hybridization with miRNAs using probes of very short lengths, such as those lengths discussed herein when discussing the collection of probes. Typically the small, non-coding RNA has a length of at most 30 residues, such as at most 29, 28, 27, 26, 25, 24, 23, 22, 21, 20, 19, or 18 residues. The small non-coding RNA typically also has a length of at least 15 residues, such as at least 16, 17, 18, 19, 20, 21, 22, 23, 24, 25, 26, 27, 28, 29 or 30 residues.

As detailed in the examples herein, the specific hybridization between the short probes of the present invention to miRNA and the fact that miRNA can be mapped to various tissue origins, hence allows for an embodiment of the uses/methods of the present invention comprising identification of the primary site of metastatic tumors of unknown origin.

It is for instance contemplated to determine, by studying miRNA expression profiles in a given tumour sample, whether this sample is derived from any of the following tumours: adenocarcinoma of breast, cervix, esophagus, gall bladder, lung, pancreas, small and large intestine, stomach; astrocytoma, skin basal cell carcinoma, cholangiocarcinoma of liver, clear cell adenocarcinoma of ovary, diffuse large B-cell lymphoma, carcinoma of the testes, endometrioid carcinoma, Ewing's sarcoma, follicular carcinoma of thyroid, gastrointestinal stromal tumour, germ cell tumour of ovary, germ-cell tumour of testes, glioblastoma multiforme, hepatocellular carcinoma of liver, Hodgkin's lymphoma, large cell carcinoma of lung, lelomyosarcoma, liposarcoma, lobular carcinoma of breast, malignant fibrous histiocytoma, medullary carcinoma of thyroid, melanoma, meningioma, mesothelioma of lung, mucinous adenocarcinoma of ovary, myofibrosarcoma, neuroendocrine entestinal tumour, oligodendroglioma, osteosarcoma, papillary carcinoma of thyroid, pheochromocytoma, renal cell carcinoma, rhabdomyosarcoma, seminoma, serous adenocarcinoma of the ovary, small cell carcinoma of lung, cervical squamous cell carcinoma, esophageal squamous cell carcinoma, laryngal squamous cell carcinoma, lung squamous cell carcinoma, skin squamous cell carcinoma, synovial sarcoma, T-Cell lymphoma, and transitional cell carcinoma of the bladder.

It is especially preferred that the metastatic tumour which is typed according to the method of the present invention is a carcinoma, such as an adenocarcinoma.

As also discussed in the examples herein, the short, but highly specific probes of the present invention allows hybridization assays to be performed on fixated embedded tissue sections, such as formalin fixated paraffine embedded sections. Hence, an embodiment of the uses/methods of the present invention are those where the molecule, which is isolated, purified, amplified, detected, identified, quantified, inhibited or captured, is DNA (single stranded such as viral DNA) or RNA present in a fixated, embedded sample such as a formalin fixated paraffine embedded sample.

Hence, the method of the present invention includes:

(a) capture and detection of naturally occurring miRNAs such as mature miRNAs and stem-loop precursor miRNAs;

(b) purification/isolation of naturally occurring miRNAs such as mature miRNAs and stem-loop precursor miRNAs;

(c) detection and assessment of expression patterns for naturally occurring miRNAs such as mature miRNAs and stem-loop precursor miRNAs by RNA in-situ hybridisation, dot blot hybridisation, reverse dot blot hybridisation, or in Northern blot analysis or expression profiling by microarrays;

So, the embodiments include the use of an LNA modified oligonucleotide probe as an aptamer in molecular diagnostics and in the construction of Taqman probes or Molecular Beacons.

The present invention also provides a kit for the identification and optional quantification/capture of miRNA to determine tissue origin of metastatic tumours having no apparent primary tumour, where the kit comprises a reaction body and one or more LNAs as defined herein. The LNAs are preferably immobilised onto said reactions body (e.g. by using the immobilising techniques described above).

For the kits according to the invention, the reaction body is preferably a solid support material, e.g. selected from borosilicate glass, soda-lime glass, polystyrene, polycarbonate, polypropylene, polyethylene, polyethyleneglycol terephthalate, polyvinylacetate, polyvinylpyrrolidinone, polymethylmethacrylate and polyvinylchloride, preferably polystyrene and polycarbonate. The reaction body may be in the form of a specimen tube, a vial, a slide, a sheet, a film, a bead, a pellet, a disc, a plate, a ring, a rod, a net, a filter, a tray, a microtitre plate, a stick, or a multi-bladed stick.

A written instruction sheet stating the optimal conditions for use of the kit typically accompanies the kits.

### FURTHER ASPECTS OF THE INVENTION

Once the appropriate target miRNA sequences have been selected, LNA substituted detection probes are preferably chemically synthesized using commercially available methods and equipment as described in the art (Tetrahedron 54: 3607-30, 1998). For example, the solid phase phosphoramidite method can be used to produce short LNA probes (Caruthers, et al., Cold Spring Harbor Symp. Quant. Biol. 47:411-418, 1982, Adams, et al., J. Am. Chem. Soc. 105: 661 (1983).

LNA-containing-probes can be labelled during synthesis. The flexibility of the phosphoramidite synthesis approach furthermore facilitates the easy production of LNAs carrying all commercially available linkers, fluorophores and labelling-molecules available for this standard chemistry. LNA-modified probes may also be labelled by enzymatic reactions e.g. by kinasing using T4 polynucleotide kinase and gamma-³²P-ATP or by using terminal deoxynucleotidyl transferase (TDT) and any given digoxygenin-conjugated nucleotide triphosphate (dNTP) or dideoxynucleotide triphosphate (ddNTP).

Detection probes according to the invention can comprise single labels or a plurality of labels. In one aspect, the plurality of labels comprise a pair of labels which interact with each other either to produce a signal or to produce a change in a signal when hybridization of the detection probe to a target sequence occurs.

In another aspect, the detection probe comprises a fluorophore moiety and a quencher moiety, positioned in such a way that the hybridized state of the probe can be distinguished from the unhybridized state of the probe by an increase in the fluorescent signal from the nucleotide. In one aspect, the detection probe comprises, in addition to the recognition element, first and second complementary sequences, which specifically hybridize to each other, when the probe is not hybridized to a recognition sequence in a target molecule, bringing the quencher molecule in sufficient proximity to said reporter molecule to quench fluorescence of the reporter molecule. Hybridization of the target molecule distances the quencher from the reporter molecule and results in a signal, which is proportional to the amount of hybridization.

In the present context, the term “label” means a reporter group, which is detectable either by itself or as a part of a detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy). Especially interesting examples are biotin, fluorescein, Texas Red, rhodamine, dinitrophenyl, digoxigenin, Ruthenium, Europium, Cy5, Cy3, etc.

Suitable samples of target RNA sequences are derived from animal cells (e.g. from blood, serum, plasma, reticulocytes, lymphocytes, urine, bone marrow tissue, cerebrospinal fluid or any product prepared from blood or lymph) or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), and archival tissue nucleic acids.

Preferably, the detection probes of the invention are modified in order to increase the binding affinity of the probes for the target sequence by at least two-fold compared to probes of the same sequence without the modification, under the same conditions for hybridization or stringent hybridization conditions. The preferred modifications include, but are not limited to, inclusion of nucleobases, nucleosidic bases or nucleotides that have been modified by a chemical moiety or replaced by an analogue to increase the binding affinity. The preferred modifications may also include attachment of duplex-stabilizing agents e.g., such as minor-groove-binders (MGB) or intercalating nucleic acids (INA). Additionally, the preferred modifications may also include addition of non-discriminatory bases e.g., such as 5-nitroindole, which are capable of stabilizing duplex formation regardless of the nucleobase at the opposing position on the target strand. Finally, multi-probes composed of a non-sugar-phosphate backbone, e.g. such as PNA, that are capable of binding sequence specifically to a target sequence are also considered as a modification. All the different binding affinity-increasing modifications mentioned above will in the following be referred to as “the stabilizing modification(s)”, and the tagging probes and the detection probes will in the following also be referred to as “modified oligonucleotide”. More preferably the binding affinity of the modified oligonucleotide is at least about 3-fold, 4-fold, 5-fold, or 20-fold higher than the binding of a probe of the same sequence but without the stabilizing modification(s).

Most preferably, the stabilizing modification(s) is inclusion of one or more LNA nucleotide analogs. Probes from 6 to 30 nucleotides according to the invention may comprise from 1 to 8 stabilizing nucleotides, such as LNA nucleotides. When at least two LNA nucleotides are included, these may be consecutive or separated by one or more non-LNA nucleotides. In one aspect, LNA nucleotides are alpha-L-LNA and/or xylo LNA nucleotides as disclosed in PCT Publications No. WO 2000/66604 and WO 2000/56748.

The problems with existing detection, quantification and knock-down of miRNAs are addressed by the use of the oligonucleotide probes described herein in combination with the method of the invention selected so as to recognize or detect a majority of all discovered and detected miRNAs, in a given cell or tissue type. In one aspect, the probe sequences comprise probes that detect mature miRNAs in mammals, e.g., such as mouse, rat, rabbit, monkey, or, preferably, human miRNAs. By providing a sensitive and specific method for detection of mature miRNAs, the present invention overcomes the limitations discussed above especially for conventional miRNA assays. The detection element of the detection probes according to the invention may be single or double labelled (e.g. by comprising a label at each end of the probe, or an internal position). In one aspect, the detection probe comprises two labels capable of interacting with each other to produce a signal or to modify a signal, such that a signal or a change in a signal may be detected when the probe hybridizes to a target sequence. A particular aspect is when the two labels comprise a quencher and a reporter molecule.

In another aspect, the probe comprises a target-specific recognition segment capable of specifically hybridizing to a target miRNA derived sequence comprising the complementary recognition sequence. A particular detection aspect of the invention referred to as a “molecular beacon with a stem region” is when the recognition segment is flanked by first and second complementary hairpin-forming sequences which may anneal to form a hairpin. A reporter label is attached to the end of one complementary sequence and a quenching moiety is attached to the end of the other complementary sequence. The stem formed when the first and second complementary sequences are hybridized (i.e., when the probe recognition segment is not hybridized to its target) keeps these two labels in close proximity to each other, causing a signal produced by the reporter to be quenched by fluorescence resonance energy transfer (FRET). The proximity of the two labels is reduced when the probe is hybridized to a target sequence and the change in proximity produces a change in the interaction between the labels. Hybridization of the probe thus, results in a signal (e.g. fluorescence) being produced by the reporter molecule, which can be detected and/or quantified.

As mentioned above, the invention also utilises a method, system and computer program embedded in a computer readable medium (“a computer program product”) for designing detection probes comprising at least one stabilizing nucleobase—this method, system and computer program is detailed in WO 2006/069584.

A preferred embodiment of the invention are kits for the detection or quantification of target miRNAs comprising libraries of detection probes. In one aspect, the kit comprises in silico protocols for their use. The detection probes contained within these kits may have any or all of the characteristics described above. In one preferred aspect, a plurality of probes comprises at least one stabilizing nucleotide, such as an LNA nucleotide. In another aspect, the plurality of probes comprises a nucleotide coupled to or stably associated with at least one chemical moiety for increasing the stability of binding of the probe. The kits according to the invention allow a user to quickly and efficiently develop an assay for different miRNA targets, siRNA targets, RNA-edited transcripts, non-coding antisense transcripts or alternative splice variants.

In one preferred aspect, the target sequence database comprises nucleic acid sequences corresponding to human, mouse, rat, Drosophila melanogaster, C. elegans, Arabidopsis thaliana, maize or rice miRNAs.

The present invention also contemplates a method of for the treatment of cancer, said method comprising
a. Isolating RNA from at least one tissue sample from a patient suffering from cancer,
b. establishing an miRNA expression profile utilising RNA isolated in step a (i.e. establishing an miRNA expression profile as detailed herein) and determining at least one feature of said cancer which conforms with the miRNA expression profile,
c. based on the identification feature determined in step b) diagnosing the physiological status of the cancer disease in said patient, and
d. selecting and applying an appropriate form of therapy for said patient based on the said diagnosis.

In this method it is preferred that said at least one feature of said cancer is selected from one or more of the group consisting of: presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy. It is according to the present invention of particular interest that the at least one feature of said cancer is determination of the origin of said cancer, especially when said cancer is a metestasis and/or a secondary cancer which is remote from the cancer of origin, such as the primary cancer. The therapeutic regimen may be any suitable therapeutic regimen established to be suitable for the treatment of the particular cancer state, and may comprise one or more of the therapies selected from the group consisting of: chemotherapy; hormone treatment; invasive surgery; radiotherapy; and adjuvant systemic therapy.

Hence, in general, the invention utilises the design of high affinity oligonucleotide probes that have duplex stabilizing properties and methods highly useful for a variety of target nucleic acid detection methods, but particularly useful for detection of miRNAs in the method of the present invention. Some of these oligonucleotide probes contain novel nucleotides created by combining specialized synthetic nucleobases with an LNA backbone, thus creating high affinity oligonucleotides with specialized properties such as reduced sequence discrimination for the complementary strand or reduced ability to form intramolecular double stranded structures.

### Examples

The invention will now be further illustrated with reference to the following examples. It will be appreciated that what follows is by way of example only and that modifications to detail may be made while still falling within the scope of the invention.

### Example 1

Synthesis, Deprotection and Purification of LNA-Substituted Oligonucleotide Probes

The LNA-substituted probes of Example 2 to 11 were prepared on an automated DNA synthesizer (Expedite 8909 DNA synthesizer, PerSeptive Biosystems, 0.2 μmol scale) using the phosphoramidite approach (Beaucage and Caruthers, Tetrahedron Lett. 22: 1859-1862, 1981) with 2-cyanoethyl protected LNA and DNA phosphoramidites, (Sinha, et al., Tetrahedron Lett. 24: 5843-5846, 1983). CPG solid supports derivatised with a suitable quencher and 5′-fluorescein phosphoramidite (GLEN Research, Sterling, Va., USA). The synthesis cycle was modified for LNA phosphoramidites (250 s coupling time) compared to DNA phosphoramidites. 1H-tetrazole or 4,5-dicyanoimidazole (Proligo, Hamburg, Germany) was used as activator in the coupling step.

The probes were deprotected using 32% aqueous ammonia (1 h at room temperature, then 2 hours at 60° C.) and purified by HPLC (Shimadzu-SpectraChrom series; Xterra™ RP18 column, 10? m 7.8×150 mm (Waters). Buffers: A: 0.05M Triethylammonium acetate pH 7.4. B. 50% acetonitrile in water. Eluent: 0-25 min: 10-80% B; 25-30 min: 80% B). The composition and purity of the probes were verified by MALDI-MS (PerSeptive Biosystem, Voyager DE-PRO) analysis.

### Example 2

List of LNA-Substituted Detection Probes for Detection of Fully Conserved Vertebrate MicroRNAs in all Vertebrates

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE A

| | | | |
|---|---|---|---|
| | | Self-comp | Calculated |
| LNA probe name | Sequence 5′-3′ | score | Tm |

| | | | |
|---|---|---|---|
| hsa-let7f/LNA | aamCtaTacAatmCtamCtamCctmCa | 16 | 67 |
| hsa-miR19b/LNA | tmCagTttTgcAtgGatTtgmCaca | 34 | 75 |
| hsa-miR17-5p/LNA | actAccTgcActGtaAgcActTtg | 39 | 74 |
| hsa-miR217/LNA | atcmCaaTcaGttmCctGatGcaGta | 49 | 75 |
| hsa-miR218/LNA | acAtgGttAgaTcaAgcAcaa | 40 | 70 |
| hsa-miR222/LNA | gaGacmCcaGtaGccAgaTgtAgct | 38 | 80 |
| hsa-let7i/LNA | agmCacAaamCtamCtamCctmCa | 18 | 71 |
| hsa-miR27b/LNA | cagAacTtaGccActGtgAa | 35 | 68 |
| hsa-miR301/LNA | gctTtgAcaAtamCtaTtgmCacTg | 36 | 70 |
| hsa-miR30b/LNA | gcTgaGtgTagGatGttTaca | 33 | 70 |
| hsa-miR100/LNA | cacAagTtcGgaTctAcgGgtt | 38 | 77 |
| hsa-miR34a/LNA | aamCaamCcaGctAagAcamCtgmCca | 27 | 80 |
| hsa-miR7/LNA | aacAaaAtcActAgtmCttmCca | 30 | 66 |
| hsa-miR125b/LNA | tcamCaaGttAggGtcTcaGgga | 35 | 77 |
| hsa-miR133a/LNA | acAgcTggTtgAagGggAccAa | 41 | 82 |
| hsa-miR101/LNA | cttmCagTtaTcamCagTacTgta | 54 | 68 |
| hsa-miR108/LNA | aatGccmCctAaaAatmCctTat | 23 | 66 |
| hsa-miR107/LNA | tGatAgcmCctGtamCaaTgcTgct | 63 | 80 |
| hsa-miR153/LNA | tcamCttTtgTgamCtaTgcAa | 35 | 68 |
| hsa-miR10b/LNA | amCaaAttmCggTtcTacAggGta | 35 | 73 |
| mmu-miR10b/LNA | acamCaaAttmCggTtcTacAggg | 27 | 73 |
| hsa-miR194/LNA | tccAcaTggAgtTgcTgtTaca | 41 | 75 |
| hsa-miR199a/LNA | gaAcaGgtAgtmCtgAacActGgg | 40 | 78 |
| hsa-miR199a*/LNA | aacmCaaTgtGcaGacTacTgta | 39 | 74 |
| hsa-miR20/LNA | ctAccTgcActAtaAgcActTta | 26 | 70 |
| hsa-miR214/LNA | ctGccTgtmCtgTgcmCtgmCtgt | 30 | 81 |
| hsa-miR219/LNA | agAatTgcGttTggAcaAtca | 35 | 70 |
| hsa-miR223/LNA | gGggTatTtgAcaAacTgamCa | 40 | 73 |
| hsa-miR23a/LNA | gGaaAtcmCctGgcAatGtgAt | 37 | 76 |
| hsa-miR24/LNA | cTgtTccTgcTgaActGagmCca | 35 | 80 |
| hsa-miR26a/LNA | agcmCtaTccTggAttActTgaa | 34 | 70 |
| hsa-miR126/LNA | gcAttAttActmCacGgtAcga | 25 | 71 |
| hsa-miR126*/LNA | cgmCgtAccAaaAgtAatAatg | 28 | 68 |
| hsa-miR128a/LNA | aaAagAgamCcgGttmCacTgtGa | 47 | 77 |
| mmu-miR7b/LNA | aamCaaAatmCacAagTctTcca | 24 | 68 |
| hsa-let7c/LNA | aamCcaTacAacmCtamCtamCctmCa | 11 | 74 |
| hsa-let7b/LNA | aamCcamCacAacmCtamCtamCctmCa | 6 | 77 |
| hsa-miR103/LNA | tmCatAgcmCctGtamCaaTgcTgct | 63 | 80 |
| hsa-miR129/LNA | agcAagmCccAgamCcgmCaaAaag | 21 | 80 |
| rno-miR129*/LNA | aTgcTttTtgGggTaaGggmCtt | 37 | 78 |
| hsa-miR130a/LNA | gcmCctTttAacAttGcamCtg | 34 | 70 |
| hsa-miR132/LNA | cgAccAtgGctGtaGacTgtTa | 48 | 76 |
| hsa-miR135a/LNA | tcamCatAggAatAaaAagmCcaTa | 22 | 69 |
| hsa-miR137/LNA | cTacGcgTatTctTaaGcaAta | 48 | 68 |
| hsa-miR200a/LNA | acaTcgTtamCcaGacAgtGtta | 39 | 72 |
| hsa-miR142-3p/LNA | tmCcaTaaAgtAggAaamCacTaca | 29 | 72 |
| hsa-miR142-5p/LNA | gtaGtgmCttTctActTtaTg | 36 | 63 |
| hsa-miR181b/LNA | aamCccAccGacAgcAatGaaTgtt | 30 | 81 |
| hsa-miR183/LNA | caGtgAatTctAccAgtGccAta | 32 | 73 |
| hsa-miR190/LNA | acmCtaAtaTatmCaaAcaTatmCa | 31 | 62 |
| hsa-miR193/LNA | ctGggActTtgTagGccAgtt | 31 | 76 |
| hsa-miR19a/LNA | tmCagTttTgcAtaGatTtgmCaca | 37 | 72 |
| hsa-miR204/LNA | cagGcaTagGatGacAaaGggAa | 25 | 78 |
| hsa-miR205/LNA | caGacTccGgtGgaAtgAagGa | 39 | 81 |
| hsa-miR216/LNA | camCagTtgmCcaGctGagAtta | 64 | 74 |
| hsa-miR221/LNA | gAaamCccAgcAgamCaaTgtAgct | 31 | 80 |
| hsa-miR25/LNA | tcaGacmCgaGacAagTgcAatg | 27 | 77 |
| hsa-miR29c/LNA | taamCcgAttTcaAatGgtGcta | 47 | 70 |
| hsa-miR29b/LNA | amCacTgaTttmCaaAtgGtgmCta | 47 | 71 |
| hsa-miR30c/LNA | gmCtgAgaGtgTagGatGttTaca | 33 | 73 |
| hsa-miR140/LNA | ctAccAtaGggTaaAacmCact | 43 | 71 |
| hsa-miR9*/LNA | acTttmCggTtaTctAgcTtta | 27 | 65 |
| hsa-miR92/LNA | amCagGccGggAcaAgtGcaAta | 36 | 81 |
| hsa-miR96/LNA | aGcaAaaAtgTgcTagTgcmCaaa | 38 | 75 |
| hsa-miR99a/LNA | cacAagAtcGgaTctAcgGgtt | 42 | 77 |
| hsa-miR145/LNA | aAggGatTccTggGaaAacTggAc | 50 | 79 |
| hsa-miR155/LNA | ccmCctAtcAcgAttAgcAttAa | 29 | 71 |
| hsa-miR29a/LNA | aamCcgAttTcaAatGgtGctAg | 47 | 75 |
| rno-miR140*/LNA | gtcmCgtGgtTctAccmCtgTggTa | 49 | 81 |
| hsa-miR206/LNA | ccamCacActTccTtamCatTcca | 11 | 73 |
| hsa-miR124a/LNA | tggmCatTcamCcgmCgtGccTtaa | 43 | 80 |
| hsa-miR122a/LNA | acAaamCacmCatTgtmCacActmCca | 25 | 78 |
| hsa-miR1/LNA | tamCatActTctTtamCatTcca | 11 | 64 |
| hsa-miR181a/LNA | acTcamCcgAcaGcgTtgAatGtt | 49 | 77 |
| hsa-miR10a/LNA | cAcaAatTcgGatmCtamCagGgta | 37 | 74 |
| hsa-miR196a/LNA | ccaAcaAcaTgaAacTacmCta | 20 | 67 |
| hsa-let7a/LNA | aamCtaTacAacmCtamCtamCctmCa | 16 | 70 |
| hsa-miR9/LNA | tcAtamCagmCtaGatAacmCaaAga | 34 | 71 |

### Example 3

List of LNA-Substituted Detection Probes for Detection of Fully Conserved Vertebrate MicroRNAs in all Vertebrates

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE B

| | | Self-compl | Calculated |
|---|---|---|---|
| Probe name | Sequence 5′-3′ | score | Tm |
| hsa-miR-210 | agcmCgcTgtmCacAcgmCacAg | 37 | 84 |
| hsa-miR-144 | taGtamCatmCatmCtaTacTgta | 37 | 64 |
| hsa-miR-338 | caAcaAaaTcamCtgAtgmCtgGa | 33 | 72 |
| hsa-miR-187 | ggcTgcAacAcaAgamCacGa | 30 | 79 |
| hsa-miR-200b | cAtcAttAccAggmCagTatTaga | 29 | 71 |
| hsa-miR-184 | cmCctTatmCagTtcTccGtcmCa | 23 | 75 |
| hsa-miR-27a | gcGgaActTagmCcamCtgTgaa | 35 | 77 |
| hsa-miR-215 | ctgTcaAttmCatAggTcat | 38 | 65 |
| hsa-miR-203 | agTggTccTaaAcaTttmCac | 23 | 68 |
| hsa-miR-16 | ccaAtaTttAcgTgcTgcTa | 30 | 68 |
| hsa-miR-152 | aAgtTctGtcAtgmCacTga | 29 | 72 |

### Example 4

List of LNA-Substituted Detection Probes for Detection of Zebra Fish MicroRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

TABLE C

| | | Self-comp | Calculated |
|---|---|---|---|
| Probe name | Sequence 5′-3′ | score | Tm |
| dre-miR-93 | ctAccTgcAcaAacAgcActTt | 26 | 73 |
| dre-miR-22 | acaGttmCttmCagmCtgGcaGctt | 62 | 76 |
| dre-miR-213 | gGtamCagTcaAcgGtcGatGgt | 63 | 80 |
| dre-miR-31 | cagmCtaTgcmCaamCatmCttGcc | 34 | 76 |
| dre-miR-189 | amCtgTtaTcaGctmCagTagGcac | 41 | 75 |
| dre-miR-18 | tatmCtgmCacTaaAtgmCacmCtta | 45 | 69 |
| dre-miR-15a | cAcaAacmCatTctGtgmCtgmCta | 35 | 74 |
| dre-miR-34b | cAatmCagmCtaAcaAcamCtgmCcta | 24 | 74 |
| dre-miR-148a | acaAagTtcTgtAatGcamCtga | 44 | 69 |
| dre-miR-125a | camCagGttAagGgtmCtcAggGa | 38 | 80 |
| dre-miR-139 | agAcamCatGcamCtgTaga | 34 | 69 |
| dre-miR-150 | cacTggTacAagGatTggGaga | 30 | 75 |
| dre-miR-192 | ggcTgtmCaaTtcAtaGgtmCa | 46 | 73 |
| dre-miR-98 | aacAacAcaActTacTacmCtca | 17 | 68 |
| dre-let-7g | amCtgTacAaamCaamCtamCctmCa | 30 | 73 |
| dre-miR-30a-5p | gctTccAgtmCggGgaTgtTtamCa | 45 | 80 |
| dre-miR-26b | aacmCtaTccTggAttActTgaa | 36 | 68 |
| dre-miR-21 | cAacAccAgtmCtgAtaAgcTa | 35 | 72 |
| dre-miR-146 | accmCttGgaAttmCagTtcTca | 40 | 72 |
| dre-miR-182 | tgtGagTtcTacmCatTgcmCaaa | 32 | 72 |
| dre-miR-182* | taGttGgcAagTctAgaAcca | 32 | 72 |
| dre-miR-220 | aAgtGtcmCgaTacGgtTgtGg | 47 | 81 |

### Example 5

List of LNA-Substituted Detection Probes for Detection of Drosophila melanogaster MicroRNAs.

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH2-C6- or a NH2-C6-hexaethylene glycol monomer or dimer group at the 5′-end or at the 3′-end of the probes during synthesis.

TABLE D

| | | Self-compl | Calculated |
|---|---|---|---|
| Probe name | Sequence 5′-3′ | score | Tm |
| dme-miR-2c | gcmCcaTcaAagmCtgGctGtgAta | 68 | 78 |
| dme-miR-6 | aaaAagAacAgcmCacTgtGata | 36 | 71 |
| dme-miR-7 | amCaamCaaAatmCacTagTctTcca | 30 | 71 |
| dme-miR-14 | tAggAgaGagAaaAagActGa | 15 | 71 |
| dme-miR-277 | tgTcgTacmCagAtaGtgmCatTta | 38 | 72 |
| dme-miR-278 | aaAcgGacGaaAgtmCccAccGa | 41 | 80 |
| dme-miR-279 | tTaaTgaGtgTggAtcTagTca | 40 | 70 |
| dme-miR-309 | tAggAcaAacTttAccmCagTgc | 37 | 74 |
| dme-miR-310 | aAagGccGggAagTgtGcaAta | 28 | 79 |
| dme-miR-318 | tgaGatAaamCaaAgcmCcaGtga | 25 | 73 |
| dme-miR- | aaTcaGctTtcAaaAtgAtcTca | 40 | 66 |
| bantam | | | |

### Example 6

List of LNA-Substituted Detection Probes for Detection of Drosophila melanogaster and Caenorhabditis elegans MicroRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE E

| | | Self-comp | Calculated |
|---|---|---|---|
| Probe name | Sequence 5′-3′ | score | Tm |
| dme_cel-miR1/LNA | cAtamCttmCttTacAttmCca | 14 | 62 |
| dme_cel-miR2/LNA | tcaAagmCtgGctGtgAta | 56 | 67 |
| cel-lin4/LNA | tcAcamCttGagGtcTcag | 50 | 68 |

### Example 7

List of LNA-Substituted Detection Probes for Detection of Arabidopsis thallana MicroRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE F

| | | Self-comp | Calculated |
|---|---|---|---|
| Probe name | Sequence 5′-3′ | score | Tm |
| ath-MIR171_LNA2 | gAtAtTgGcGcGgmCtmCaAtmCa | 64 | 83 |
| ath-MIR171_LNA3 | gAtaTtgGcgmCggmCtcAatmCa | 54 | 78 |
| ath-MIR159_LNA2 | tAgAgmCtmCcmCtTcAaTcmCaAa | 46 | 79 |
| ath-MIR159_LNA3 | tAgaGctmCccTtcAatmCcaAa | 43 | 72 |
| ath-MIR161LNA3 | cmCccGatGtaGtcActTtcAa | 34 | 73 |
| ath-MIR167LNA3 | tAgaTcaTgcTggmCagmCttmCa | 53 | 79 |
| ath-MIR319LNA3 | ggGagmCtcmCctTcaGtcmCaa | 70 | 78 |

### Example 8

List of LNA-Substituted Detection Probes for Detection of Arabidopsis thaliana MicroRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE G

| | | Predicted |
|---|---|---|
| Oligo name | Sequence 5′-3′ | Tm ° C. |
| ath-miR159a/LNA | tAgaGctmCccTtcAatmCcaAa | 145 |
| ath-miR319a/LNA | ggGagmCtcmCctTcaGtcmCaa | 183 |
| ath-miR396a/LNA | gTtcAagAaaGctGtgGaa | 242 |
| ath-miR156a/LNA | gtgmCtcActmCtcTtcTgtmCa | 235 |
| ath-miR172a/LNA | atgmCagmCatmCatmCaaGat | 228 |
| | Tct | |

### Example 9

List of LNA-Substituted Detection Probes Useful as Controls in Detection of Vertebrate MicroRNAs

LNA nucleotides are depicted by capital letters, DNA nucleotides by lowercase letters, mC denotes LNA methyl-cytosine. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE H

| | | Self- |
|---|---|---|
| | | comp |
| Probe name | Sequence 5′-3′ | score |
| hsa-miR206/ | ccamCacActmCtcTtamCatTcca | 8 |
| LNA/2MM | | |
| hsa-miR206/ | ccamCacActmCccTtamCatTcca | 8 |
| LNA/MM10 | | |
| hsa-miR124a/ | tggmCatTcaAagmCgtGccTtaa | 60 |
| LNA/2MM | | |
| hsa-miR124a/ | tggmCatTcaAcgmCgtGccTtaa | 60 |
| LNA/MM10 | | |
| hsa-miR122a/ | acAaamCacmCacmCgtmCacActmCca | 18 |
| LNA/2MM | | |
| hsa-miR122a/ | acAaamCacmCatmCgtmCacActmCca | 18 |
| LNA/MM11 | | |

### Example 10

List of LNA-Substituted Detection Probes for Detection of Human MicroRNAs

LNA nucleotides are depicted by capital letters, DNA nucleotides by lowercase letters, mC denotes LNA methyl-cytosine, PM perfect match to the miRNA, MM one mismatch at the central position of the probe sequence. The detection probes can be used to detect and analyze conserved vertebrate miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE I

| Probe name | Sequence 5′-3′ |
|---|---|
| hsa-let7a/LNA_PM | aamCtaTacAacmCtamCtamCctmCa |
| hsa-let7f/LNA_PM | aamCtaTacAatmCtamCtamCctmCa |
| hsa-miR143LNA_PM | tGagmCtamCagTgcTtcAtcTca |
| hsa-miR145/LNA_PM | aAggGatTccTggGaaAacTggAc |
| hsa-miR320/LNA_PM | tTcgmCccTctmCaamCccAgcTttt |
| hsa-miR26a/LNA_PM | agcmCtaTccTggAttActTgaa |
| hsa-miR99a/LNA_PM | cacAagAtcGgaTctAcgGgtt |
| hsa-miR15a/LNA_PM | cAcaAacmCatTatGtgmCtgmCta |
| hsa-miR16-1/LNA_PM | cgmCcaAtaTttAcgTgcTgcTa |
| hsa-miR24/LNA_PM | cTgtTccTgcTgaActGagmCca |
| hsa-let7g/LNA_PM | amCtgTacAaamCtamCtamCctmCa |
| hsa-let7a/LNA_MM | aamCtaTacAacAtamCtamCctmCa |
| hsa-let7f/LNA_MM | aamCtaTacAatAtamCtamCctmCa |
| hsa-miR143LNA_MM | tGagmCtamCagmCgcTtcAtcTca |
| hsa-miR145/LNA_MM | aAggGatTccTcgGaaAacTggAc |
| hsa-miR320/LNA_MM | tTcgmCccTctAaamCccAgcTttt |
| hsa-miR26a/LNA_MM | agcmCtaTccTcgAttActTgaa |
| hsa-miR99a/LNA_MM | cacAagAtcGcaTctAcgGgtt |
| hsa-miR15a/LNA_MM | cAcaAacmCatmCatGtgmCtgmCta |
| hsa-miR16-1/LNA_MM | cgmCcaAtaTttTcgTgcTgcTa |
| hsa-miR24/LNA_MM | cTgtTccTgcmCgaActGagmCca |
| hsa-let7g/LNA_MM | amCtgTacAaaAtamCtamCctmCa |

### Example 11

List of LNA-Substituted Detection Probes for Expression Profiling of Human and Mouse MicroRNAs by Oligonucleotide Microarrays

LNA nucleotides are depicted by capital letters, DNA nucleotides by lowercase letters, mC denotes LNA methyl-cytosine, PM perfect match to the miRNA, MM one mismatch at the central position of the probe sequence, dir denotes the probe sequence corresponding to the mature miRNA sequence, rev denotes the probe sequence complementary to the mature miRNA sequence in question. The detection probes can be used t as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis.

TABLE J

| | | Self-comp |
|---|---|---|
| Probe name | Sequence 5′-3′ | score |
| mmu-let7adirPM/LNA | tgaGgtAgtAggTtgTatAgtt | 30 |
| mmu-miR1dirPM/LNA | tgGaaTgtAaaGaaGtaTgta | 18 |
| mmu-miR16dirPM/LNA | tagmCagmCacGtaAatAttGgcg | 46 |
| mmu-miR22dirPM/LNA | aagmCtgmCcaGttGaaGaamCtgt | 48 |
| mmu-miR26bdirPM/LNA | tTcaAgtAatTcaGgaTagGtt | 35 |
| mmu-miR30cdirPM/LNA | tgtAaamCatmCctAcamCtcTcaGc | 27 |
| mmu-miR122adirPM/LNA | tggAgtGtgAcaAtgGtgTttg | 32 |
| mmu-miR126stardirPM/LNA | catTatTacTttTggTacGcg | 28 |
| mmu-miR126dirPM/LNA | tcgTacmCgtGagTaaTaaTgc | 32 |
| mmu-miR133dirPM/LNA | tTggTccmCctTcaAccAgcTgt | 37 |
| mmu-miR143dirPM/LNA | tGagAtgAagmCacTgtAgcTca | 49 |
| mmu-miR144dirPM/LNA | tAcaGtaTagAtgAtgTacTag | 41 |
| mmu-let7arevPM/LNA | aamCtaTacAacmCtamCtamCctmCa | 16 |
| mmu-miR1revPM/LNA | tamCatActTctTtamCatTcca | 11 |
| mmu-miR16revPM/LNA | cgmCcaAtaTttAcgTgcTgcTa | 34 |
| mmu-miR22revPM/LNA | acaGttmCttmCaamCtgGcaGctt | 48 |
| mmu-miR26brevPM/LNA | aacmCtaTccTgaAttActTgaa | 28 |
| mmu-miR30crevPM/LNA | gmCtgAgaGtgTagGatGttTaca | 33 |
| mmu-miR122arevPM/LNA | cAaamCacmCatTgtmCacActmCca | 25 |
| mmu-miR126starrevPM/LNA | cgmCgtAccAaaAgtAatAatg | 28 |
| mmu-miR126revPM/LNA | gcAttAttActmCacGgtAcga | 25 |
| mmu-miR133revPM/LNA | acAgcTggTtgAagGggAccAa | 41 |
| mmu-miR143revPM/LNA | tGagmCtamCagTgcTtcAtcTca | 56 |
| mmu-miR144revPM/LNA | ctaGtamCatmCatmCtaTacTgta | 37 |
| mmu-let7adirMM/LNA | tgaGgtAgtAagTtgTatAgtt | 34 |
| mmu-miR1dirMM/LNA | tgGaaTgtAagGaaGtaTgta | 18 |
| mmu-miR16dirMM/LNA | tAgcAgcAcgGaaAtaTtgGcg | 33 |
| mmu-miR22dirMM/LNA | aaGctGccAggTgaAgaActGt | 35 |
| mmu-miR26bdirMM/LNA | tTcaAgtAatGcaGgaTagGtt | 27 |
| mmu-miR30cdirMM/LNA | tgtAaamCatmCatAcamCtcTcaGc | 27 |
| mmu-miR122adirMM/LNA | tggAgtGtgAaaAtgGtgTttg | 29 |
| mmu-miR126stardirMM/LNA | catTatTacTgtTggTacGcg | 35 |
| mmu-miR126dirMM/LNA | tmCgtAccGtgGgtAatAatGc | 39 |
| mmu-miR133dirMM/LNA | ttgGtcmCccTgcAacmCagmCtgt | 42 |
| mmu-miR143dirMM/LNA | tGagAtgAagAacTgtAgcTca | 49 |
| mmu-miR144dirMM/LNA | tAcaGtaTagGtgAtgTacTag | 41 |
| mmu-let7arevMM/LNA | aActAtamCaamCttActAccTca | 17 |
| mmu-miR1revMM/LNA | tacAtamCttmCctTacAttmCca | 11 |
| mmu-miR16revMM/LNA | cgmCcaAtaTttmCcgTgcTgcTa | 34 |
| mmu-miR22revMM/LNA | amCagTtcTtcAccTggmCagmCtt | 35 |
| mmu-miR26brevMM/LNA | aamCctAtcmCtgmCatTacTtgAa | 24 |
| mmu-miR30crevMM/LNA | gmCtgAgaGtgTatGatGttTaca | 29 |
| mmu-miR122arevMM/LNA | cAaamCacmCatTttmCacActmCca | 13 |
| mmu-miR126starrevMM/LNA | cgmCgtAccAacAgtAatAatg | 31 |
| mmu-miR126revMM/LNA | gmCatTatTacmCcamCggTacGa | 39 |
| mmu-miR133revMM/LNA | acaGctGgtTgcAggGgamCcaa | 45 |
| mmu-miR143revMM/LNA | tgAgcTacAgtTctTcaTctmCa | 49 |
| mmu-miR144revMM/LNA | ctAgtAcaTcamCctAtamCtgTa | 31 |

### Example 12

List of LNA-Substituted Detection Probes for Detection of All MicroRNAs Listed in the miRNA Registry Database Release 5.1 from December 2004 at http://www.sanger.ac.uk/Software/Rfam/mirna/index.shtml

LNA nucleotides are depicted by capital letters, DNA nucleotides by lowercase letters, mC denotes LNA methyl-cytosine. The detection probes can be used to detect and analyze miRNAs by RNA in situ hybridization, Northern blot analysis and by silencing using the oligonucleotides as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group, or a NH₂—C₆-random N₂₀ sequence at the 5′-end or at the 3′-end of the probes during synthesis. Ath, Arabidopsis thaliana; cbr, Caenorhabditis briggsae; cel, Caenorhabditis elegans; dme, Drosophila melanogaster, dps, Drosophila pseudoobscura; dre, Danio rerio; ebr, Eppstein Barr Virus; gga, Gallus gallus; has, Homo sapiens; mmu, Mus musculus; osa, Oryza sativa; mo, Rattus norvegicus; zma, Zea mays.

TABLE K

| | | | |
|---|---|---|---|
| | | Calc Tm | Self-complem. |
| Probe name | Probe sequence (5′-3′) | ° C. | score |

| | | | |
|---|---|---|---|
| ath-miR156a | gtgmCtcActmCtcTtcTgtmCa | 71 | 25 |
| ath-miR156b | gtgmCtcActmCtcTtcTgtmCa | 71 | 25 |
| ath-miR156c | gtgmCtcActmCtcTtcTgtmCa | 71 | 25 |
| ath-miR156d | gtgmCtcActmCtcTtcTgtmCa | 71 | 25 |
| ath-miR156e | gtgmCtcActmCtcTtcTgtmCa | 71 | 25 |
| ath-miR156f | gtgmCtcActmCtcTtcTgtmCa | 71 | 25 |
| ath-miR156g | tgTgcTcamCtcTctTctGtcg | 74 | 31 |
| ath-miR156h | gtgmCtcTctTtcTtcTgtmCaa | 68 | 25 |
| ath-miR157a | gtgmCtcTctAtcTtcTgtmCaa | 68 | 25 |
| ath-miR157b | gtgmCtcTctAtcTtcTgtmCaa | 68 | 25 |
| ath-miR157c | gtgmCtcTctAtcTtcTgtmCaa | 68 | 25 |
| ath-miR157d | gTgcTctmCtaTctTctGtca | 69 | 21 |
| ath-miR158a | tgmCttTgtmCtamCatTtgGga | 71 | 28 |
| ath-miR158b | tgmCttTgtmCtamCatTtgGgg | 72 | 28 |
| ath-miR159a | tagAgcTccmCttmCaaTccAaa | 71 | 36 |
| ath-miR159b | aagAgcTccmCttmCaaTccAaa | 72 | 36 |
| ath-miR159c | aggAgcTccmCttmCaaTccAaa | 74 | 46 |
| ath-miR160a | tggmCatAcaGggAgcmCagGca | 85 | 49 |
| ath-miR160b | tggmCatAcaGggAgcmCagGca | 85 | 49 |
| ath-miR160c | tggmCatAcaGggAgcmCagGca | 85 | 49 |
| ath-miR161 | cccmCgaTgtAgtmCacTttmCaa | 75 | 27 |
| ath-miR162a | ctgGatGcaGagGttTatmCga | 73 | 34 |
| ath-miR162b | ctgGatGcaGagGttTatmCga | 73 | 34 |
| ath-miR163 | aTcgAagTtcmCaaGtcmCtcTtcAa | 74 | 29 |
| ath-miR164a | tgcAcgTgcmCctGctTctmCca | 82 | 46 |
| ath-miR164b | tgcAcgTgcmCctGctTctmCca | 82 | 46 |
| ath-miR164c | cgcAcgTgcmCctGctTctmCca | 83 | 46 |
| ath-miR165a | gggGgaTgaAgcmCtgGtcmCga | 84 | 46 |
| ath-miR165b | gggGgaTgaAgcmCtgGtcmCga | 84 | 46 |
| ath-miR166a | gGggAatGaaGccTggTccGa | 84 | 33 |
| ath-miR166b | gGggAatGaaGccTggTccGa | 84 | 33 |
| ath-miR166c | gGggAatGaaGccTggTccGa | 84 | 33 |
| ath-miR166d | gGggAatGaaGccTggTccGa | 84 | 33 |
| ath-miR166e | gGggAatGaaGccTggTccGa | 84 | 33 |
| ath-miR166f | gGggAatGaaGccTggTccGa | 84 | 33 |
| ath-miR166g | gGggAatGaaGccTggTccGa | 84 | 33 |
| ath-miR167a | tAgaTcaTgcTggmCagmCttmCa | 79 | 53 |
| ath-miR167b | tAgaTcaTgcTggmCagmCttmCa | 79 | 53 |
| ath-miR167c | aAgaTcaTgcTggmCagmCttAa | 76 | 53 |
| ath-miR167d | ccAgaTcaTgcTggmCagmCttmCa | 82 | 53 |
| ath-miR168a | ttcmCcgAccTgcAccAagmCga | 82 | 26 |
| ath-miR168b | ttcmCcgAccTgcAccAagmCga | 82 | 26 |
| ath-miR169a | tcGgcAagTcaTccTtgGctg | 78 | 40 |
| ath-miR169b | ccGgcAagTcaTccTtgGctg | 79 | 40 |
| ath-miR169c | ccGgcAagTcaTccTtgGctg | 79 | 40 |
| ath-miR169d | cGgcAagTcaTccTtgGctmCa | 80 | 35 |
| ath-miR169e | cGgcAagTcaTccTtgGctmCa | 80 | 35 |
| ath-miR169f | cGgcAagTcaTccTtgGctmCa | 80 | 35 |
| ath-miR169g | cGgcAagTcaTccTtgGctmCa | 80 | 35 |
| ath-miR169g* | aGccAagGtcAacTtgmCcgGa | 81 | 45 |
| ath-miR169h | caGgcAagTcaTccTtgGcta | 76 | 41 |
| ath-miR169i | caGgcAagTcaTccTtgGcta | 76 | 41 |
| ath-miR169j | caGgcAagTcaTccTtgGcta | 76 | 41 |
| ath-miR169k | caGgcAagTcaTccTtgGcta | 76 | 41 |
| ath-miR169l | caGgcAagTcaTccTtgGcta | 76 | 41 |
| ath-miR169m | caGgcAagTcaTccTtgGcta | 76 | 41 |
| ath-miR169n | caGgcAagTcaTccTtgGcta | 76 | 41 |
| ath-miR170 | gAtaTtgAcamCggmCtcAatmCa | 72 | 52 |
| ath-miR171a | gAtaTtgGcgmCggmCtcAatmCa | 78 | 54 |
| ath-miR171b | cGtgAtaTtgGcamCggmCtcAa | 77 | 43 |
| ath-miR171c | cGtgAtaTtgGcamCggmCtcAa | 77 | 43 |
| ath-miR172a | atgmCagmCatmCatmCaaGatTct | 73 | 45 |
| ath-miR172b | atgmCagmCatmCatmCaaGatTct | 73 | 45 |
| ath-miR172b* | gtgAatmCttAatGgtGctGc | 72 | 33 |
| ath-miR172c | ctgmCagmCatmCatmCaaGatTct | 73 | 39 |
| ath-miR172d | ctgmCagmCatmCatmCaaGatTct | 73 | 39 |
| ath-miR172e | aTgcAgcAtcAtcAagAttmCc | 74 | 39 |
| ath-miR173 | gtgAttTctmCtcTgcAagmCgaa | 72 | 38 |
| ath-miR319a | gggAgcTccmCttmCagTccAa | 77 | 64 |
| ath-miR319b | gggAgcTccmCttmCagTccAa | 77 | 64 |
| ath-miR319c | aggAgcTccmCttmCagTccAa | 76 | 46 |
| ath-miR393a | gAtcAatGcgAtcmCctTtgGa | 74 | 56 |
| ath-miR393b | gAtcAatGcgAtcmCctTtgGa | 74 | 56 |
| ath-miR394a | gGagGtgGacAgaAtgmCcaa | 77 | 29 |
| ath-miR394b | gGagGtgGacAgaAtgmCcaa | 77 | 29 |
| ath-miR395a | gAgtTccmCccAaamCacTtcAg | 77 | 28 |
| ath-miR395b | gagTccmCccmCaaAcamCttmCag | 77 | 21 |
| ath-miR395c | gagTccmCccmCaaAcamCttmCag | 77 | 21 |
| ath-miR395d | gAgtTccmCccAaamCacTtcAg | 77 | 28 |
| ath-miR395e | gAgtTccmCccAaamCacTtcAg | 77 | 28 |
| ath-miR395f | gagTccmCccmCaaAcamCttmCag | 77 | 21 |
| ath-miR396a | cagTtcAagAaaGctGtgGaa | 70 | 35 |
| ath-miR396b | aagTtcAagAaaGctGtgGaa | 69 | 24 |
| ath-miR397a | caTcaAcgmCtgmCacTcaAtga | 73 | 39 |
| ath-miR397b | caTcaAcgAtgmCacTcaAtga | 70 | 35 |
| ath-miR398a | aagGggTgamCctGagAacAca | 80 | 39 |
| ath-miR398b | cagGggTgamCctGagAacAca | 81 | 51 |
| ath-miR398c | cagGggTgamCctGagAacAca | 81 | 51 |
| ath-miR399a | cAggGcaAatmCtcmCttTggmCa | 78 | 48 |
| ath-miR399b | caGggmCaamCtcTccTttGgca | 81 | 39 |
| ath-miR399c | caGggmCaamCtcTccTttGgca | 81 | 39 |
| ath-miR399d | cggGgcAaaTctmCctTtgGca | 79 | 47 |
| ath-miR399e | cgaGgcAaaTctmCctTtgGca | 76 | 41 |
| ath-miR399f | cmCggGcaAatmCtcmCttTggmCa | 80 | 41 |
| ath-miR400 | gTgamCttAtaAtamCtcTcaTa | 63 | 32 |
| ath-miR401 | tgtmCggTcgAcamCcaGttTcg | 78 | 59 |
| ath-miR402 | cAgaGgtTtaAtaGgcmCtcGaa | 76 | 68 |
| ath-miR403 | cgAgtTtgTgcGtgAatmCtaa | 71 | 46 |
| ath-miR404 | gmCtgmCcgmCaamCcgmCcaGcgTtaAt | 88 | 55 |
| ath-miR405a | agTtaTggGttAgamCccAacTcat | 74 | 64 |
| ath-miR405b | agTtaTggGttAgamCccAacTcat | 74 | 64 |
| ath-miR405d | agTtaTggGttAgamCccAacTcat | 74 | 64 |
| ath-miR406 | ctGgaTtamCaaTagmCatTcta | 67 | 38 |
| ath-miR407 | amCcaAaaGtaTatGatTtaAa | 61 | 36 |
| ath-miR408 | gmCcaGggAagAggmCagTgcAt | 87 | 35 |
| ath-miR413 | gtgmCagAacAagAgaAacTat | 69 | 24 |
| ath-miR414 | tGacGatGatGatGaaGatGa | 75 | 22 |
| ath-miR415 | atgTtcTgtTtcTgcTctGtt | 68 | 15 |
| ath-miR416 | tGaamCagTgtAcgTacGaamCc | 78 | 52 |
| ath-miR417 | tcgAacAaaTtcActAccTtc | 65 | 21 |
| ath-miR418 | ggtmCagTtcAtcAtcAcaTta | 66 | 22 |
| ath-miR419 | caamCatmCctmCagmCatTcaTaa | 71 | 18 |
| ath-miR420 | tGcaTttmCcgTgaTtaGttTa | 68 | 27 |
| ath-miR426 | cgTaaGgamCaaAttTccAaaa | 68 | 31 |
| cbr-let-7 | aamCtaTacAacmCtamCtamCctmCa | 70 | 16 |
| cbr-lin-4 | tcamCacTtgAggTctmCagGga | 78 | 70 |
| cbr-l6 | cggAatGcgTctmCatAcaAaa | 71 | 40 |
| cbr-miR-1 | tamCatActTctTtamCatTcca | 64 | 11 |
| cbr-miR-124 | tggmCatTcamCcgmCgtGccTta | 80 | 43 |
| cbr-miR-228 | ccGtgAatTcaTgcAgtGccAtt | 78 | 56 |
| cbr-miR-230 | ttTccTggTcgmCacAacTaaTac | 74 | 27 |
| cbr-miR-231 | tccTgcmCtgTtgTtcAcgAgcTta | 77 | 39 |
| cbr-miR-232 | tcAccGcaGttAagAtgmCatTta | 71 | 44 |
| cbr-miR-233 | tccmCgcAcaTgcGcaTtgmCtcAa | 83 | 59 |
| cbr-miR-234 | aAggGtaTtcTcgAgcAatAa | 70 | 46 |
| cbr-miR-236 | agmCgtmCatTacmCtgAcaGtaTta | 71 | 36 |
| cbr-miR-239a | cmCagTacmCtaAttGtaGtamCaaa | 68 | 44 |
| cbr-miR-239b | caGtamCttTtgTgcAgtAcaa | 68 | 51 |
| cbr-miR-240 | agcGaaAatTtgGagGccAgta | 74 | 33 |
| cbr-miR-241 | tmCatTtcTcamCacmCtamCctmCa | 74 | 7 |
| cbr-miR-244 | catAccActTtgTacAacmCaaAga | 70 | 40 |
| cbr-miR-245 | gaGctActTggAggGgamCcaAt | 80 | 33 |
| cbr-miR-246 | aGctmCctAccmCaaTacAtgTaa | 73 | 40 |
| cbr-miR-248 | tGagmCgtTatmCcgAgcAcgTgta | 82 | 59 |
| cbr-miR-249 | gmCaamCacTcaAaaAtcmCtgTga | 73 | 23 |
| cbr-miR-250 | cmCgtGccAacAgtTgamCtgTga | 81 | 58 |
| cbr-miR-251 | aatAagAgcGgcAccActActTaa | 74 | 41 |
| cbr-miR-252 | gttAccTgcGgcActActActTa | 75 | 28 |
| cbr-miR-253 | agtTagTgtTagTgaGgtGtg | 72 | 32 |
| cbr-miR-254 | tAtamCagTtgmCaaAagAttTgca | 69 | 51 |
| cbr-miR-259 | aacmCagAttAggAtgAgaTtt | 67 | 31 |
| cbr-miR-268 | amCcaAaamCtgmCttmCtaAttmCttGcc | 73 | 23 |
| cbr-miR-34 | cAacmCagmCtaAccAcamCtgmCct | 80 | 24 |
| cbr-miR-35 | cTtgmCaaGttTtcAccmCggTga | 77 | 52 |
| cbr-miR-353 | gaTacmCaamCacAtgAtamCttg | 68 | 23 |
| cbr-miR-354 | aggAgcAgcAacAaamCaaGgt | 79 | 23 |
| cbr-miR-355 | catAgcTcaGgcTaaAacAaa | 70 | 45 |
| cbr-miR-356 | ggAttTgtTcgmCgtTgcTcat | 74 | 29 |
| cbr-miR-357 | tccGtcAatGacTggmCatTtt | 73 | 52 |
| cbr-miR-358 | ccamCgamCtaAggAtamCcaAttg | 72 | 26 |
| cbr-miR-36 | aTtgmCgaAttTtcAccmCggTga | 76 | 44 |
| cbr-miR-360 | ttGtgAacGggAttAcgGtca | 75 | 46 |
| cbr-miR-38 | aTacmCagGttGtcTccmCggTga | 80 | 53 |
| cbr-miR-39 | cTaamCcgTttTtcAccmCggTga | 76 | 49 |
| cbr-miR-40 | ctAgcTgaTtgAcamCccGgtGa | 81 | 57 |
| cbr-miR-41 | tggGagTttTtcAccmCggTga | 76 | 44 |
| cbr-miR-42 | cTgtAgaTgtTaamCccGgtg | 76 | 39 |
| cbr-miR-43 | gcGacAgcAagTaaActGtgAta | 74 | 32 |
| cbr-miR-44 | agcTgaAtgTgtmCtcTagTca | 70 | 30 |
| cbr-miR-45 | agcTgaAtgTgtmCtcTagTca | 70 | 30 |
| cbr-miR-46 | tgAagAgaGcgActmCcaTgamCa | 79 | 33 |
| cbr-miR-47 | tGaaGagAgcGccTccAtgAca | 80 | 38 |
| cbr-miR-48 | tcgmCatmCtamCtgAgcmCtamCctmCa | 79 | 31 |
| cbr-miR-49 | tcTgcAgcTtcTcgTggTgcTt | 80 | 36 |
| cbr-miR-50 | aamCccAagAatAtcAgamCatAtca | 71 | 23 |
| cbr-miR-51 | aacAtgGcaAggAgcTacGggTa | 80 | 34 |
| cbr-miR-52 | agmCacGgaAacAtaTgtAcgGgtg | 81 | 44 |
| cbr-miR-55 | ctcGgcAgaAaaAtaTacGggTa | 75 | 32 |
| cbr-miR-57 | acamCacAgcTcgAtcTacAggGta | 78 | 47 |
| cbr-miR-58 | aTtgmCcgTacTgaAcgAtcTca | 75 | 32 |
| cbr-miR-60 | tgGacTagAaaAtgTgcAtaAta | 67 | 34 |
| cbr-miR-61 | gAgcAgaGtcAagGttmCtaGtca | 74 | 53 |
| cbr-miR-62 | ctgTaaGctAgaTtamCatAtca | 65 | 60 |
| cbr-miR-64 | tccGtamCacGctTcaGtgTcaTg | 79 | 41 |
| cbr-miR-67 | tctActmCttTctAggAggTtgTga | 73 | 54 |
| cbr-miR-70 | ctGggAacAccAatmCacGtaTta | 74 | 29 |
| cbr-miR-71 | tcamCtamCccAtgTctTtca | 67 | 20 |
| cbr-miR-72 | gmCtaTgcmCaamCatmCtgmCct | 77 | 29 |
| cbr-miR-73 | amCtgAacTgcmCaamCatmCttGcca | 79 | 44 |
| cbr-miR-74 | tctAgamCtgmCcaTttmCttGcca | 74 | 28 |
| cbr-miR-75 | tGaaGgcGgtTggTagmCttTaa | 79 | 48 |
| cbr-miR-77 | tggAcaGctAtgGccTgaTgaa | 76 | 48 |
| cbr-miR-79 | aGctTtgGtaAccTagmCttTat | 67 | 52 |
| cbr-miR-80 | tcGgcTttmCaamCtaAtgAtcTca | 72 | 27 |
| cbr-miR-81 | acTagmCttTcamCgaTgaTctmCa | 73 | 27 |
| cbr-miR-82 | amCtgGctTtcAcgAtgAtcTca | 73 | 30 |
| cbr-miR-83 | acamCtgAatTtaTatGgtGcta | 67 | 47 |
| cbr-miR-84 | gacAgcAttGcaAacTacmCtca | 73 | 36 |
| cbr-miR-85 | gmCacGccTttTcaAatActTtgTa | 71 | 33 |
| cbr-miR-86 | gActGtgGcaAagmCatTcamCtta | 73 | 44 |
| cbr-miR-87 | amCacmCtgAaamCttTgcTcac | 72 | 20 |
| cbr-miR-90 | gGggmCatTcaAacAacAtaTca | 73 | 23 |
| cel-let-7 | aamCtaTacAacmCtamCtamCctmCa | 70 | 16 |
| cel-lin-4 | tcamCacTtgAggTctmCagGga | 78 | 70 |
| cel-16 | cgaAatGcgTctmCatAcaAaa | 69 | 44 |
| cel-miR-1 | tamCatActTctTtamCatTcca | 64 | 11 |
| cel-miR-124 | tggmCatTcamCcgmCgtGccTta | 80 | 43 |
| cel-miR-2 | gcAcaTcaAagmCtgGctGtgAta | 75 | 68 |
| cel-miR-227 | gttmCagAatmCatGtcGaaAgct | 71 | 34 |
| cel-miR-228 | ccGtgAatTcaTgcAgtGccAtt | 78 | 56 |
| cel-miR-229 | acgAtgGaaAagAtaAccAgtGtcAtt | 74 | 43 |
| cel-miR-230 | tcTccTggTcgmCacAacTaaTac | 76 | 27 |
| cel-miR-231 | ttcTgcmCtgTtgAtcAcgAgcTta | 75 | 46 |
| cel-miR-232 | tcAccGcaGttAagAtgmCatTta | 71 | 44 |
| cel-miR-233 | tccmCgcAcaTgcGcaTtgmCtcAa | 83 | 59 |
| cel-miR-234 | aAggGtaTtcTcgAgcAatAa | 70 | 46 |
| cel-miR-235 | tcAggmCcgGggAgaGtgmCaaTa | 85 | 39 |
| cel-miR-236 | agmCgtmCatTacmCtgAcaGtaTta | 71 | 36 |
| cel-miR-237 | aAgcTgtTcgAgaAttmCtcAggGa | 78 | 54 |
| cel-miR-238 | tcTgaAtgGcaTcgGagTacAaa | 75 | 34 |
| cel-miR-239a | ccaGtamCctAtgTgtAgtAcaAa | 71 | 50 |
| cel-miR-239b | cAgtActTttGtgTagTacAa | 68 | 45 |
| cel-miR-240 | agcGaaGatTtgGggGccAgta | 80 | 33 |
| cel-miR-241 | tmCatTtcTcgmCacmCtamCctmCa | 76 | 18 |
| cel-miR-242 | tmCgaAgcAaaGgcmCtamCgcAa | 82 | 49 |
| cel-miR-243 | gatAtcmCcgmCcgmCgaTcgTacmCg | 84 | 58 |
| cel-miR-244 | catAccActTtgTacAacmCaaAga | 70 | 40 |
| cel-miR-245 | gaGctActTggAggGgamCcaAt | 80 | 33 |
| cel-miR-246 | aGctmCctAccmCgaAacAtgTaa | 75 | 30 |
| cel-miR-247 | aAgaAgaGaaTagGctmCtaGtca | 71 | 50 |
| cel-miR-248 | tGagmCgtTatmCcgTgcAcgTgta | 82 | 48 |
| cel-miR-249 | gcaAcgmCtcAaaAgtmCctGtga | 74 | 35 |
| cel-miR-250 | cmCatGccAacAgtTgamCtgTga | 79 | 58 |
| cel-miR-251 | aatAagAgcGgcAccActActTaa | 74 | 41 |
| cel-miR-252 | gttAccTgcGgcActActActTa | 75 | 28 |
| cel-miR-253 | ggTcaGtgTtaGtgAggTgtg | 74 | 20 |
| cel-miR-254 | cmCtamCagTcgmCgaAagAttTgca | 76 | 44 |
| cel-miR-256 | tacAgtmCttmCtaTgcAttmCca | 69 | 32 |
| cel-miR-257 | tcActGggTacTccTgaTacTc | 76 | 42 |
| cel-miR-258 | aaaAggAttmCctmCtcAaaAcc | 67 | 45 |
| cel-miR-259 | tacmCagAttAggAtgAgaTtt | 67 | 30 |
| cel-miR-260 | ctamCaaGagTtcGacAtcAc | 70 | 34 |
| cel-miR-261 | cgtGaaAacTaaAaaGcta | 61 | 24 |
| cel-miR-262 | aTcaGaaAacAtcGagAaac | 67 | 25 |
| cel-miR-264 | catAacAacAacmCacmCcgmCc | 77 | 18 |
| cel-miR-265 | atamCcamCccTtcmCtcmCctmCa | 77 | 6 |
| cel-miR-266 | gctTtgmCcaAagTctTgcmCt | 74 | 44 |
| cel-miR-267 | tgcAgcAgamCacTtcAcgGg | 81 | 29 |
| cel-miR-268 | amCcaAacTgcTtcTaaTtcTtgmCc | 74 | 19 |
| cel-miR-269 | aGttTtgmCcaGagTctTgcc | 74 | 49 |
| cel-miR-270 | cTccActGctAcaTcaTgcc | 75 | 27 |
| cel-miR-271 | aaTgcTttmCccAccmCggmCga | 82 | 33 |
| cel-miR-272 | cAaamCacmCcaTgcmCtamCa | 75 | 20 |
| cel-miR-273 | cAgcmCgamCacAgtAcgGgca | 85 | 37 |
| cel-miR-34 | cAacmCagmCtaAccAcamCtgmCct | 80 | 24 |
| cel-miR-35 | amCtgmCtaGttTccAccmCggTga | 80 | 39 |
| cel-miR-353 | aaTacmCaamCacAtgGcaAttg | 70 | 33 |
| cel-miR-354 | aggAgcAgcAacAaamCaaGgt | 79 | 23 |
| cel-miR-355 | catAgcTcaGgcTaaAacAaa | 70 | 45 |
| cel-miR-356 | tgAttTgtTcgmCgtTgcTcaa | 73 | 29 |
| cel-miR-357 | tmCctGcaAcgActGgcAttTa | 77 | 33 |
| cel-miR-358 | ccTtgAcaGggAtamCcaAttg | 72 | 42 |
| cel-miR-359 | tmCgtmCagAgaAagAccAgtGa | 78 | 25 |
| cel-miR-36 | cAtgmCgaAttTtcAccmCggTga | 77 | 44 |
| cel-miR-360 | ttGtgAacGggAttAcgGtca | 75 | 46 |
| cel-miR-37 | amCtgmCaaGtgTtcAccmCggTga | 82 | 46 |
| cel-miR-38 | amCtcmCagTttTtcTccmCggTga | 77 | 28 |
| cel-miR-39 | cAagmCtgAttTacAccmCggTga | 77 | 38 |
| cel-miR-392 | tcAtcAcamCgtGatmCgaTgaTa | 75 | 59 |
| cel-miR-40 | tTagmCtgAtgTacAccmCggTga | 78 | 52 |
| cel-miR-41 | tAggTgaTttTtcAccmCggTga | 76 | 44 |
| cel-miR-42 | cTgtAgaTgtTaamCccGgtg | 76 | 39 |
| cel-miR-43 | gcGacAgcAagTaaActGtgAta | 74 | 32 |
| cel-miR-44 | agcTgaAtgTgtmCtcTagTca | 70 | 30 |
| cel-miR-45 | agcTgaAtgTgtmCtcTagTca | 70 | 30 |
| cel-miR-46 | tgAagAgaGcgActmCcaTgamCa | 79 | 33 |
| cel-miR-47 | tGaaGagAgcGccTccAtgAca | 80 | 38 |
| cel-miR-48 | tcgmCatmCtamCtgAgcmCtamCctmCa | 79 | 31 |
| cel-miR-49 | tcTgcAgcTtcTcgTggTgcTt | 80 | 36 |
| cel-miR-50 | aamCccAagAatAccAgamCatAtca | 73 | 16 |
| cel-miR-51 | aacAtgGatAggAgcTacGggTa | 79 | 31 |
| cel-miR-52 | agmCacGgaAacAtaTgtAcgGgtg | 81 | 44 |
| cel-miR-53 | agmCacGgaAacAaaTgtAcgGgtg | 82 | 33 |
| cel-miR-54 | cTcgGatTatGaaGatTacGggTa | 75 | 35 |
| cel-miR-55 | ctcAgcAgaAacTtaTacGggTa | 74 | 33 |
| cel-miR-56 | ctcAgcGgaAacAttAcgGgta | 77 | 25 |
| cel-miR-56* | tacAacmCcaAaaTggAtcmCgcmCa | 78 | 42 |
| cel-miR-57 | acamCacAgcTcgAtcTacAggGta | 78 | 47 |
| cel-miR-58 | aTtgmCcgTacTgaAcgAtcTca | 75 | 32 |
| cel-miR-59 | cAtcAtcmCtgAtaAacGatTcga | 70 | 35 |
| cel-miR-60 | tgAacTagAaaAtgTgcAtaAta | 65 | 34 |
| cel-miR-61 | gagAtgAgtAacGgtTctAgtmCa | 75 | 52 |
| cel-miR-62 | ctgTaaGctAgaTtamCatAtca | 65 | 60 |
| cel-miR-63 | ttTccAacTcgmCttmCagTgtmCata | 75 | 31 |
| cel-miR-64 | ttcGgtAacGctTcaGtgTcaTa | 76 | 41 |
| cel-miR-65 | ttcGgtTacGctTcaGtgTcaTa | 75 | 41 |
| cel-miR-66 | tmCacAtcmCctAatmCagTgtmCatg | 75 | 27 |
| cel-miR-67 | tctActmCttTctAggAggTtgTga | 73 | 54 |
| cel-miR-68 | tmCtamCacTttTgaGtcTtcGa | 69 | 33 |
| cel-miR-69 | tcTacActTttTaaTttTcga | 59 | 20 |
| cel-miR-70 | atgGaaAcamCcaAcgAcgTatTa | 73 | 33 |
| cel-miR-71 | tcamCtamCccAtgTctTtca | 67 | 20 |
| cel-miR-72 | gmCtaTgcmCaamCatmCttGcct | 76 | 34 |
| cel-miR-73 | actGaamCtgmCctAcaTctTgcmCa | 79 | 28 |
| cel-miR-74 | tgTagActGccAttTctTgcmCa | 76 | 43 |
| cel-miR-75 | tgAagmCcgGttGgtAgcTttAa | 77 | 48 |
| cel-miR-76 | tcaAggmCttmCatmCaamCaamCgaa | 75 | 31 |
| cel-miR-77 | tggAcaGctAtgGccTgaTgaa | 76 | 48 |
| cel-miR-78 | gcamCaaAcaAccAggmCctmCca | 79 | 38 |
| cel-miR-79 | aGctTtgGtaAccTagmCttTat | 67 | 52 |
| cel-miR-80 | tcGgcTttmCaamCtaAtgAtcTca | 72 | 27 |
| cel-miR-81 | acTagmCttTcamCgaTgaTctmCa | 73 | 27 |
| cel-miR-82 | amCtgGctTtcAcgAtgAtcTca | 73 | 30 |
| cel-miR-83 | ttamCtgAatTtaTatGgtGcta | 65 | 33 |
| cel-miR-84 | tamCaaTatTacAtamCtamCctmCa | 66 | 26 |
| cel-miR-85 | gmCacGacTttTcaAatActTtgTa | 70 | 35 |
| cel-miR-86 | gActGtgGcaAagmCatTcamCtta | 73 | 44 |
| cel-miR-87 | amCacmCtgAaamCttTgcTcac | 72 | 20 |
| cel-miR-90 | gGggmCatTcaAacAacAtaTca | 73 | 23 |
| dme-bantam | aaTcaGctTtcAaaAtgAtcTca | 66 | 40 |
| dme-let-7 | amCtaTacAacmCtamCtamCctmCa | 71 | 16 |
| dme-miR-1 | ctcmCatActTctTtamCatTcca | 67 | 11 |
| dme-miR-10 | acaAatTcgGatmCtamCagGgt | 73 | 37 |
| dme-miR-100 | cAcaAgtTcgGatTtamCggGtt | 74 | 48 |
| dme-miR-11 | gcaAgaActmCagActGtgAtg | 71 | 40 |
| dme-miR-12 | accAgtAccTgaTgtAatActmCa | 73 | 33 |
| dme-miR-124 | ctTggmCatTcamCcgmCgtGccTta | 81 | 43 |
| dme-miR-125 | tcamCaaGttAggGtcTcaGgga | 77 | 35 |
| dme-miR-133 | acAgcTggTtgAagGggAccAa | 82 | 41 |
| dme-miR-13a | acTcaTcaAaaTggmCtgTgaTa | 72 | 34 |
| dme-miR-13b | acTcgTcaAaaTggmCtgTgaTa | 74 | 34 |
| dme-miR-14 | tAggAgaGagAaaAagActGa | 71 | 15 |
| dme-miR-184 | gcmCctTatmCagTtcTccGtcmCa | 77 | 23 |
| dme-miR-184* | cGggGcgAgaGaaTgaTaaGg | 83 | 19 |
| dme-miR-210 | tAgcmCgcTgtmCacAcgmCacAa | 84 | 37 |
| dme-miR-219 | cAagAatTgcGttTggAcaAtca | 72 | 35 |
| dme-miR-263a | gtgAatTctTccAgtGccAttAac | 72 | 37 |
| dme-miR-263b | gTgaAttmCtcmCcaGtgmCcaAg | 77 | 34 |
| dme-miR-274 | aTtamCccGttAgtGtcGgtmCacAaaa | 79 | 51 |
| dme-miR-275 | cGcgmCgcTacTtcAggTacmCtga | 82 | 64 |
| dme-miR-276a | agAgcAcgGtaTgaAgtTccTa | 75 | 33 |
| dme-miR-276a* | cgtAggAacTctAtamCctmCgcTg | 76 | 30 |
| dme-miR-276b | agAgcAcgGtaTtaAgtTccTa | 71 | 40 |
| dme-miR-276b* | cgtAggAacTctAtamCctmCgcTg | 76 | 30 |
| dme-miR-277 | tgTcgTacmCagAtaGtgmCatTta | 72 | 38 |
| dme-miR-278 | aaAcgGacGaaAgtmCccAccGa | 80 | 41 |
| dme-miR-279 | tTaaTgaGtgTggAtcTagTca | 70 | 40 |
| dme-miR-280 | tAtcAttTcaTatGcaAcgTaaAtamCa | 70 | 40 |
| dme-miR-281 | acAaaGagAgcAatTccAtgAca | 74 | 26 |
| dme-miR-281-1* | actGtcGacGgamCagmCtcTctt | 80 | 56 |
| dme-miR-281-2* | actGtcGacGgaTagmCtcTctt | 77 | 56 |
| dme-miR-282 | amCagAcaAagmCctAgtAgaGgcTagAtt | 80 | 49 |
| dme-miR-283 | aGaaTtamCcaGctGatAttTa | 67 | 54 |
| dme-miR-284 | caAttGctGgaAtcAagTtgmCtgActTca | 78 | 45 |
| dme-miR-285 | gcamCtgAttTcgAatGgtGcta | 74 | 55 |
| dme-miR-286 | agcAcgAgtGttmCggTctAgtmCa | 80 | 46 |
| dme-miR-287 | gtgmCaaAcgAttTtcAacAca | 68 | 27 |
| dme-miR-288 | caTgaAatGaaAtcGacAtgAaa | 68 | 27 |
| dme-miR-289 | agtmCgcAggmCtcmCacTtaAatAttTa | 74 | 42 |
| dme-miR-2a | gcTcaTcaAagmCtgGctGtgAta | 75 | 68 |
| dme-miR-2b | gcTccTcaAagmCtgGctGtgAta | 76 | 62 |
| dme-miR-2c | gcmCcaTcaAagmCtgGctGtgAta | 78 | 68 |
| dme-miR-3 | tgaGacAcamCttTgcmCcaGtga | 77 | 45 |
| dme-miR-303 | accAgtTtcmCtgTgaAacmCtaAa | 72 | 45 |
| dme-miR-304 | ctcAcaTttAcaAatTgaGatTa | 64 | 55 |
| dme-miR-305 | cagAgcAccTgaTgaAgtAcaAt | 74 | 31 |
| dme-miR-306 | tTgaGagTcamCtaAgtAccTga | 72 | 42 |
| dme-miR-306* | gmCacAggmCacAgaGtgAccmCcc | 86 | 37 |
| dme-miR-307 | ctmCacTcaAggAggTtgTga | 74 | 33 |
| dme-miR-308 | cTcamCagTatAatmCctGtgAtt | 69 | 64 |
| dme-miR-309 | tAggAcaAacTttAccmCagTgc | 74 | 37 |
| dme-miR-310 | aAagGccGggAagTgtGcaAta | 79 | 28 |
| dme-miR-311 | tmCagGccGgtGaaTgtGcaAta | 81 | 36 |
| dme-miR-312 | tmCagGccGtcTcaAgtGcaAta | 77 | 39 |
| dme-miR-313 | tcgGgcTgtGaaAagTgcAata | 77 | 29 |
| dme-miR-314 | cmCgaActTatTggmCtcGaaTa | 72 | 30 |
| dme-miR-315 | gmCttTctGagmCaamCaaTcaAaa | 72 | 37 |
| dme-miR-316 | cgcmCagTaaGcgGaaAaaGaca | 76 | 35 |
| dme-miR-317 | amCtgGatAccAccAgcTgtGttmCa | 82 | 47 |
| dme-miR-318 | tgaGatAaamCaaAgcmCcaGtga | 73 | 25 |
| dme-miR-31a | tcaGctAtgmCcgAcaTctTgcmCa | 80 | 45 |
| dme-miR-31b | cagmCtaTtcmCgamCatmCttGcca | 75 | 31 |
| dme-miR-33 | cAatGcgActAcaAtgmCacmCt | 75 | 26 |
| dme-miR-34 | cAacmCagmCtaAccAcamCtgmCca | 80 | 24 |
| dme-miR-4 | tcAatGgtTgtmCtaGctTtat | 67 | 34 |
| dme-miR-5 | catAtcAcaAcgAtcGttmCctTt | 69 | 54 |
| dme-miR-6 | aaaAagAacAgcmCacTgtGata | 71 | 36 |
| dme-miR-7 | amCaamCaaAatmCacTagTctTcca | 71 | 30 |
| dme-miR-79 | atgmCttTggTaaTctAgcTtta | 66 | 34 |
| dme-miR-8 | gamCatmCttTacmCtgAcaGtaTta | 67 | 36 |
| dme-miR-87 | camCacmCtgAaaTttTgcTcaa | 69 | 32 |
| dme-miR-92a | aTagGccGggAcaAgtGcaAtg | 80 | 28 |
| dme-miR-92b | gmCagGccGggActAgtGcaAtt | 83 | 36 |
| dme-miR-9a | tcAtamCagmCtaGatAacmCaaAga | 71 | 34 |
| dme-miR-9b | catAcaGctAaaAtcAccAaaGa | 69 | 24 |
| dme-miR-9c | tctAcaGctAgaAtamCcaAaga | 68 | 27 |
| dme-miR-iab-4-3p | gttAcgTatActGaaGgtAtamCcg | 73 | 59 |
| dme-miR-iab-4-5p | tmCagGatAcaTtcAgtAtamCgt | 72 | 34 |
| dps-bantam | aaTcaGctTtcAaaAtgAtcTca | 66 | 40 |
| dps-let-7 | amCtaTacAacmCtamCtamCctmCa | 71 | 16 |
| dps-miR-1 | ctcmCatActTctTtamCatTcca | 67 | 11 |
| dps-miR-10 | acaAatTcgGatmCtamCagGgt | 73 | 37 |
| dps-miR-100 | cacAagTtcGgaAttAcgGgtt | 74 | 50 |
| dps-miR-11 | gcaAgaActmCagActGtgAtg | 71 | 40 |
| dps-miR-12 | accAgtAccTgaTgtAatActmCa | 73 | 33 |
| dps-miR-124 | ctTggmCatTcamCcgmCgtGccTta | 81 | 43 |
| dps-miR-125 | tcamCaaGttAggGtcTcaGgga | 77 | 35 |
| dps-miR-133 | acAgcTggTtgAagGggAccAa | 82 | 41 |
| dps-miR-13a | acTcaTcaAaaTggmCtgTgaTa | 72 | 34 |
| dps-miR-13b | acTcgTcaAaaTggmCtgTgaTa | 74 | 34 |
| dps-miR-14 | tAggAgaGagAaaAagActGa | 71 | 15 |
| dps-miR-184 | gcmCctTatmCagTtcTccGtcmCa | 77 | 23 |
| dps-miR-210 | tAgcmCgcTgtmCacAcgmCacAa | 84 | 37 |
| dps-miR-219 | cAagAatTgcGttTggAcaAtca | 72 | 35 |
| dps-miR-263a | gtgAatTctTccAgtGccAttAac | 72 | 37 |
| dps-miR-263b | gTgaAttmCtcmCcaGtgmCcaAg | 77 | 34 |
| dps-miR-274 | aTtamCccGttAgtGtcGgtmCacAaaa | 79 | 51 |
| dps-miR-275 | cGcgmCgcTacTtcAggTacmCtga | 82 | 64 |
| dps-miR-276a | agAgcAcgGtaTgaAgtTccTa | 75 | 33 |
| dps-miR-276b | agAgcAcgGtaTtaAgtTccTa | 71 | 40 |
| dps-miR-277 | tgTcgTacmCagAtaGtgmCatTta | 72 | 38 |
| dps-miR-278 | aAacGgamCgaAagTccmCtcmCga | 81 | 53 |
| dps-miR-279 | tTaaTgaGtgTggAtcTagTca | 70 | 40 |
| dps-miR-280 | tAtcAttTcaTatGcaAcgTaaAtamCa | 70 | 40 |
| dps-miR-281 | acAaaGagAgcAatTccAtgAca | 74 | 26 |
| dps-miR-282 | amCagAcaAagmCctAgtAgaGgcTagAtt | 80 | 49 |
| dps-miR-283 | aGaaTtamCcaGctGatAttTa | 67 | 54 |
| dps-miR-284 | caAttGctGgaAtcAagTtgmCtgActTca | 78 | 45 |
| dps-miR-285 | gcamCtgAttTcgAatGgtGcta | 74 | 55 |
| dps-miR-286 | agcAcgAgtGttmCggTctAgtmCa | 80 | 46 |
| dps-miR-287 | gtgmCaaAcgAttTtcAacAca | 68 | 27 |
| dps-miR-288 | caTgaAatGaaAtcGacAtgAaa | 68 | 27 |
| dps-miR-289 | agtmCgcAggmCtcmCacTtaAatAttTa | 74 | 42 |
| dps-miR-2a | gcTcaTcaAagmCtgGctGtgAta | 75 | 68 |
| dps-miR-2b | gcTccTcaAagmCtgGctGtgAta | 76 | 62 |
| dps-miR-2c | gcmCcaTcaAagmCtgGctGtgAta | 78 | 68 |
| dps-miR-3 | tgaGacAcamCttTgcmCcaGtga | 77 | 45 |
| dps-miR-304 | ctcAcaTttAcaAatTgaGatTa | 64 | 55 |
| dps-miR-305 | cagAgcAccTgaTgaAgtAcaAt | 74 | 31 |
| dps-miR-306 | tTgaGagTcamCtaAgtAccTga | 72 | 42 |
| dps-miR-307 | ctmCacTcaAggAggTtgTga | 74 | 33 |
| dps-miR-308 | cTcamCagTatAatmCctGtgAtt | 69 | 64 |
| dps-miR-309 | tAagAcaAacTtcAccmCagTgc | 74 | 29 |
| dps-miR-314 | cmCgaActTatTggmCtcGaaTa | 72 | 30 |
| dps-miR-315 | gmCttTctGagmCaamCaaTcaAaa | 72 | 37 |
| dps-miR-316 | cgcmCagTaaGcgGaaAaaGaca | 76 | 35 |
| dps-miR-317 | aTtgGatAccAccAgcTgtGttmCa | 79 | 47 |
| dps-miR-318 | tgaGatAaamCaaAgcmCcaGtga | 73 | 25 |
| dps-miR-31a | tcaGctAtgmCcgAcaTctTgcmCa | 80 | 45 |
| dps-miR-31b | tcaGctAttmCcgAcaTctTgcmCa | 77 | 31 |
| dps-miR-33 | cAatGcgActAcaAtgmCacmCt | 75 | 26 |
| dps-miR-34 | cAacmCagmCtaAccAcamCtgmCca | 80 | 24 |
| dps-miR-4 | tcAatGgtTgtmCtaGctTtat | 67 | 34 |
| dps-miR-5 | catAtcAcaAcgAtcGttmCctTt | 69 | 54 |
| dps-miR-6 | aaaAagAacAgcmCacTgtGata | 71 | 36 |
| dps-miR-7 | amCaamCaaAatmCacTagTctTcca | 71 | 30 |
| dps-miR-79 | atgmCttTggTaaTctAgcTtta | 66 | 34 |
| dps-miR-8 | gamCatmCttTacmCtgAcaGtaTta | 67 | 36 |
| dps-miR-87 | camCacmCtgAaaTttTgcTcaa | 69 | 32 |
| dps-miR-92a | aTagGccGggAcaAgtGcaAtg | 80 | 28 |
| dps-miR-92b | gmCagGccGggActAgtGcaAtt | 83 | 36 |
| dps-miR-9a | tcAtamCagmCtaGatAacmCaaAga | 71 | 34 |
| dps-miR-9b | catAcaGctAaaAtcAccAaaGa | 69 | 24 |
| dps-miR-9c | tctAcaGctAgaAtamCcaAaga | 68 | 27 |
| dps-miR-iab-4-3p | gttAcgTatActGaaGgtAtamCcg | 73 | 59 |
| dps-miR-iab-4-5p | tmCagGatAcaTtcAgtAtamCgt | 72 | 34 |
| dre-miR-10a | cAcaAatTcgGatmCtamCagGgta | 74 | 37 |
| dre-miR-10b | amCaaAttmCggTtcTacAggGta | 73 | 35 |
| dre-miR-181b | cccAccGacAgcAatGaaTgtt | 78 | 30 |
| dre-miR-182 | tgtGagTtcTacmCatTgcmCaaa | 72 | 32 |
| dre-miR-182* | taGttGgcAagTctAgaAcca | 72 | 32 |
| dre-miR-183 | caGtgAatTctAccAgtGccAta | 73 | 32 |
| dre-miR-187 | ggcTgcAacAcaAgamCacGa | 79 | 30 |
| dre-miR-192 | ggcTgtmCaaTtcAtaGgtmCat | 72 | 46 |
| dre-miR-196a | ccaAcaAcaTgaAacTacmCta | 67 | 20 |
| dre-miR-199a | gaAcaGgtAgtmCtgAacActGgg | 78 | 40 |
| dre-miR-203 | cAagTggTccTaaAcaTttmCac | 70 | 31 |
| dre-miR-204 | aggmCatAggAtgAcaAagGgaa | 75 | 25 |
| dre-miR-205 | caGacTccGgtGgaAtgAagGa | 81 | 39 |
| dre-miR-210 | ttAgcmCgcTgtmCacAcgmCacAg | 85 | 37 |
| dre-miR-213 | gGtamCaaTcaAcgGtcAatGgt | 75 | 43 |
| dre-miR-214 | ctGccTgtmCtgTgcmCtgmCtgt | 81 | 30 |
| dre-miR-216 | camCagTtgmCcaGctGagAtta | 74 | 64 |
| dre-miR-217 | atcmCaaTcaGttmCctGatGcaGta | 75 | 49 |
| dre-miR-219 | agAatTgcGttTggAcaAtca | 70 | 35 |
| dre-miR-220 | aAgtGtcmCgaTacGgtTgtGg | 81 | 47 |
| dre-miR-221 | gAaamCccAgcAgamCaaTgtAgct | 80 | 31 |
| dre-miR-222 | gaGacmCcaGtaGccAgaTgtAgct | 80 | 38 |
| dre-miR-223 | gGggTatTtgAcaAacTgamCa | 73 | 40 |
| dre-miR-34a | aamCaamCcaGctAagAcamCtgmCca | 80 | 27 |
| dre-miR-7 | caamCaaAatmCacTagTctTcca | 69 | 30 |
| dre-miR-7b | aamCaaAatmCacAagTctTcca | 68 | 24 |
| ebv-miR-B | aGcamCgtmCacTtcmCacTaaGa | 77 | 25 |
| ebv-miR-B | gcAagGgcGaaTgcAgaAaaTa | 78 | 27 |
| ebv-miR-BHRF1-1 | aacTccGggGctGatmCagGtta | 80 | 50 |
| ebv-miR-BHRF1-2 | tTcaAttTctGccGcaAaaGata | 70 | 52 |
| ebv-miR-BHRF1-2* | gctAtcTgcTgcAacAgaAttt | 71 | 62 |
| ebv-miR-BHRF1-3 | gtGtgmCttAcamCacTtcmCcgTta | 76 | 47 |
| gga-let-7a | aamCtaTacAacmCtamCtamCctmCa | 70 | 16 |
| gga-let-7b | aamCcamCacAacmCtamCtamCctmCa | 77 | 6 |
| gga-let-7c | aamCcaTacAacmCtamCtamCctmCa | 74 | 11 |
| gga-let-7d | actAtgmCaamCccActAccTct | 74 | 24 |
| gga-let-7f | aamCtaTacAatmCtamCtamCctmCa | 67 | 16 |
| gga-let-7g | amCtgTacAaamCtamCtamCctmCa | 71 | 30 |
| gga-let-7i | amCagmCacAaamCtamCtamCctmCa | 76 | 18 |
| gga-let-7j | aamCtaTacAacmCtamCtamCctmCa | 70 | 16 |
| gga-let-7k | aActAttmCaaTctActAccTca | 67 | 22 |
| gga-miR-1 | tamCatActTctTtamCatTcca | 64 | 11 |
| gga-miR-100 | cacAagTtcGgaTctAcgGgtt | 77 | 38 |
| gga-miR-101 | cttmCagTtaTcamCagTacTgta | 68 | 54 |
| gga-miR-103 | tmCatAgcmCctGtamCaaTgcTgct | 80 | 63 |
| gga-miR-106 | tacmCtgmCacTgtAagmCacTttt | 72 | 37 |
| gga-miR-107 | tGatAgcmCctGtamCaaTgcTgct | 80 | 63 |
| gga-miR-10b | amCaaAttmCggTtcTacAggGta | 73 | 35 |
| gga-miR-122a | acAaamCacmCatTgtmCacActmCca | 78 | 25 |
| gga-miR-124a | tggmCatTcamCcgmCgtGccTtaa | 80 | 43 |
| gga-miR-124b | tggmCatTcamCtgmCgtGccTtaa | 77 | 48 |
| gga-miR-125b | tcamCaaGttAggGtcTcaGgga | 77 | 35 |
| gga-miR-126 | gcAttAttActmCacGgtAcga | 71 | 25 |
| gga-miR-128a | aaAagAgamCcgGttmCacTgtGa | 77 | 47 |
| gga-miR-128b | aaAagAgamCcgGttmCacTgtGa | 77 | 47 |
| gga-miR-130a | atgmCccTttTaaTatTgcActg | 68 | 42 |
| gga-miR-130b | acGccmCttTcaTtaTtgmCacTg | 75 | 26 |
| gga-miR-133a | acAgcTggTtgAagGggAccAa | 82 | 41 |
| gga-miR-133b | taGctGgtTgaAggGgamCcaa | 81 | 37 |
| gga-miR-133c | gcAgcTggTtgAagGggAccAa | 83 | 41 |
| gga-miR-135a | tcamCatAggAatAaaAagmCcaTa | 69 | 22 |
| gga-miR-137 | cTacGcgTatTctTaaGcaAta | 68 | 48 |
| gga-miR-138 | gatTcamCaamCacmCagmCt | 70 | 24 |
| gga-miR-140 | ctAccAtaGggTaaAacmCact | 71 | 43 |
| gga-miR-142-3p | tmCcaTaaAgtAggAaamCacTaca | 72 | 29 |
| gga-miR-142-5p | gtaGtgmCttTctActTtaTg | 63 | 36 |
| gga-miR-146 | aAccmCatGgaAttmCagTtcTca | 73 | 44 |
| gga-miR-148a | acaAagTtcTgtAgtGcamCtga | 72 | 54 |
| gga-miR-153 | tcamCttTtgTgamCtaTgcAa | 68 | 35 |
| gga-miR-155 | ccmCctAtcAcgAttAgcAttAa | 71 | 29 |
| gga-miR-15a | cAcaAacmCatTatGtgmCtgmCta | 73 | 35 |
| gga-miR-15b | tgcAaamCcaTgaTgtGctGcta | 77 | 52 |
| gga-miR-16 | cacmCaaTatTtamCgtGctGcta | 71 | 38 |
| gga-miR-17-3p | acAagTgcmCttmCacTgcAgt | 77 | 47 |
| gga-miR-17-5p | actAccTgcActGtaAgcActTtg | 74 | 39 |
| gga-miR-181a | acTcamCcgAcaGcgTtgAatGtt | 77 | 49 |
| gga-miR-181b | cccAccGacAgcAatGaaTgtt | 78 | 30 |
| gga-miR-183 | caGtgAatTctAccAgtGccAta | 73 | 32 |
| gga-miR-184 | acmCctTatmCagTtcTccGtcmCa | 76 | 23 |
| gga-miR-187 | ggcTgcAacAcaAgamCacGa | 79 | 30 |
| gga-miR-18a | tatmCtgmCacTagAtgmCacmCtta | 71 | 40 |
| gga-miR-18b | taamCtgmCacTagAtgmCacmCtta | 72 | 40 |
| gga-miR-190 | acmCtaAtaTatmCaaAcaTatmCa | 62 | 31 |
| gga-miR-194 | tccAcaTggAgtTgcTgtTaca | 75 | 41 |
| gga-miR-196 | ccaAcaAcaTgaAacTacmCta | 67 | 20 |
| gga-miR-199a | gaAcaGgtAgtmCtgAacActGgg | 78 | 40 |
| gga-miR-19a | tmCagTttTgcAtaGatTtgmCaca | 72 | 37 |
| gga-miR-19b | tmCagTttTgcAtgGatTtgmCaca | 75 | 34 |
| gga-miR-1b | tacAtamCttmCttAacAttmCca | 64 | 16 |
| gga-miR-20 | ctAccTgcActAtaAgcActTta | 70 | 26 |
| gga-miR-200a | acaTcgTtamCcaGacAgtGtta | 72 | 39 |
| gga-miR-200b | atcAtcAttAccAggmCagTatTa | 70 | 29 |
| gga-miR-203 | cAagTggTccTaaAcaTttmCac | 70 | 31 |
| gga-miR-204 | aggmCatAggAtgAcaAagGgaa | 75 | 25 |
| gga-miR-205a | caGacTccGgtGgaAtgAagGa | 81 | 39 |
| gga-miR-205b | cAgaTtcmCggTggAatGaaGgg | 80 | 55 |
| gga-miR-206 | ccamCacActTccTtamCatTcca | 73 | 11 |
| gga-miR-213 | gGtamCaaTcaAcgGtcGatGgt | 79 | 67 |
| gga-miR-215 | gtcTgtmCaaTtcAtaGgtmCat | 70 | 50 |
| gga-miR-216 | camCagTtgmCcaGctGagAtta | 74 | 64 |
| gga-miR-217 | atcmCaaTcaGttmCctGatGcaGta | 75 | 49 |
| gga-miR-218 | acAtgGttAgaTcaAgcAcaa | 70 | 40 |
| gga-miR-219 | agAatTgcGttTggAcaAtca | 70 | 35 |
| gga-miR-221 | gAaamCccAgcAgamCaaTgtAgct | 80 | 31 |
| gga-miR-222a | gaGacmCcaGtaGccAgaTgtAgct | 80 | 38 |
| gga-miR-222b | gAgamCccAgtAgcmCagAtgTagTt | 80 | 28 |
| gga-miR-223 | gGggTatTtgAcaAacTgamCa | 73 | 40 |
| gga-miR-23b | ggtAatmCccTggmCaaTgtGat | 76 | 38 |
| gga-miR-24 | cTgtTccTgcTgaActGagmCca | 80 | 35 |
| gga-miR-26a | gcmCtaTccTggAttActTgaa | 70 | 34 |
| gga-miR-27b | gcAgaActTagmCcamCtgTgaa | 74 | 38 |
| gga-miR-29a | aamCcgAttTcaAatGgtGcta | 71 | 47 |
| gga-miR-29b | aamCacTgaTttmCaaAtgGtgmCta | 71 | 47 |
| gga-miR-29c | amCcgAttTcaAatGgtGcta | 71 | 47 |
| gga-miR-301 | atGctTtgAcaAtaTtaTtgmCacTg | 70 | 45 |
| gga-miR-302 | tcActAaaAcaTggAagmCacTt | 71 | 23 |
| gga-miR-30a-3p | gctGcaAacAtcmCgamCtgAaag | 74 | 28 |
| gga-miR-30a-5p | cTtcmCagTcgAggAtgTttAca | 73 | 31 |
| gga-miR-30b | agcTgaGtgTagGatGttTaca | 71 | 33 |
| gga-miR-30c | gmCtgAgaGtgTagGatGttTaca | 73 | 33 |
| gga-miR-30d | cttmCcaGtcGggGatGttTaca | 76 | 44 |
| gga-miR-30e | tcmCagTcaAggAtgTttAca | 69 | 30 |
| gga-miR-31 | cagmCtaTgcmCaamCatmCttGcct | 77 | 34 |
| gga-miR-32 | gcaActTagTaaTgtGcaAta | 65 | 43 |
| gga-miR-33 | cAatGcaActAcaAtgmCac | 68 | 30 |
| gga-miR-34a | aamCaamCcaGctAagAcamCtgmCca | 80 | 27 |
| gga-miR-34b | caAtcAgcTaamCtamCacTgcmCtg | 75 | 32 |
| gga-miR-34c | gcAatmCagmCtaActAcamCtgmCct | 76 | 31 |
| gga-miR-7 | caamCaaAatmCacTagTctTcca | 69 | 30 |
| gga-miR-7b | aacAaaAatmCacTagTctTcca | 66 | 30 |
| gga-miR-9 | tcAtamCagmCtaGatAacmCaaAga | 71 | 34 |
| gga-miR-92 | cagGccGggAcaAgtGcaAta | 79 | 28 |
| gga-miR-99a | cacAagAtcGgaTctAcgGgtt | 77 | 42 |
| hsa-let-7a | aamCtaTacAacmCtamCtamCctmCa | 70 | 16 |
| hsa-let-7b | aamCcamCacAacmCtamCtamCctmCa | 77 | 6 |
| hsa-let-7c | aamCcaTacAacmCtamCtamCctmCa | 74 | 11 |
| hsa-let-7d | actAtgmCaamCctActAccTct | 71 | 24 |
| hsa-let-7e | actAtamCaamCctmCctAccTca | 71 | 16 |
| hsa-let-7f | aamCtaTacAatmCtamCtamCctmCa | 67 | 16 |
| hsa-let-7g | amCtgTacAaamCtamCtamCctmCa | 71 | 30 |
| hsa-let-7i | amCagmCacAaamCtamCtamCctmCa | 76 | 18 |
| hsa-miR-1 | tamCatActTctTtamCatTcca | 64 | 11 |
| hsa-miR-100 | cacAagTtcGgaTctAcgGgtt | 77 | 38 |
| hsa-miR-101 | cttmCagTtaTcamCagTacTgta | 68 | 54 |
| hsa-miR-103 | tmCatAgcmCctGtamCaaTgcTgct | 80 | 63 |
| hsa-miR-105 | acAggAgtmCtgAgcAttTga | 73 | 33 |
| hsa-miR-106a | gctAccTgcActGtaAgcActTtt | 75 | 37 |
| hsa-miR-106b | atcTgcActGtcAgcActTta | 72 | 35 |
| hsa-miR-107 | tGatAgcmCctGtamCaaTgcTgct | 80 | 63 |
| hsa-miR-108 | aatGccmCctAaaAatmCctTat | 66 | 23 |
| hsa-miR-10a | cAcaAatTcgGatmCtamCagGgta | 74 | 37 |
| hsa-miR-10b | amCaaAttmCggTtcTacAggGta | 73 | 35 |
| hsa-miR-122a | acAaamCacmCatTgtmCacActmCca | 78 | 25 |
| hsa-miR-124a | tggmCatTcamCcgmCgtGccTtaa | 80 | 43 |
| hsa-miR-125a | cAcaGgtTaaAggGtcTcaGgga | 79 | 35 |
| hsa-miR-125b | tcamCaaGttAggGtcTcaGgga | 77 | 35 |
| hsa-miR-126 | gcAttAttActmCacGgtAcga | 71 | 25 |
| hsa-miR-126* | cgmCgtAccAaaAgtAatAatg | 68 | 28 |
| hsa-miR-127 | agcmCaaGctmCagAcgGatmCcga | 81 | 54 |
| hsa-miR-128a | aaAagAgamCcgGttmCacTgtGa | 77 | 47 |
| hsa-miR-128b | gaAagAgamCcgGttmCacTgtGa | 78 | 47 |
| hsa-miR-129 | gcAagmCccAgamCcgmCaaAaag | 80 | 21 |
| hsa-miR-130a | aTgcmCctTttAacAttGcamCtg | 74 | 42 |
| hsa-miR-130b | aTgcmCctTtcAtcAttGcamCtg | 75 | 34 |
| hsa-miR-132 | cgAccAtgGctGtaGacTgtTa | 76 | 48 |
| hsa-miR-133a | acAgcTggTtgAagGggAccAa | 82 | 41 |
| hsa-miR-133b | taGctGgtTgaAggGgamCcaa | 81 | 37 |
| hsa-miR-134 | ccmCtcTggTcaAccAgtmCaca | 77 | 57 |
| hsa-miR-135a | tcamCatAggAatAaaAagmCcaTa | 69 | 22 |
| hsa-miR-135b | cacAtaGgaAtgAaaAgcmCata | 70 | 22 |
| hsa-miR-136 | tccAtcAtcAaaAcaAatGgaGt | 71 | 25 |
| hsa-miR-137 | cTacGcgTatTctTaaGcaAta | 68 | 48 |
| hsa-miR-138 | gatTcamCaamCacmCagmCt | 70 | 24 |
| hsa-miR-139 | aGacAcgTgcActGtaGa | 75 | 42 |
| hsa-miR-140 | ctAccAtaGggTaaAacmCact | 71 | 43 |
| hsa-miR-141 | cmCatmCttTacmCagAcaGtgTta | 70 | 33 |
| hsa-miR-142-3p | tmCcaTaaAgtAggAaamCacTaca | 72 | 29 |
| hsa-miR-142-5p | gtaGtgmCttTctActTtaTg | 63 | 36 |
| hsa-miR-143 | tGagmCtamCagTgcTtcAtcTca | 75 | 56 |
| hsa-miR-144 | ctaGtamCatmCatmCtaTacTgta | 64 | 37 |
| hsa-miR-145 | aAggGatTccTggGaaAacTggAc | 79 | 50 |
| hsa-miR-146 | aAccmCatGgaAttmCagTtcTca | 73 | 44 |
| hsa-miR-147 | gcAgaAgcAttTccAcamCac | 74 | 25 |
| hsa-miR-148a | acaAagTtcTgtAgtGcamCtga | 72 | 54 |
| hsa-miR-148b | acaAagTtcTgtGatGcamCtga | 72 | 39 |
| hsa-miR-149 | ggaGtgAagAcamCggAgcmCaga | 80 | 31 |
| hsa-miR-150 | cacTggTacAagGgtTggGaga | 78 | 30 |
| hsa-miR-151 | ccTcaAggAgcTtcAgtmCtaGt | 75 | 45 |
| hsa-miR-152 | cmCcaAgtTctGtcAtgmCacTga | 78 | 36 |
| hsa-miR-153 | tcamCttTtgTgamCtaTgcAa | 68 | 35 |
| hsa-miR-154 | cGaaGgcAacAcgGatAacmCta | 78 | 40 |
| hsa-miR-154* | aAtaGgtmCaamCcgTgtAtgAtt | 74 | 40 |
| hsa-miR-155 | ccmCctAtcAcgAttAgcAttAa | 71 | 29 |
| hsa-miR-15a | cAcaAacmCatTatGtgmCtgmCta | 73 | 35 |
| hsa-miR-15b | tgtAaamCcaTgaTgtGctGcta | 74 | 38 |
| hsa-miR-16 | cgmCcaAtaTttAcgTgcTgcTa | 74 | 34 |
| hsa-miR-17-3p | acAagTgcmCttmCacTgcAgt | 77 | 47 |
| hsa-miR-17-5p | actAccTgcActGtaAgcActTtg | 74 | 39 |
| hsa-miR-18 | tatmCtgmCacTagAtgmCacmCtta | 71 | 40 |
| hsa-miR-181a | acTcamCcgAcaGcgTtgAatGtt | 77 | 49 |
| hsa-miR-181b | cccAccGacAgcAatGaaTgtt | 78 | 30 |
| hsa-miR-181c | amCtcAccGacAggTtgAatGtt | 76 | 33 |
| hsa-miR-182 | tgtGagTtcTacmCatTgcmCaaa | 72 | 32 |
| hsa-miR-182* | taGttGgcAagTctAgaAcca | 72 | 32 |
| hsa-miR-183 | caGtgAatTctAccAgtGccAta | 73 | 32 |
| hsa-miR-184 | acmCctTatmCagTtcTccGtcmCa | 76 | 23 |
| hsa-miR-185 | gAacTgcmCttTctmCtcmCa | 70 | 27 |
| hsa-miR-186 | aaGccmCaaAagGagAatTctTtg | 71 | 48 |
| hsa-miR-187 | cGgcTgcAacAcaAgamCacGa | 84 | 31 |
| hsa-miR-188 | amCccTccAccAtgmCaaGggAtg | 83 | 42 |
| hsa-miR-189 | actGatAtcAgcTcaGtaGgcAc | 77 | 54 |
| hsa-miR-190 | acmCtaAtaTatmCaaAcaTatmCa | 62 | 31 |
| hsa-miR-191 | agcTgcTttTggGatTccGttg | 74 | 42 |
| hsa-miR-192 | gGctGtcAatTcaTagGtcAg | 73 | 46 |
| hsa-miR-193 | ctGggActTtgTagGccAgtt | 76 | 31 |
| hsa-miR-194 | tccAcaTggAgtTgcTgtTaca | 75 | 41 |
| hsa-miR-195 | gmCcaAtaTttmCtgTgcTgcTa | 73 | 28 |
| hsa-miR-196a | ccaAcaAcaTgaAacTacmCta | 67 | 20 |
| hsa-miR-196b | cmCaamCaamCagGaaActAccTa | 73 | 27 |
| hsa-miR-197 | gctGggTggAgaAggTggTgaa | 84 | 19 |
| hsa-miR-198 | cmCtaTctmCccmCtcTggAcc | 75 | 25 |
| hsa-miR-199a | gaAcaGgtAgtmCtgAacActGgg | 78 | 40 |
| hsa-miR-199a* | aacmCaaTgtGcaGacTacTgta | 74 | 39 |
| hsa-miR-199b | gAacAgaTagTctAaamCacTggg | 73 | 32 |
| hsa-miR-19a | tmCagTttTgcAtaGatTtgmCaca | 72 | 37 |
| hsa-miR-19b | tmCagTttTgcAtgGatTtgmCaca | 75 | 34 |
| hsa-miR-20 | ctAccTgcActAtaAgcActTta | 70 | 26 |
| hsa-miR-200a | acaTcgTtamCcaGacAgtGtta | 72 | 39 |
| hsa-miR-200b | gtcAtcAttAccAggmCagTatTa | 71 | 31 |
| hsa-miR-200c | ccAtcAttAccmCggmCagTatTa | 74 | 38 |
| hsa-miR-203 | cTagTggTccTaaAcaTttmCac | 69 | 23 |
| hsa-miR-204 | aggmCatAggAtgAcaAagGgaa | 75 | 25 |
| hsa-miR-205 | caGacTccGgtGgaAtgAagGa | 81 | 39 |
| hsa-miR-206 | ccamCacActTccTtamCatTcca | 73 | 11 |
| hsa-miR-208 | acAagmCttTttGctmCgtmCttAt | 71 | 34 |
| hsa-miR-21 | tmCaamCatmCagTctGatAagmCta | 72 | 48 |
| hsa-miR-210 | tcAgcmCgcTgtmCacAcgmCacAg | 87 | 38 |
| hsa-miR-211 | aggmCgaAggAtgAcaAagGgaa | 77 | 18 |
| hsa-miR-212 | gGccGtgActGgaGacTgtTa | 81 | 37 |
| hsa-miR-213 | gGtamCaaTcaAcgGtcGatGgt | 79 | 67 |
| hsa-miR-214 | ctGccTgtmCtgTgcmCtgmCtgt | 81 | 30 |
| hsa-miR-215 | gtcTgtmCaaTtcAtaGgtmCat | 70 | 50 |
| hsa-miR-216 | camCagTtgmCcaGctGagAtta | 74 | 64 |
| hsa-miR-217 | atcmCaaTcaGttmCctGatGcaGta | 75 | 49 |
| hsa-miR-218 | acAtgGttAgaTcaAgcAcaa | 70 | 40 |
| hsa-miR-219 | agAatTgcGttTggAcaAtca | 70 | 35 |
| hsa-miR-22 | acaGttmCttmCaamCtgGcaGctt | 74 | 48 |
| hsa-miR-220 | aaAgtGtcAgaTacGgtGtgg | 75 | 32 |
| hsa-miR-221 | gAaamCccAgcAgamCaaTgtAgct | 80 | 31 |
| hsa-miR-222 | gaGacmCcaGtaGccAgaTgtAgct | 80 | 38 |
| hsa-miR-223 | gGggTatTtgAcaAacTgamCa | 73 | 40 |
| hsa-miR-224 | tAaamCggAacmCacTagTgamCttg | 75 | 49 |
| hsa-miR-23a | gGaaAtcmCctGgcAatGtgAt | 76 | 37 |
| hsa-miR-23b | ggtAatmCccTggmCaaTgtGat | 76 | 38 |
| hsa-miR-24 | cTgtTccTgcTgaActGagmCca | 80 | 35 |
| hsa-miR-25 | tcaGacmCgaGacAagTgcAatg | 77 | 27 |
| hsa-miR-26a | gcmCtaTccTggAttActTgaa | 70 | 34 |
| hsa-miR-26b | aacmCtaTccTgaAttActTgaa | 65 | 28 |
| hsa-miR-27a | gcGgaActTagmCcamCtgTgaa | 77 | 35 |
| hsa-miR-27b | gcAgaActTagmCcamCtgTgaa | 74 | 38 |
| hsa-miR-28 | ctmCaaTagActGtgAgcTccTt | 73 | 43 |
| hsa-miR-296 | acAggAttGagGggGggmCcct | 88 | 48 |
| hsa-miR-299 | aTgtAtgTggGacGgtAaamCca | 80 | 35 |
| hsa-miR-29a | aamCcgAttTcaGatGgtGcta | 75 | 43 |
| hsa-miR-29b | aamCacTgaTttmCaaAtgGtgmCta | 71 | 47 |
| hsa-miR-29c | amCcgAttTcaAatGgtGcta | 71 | 47 |
| hsa-miR-301 | gctTtgAcaAtamCtaTtgmCacTg | 70 | 36 |
| hsa-miR-302a | tcAccAaaAcaTggAagmCacTta | 72 | 25 |
| hsa-miR-302a* | aaaGcaAgtAcaTccAcgTtta | 69 | 32 |
| hsa-miR-302b | ctActAaaAcaTggAagmCacTta | 69 | 23 |
| hsa-miR-302b* | agAaaGcamCttmCcaTgtTaaAgt | 72 | 36 |
| hsa-miR-302c | ccActGaaAcaTggAagmCacTta | 74 | 28 |
| hsa-miR-302c* | cagmCagGtamCccmCcaTgtTaaa | 76 | 44 |
| hsa-miR-302d | acActmCaaAcaTggAagmCacTta | 73 | 23 |
| hsa-miR-30a-3p | gctGcaAacAtcmCgamCtgAaag | 74 | 28 |
| hsa-miR-30a-5p | cTtcmCagTcgAggAtgTttAca | 73 | 31 |
| hsa-miR-30b | agcTgaGtgTagGatGttTaca | 71 | 33 |
| hsa-miR-30c | gmCtgAgaGtgTagGatGttTaca | 73 | 33 |
| hsa-miR-30d | cttmCcaGtcGggGatGttTaca | 76 | 44 |
| hsa-miR-30e-3p | gmCtgTaaAcaTccGacTgaAag | 73 | 27 |
| hsa-miR-30e-5p | tcmCagTcaAggAtgTttAca | 69 | 30 |
| hsa-miR-31 | cagmCtaTgcmCagmCatmCttGcc | 78 | 38 |
| hsa-miR-32 | gcaActTagTaaTgtGcaAta | 65 | 43 |
| hsa-miR-320 | tTcgmCccTctmCaamCccAgcTttt | 80 | 26 |
| hsa-miR-323 | agAggTcgAccGtgTaaTgtGc | 80 | 46 |
| hsa-miR-324-3p | ccAgcAgcAccTggGgcAgtGg | 92 | 41 |
| hsa-miR-324-5p | acAccAatGccmCtaGggGatGcg | 84 | 54 |
| hsa-miR-325 | amCacTtamCtgGacAccTacTagg | 74 | 39 |
| hsa-miR-326 | ctgGagGaaGggmCccAgaGg | 87 | 46 |
| hsa-miR-328 | acGgaAggGcaGagAggGccAg | 87 | 31 |
| hsa-miR-33 | cAatGcaActAcaAtgmCac | 68 | 30 |
| hsa-miR-330 | tmCtcTgcAggmCcgTgtGctTtgc | 84 | 53 |
| hsa-miR-331 | tTctAggAtaGgcmCcaGggGc | 84 | 51 |
| hsa-miR-335 | amCatTttTcgTtaTtgmCtcTtga | 67 | 26 |
| hsa-miR-337 | aaaGgcAtcAtaTagGagmCtgGa | 76 | 34 |
| hsa-miR-338 | tcaAcaAaaTcamCtgAtgmCtgGa | 73 | 33 |
| hsa-miR-339 | tgAgcTccTggAggAcaGgga | 83 | 47 |
| hsa-miR-340 | ggcTatAaaGtaActGagAcgGa | 72 | 34 |
| hsa-miR-342 | gacGggTgcGatTtcTgtGtgAga | 82 | 34 |
| hsa-miR-345 | gmCccTggActAggAgtmCagmCa | 84 | 40 |
| hsa-miR-346 | agaGgcAggmCatGcgGgcAgamCa | 92 | 50 |
| hsa-miR-34a | aamCaamCcaGctAagAcamCtgmCca | 80 | 27 |
| hsa-miR-34b | cAatmCagmCtaAtgAcamCtgmCcta | 74 | 30 |
| hsa-miR-34c | gcAatmCagmCtaActAcamCtgmCct | 76 | 31 |
| hsa-miR-361 | gTacmCccTggAgaTtcTgaTaa | 73 | 29 |
| hsa-miR-367 | tmCacmCatTgcTaaAgtGcaAtt | 72 | 41 |
| hsa-miR-368 | aaamCgtGgaAttTccTctAtgt | 70 | 45 |
| hsa-miR-369 | aaAgaTcaAccAtgTatTatt | 62 | 24 |
| hsa-miR-370 | ccAggTtcmCacmCccAgcAggc | 86 | 29 |
| hsa-miR-371 | acActmCaaAagAtgGcgGcac | 76 | 33 |
| hsa-miR-372 | acgmCtcAaaTgtmCgcAgcActTt | 79 | 38 |
| hsa-miR-373 | acamCccmCaaAatmCgaAgcActTc | 77 | 33 |
| hsa-miR-373* | ggaAagmCgcmCccmCatTttGagt | 78 | 31 |
| hsa-miR-374 | cacTtaTcaGgtTgtAttAtaa | 63 | 35 |
| hsa-miR-375 | tcAcgmCgaGccGaamCgaAcaAa | 81 | 39 |
| hsa-miR-376a | acgTggAttTtcmCtcTatGat | 68 | 39 |
| hsa-miR-377 | acAaaAgtTgcmCttTgtGtgAt | 73 | 48 |
| hsa-miR-378 | amCacAggAccTggAgtmCagGag | 84 | 51 |
| hsa-miR-379 | tacGttmCcaTagTctAcca | 66 | 25 |
| hsa-miR-380-3p | aagAtgTggAccAtaTtamCata | 66 | 54 |
| hsa-miR-380-5p | gmCgcAtgTtcTatGgtmCaamCca | 80 | 41 |
| hsa-miR-381 | amCagAgaGctTgcmCctTgtAta | 76 | 37 |
| hsa-miR-382 | cgaAtcmCacmCacGaamCaamCttc | 75 | 23 |
| hsa-miR-383 | agcmCacAatmCacmCttmCtgAtct | 74 | 27 |
| hsa-miR-384 | tAtgAacAatTtcTagGaat | 61 | 46 |
| hsa-miR-422a | ggcmCttmCtgAccmCtaAgtmCcag | 76 | 45 |
| hsa-miR-422b | ggmCctTctGacTccAagTccAg | 80 | 39 |
| hsa-miR-423 | ctgAggGgcmCtcAgamCcgAgct | 87 | 61 |
| hsa-miR-424 | ttcAaaAcaTgaAttGctGctg | 69 | 40 |
| hsa-miR-425 | ggmCggAcamCgamCatTccmCgat | 83 | 43 |
| hsa-miR-7 | caamCaaAatmCacTagTctTcca | 69 | 30 |
| hsa-miR-9 | tcAtamCagmCtaGatAacmCaaAga | 71 | 34 |
| hsa-miR-9* | acTttmCggTtaTctAgcTtta | 65 | 27 |
| hsa-miR-92 | cagGccGggAcaAgtGcaAta | 79 | 28 |
| hsa-miR-93 | ctAccTgcAcgAacAgcActTt | 76 | 31 |
| hsa-miR-95 | tgcTcaAtaAatAccmCgtTgaa | 68 | 36 |
| hsa-miR-96 | gcaAaaAtgTgcTagTgcmCaaa | 72 | 38 |
| hsa-miR-98 | aAcaAtamCaamCttActAccTca | 67 | 17 |
| hsa-miR-99a | cacAagAtcGgaTctAcgGgtt | 77 | 42 |
| hsa-miR-99b | cgcAagGtcGgtTctAcgGgtg | 82 | 42 |
| mmu-let-7a | amCtaTacAacmCtamCtamCctmCa | 71 | 16 |
| mmu-let-7b | aamCcamCacAacmCtamCtamCctmCa | 77 | 6 |
| mmu-let-7c | aamCcaTacAacmCtamCtamCctmCa | 74 | 11 |
| mmu-let-7d | actAtgmCaamCctActAccTct | 71 | 24 |
| mmu-let-7d* | agAaaGgcAgcAggTcgTatAg | 79 | 23 |
| mmu-let-7e | actAtamCaamCctmCctAccTca | 71 | 16 |
| mmu-let-7f | amCtaTacAatmCtamCtamCctmCa | 68 | 16 |
| mmu-let-7g | amCtgTacAaamCtamCtamCctmCa | 71 | 30 |
| mmu-let-7i | amCagmCacAaamCtamCtamCctmCa | 76 | 18 |
| mmu-miR-1 | tamCatActTctTtamCatTcca | 64 | 11 |
| mmu-miR-100 | cacAagTtcGgaTctAcgGgtt | 77 | 38 |
| mmu-miR-101a | cttmCagTtaTcamCagTacTgta | 68 | 54 |
| mmu-miR-101b | cttmCagmCtaTcamCagTacTgta | 70 | 54 |
| mmu-miR-103 | tmCatAgcmCctGtamCaaTgcTgct | 80 | 63 |
| mmu-miR-106a | tacmCtgmCacTgtTagmCacTttg | 73 | 44 |
| mmu-miR-106b | atcTgcActGtcAgcActTta | 72 | 35 |
| mmu-miR-107 | tGatAgcmCctGtamCaaTgcTgct | 80 | 63 |
| mmu-miR-10a | cAcaAatTcgGatmCtamCagGgta | 74 | 37 |
| mmu-miR-10b | acamCaaAttmCggTtcTacAggg | 73 | 27 |
| mmu-miR-122a | acAaamCacmCatTgtmCacActmCca | 78 | 25 |
| mmu-miR-124a | ggmCatTcamCcgmCgtGccTta | 80 | 43 |
| mmu-miR-125a | cAcaGgtTaaAggGtcTcaGgga | 79 | 35 |
| mmu-miR-125b | tcamCaaGttAggGtcTcaGgga | 77 | 35 |
| mmu-miR-126-3p | gcAttAttActmCacGgtAcga | 71 | 25 |
| mmu-miR-126-5p | cgmCgtAccAaaAgtAatAatg | 68 | 28 |
| mmu-miR-127 | gcmCaaGctmCagAcgGatmCcga | 80 | 54 |
| mmu-miR-128a | aaAagAgamCcgGttmCacTgtGa | 77 | 47 |
| mmu-miR-128b | gaAagAgamCcgGttmCacTgtGa | 78 | 47 |
| mmu-miR-129 | agcAagmCccAgamCcgmCaaAaag | 80 | 21 |
| mmu-miR-129-3p | aTgcTttTtgGggTaaGggmCtt | 78 | 37 |
| mmu-miR-129-5p | agcAagmCccAgamCcgmCaaAaag | 80 | 21 |
| mmu-miR-130a | aTgcmCctTttAacAttGcamCtg | 74 | 42 |
| mmu-miR-130b | aTgcmCctTtcAtcAttGcamCtg | 75 | 34 |
| mmu-miR-132 | cgAccAtgGctGtaGacTgtTa | 76 | 48 |
| mmu-miR-133a | acAgcTggTtgAagGggAccAa | 82 | 41 |
| mmu-miR-133b | taGctGgtTgaAggGgamCcaa | 81 | 37 |
| mmu-miR-134 | cccmCtcTggTcaAccAgtmCaca | 79 | 57 |
| mmu-miR-135a | tcamCatAggAatAaaAagmCcaTa | 69 | 22 |
| mmu-miR-135b | cacAtaGgaAtgAaaAgcmCata | 70 | 22 |
| mmu-miR-136 | tccAtcAtcAaaAcaAatGgaGt | 71 | 25 |
| mmu-miR-137 | cTacGcgTatTctTaaGcaAtaa | 67 | 48 |
| mmu-miR-138 | gatTcamCaamCacmCagmCt | 70 | 24 |
| mmu-miR-139 | aGacAcgTgcActGtaGa | 75 | 42 |
| mmu-miR-140 | ctamCcaTagGgtAaaAccActg | 71 | 56 |
| mmu-miR-140* | tcmCgtGgtTctAccmCtgTggTa | 81 | 49 |
| mmu-miR-141 | cmCatmCttTacmCagAcaGtgTta | 70 | 33 |
| mmu-miR-142-3p | ccaTaaAgtAggAaamCacTaca | 69 | 29 |
| mmu-miR-142-5p | gtaGtgmCttTctActTtaTg | 63 | 36 |
| mmu-miR-143 | tGagmCtamCagTgcTtcAtcTca | 75 | 56 |
| mmu-miR-144 | ctaGtamCatmCatmCtaTacTgta | 64 | 37 |
| mmu-miR-145 | aAggGatTccTggGaaAacTggAc | 79 | 50 |
| mmu-miR-146 | aAccmCatGgaAttmCagTtcTca | 73 | 44 |
| mmu-miR-148a | acaAagTtcTgtAgtGcamCtga | 72 | 54 |
| mmu-miR-148b | acaAagTtcTgtGatGcamCtga | 72 | 39 |
| mmu-miR-149 | ggaGtgAagAcamCggAgcmCaga | 80 | 31 |
| mmu-miR-150 | cacTggTacAagGgtTggGaga | 78 | 30 |
| mmu-miR-151 | cmCtcAagGagmCctmCagTctAg | 78 | 42 |
| mmu-miR-152 | cmCcaAgtTctGtcAtgmCacTga | 78 | 36 |
| mmu-miR-153 | gatmCacTttTgtGacTatGcaa | 69 | 36 |
| mmu-miR-154 | cGaaGgcAacAcgGatAacmCta | 78 | 40 |
| mmu-miR-155 | ccmCctAtcAcaAttAgcAttAa | 69 | 21 |
| mmu-miR-15a | cAcaAacmCatTatGtgmCtgmCta | 73 | 35 |
| mmu-miR-15b | tgtAaamCcaTgaTgtGctGcta | 74 | 38 |
| mmu-miR-16 | cgmCcaAtaTttAcgTgcTgcTa | 74 | 34 |
| mmu-miR-17-3p | tacAagTgcmCctmCacTgcAgt | 79 | 42 |
| mmu-miR-17-5p | actAccTgcActGtaAgcActTtg | 74 | 39 |
| mmu-miR-18 | tatmCtgmCacTagAtgmCacmCtta | 71 | 40 |
| mmu-miR-181a | acTcamCcgAcaGcgTtgAatGtt | 77 | 49 |
| mmu-miR-181b | cccAccGacAgcAatGaaTgtt | 78 | 30 |
| mmu-miR-181c | amCtcAccGacAggTtgAatGtt | 76 | 33 |
| mmu-miR-182 | tgtGagTtcTacmCatTgcmCaaa | 72 | 32 |
| mmu-miR-183 | caGtgAatTctAccAgtGccAta | 73 | 32 |
| mmu-miR-184 | acmCctTatmCagTtcTccGtcmCa | 76 | 23 |
| mmu-miR-185 | gAacTgcmCttTctmCtcmCa | 70 | 27 |
| mmu-miR-186 | aaGccmCaaAagGagAatTctTtg | 71 | 48 |
| mmu-miR-187 | ccGgcTgcAacAcaAgamCacGa | 85 | 31 |
| mmu-miR-188 | amCccTccAccAtgmCaaGggAtg | 83 | 42 |
| mmu-miR-189 | actGatAtcAgcTcaGtaGgcAc | 77 | 54 |
| mmu-miR-190 | acmCtaAtaTatmCaaAcaTatmCa | 62 | 31 |
| mmu-miR-191 | agcTgcTttTggGatTccGttg | 74 | 42 |
| mmu-miR-192 | tgTcaAttmCatAggTcag | 64 | 28 |
| mmu-miR-193 | ctGggActTtgTagGccAgtt | 76 | 31 |
| mmu-miR-194 | tccAcaTggAgtTgcTgtTaca | 75 | 41 |
| mmu-miR-195 | gmCcaAtaTttmCtgTgcTgcTa | 73 | 28 |
| mmu-miR-196a | ccaAcaAcaTgaAacTacmCta | 67 | 20 |
| mmu-miR-196b | cmCaamCaamCagGaaActAccTa | 73 | 27 |
| mmu-miR-199a | gaAcaGgtAgtmCtgAacActGgg | 78 | 40 |
| mmu-miR-199a* | aacmCaaTgtGcaGacTacTgta | 74 | 39 |
| mmu-miR-199b | gaAcaGgtAgtmCtaAacActGgg | 76 | 31 |
| mmu-miR-19a | tmCagTttTgcAtaGatTtgmCaca | 72 | 37 |
| mmu-miR-19b | tmCagTttTgcAtgGatTtgmCaca | 75 | 34 |
| mmu-miR-20 | ctAccTgcActAtaAgcActTta | 70 | 26 |
| mmu-miR-200a | acaTcgTtamCcaGacAgtGtta | 72 | 39 |
| mmu-miR-200b | gtcAtcAttAccAggmCagTatTa | 71 | 31 |
| mmu-miR-200c | ccAtcAttAccmCggmCagTatTa | 74 | 38 |
| mmu-miR-201 | agAacAatGccTtamCtgAgta | 69 | 37 |
| mmu-miR-202 | tmCttmCccAtgmCgcTatAccTct | 76 | 28 |
| mmu-miR-203 | cTagTggTccTaaAcaTttmCa | 68 | 23 |
| mmu-miR-204 | cagGcaTagGatGacAaaGggAa | 78 | 25 |
| mmu-miR-205 | caGacTccGgtGgaAtgAagGa | 81 | 39 |
| mmu-miR-206 | ccamCacActTccTtamCatTcca | 73 | 11 |
| mmu-miR-207 | gaGggAggAgaGccAggAgaAgc | 86 | 18 |
| mmu-miR-208 | acAagmCttTttGctmCgtmCttAt | 71 | 34 |
| mmu-miR-21 | tmCaamCatmCagTctGatAagmCta | 72 | 48 |
| mmu-miR-210 | tcAgcmCgcTgtmCacAcgmCacAg | 87 | 38 |
| mmu-miR-211 | aggmCaaAggAtgAcaAagGgaa | 75 | 18 |
| mmu-miR-212 | gGccGtgActGgaGacTgtTa | 81 | 37 |
| mmu-miR-213 | gGtamCaaTcaAcgGtcGatGgt | 79 | 67 |
| mmu-miR-214 | ctGccTgtmCtgTgcmCtgmCtgt | 81 | 30 |
| mmu-miR-215 | gtmCtgTcaAatmCatAggTcat | 68 | 35 |
| mmu-miR-216 | camCagTtgmCcaGctGagAtta | 74 | 64 |
| mmu-miR-217 | atmCcaGtcAgtTccTgaTgcAgta | 77 | 43 |
| mmu-miR-218 | acAtgGttAgaTcaAgcAcaa | 70 | 40 |
| mmu-miR-219 | agAatTgcGttTggAcaAtca | 70 | 35 |
| mmu-miR-22 | acaGttmCttmCaamCtgGcaGctt | 74 | 48 |
| mmu-miR-221 | aaamCccAgcAgamCaaTgtAgct | 79 | 31 |
| mmu-miR-222 | gaGacmCcaGtaGccAgaTgtAgct | 80 | 38 |
| mmu-miR-223 | gGggTatTtgAcaAacTgamCa | 73 | 40 |
| mmu-miR-224 | tAaamCggAacmCacTagTgamCtta | 74 | 49 |
| mmu-miR-23a | gGaaAtcmCctGgcAatGtgAt | 76 | 37 |
| mmu-miR-23b | ggtAatmCccTggmCaaTgtGat | 76 | 38 |
| mmu-miR-24 | cTgtTccTgcTgaActGagmCca | 80 | 35 |
| mmu-miR-25 | tcaGacmCgaGacAagTgcAatg | 77 | 27 |
| mmu-miR-26a | gcmCtaTccTggAttActTgaa | 70 | 34 |
| mmu-miR-26b | aacmCtaTccTgaAttActTgaa | 65 | 28 |
| mmu-miR-27a | gcGgaActTagmCcamCtgTgaa | 77 | 35 |
| mmu-miR-27b | gcAgaActTagmCcamCtgTgaa | 74 | 38 |
| mmu-miR-28 | ctmCaaTagActGtgAgcTccTt | 73 | 43 |
| mmu-miR-290 | aaaAagTgcmCccmCatAgtTtgAg | 75 | 29 |
| mmu-miR-291-3p | gGcamCacAaaGtgGaaGcamCttt | 78 | 52 |
| mmu-miR-291-5p | aGagAggGccTccActTtgAtg | 77 | 46 |
| mmu-miR-292-3p | acActmCaaAacmCtgGcgGcamCtt | 80 | 33 |
| mmu-miR-292-5p | caaAagAgcmCccmCagTttGagt | 76 | 32 |
| mmu-miR-293 | amCacTacAaamCtcTgcGgcAct | 81 | 30 |
| mmu-miR-294 | acAcamCaaAagGgaAgcActTt | 75 | 25 |
| mmu-miR-295 | agamCtcAaaAgtAgtAgcActTt | 70 | 44 |
| mmu-miR-296 | acAggAttGagGggGggmCcct | 88 | 48 |
| mmu-miR-297 | cAtgmCacAtgmCacAcaTacAt | 75 | 41 |
| mmu-miR-298 | gGaaGaamCagmCccTccTctGcc | 82 | 53 |
| mmu-miR-299 | aTgtAtgTggGacGgtAaamCca | 80 | 35 |
| mmu-miR-29a | aamCcgAttTcaGatGgtGcta | 75 | 43 |
| mmu-miR-29b | aamCacTgaTttmCaaAtgGtgmCta | 71 | 47 |
| mmu-miR-29c | amCcgAttTcaAatGgtGcta | 71 | 47 |
| mmu-miR-300 | gAagAgaGctTgcmCctTgcAta | 77 | 35 |
| mmu-miR-301 | gctTtgAcaAtamCtaTtgmCacTg | 70 | 36 |
| mmu-miR-302 | tcAccAaaAcaTggAagmCacTta | 72 | 25 |
| mmu-miR-30a-3p | gctGcaAacAtcmCgamCtgAaag | 74 | 28 |
| mmu-miR-30a-5p | cTtcmCagTcgAggAtgTttAca | 73 | 31 |
| mmu-miR-30b | agcTgaGtgTagGatGttTaca | 71 | 33 |
| mmu-miR-30c | gmCtgAgaGtgTagGatGttTaca | 73 | 33 |
| mmu-miR-30d | cttmCcaGtcGggGatGttTaca | 76 | 44 |
| mmu-miR-30e | tcmCagTcaAggAtgTttAca | 69 | 30 |
| mmu-miR-30e* | ctgTaaAcaTccGacTgaAag | 69 | 27 |
| mmu-miR-31 | cagmCtaTgcmCagmCatmCttGcct | 79 | 38 |
| mmu-miR-32 | gcaActTagTaaTgtGcaAta | 65 | 43 |
| mmu-miR-320 | tTcgmCccTctmCaamCccAgcTttt | 80 | 26 |
| mmu-miR-322-3p | tgtTgcAgcGctTcaTgtTt | 74 | 48 |
| mmu-miR-322-5p | tccAaaAcaTgaAttGctGctg | 71 | 40 |
| mmu-miR-323 | agAggTcgAccGtgTaaTgtGc | 80 | 46 |
| mmu-miR-324-3p | ccAgcAgcAccTggGgcAgtGg | 92 | 41 |
| mmu-miR-324-5p | cAccAatGccmCtaGggGatGcg | 83 | 54 |
| mmu-miR-325 | acamCttActGagmCacmCtamCtaGg | 78 | 42 |
| mmu-miR-326 | actGgaGgaAggGccmCagAgg | 86 | 46 |
| mmu-miR-328 | acGgaAggGcaGagAggGccAg | 87 | 31 |
| mmu-miR-329 | aAaaAggTtaGctGggTgtGtt | 75 | 32 |
| mmu-miR-33 | cAatGcaActAcaAtgmCac | 68 | 30 |
| mmu-miR-330 | tmCtcTgcAggmCccTgtGctTtgc | 83 | 52 |
| mmu-miR-331 | tTctAggAtaGgcmCcaGggGc | 84 | 51 |
| mmu-miR-335 | amCatTttTcgTtaTtgmCtcTtga | 67 | 26 |
| mmu-miR-337 | aaaGgcAtcAtaTagGagmCtgAa | 74 | 35 |
| mmu-miR-338 | tcaAcaAaaTcamCtgAtgmCtgGa | 73 | 33 |
| mmu-miR-339 | tgAgcTccTggAggAcaGgga | 83 | 47 |
| mmu-miR-340 | ggcTatAaaGtaActGagAcgGa | 72 | 34 |
| mmu-miR-341 | amCtgAccGacmCgamCcgAtcGa | 84 | 53 |
| mmu-miR-342 | gacGggTgcGatTtcTgtGtgAga | 82 | 34 |
| mmu-miR-344 | amCagTcaGgcTttGgcTagAtca | 79 | 53 |
| mmu-miR-345 | gcActGgamCtaGggGtcAgca | 83 | 43 |
| mmu-miR-346 | agaGgcAggmCacTcgGgcAgamCa | 91 | 38 |
| mmu-miR-34a | aamCaamCcaGctAagAcamCtgmCca | 80 | 27 |
| mmu-miR-34b | caaTcaGctAatTacActGccTa | 71 | 40 |
| mmu-miR-34c | gcAatmCagmCtaActAcamCtgmCct | 76 | 31 |
| mmu-miR-350 | tgaAagTgtAtgGgcTttGtgAa | 73 | 42 |
| mmu-miR-351 | cagGctmCaaAggGctmCctmCagGga | 84 | 59 |
| mmu-miR-361 | gTacmCccTggAgaTtcTgaTaa | 73 | 29 |
| mmu-miR-370 | aAccAggTtcmCacmCccAgcAggc | 86 | 34 |
| mmu-miR-375 | tcAcgmCgaGccGaamCgaAcaAa | 81 | 39 |
| mmu-miR-376a | acgTggAttTtcmCtcTacGat | 71 | 47 |
| mmu-miR-376b | aAagTggAtgTtcmCtcTatGat | 70 | 39 |
| mmu-miR-377 | acAaaAgtTgcmCttTgtGtgAt | 73 | 48 |
| mmu-miR-378 | amCacAggAccTggAgtmCagGag | 84 | 51 |
| mmu-miR-379 | cctAcgTtcmCatAgtmCtamCca | 72 | 33 |
| mmu-miR-380-3p | aagAtgTggAccAtamCtamCata | 69 | 49 |
| mmu-miR-380-5p | gmCgcAtgTtcTatGgtmCaamCca | 80 | 41 |
| mmu-miR-381 | amCagAgaGctTgcmCctTgtAta | 76 | 37 |
| mmu-miR-382 | cgaAtcmCacmCacGaamCaamCttc | 75 | 23 |
| mmu-miR-383 | agcmCacAgtmCacmCttmCtgAtct | 76 | 25 |
| mmu-miR-384 | tGtgAacAatTtcTagGaat | 64 | 46 |
| mmu-miR-409 | aAggGgtTcamCcgAgcAacAttc | 80 | 35 |
| mmu-miR-410 | aacAggmCcaTctGtgTtaTatt | 70 | 39 |
| mmu-miR-411 | actGagGgtTagTggAccGtgTt | 80 | 40 |
| mmu-miR-412 | acgGctAgtGgamCcaGgtGaaGt | 86 | 53 |
| mmu-miR-425 | ggmCggAcamCgamCatTccmCgat | 83 | 43 |
| mmu-miR-7 | caamCaaAatmCacTagTctTcca | 69 | 30 |
| mmu-miR-7b | aamCaaAatmCacAagTctTcca | 68 | 24 |
| mmu-miR-9 | cAtamCagmCtaGatAacmCaaAga | 70 | 34 |
| mmu-miR-9* | acTttmCggTtaTctAgcTtta | 65 | 27 |
| mmu-miR-92 | cagGccGggAcaAgtGcaAta | 79 | 28 |
| mmu-miR-93 | ctAccTgcAcgAacAgcActTtg | 77 | 31 |
| mmu-miR-96 | aGcaAaaAtgTgcTagTgcmCaaa | 75 | 38 |
| mmu-miR-98 | aAcaAtamCaamCttActAccTca | 67 | 17 |
| mmu-miR-99a | acAagAtcGgaTctAcgGgt | 77 | 40 |
| mmu-miR-99b | cgcAagGtcGgtTctAcgGgtg | 82 | 42 |
| osa-miR156a | gtgmCtcActmCtcTtcTgtmCa | 71 | 25 |
| osa-miR156b | gtgmCtcActmCtcTtcTgtmCa | 71 | 25 |
| osa-miR156c | gtgmCtcActmCtcTtcTgtmCa | 71 | 25 |
| osa-miR156d | gtgmCtcActmCtcTtcTgtmCa | 71 | 25 |
| osa-miR156e | gtgmCtcActmCtcTtcTgtmCa | 71 | 25 |
| osa-miR156f | gtgmCtcActmCtcTtcTgtmCa | 71 | 25 |
| osa-miR156g | gtgmCtcActmCtcTtcTgtmCa | 71 | 25 |
| osa-miR156h | gtgmCtcActmCtcTtcTgtmCa | 71 | 25 |
| osa-miR156i | gtgmCtcActmCtcTtcTgtmCa | 71 | 25 |
| osa-miR156j | gtgmCtcActmCtcTtcTgtmCa | 71 | 25 |
| osa-miR156k | tgTgcTctmCtcTctTctGtca | 72 | 21 |
| osa-miR156l | taTgcTcamCtcTctTctGtcg | 71 | 17 |
| osa-miR159a | cagAgcTccmCttmCaaTccAaa | 73 | 36 |
| osa-miR159b | cagAgcTccmCttmCaaTccAaa | 73 | 36 |
| osa-miR159c | tggAgcTccmCttmCaaTccAat | 74 | 46 |
| osa-miR159d | cggAgcTccmCttmCaaTccAat | 75 | 46 |
| osa-miR159e | aggAgcTccmCttmCaaTccAat | 74 | 46 |
| osa-miR159f | tagAgcTccmCttmCaaTccAag | 72 | 36 |
| osa-miR160a | tggmCatAcaGggAgcmCagGca | 85 | 49 |
| osa-miR160b | tggmCatAcaGggAgcmCagGca | 85 | 49 |
| osa-miR160c | tggmCatAcaGggAgcmCagGca | 85 | 49 |
| osa-miR160d | tggmCatAcaGggAgcmCagGca | 85 | 49 |
| osa-miR160e | cggmCatAcaGggAgcmCagGca | 85 | 43 |
| osa-miR160f | tgGcaTtcAggGagmCcaGgca | 84 | 60 |
| osa-miR162a | ctgGatGcaGagGttTatmCga | 73 | 34 |
| osa-miR162b | ctgGatGcaGagGctTatmCga | 76 | 36 |
| osa-miR164a | tgcAcgTgcmCctGctTctmCca | 82 | 46 |
| osa-miR164b | tgcAcgTgcmCctGctTctmCca | 82 | 46 |
| osa-miR164c | tGcamCgtAccmCtgmCttmCtcmCa | 82 | 32 |
| osa-miR164d | agcAcgTgcmCctGctTctmCca | 82 | 47 |
| osa-miR164e | ctcAcgTgcmCctGctTctmCca | 80 | 36 |
| osa-miR166a | gGggAatGaaGccTggTccGa | 84 | 33 |
| osa-miR166b | gGggAatGaaGccTggTccGa | 84 | 33 |
| osa-miR166c | gGggAatGaaGccTggTccGa | 84 | 33 |
| osa-miR166d | gGggAatGaaGccTggTccGa | 84 | 33 |
| osa-miR166e | gGggAatGaaGccTggTccGa | 84 | 33 |
| osa-miR166f | gGggAatGaaGccTggTccGa | 84 | 33 |
| osa-miR166g | gAggAatGaaGccTggTccGa | 80 | 29 |
| osa-miR166h | gAggAatGaaGccTggTccGa | 80 | 29 |
| osa-miR166i | gAggAatGaaGccTgaTccGa | 78 | 29 |
| osa-miR166j | gAggAatGaaGccTgaTccGa | 78 | 29 |
| osa-miR166k | aGggAttGaaGccTggTccGa | 83 | 37 |
| osa-miR166l | aGggAttGaaGccTggTccGa | 83 | 37 |
| osa-miR167a | tAgaTcaTgcTggmCagmCttmCa | 79 | 53 |
| osa-miR167b | tAgaTcaTgcTggmCagmCttmCa | 79 | 53 |
| osa-miR167c | tAgaTcaTgcTggmCagmCttmCa | 79 | 53 |
| osa-miR167d | cAgaTcaTgcTggmCagmCttmCa | 80 | 53 |
| osa-miR167e | cAgaTcaTgcTggmCagmCttmCa | 80 | 53 |
| osa-miR167f | cAgaTcaTgcTggmCagmCttmCa | 80 | 53 |
| osa-miR167g | cAgaTcaTgcTggmCagmCttmCa | 80 | 53 |
| osa-miR167h | cAgaTcaTgcTggmCagmCttmCa | 80 | 53 |
| osa-miR167i | cAgaTcaTgcTggmCagmCttmCa | 80 | 53 |
| osa-miR168a | gtmCccGatmCtgmCacmCaaGcga | 82 | 38 |
| osa-miR168b | ttcmCcgAgcTgcAccAagmCct | 83 | 30 |
| osa-miR169a | tcGgcAagTcaTccTtgGctg | 78 | 40 |
| osa-miR169b | ccGgcAagTcaTccTtgGctg | 79 | 40 |
| osa-miR169c | ccGgcAagTcaTccTtgGctg | 79 | 40 |
| osa-miR169d | ccGgcAatTcaTccTtgGcta | 76 | 33 |
| osa-miR169e | ccGgcAagTcaTccTtgGcta | 78 | 35 |
| osa-miR169f | taGgcAagTcaTccTtgGcta | 74 | 47 |
| osa-miR169g | taGgcAagTcaTccTtgGcta | 74 | 47 |
| osa-miR169h | caGgcAagTcaTccTtgGcta | 76 | 41 |
| osa-miR169i | caGgcAagTcaTccTtgGcta | 76 | 41 |
| osa-miR169j | caGgcAagTcaTccTtgGcta | 76 | 41 |
| osa-miR169k | caGgcAagTcaTccTtgGcta | 76 | 41 |
| osa-miR169l | caGgcAagTcaTccTtgGcta | 76 | 41 |
| osa-miR169m | caGgcAagTcaTccTtgGcta | 76 | 41 |
| osa-miR169n | taGgcAagTcaTtcTtgGcta | 71 | 47 |
| osa-miR169o | taGgcAagTcaTtcTtgGcta | 71 | 47 |
| osa-miR169p | ccgGcaAgtTtgTccTtgGcta | 76 | 52 |
| osa-miR169q | caTggGcaGtcTccTtgGcta | 75 | 47 |
| osa-miR171a | gAtaTtgGcgmCggmCtcAatmCa | 78 | 54 |
| osa-miR171b | gaTatTggmCacGgcTcaAtca | 75 | 46 |
| osa-miR171c | gaTatTggmCacGgcTcaAtca | 75 | 46 |
| osa-miR171d | gaTatTggmCacGgcTcaAtca | 75 | 46 |
| osa-miR171e | gaTatTggmCacGgcTcaAtca | 75 | 46 |
| osa-miR171f | gaTatTggmCacGgcTcaAtca | 75 | 46 |
| osa-miR171g | gaTatTggmCtcGgcTcamCctc | 78 | 34 |
| osa-miR171h | agTgaTatTggTtcGgcTcac | 74 | 34 |
| osa-miR172a | atgmCagmCatmCatmCaaGatTct | 73 | 45 |
| osa-miR172b | aTgcAgcAtcAtcAagAttmCc | 74 | 39 |
| osa-miR172c | gTgcAgcAtcAtcAagAttmCa | 74 | 39 |
| osa-miR319a | gggAgcAccmCttmCagTccAa | 78 | 39 |
| osa-miR319b | gggAgcAccmCttmCagTccAa | 78 | 39 |
| osa-miR393 | gAtcAatGcgAtcmCctTtgGa | 74 | 56 |
| osa-miR393b | agaTcaAtgmCgaTccmCttTgga | 73 | 56 |
| osa-miR394 | gGagGtgGacAgaAtgmCcaa | 77 | 29 |
| osa-miR395a | gagTtcmCccmCaaAtamCttmCac | 71 | 23 |
| osa-miR395b | gAgtTccmCccAaamCacTtcAc | 75 | 28 |
| osa-miR395c | gAgtTccmCccAagmCacTtcAc | 78 | 28 |
| osa-miR395d | gAgtTccmCccAaamCacTtcAc | 75 | 28 |
| osa-miR395e | gAgtTccmCccAaamCacTtcAc | 75 | 28 |
| osa-miR395f | gatTtcmCccmCaaAcgmCttmCac | 74 | 22 |
| osa-miR395g | gAgtTccmCccAaamCacTtcAc | 75 | 28 |
| osa-miR395h | gAgtTccmCccAaamCacTtcAc | 75 | 28 |
| osa-miR395i | gAgtTccmCccAaamCacTtcAc | 75 | 28 |
| osa-miR395j | gAgtTccmCccAaamCacTtcAc | 75 | 28 |
| osa-miR395k | gAgtTccmCccAaamCacTtcAc | 75 | 28 |
| osa-miR395l | gAgtTccmCccAaamCacTtcAc | 75 | 28 |
| osa-miR395m | gAgtTccmCccAaamCacTtcAc | 75 | 28 |
| osa-miR395n | gAgtTtcmCccAaamCacTtcAc | 73 | 35 |
| osa-miR395o | gAgtTtcmCccAaamCacTtcAc | 73 | 35 |
| osa-miR395p | gatTtcmCccmCaaAcgmCttmCac | 74 | 22 |
| osa-miR395q | gAgtTccTccAaamCacTtcAc | 72 | 29 |
| osa-miR395r | gAgtTtcmCccAaamCacTtcAc | 73 | 35 |
| osa-miR395s | gatTtcmCccmCaaAcgmCttmCac | 74 | 22 |
| osa-miR396a | cagTtcAagAaaGctGtgGaa | 70 | 35 |
| osa-miR396b | cagTtcAagAaaGctGtgGaa | 70 | 35 |
| osa-miR396c | aagTtcAagAaaGctGtgGaa | 69 | 24 |
| osa-miR397a | caTcaAcgmCtgmCacTcaAtga | 73 | 39 |
| osa-miR397b | caTcaAcgmCtgmCacTcaAtaa | 71 | 35 |
| osa-miR398a | aagGggTgamCctGagAacAca | 80 | 39 |
| osa-miR398b | caGggGcgAccTgaGaamCaca | 83 | 43 |
| osa-miR399a | cAggGcaAttmCtcmCttTggmCa | 78 | 48 |
| osa-miR399b | cAggGcaAttmCtcmCttTggmCa | 78 | 48 |
| osa-miR399c | cAggGcaAttmCtcmCttTggmCa | 78 | 48 |
| osa-miR399d | caGggmCaamCtcTccTttGgca | 81 | 39 |
| osa-miR399e | cTggGcaAatmCtcmCttTggmCa | 77 | 41 |
| osa-miR399f | cTggGcaAatmCtcmCttTggmCa | 77 | 41 |
| osa-miR399g | cmCggGcaAatmCtcmCttTggmCa | 80 | 41 |
| osa-miR399h | cTggGcaAgtmCtcmCttTggmCa | 80 | 37 |
| osa-miR399i | caGggmCagmCtcTccTttGgca | 83 | 63 |
| osa-miR399j | taGggmCaamCtcTccTttGgca | 80 | 39 |
| osa-miR399k | cggGgcAaaTttmCctTtgGca | 76 | 53 |
| osa-miR408 | gmCcaGggAagAggmCagTgcAg | 88 | 35 |
| osa-miR413 | gtgmCagAacAagTgaAacTag | 70 | 24 |
| osa-miR414 | gGacGatGatGatGagGatGa | 77 | 21 |
| osa-miR415 | ctgmCtcTgcTtcTgtTctGtt | 71 | 19 |
| osa-miR416 | tgAacAgtGtamCggAcgAaca | 75 | 42 |
| osa-miR417 | tgGaamCaaAttmCacTacAttc | 66 | 26 |
| osa-miR418 | cgTcaTttmCatmCatmCacAtta | 67 | 16 |
| osa-miR419 | caamCatmCgtmCagmCatTcaTca | 74 | 18 |
| osa-miR420 | atcAttTccGtgAttAatTta | 60 | 32 |
| osa-miR426 | cgtAagGacAaamCttmCcaAaa | 69 | 31 |
| rno-let-7a | aamCtaTacAacmCtamCtamCctmCa | 70 | 16 |
| rno-let-7b | aamCcamCacAacmCtamCtamCctmCa | 77 | 6 |
| rno-let-7c | aamCcaTacAacmCtamCtamCctmCa | 74 | 11 |
| rno-let-7d | actAtgmCaamCctActAccTct | 71 | 24 |
| rno-let-7d* | agAaaGgcAgcAggTcgTatAg | 79 | 23 |
| rno-let-7e | actAtamCaamCctmCctAccTca | 71 | 16 |
| rno-let-7f | aamCtaTacAatmCtamCtamCctmCa | 67 | 16 |
| rno-let-7i | amCagmCacAaamCtamCtamCctmCa | 76 | 18 |
| rno-miR-100 | cacAagTtcGgaTctAcgGgtt | 77 | 38 |
| rno-miR-101 | cttmCagTtaTcamCagTacTgta | 68 | 54 |
| rno-miR-101b | cttmCagmCtaTcamCagTacTgta | 70 | 54 |
| rno-miR-103 | tmCatAgcmCctGtamCaaTgcTgct | 80 | 63 |
| rno-miR-106b | atcTgcActGtcAgcActTta | 72 | 35 |
| rno-miR-107 | tGatAgcmCctGtamCaaTgcTgct | 80 | 63 |
| rno-miR-10a | cAcaAatTcgGatmCtamCagGgta | 74 | 37 |
| rno-miR-10b | acamCaaAttmCggTtcTacAggg | 73 | 27 |
| rno-miR-122a | acAaamCacmCatTgtmCacActmCca | 78 | 25 |
| rno-miR-124a | tggmCatTcamCcgmCgtGccTtaa | 80 | 43 |
| rno-miR-125a | cAcaGgtTaaAggGtcTcaGgga | 79 | 35 |
| rno-miR-125b | tcamCaaGttAggGtcTcaGgga | 77 | 35 |
| rno-miR-126 | gcAttAttActmCacGgtAcga | 71 | 25 |
| rno-miR-126* | cgmCgtAccAaaAgtAatAatg | 68 | 28 |
| rno-miR-127 | agcmCaaGctmCagAcgGatmCcga | 81 | 54 |
| rno-miR-128a | aaAagAgamCcgGttmCacTgtGa | 77 | 47 |
| rno-miR-128b | gaAagAgamCcgGttmCacTgtGa | 78 | 47 |
| rno-miR-129 | agcAagmCccAgamCcgmCaaAaag | 80 | 21 |
| rno-miR-129* | aTgcTttTtgGggTaaGggmCtt | 78 | 37 |
| rno-miR-130a | aTgcmCctTttAacAttGcamCtg | 74 | 42 |
| rno-miR-130b | aTgcmCctTtcAtcAttGcamCtg | 75 | 34 |
| rno-miR-132 | cgAccAtgGctGtaGacTgtTa | 76 | 48 |
| rno-miR-133a | acAgcTggTtgAagGggAccAa | 82 | 41 |
| rno-miR-134 | ccmCtcTggTcaAccAgtmCaca | 77 | 57 |
| rno-miR-135a | tcamCatAggAatAaaAagmCcaTa | 69 | 22 |
| rno-miR-135b | cacAtaGgaAtgAaaAgcmCata | 70 | 22 |
| rno-miR-136 | tccAtcAtcAaaAcaAatGgaGt | 71 | 25 |
| rno-miR-137 | cTacGcgTatTctTaaGcaAta | 68 | 48 |
| rno-miR-138 | gatTcamCaamCacmCagmCt | 70 | 24 |
| rno-miR-139 | aGacAcgTgcActGtaGa | 75 | 42 |
| rno-miR-140 | ctAccAtaGggTaaAacmCact | 71 | 43 |
| rno-miR-140* | tgtmCcgTggTtcTacmCctGtgGta | 80 | 50 |
| rno-miR-141 | cmCatmCttTacmCagAcaGtgTta | 70 | 33 |
| rno-miR-142-3p | tmCcaTaaAgtAggAaamCacTaca | 72 | 29 |
| rno-miR-142-5p | gtaGtgmCttTctActTtaTg | 63 | 36 |
| rno-miR-143 | tGagmCtamCagTgcTtcAtcTca | 75 | 56 |
| rno-miR-144 | ctaGtamCatmCatmCtaTacTgta | 64 | 37 |
| rno-miR-145 | aAggGatTccTggGaaAacTggAc | 79 | 50 |
| rno-miR-146 | aAccmCatGgaAttmCagTtcTca | 73 | 44 |
| rno-miR-148b | acaAagTtcTgtGatGcamCtga | 72 | 39 |
| rno-miR-150 | cacTggTacAagGgtTggGaga | 78 | 30 |
| rno-miR-151 | cmCtcAagGagmCctmCagTctAgt | 78 | 42 |
| rno-miR-151* | tacTagActGtgAgcTccTcga | 74 | 42 |
| rno-miR-152 | cmCcaAgtTctGtcAtgmCacTga | 78 | 36 |
| rno-miR-153 | tcamCttTtgTgamCtaTgcAa | 68 | 35 |
| rno-miR-154 | cGaaGgcAacAcgGatAacmCta | 78 | 40 |
| rno-miR-15b | tgtAaamCcaTgaTgtGctGcta | 74 | 38 |
| rno-miR-16 | cgmCcaAtaTttAcgTgcTgcTa | 74 | 34 |
| rno-miR-17 | actAccTgcActGtaAgcActTtg | 74 | 39 |
| rno-miR-18 | tatmCtgmCacTagAtgmCacmCtta | 71 | 40 |
| rno-miR-181a | acTcamCcgAcaGcgTtgAatGtt | 77 | 49 |
| rno-miR-181b | cccAccGacAgcAatGaaTgtt | 78 | 30 |
| rno-miR-181c | amCtcAccGacAggTtgAatGtt | 76 | 33 |
| rno-miR-183 | caGtgAatTctAccAgtGccAta | 73 | 32 |
| rno-miR-184 | acmCctTatmCagTtcTccGtcmCa | 76 | 23 |
| rno-miR-185 | gAacTgcmCttTctmCtcmCa | 70 | 27 |
| rno-miR-186 | aaGccmCaaAagGagAatTctTtg | 71 | 48 |
| rno-miR-187 | cGgcTgcAacAcaAgamCacGa | 84 | 31 |
| rno-miR-190 | acmCtaAtaTatmCaaAcaTatmCa | 62 | 31 |
| rno-miR-191 | agcTgcTttTggGatTccGttg | 74 | 42 |
| rno-miR-192 | gGctGtcAatTcaTagGtcAg | 73 | 46 |
| rno-miR-193 | ctGggActTtgTagGccAgtt | 76 | 31 |
| rno-miR-194 | tccAcaTggAgtTgcTgtTaca | 75 | 41 |
| rno-miR-195 | gmCcaAtaTttmCtgTgcTgcTa | 73 | 28 |
| rno-miR-196a | ccaAcaAcaTgaAacTacmCta | 67 | 20 |
| rno-miR-196b | cmCaamCaamCagGaaActAccTa | 73 | 27 |
| rno-miR-199a | gaAcaGgtAgtmCtgAacActGgg | 78 | 40 |
| rno-miR-19a | tmCagTttTgcAtaGatTtgmCaca | 72 | 37 |
| rno-miR-19b | tmCagTttTgcAtgGatTtgmCaca | 75 | 34 |
| rno-miR-20 | ctAccTgcActAtaAgcActTta | 70 | 26 |
| rno-miR-20* | tgtAagTgcTcgTaaTgcAgt | 74 | 26 |
| rno-miR-200a | acaTcgTtamCcaGacAgtGtta | 72 | 39 |
| rno-miR-200b | gtcAtcAttAccAggmCagTatTa | 71 | 31 |
| rno-miR-200c | ccAtcAttAccmCggmCagTatTa | 74 | 38 |
| rno-miR-203 | cTagTggTccTaaAcaTttmCac | 69 | 23 |
| rno-miR-204 | aggmCatAggAtgAcaAagGgaa | 75 | 25 |
| rno-miR-205 | caGacTccGgtGgaAtgAagGa | 81 | 39 |
| rno-miR-206 | ccamCacActTccTtamCatTcca | 73 | 11 |
| rno-miR-208 | acAagmCttTttGctmCgtmCttAt | 71 | 34 |
| rno-miR-21 | tmCaamCatmCagTctGatAagmCta | 72 | 48 |
| rno-miR-210 | tcAgcmCgcTgtmCacAcgmCacAg | 87 | 38 |
| rno-miR-211 | aggmCaaAggAtgAcaAagGgaa | 75 | 18 |
| rno-miR-212 | gGccGtgActGgaGacTgtTa | 81 | 37 |
| rno-miR-213 | gGtamCaaTcaAcgGtcGatGgt | 79 | 67 |
| rno-miR-214 | ctGccTgtmCtgTgcmCtgmCtgt | 81 | 30 |
| rno-miR-216 | camCagTtgmCcaGctGagAtta | 74 | 64 |
| rno-miR-217 | atmCcaGtcAgtTccTgaTgcAgta | 77 | 43 |
| rno-miR-218 | acAtgGttAgaTcaAgcAcaa | 70 | 40 |
| rno-miR-219 | agAatTgcGttTggAcaAtca | 70 | 35 |
| rno-miR-22 | acaGttmCttmCaamCtgGcaGctt | 74 | 48 |
| rno-miR-221 | gAaamCccAgcAgamCaaTgtAgct | 80 | 31 |
| rno-miR-222 | gaGacmCcaGtaGccAgaTgtAgct | 80 | 38 |
| rno-miR-223 | gGggTatTtgAcaAacTgamCa | 73 | 40 |
| rno-miR-23a | gGaaAtcmCctGgcAatGtgAt | 76 | 37 |
| rno-miR-23b | ggtAatmCccTggmCaaTgtGat | 76 | 38 |
| rno-miR-24 | cTgtTccTgcTgaActGagmCca | 80 | 35 |
| rno-miR-25 | tcaGacmCgaGacAagTgcAatg | 77 | 27 |
| rno-miR-26a | gcmCtaTccTggAttActTgaa | 70 | 34 |
| rno-miR-26b | aacmCtaTccTgaAttActTgaa | 65 | 28 |
| rno-miR-27a | gcGgaActTagmCcamCtgTgaa | 77 | 35 |
| rno-miR-27b | gcAgaActTagmCcamCtgTgaa | 74 | 38 |
| rno-miR-28 | ctmCaaTagActGtgAgcTccTt | 73 | 43 |
| rno-miR-290 | aaaAagTgcmCccmCatAgtTtgAg | 75 | 29 |
| rno-miR-291-3p | gGcamCacAaaGtgGaaGcamCttt | 78 | 52 |
| rno-miR-291-5p | aGagAggGccTccActTtgAtg | 77 | 46 |
| rno-miR-292-3p | acActmCaaAacmCtgGcgGcamCtt | 80 | 33 |
| rno-miR-292-5p | caaAagAgcmCccmCagTttGagt | 76 | 32 |
| rno-miR-296 | acAggAttGagGggGggmCcct | 88 | 48 |
| rno-miR-297 | cAtgmCatAcaTgcAcamCatAcat | 74 | 47 |
| rno-miR-298 | gGaaGaamCagmCccTccTctGcc | 82 | 53 |
| rno-miR-299 | aTgtAtgTggGacGgtAaamCca | 80 | 35 |
| rno-miR-29a | aamCcgAttTcaGatGgtGcta | 75 | 43 |
| rno-miR-29b | aamCacTgaTttmCaaAtgGtgmCta | 71 | 47 |
| rno-miR-29c | amCcgAttTcaAatGgtGcta | 71 | 47 |
| rno-miR-300 | gAagAgaGctTgcmCctTgcAta | 77 | 35 |
| rno-miR-301 | atGctTtgAcaAtamCtaTtgmCacTg | 72 | 42 |
| rno-miR-30a-3p | gctGcaAacAtcmCgamCtgAaag | 74 | 28 |
| rno-miR-30a-5p | cTtcmCagTcgAggAtgTttAca | 73 | 31 |
| rno-miR-30b | agcTgaGtgTagGatGttTaca | 71 | 33 |
| rno-miR-30c | gmCtgAgaGtgTagGatGttTaca | 73 | 33 |
| rno-miR-30d | cttmCcaGtcGggGatGttTaca | 76 | 44 |
| rno-miR-30e | tcmCagTcaAggAtgTttAca | 69 | 30 |
| rno-miR-31 | cagmCtaTgcmCagmCatmCttGcct | 79 | 38 |
| rno-miR-32 | gcaActTagTaaTgtGcaAta | 65 | 43 |
| rno-miR-320 | tTcgmCccTctmCaamCccAgcTttt | 80 | 26 |
| rno-miR-322 | tgtTgcAgcGctTcaTgtTt | 74 | 48 |
| rno-miR-323 | agAggTcgAccGtgTaaTgtGc | 80 | 46 |
| rno-miR-324-3p | ccAgcAgcAccTggGgcAgtGg | 92 | 41 |
| rno-miR-324-5p | acAccAatGccmCtaGggGatGcg | 84 | 54 |
| rno-miR-325 | acamCttActGagmCacmCtamCtaGg | 78 | 42 |
| rno-miR-326 | actGgaGgaAggGccmCagAgg | 86 | 46 |
| rno-miR-327 | accmCtcAtgmCccmCtcAagg | 76 | 27 |
| rno-miR-328 | acGgaAggGcaGagAggGccAg | 87 | 31 |
| rno-miR-329 | aAaaAggTtaGctGggTgtGtt | 75 | 32 |
| rno-miR-33 | cAatGcaActAcaAtgmCac | 68 | 30 |
| rno-miR-330 | tmCtcTgcAggmCccTgtGctTtgc | 83 | 52 |
| rno-miR-331 | tTctAggAtaGgcmCcaGggGc | 84 | 51 |
| rno-miR-333 | aaaAgtAacTagmCacAccAc | 69 | 24 |
| rno-miR-335 | amCatTttTcgTtaTtgmCtcTtga | 67 | 26 |
| rno-miR-336 | aGacTagAtaTggAagGgtGa | 75 | 28 |
| rno-miR-337 | aaaGgcAtcAtaTagGagmCtgAa | 74 | 35 |
| rno-miR-338 | tcaAcaAaaTcamCtgAtgmCtgGa | 73 | 33 |
| rno-miR-339 | tgAgcTccTggAggAcaGgga | 83 | 47 |
| rno-miR-340 | ggcTatAaaGtaActGagAcgGa | 72 | 34 |
| rno-miR-341 | amCtgAccGacmCgamCcgAtcGa | 84 | 53 |
| rno-miR-342 | gacGggTgcGatTtcTgtGtgAga | 82 | 34 |
| rno-miR-343 | tctGggmCacAcgGagGgaGa | 87 | 40 |
| rno-miR-344 | amCggTcaGgcTttGgcTagAtca | 81 | 63 |
| rno-miR-345 | gcActGgamCtaGggGtcAgca | 83 | 43 |
| rno-miR-346 | aGagGcaGgcActmCagGcaGaca | 86 | 37 |
| rno-miR-347 | tggGcgAccmCagAggGaca | 82 | 43 |
| rno-miR-349 | agaGgtTaaGacAgcAggGctg | 79 | 39 |
| rno-miR-34a | aamCaamCcaGctAagAcamCtgmCca | 80 | 27 |
| rno-miR-34b | caaTcaGctAatTacActGccTa | 71 | 40 |
| rno-miR-34c | gcAatmCagmCtaActAcamCtgmCct | 76 | 31 |
| rno-miR-350 | gTgaAagTgtAtgGgcTttGtgAa | 76 | 42 |
| rno-miR-351 | cagGctmCaaAggGctmCctmCagGga | 84 | 59 |
| rno-miR-352 | tamCtaTgcAacmCtamCtamCtct | 68 | 26 |
| rno-miR-421 | caAcaAacAttTaaTgaGgcc | 68 | 30 |
| rno-miR-7 | aacAaaAtcActAgtmCttmCca | 66 | 30 |
| rno-miR-7* | tatGgcAgamCtgTgaTttGttg | 73 | 45 |
| rno-miR-7b | aamCaaAatmCacAagTctTcca | 68 | 24 |
| rno-miR-9 | tcAtamCagmCtaGatAacmCaaAga | 71 | 34 |
| rno-miR-92 | cagGccGggAcaAgtGcaAta | 79 | 28 |
| rno-miR-93 | ctAccTgcAcgAacAgcActTtg | 77 | 31 |
| rno-miR-96 | aGcaAaaAtgTgcTagTgcmCaaa | 75 | 38 |
| rno-miR-98 | aAcaAtamCaamCttActAccTca | 67 | 17 |
| rno-miR-99a | cacAagAtcGgaTctAcgGgtt | 77 | 42 |
| rno-miR-99b | cgcAagGtcGgtTctAcgGgtg | 82 | 42 |
| zma-miR156a | gtgmCtcActmCtcTtcTgtmCa | 71 | 25 |
| zma-miR156b | gtgmCtcActmCtcTtcTgtmCa | 71 | 25 |
| zma-miR156c | gtgmCtcActmCtcTtcTgtmCa | 71 | 25 |
| zma-miR156d | gtgmCtcActmCtcTtcTgtmCa | 71 | 25 |
| zma-miR156e | gtgmCtcActmCtcTtcTgtmCa | 71 | 25 |
| zma-miR156f | gtgmCtcActmCtcTtcTgtmCa | 71 | 25 |
| zma-miR156g | gtgmCtcActmCtcTtcTgtmCa | 71 | 25 |
| zma-miR156h | gtgmCtcActmCtcTtcTgtmCa | 71 | 25 |
| zma-miR156i | gtgmCtcActmCtcTtcTgtmCa | 71 | 25 |
| zma-miR160a | tggmCatAcaGggAgcmCagGca | 85 | 49 |
| zma-miR160b | tggmCatAcaGggAgcmCagGca | 85 | 49 |
| zma-miR160c | tggmCatAcaGggAgcmCagGca | 85 | 49 |
| zma-miR160d | tggmCatAcaGggAgcmCagGca | 85 | 49 |
| zma-miR160e | tggmCatAcaGggAgcmCagGca | 85 | 49 |
| zma-miR162 | tggAtgmCagAggTttAtcGa | 73 | 28 |
| zma-miR164a | tgcAcgTgcmCctGctTctmCca | 82 | 46 |
| zma-miR164b | tgcAcgTgcmCctGctTctmCca | 82 | 46 |
| zma-miR164c | tgcAcgTgcmCctGctTctmCca | 82 | 46 |
| zma-miR164d | tgcAcgTgcmCctGctTctmCca | 82 | 46 |
| zma-miR166a | gGggAatGaaGccTggTccGa | 84 | 33 |
| zma-miR166b | gggAatGaaGccTggTccGa | 79 | 29 |
| zma-miR166c | gggAatGaaGccTggTccGa | 79 | 29 |
| zma-miR166d | gggAatGaaGccTggTccGa | 79 | 29 |
| zma-miR166e | gggAatGaaGccTggTccGa | 79 | 29 |
| zma-miR166f | gggAatGaaGccTggTccGa | 79 | 29 |
| zma-miR166g | gggAatGaaGccTggTccGa | 79 | 29 |
| zma-miR166h | gggAatGaaGccTggTccGa | 79 | 29 |
| zma-miR166i | gggAatGaaGccTggTccGa | 79 | 29 |
| zma-miR167a | tAgaTcaTgcTggmCagmCttmCa | 79 | 53 |
| zma-miR167b | tAgaTcaTgcTggmCagmCttmCa | 79 | 53 |
| zma-miR167c | tAgaTcaTgcTggmCagmCttmCa | 79 | 53 |
| zma-miR167d | tAgaTcaTgcTggmCagmCttmCa | 79 | 53 |
| zma-miR169a | tcGgcAagTcaTccTtgGctg | 78 | 40 |
| zma-miR169b | tcGgcAagTcaTccTtgGctg | 78 | 40 |
| zma-miR171a | ataTtgGcgmCggmCtcAatmCa | 76 | 46 |
| zma-miR171b | gtgAtaTtgGcamCggmCtcAa | 74 | 43 |
| zma-miR172a | tgmCagmCatmCatmCaaGatTct | 73 | 39 |
| zma-miR172b | tgmCagmCatmCatmCaaGatTct | 73 | 39 |
| zma-miR172c | tgmCagmCatmCatmCaaGatTct | 73 | 39 |
| zma-miR172d | tgmCagmCatmCatmCaaGatTct | 73 | 39 |

### Example 13

Determination of MicroRNA Expression in Zebrafish Embryonic Development By Whole Mount in situ Hybridization of Embryos Using LNA-Substituted miRNA Detection Probes

Zebrafish

Zebrafish were kept under standard conditions (M. Westerfield, The zebrafish book (University of Oregon Press, 1993). Embryos were staged according to (C. B. Kimmel, W. W. Ballard, S. R. Kimmel, B. Ullmann, T. F. Schilling, Dev Dyn 203, 253-310 (1995). Homozygous albino embryos and larvae were used for the in situ hybridizations.

LNA-Substituted MicroRNA Probes

The sequences of the LNA-substituted microRNA probes are listed below. The LNA probes were labeled with digoxigenin (DIG) using a DIG 3′-end labeling kit (Roche) and purified using Sephadex G25 MicroSpin columns (Amersham). For in situ hybridizations approximately 1-2 pmol of labeled probe was used.

TABLE 1
List of LNA-substituted detection probes for
determination of microRNA expression in
zebrafish embryonic development by whole
mount in situ hybridization of embryos

| | | Calc |
|---|---|---|
| | | Tm |
| Probe name | Probe sequence 5′-3′ | ° C. |
| hsa-let7f/LNA | aamCtaTacAatmCtamCtamCctmCa | 67 |
| hsa-miR19b/LNA | tmCagTttTgcAtgGatTtgmCaca | 75 |
| hsa-miR17-5p/LNA | actAccTgcActGtaAgcActTtg | 74 |
| hsa-miR217/LNA | atcmCaaTcaGttmCctGatGcaGta | 75 |
| hsa-miR218/LNA | acAtgGttAgaTcaAgcAcaa | 70 |
| hsa-miR222/LNA | gaGacmCcaGtaGccAgaTgtAgct | 80 |
| hsa-let7i/LNA | agmCacAaamCtamCtamCctmCa | 71 |
| hsa-miR27b/LNA | cagAacTtaGccActGtgAa | 68 |
| hsa-miR301/LNA | gctTtgAcaAtamCtaTtgmCacTg | 70 |
| hsa-miR30b/LNA | gcTgaGtgTagGatGttTaca | 70 |
| hsa-miR100/LNA | cacAagTtcGgaTctAcgGgtt | 77 |
| hsa-miR34a/LNA | aamCaamCcaGctAagAcamCtgmCca | 80 |
| hsa-miR7/LNA | aacAaaAtcActAgtmCttmCca | 66 |
| hsa-miR125b/LNA | tcamCaaGttAggGtcTcaGgga | 77 |
| hsa-miR133a/LNA | acAgcTggTtgAagGggAccAa | 82 |
| hsa-miR101/LNA | cttmCagTtaTcamCagTacTgta | 68 |
| hsa-miR108/LNA | aatGccmCctAaaAatmCctTat | 66 |
| hsa-miR107/LNA | tGatAgcmCctGtamCaaTgcTgct | 80 |
| hsa-miR153/LNA | tcamCttTtgTgamCtaTgcAa | 68 |
| hsa-miR10b/LNA | amCaaAttmCggTtcTacAggGta | 73 |
| mmu-miR10b/LNA | acamCaaAttmCggTtcTacAggg | 73 |
| hsa-miR194/LNA | tccAcaTggAgtTgcTgtTaca | 75 |
| hsa-miR199a/LNA | gaAcaGgtAgtmCtgAacActGgg | 78 |
| hsa-miR199a*/LNA | aacmCaaTgtGcaGacTacTgta | 74 |
| hsa-miR20/LNA | ctAccTgcActAtaAgcActTta | 70 |
| hsa-miR214/LNA | ctGccTgtmCtgTgcmCtgmCtgt | 81 |
| hsa-miR219/LNA | agAatTgcGttTggAcaAtca | 70 |
| hsa-miR223/LNA | gGggTatTtgAcaAacTgamCa | 73 |
| hsa-miR23a/LNA | gGaaAtcmCctGgcAatGtgAt | 76 |
| hsa-miR24/LNA | cTgtTccTgcTgaActGagmCca | 80 |
| hsa-miR26a/LNA | agcmCtaTccTggAttActTgaa | 70 |
| hsa-miR126/LNA | gcAttAttActmCacGgtAcga | 71 |
| hsa-miR126*/LNA | cgmCgtAccAaaAgtAatAatg | 68 |
| hsa-miR128a/LNA | aaAagAgamCcgGttmCacTgtGa | 77 |
| mmu-miR7b/LNA | aamCaaAatmCacAagTctTcca | 68 |
| hsa-let7c/LNA | aamCcaTacAacmCtamCtamCctmCa | 74 |
| hsa-let7b/LNA | aamCcamCacAacmCtamCtamCctmCa | 77 |
| hsa-miR103/LNA | tmCatAgcmCctGtamCaaTgcTgct | 80 |
| hsa-miR129/LNA | agcAagmCccAgamCcgmCaaAaag | 80 |
| rno-miR129*/LNA | aTgcTttTtgGggTaaGggmCtt | 78 |
| hsa-miR130a/LNA | gcmCctTttAacAttGcamCtg | 70 |
| hsa-miR132/LNA | cgAccAtgGctGtaGacTgtTa | 76 |
| hsa-miR135a/LNA | tcamCatAggAatAaaAagmCcaTa | 69 |
| hsa-miR137/LNA | cTacGcgTatTctTaaGcaAta | 68 |
| hsa-miR200a/LNA | acaTcgTtamCcaGacAgtGtta | 72 |
| hsa-miR142-3p/ | tmCcaTaaAgtAggAaamCacTaca | 72 |
| LNA | | |
| hsa-miR142-5p/ | gtaGtgmCttTctActTtaTg | 63 |
| LNA | | |
| hsa-miR181b/LNA | aamCccAccGacAgcAatGaaTgtt | 81 |
| hsa-miR183/LNA | caGtgAatTctAccAgtGccAta | 73 |
| hsa-miR190/LNA | acmCtaAtaTatmCaaAcaTatmCa | 62 |
| hsa-miR193/LNA | ctGggActTtgTagGccAgtt | 76 |
| hsa-miR19a/LNA | tmCagTttTgcAtaGatTtgmCaca | 72 |
| hsa-miR204/LNA | cagGcaTagGatGacAaaGggAa | 78 |
| hsa-miR205/LNA | caGacTccGgtGgaAtgAagGa | 81 |
| hsa-miR216/LNA | camCagTtgmCcaGctGagAtta | 74 |
| hsa-miR221/LNA | gAaamCccAgcAgamCaaTgtAgct | 80 |
| hsa-miR25/LNA | tcaGacmCgaGacAagTgcAatg | 77 |
| hsa-miR29c/LNA | taamCcgAttTcaAatGgtGcta | 70 |
| hsa-miR29b/LNA | amCacTgaTttmCaaAtgGtgmCta | 71 |
| hsa-miR30c/LNA | gmCtgAgaGtgTagGatGttTaca | 73 |
| hsa-miR140/LNA | ctAccAtaGggTaaAacmCact | 71 |
| hsa-miR9*/LNA | acTttmCggTtaTctAgcTtta | 65 |
| hsa-miR92/LNA | amCagGccGggAcaAgtGcaAta | 81 |
| hsa-miR96/LNA | aGcaAaaAtgTgcTagTgcmCaaa | 75 |
| hsa-miR99a/LNA | cacAagAtcGgaTctAcgGgtt | 77 |
| hsa-miR145/LNA | aAggGatTccTggGaaAacTggAc | 79 |
| hsa-miR155/LNA | ccmCctAtcAcgAttAgcAttAa | 71 |
| hsa-miR29a/LNA | aamCcgAttTcaAatGgtGctAg | 75 |
| rno-miR140*/LNA | gtcmCgtGgtTctAccmCtgTggTa | 81 |
| hsa-miR206/LNA | ccamCacActTccTtamCatTcca | 73 |
| hsa-miR124a/LNA | tggmCatTcamCcgmCgtGccTtaa | 80 |
| hsa-miR122a/LNA | acAaamCacmCatTgtmCacActmCca | 78 |
| hsa-miR1/LNA | tamCatActTctTtamCatTcca | 64 |
| hsa-miR181a/LNA | acTcamCcgAcaGcgTtgAatGtt | 77 |
| hsa-miR10a/LNA | cAcaAatTcgGatmCtamCagGgta | 74 |
| hsa-miR196a/LNA | ccaAcaAcaTgaAacTacmCta | 67 |
| hsa-let7a/LNA | aamCtaTacAacmCtamCtamCctmCa | 70 |
| hsa-miR9/LNA | tcAtamCagmCtaGatAacmCaaAga | 71 |
| hsa-miR210/LNA | agcmCgcTgtmCacAcgmCacAg | 84 |
| hsa-miR144/LNA | taGtamCatmCatmCtaTacTgta | 64 |
| hsa-miR338/LNA | caAcaAaaTcamCtgAtgmCtgGa | 72 |
| hsa-miR187/LNA | ggcTgcAacAcaAgamCacGa | 79 |
| hsa-miR200b/LNA | cAtcAttAccAggmCagTatTaga | 71 |
| hsa-miR184/LNA | cmCctTatmCagTtcTccGtcmCa | 75 |
| hsa-miR27a/LNA | gcGgaActTagmCcamCtgTgaa | 77 |
| hsa-miR215/LNA | ctgTcaAttmCatAggTcat | 65 |
| hsa-miR203/LNA | agTggTccTaaAcaTttmCac | 68 |
| hsa-miR16/LNA | ccaAtaTttAcgTgcTgcTa | 68 |
| hsa-miR152/LNA | aAgtTctGtcAtgmCacTga | 72 |
| hsa-miR138/LNA | gatTcamCaamCacmCagmCt | 70 |
| hsa-miR143/LNA | gagmCtamCagTgcTtcAtcTca | 72 |
| hsa-miR195/LNA | gmCcaAtaTttmCtgTgcTgcTa | 73 |
| hsa-mir375/LNA | tAacGcgAgcmCgaAcgAacAaa | 79 |
| dre-miR93/LNA | ctAccTgcAcaAacAgcActTt | 73 |
| dre-miR22/LNA | acaGttmCttmCagmCtgGcaGctt | 76 |
| dre-miR213/LNA | gGtamCagTcaAcgGtcGatGgt | 80 |
| dre-miR31/LNA | cagmCtaTgcmCaamCatmCttGcc | 76 |
| dre-miR189/LNA | amCtgTtaTcaGctmCagTagGcac | 75 |
| dre-miR18/LNA | tatmCtgmCacTaaAtgmCacmCtta | 69 |
| dre-miR15a/LNA | cAcaAacmCatTctGtgmCtgmCta | 74 |
| dre-miR34b/LNA | cAatmCagmCtaAcaAcamCtgmCcta | 74 |
| dre-miR148a/LNA | acaAagTtcTgtAatGcamCtga | 69 |
| dre-miR125a/LNA | camCagGttAagGgtmCtcAggGa | 80 |
| dre-miR139/LNA | agAcamCatGcamCtgTaga | 69 |
| dre-miR150/LNA | cacTggTacAagGatTggGaga | 75 |
| dre-miR192/LNA | ggcTgtmCaaTtcAtaGgtmCa | 73 |
| dre-miR98/LNA | aacAacAcaActTacTacmCtca | 68 |
| dre-let7g/LNA | amCtgTacAaamCaamCtamCctmCa | 73 |
| dre-miR30a-5p/ | gctTccAgtmCggGgaTgtTtamCa | 80 |
| LNA | | |
| dre-miR26b/LNA | aacmCtaTccTggAttActTgaa | 68 |
| dre-miR21/LNA | cAacAccAgtmCtgAtaAgcTa | 72 |
| dre-miR146/LNA | accmCttGgaAttmCagTtcTca | 72 |
| dre-miR182/LNA | tgtGagTtcTacmCatTgcmCaaa | 72 |
| dre-miR182*/LNA | taGttGgcAagTctAgaAcca | 72 |
| dre-miR220/LNA | aAgtGtcmCgaTacGgtTgtGg | 81 |
| hsa-miR138/LNA | gatTcamCaamCacmCagmCt | 70 |
| dre-miR141/LNA | gcaTcgTtamCcaGacAgtGtt | 74 |
| hsa-miR143/LNA | gagmCtamCagTgcTtcAtcTca | 72 |
| hsa-miR195/LNA | gmCcaAtaTttmCtgTgcTgcTa | 73 |
| dre-mir-30a-3p/ | acaGcaAacAtcmCaamCtgAaag | 72 |
| LNA | | |
| hsa-mir375/LNA | tAacGcgAgcmCgaAcgAacAaa | 79 |

LNA nucleotides are depicted by capital letters,
DNA nucleotides by lowercase letters,
mC denotes LNA methyl-cytosine.

Whole-Mount in situ Hybridizations

Whole-mount in situ hybridizations were performed essentially as described (B. Thisse et al., Methods Cell Biol 77, 505-19 (2004).), with the following modifications: Hybridization, washing and incubation steps were done in 2.0 ml eppendorf tubes. All PBS and SSC solutions contained 0.1% Tween (PBST and SSCT). Embryos of 12, 16, 24, 48, 72 and 120 hpf were treated with proteinase K for 2, 5, 10, 30, 45 and 90 min, respectively. After proteinase K treatment and refixation with 4% paraformaldehyde, endogenous alkaline phosphatase activity was blocked by incubation of the embryos in 0.1 M ethanolamine and 2.5% acetic anhydride for 10 min, followed by extensive washing with PBST. Hybridizations were performed in 200 μl of hybridization mix. The temperature of hybridization and subsequent washing steps was adjusted to approximately 22° C. below the predicted melting temperatures of the LNA-modified probes. Staining with NBT/BCIP was done overnight at 4° C. After staining, the embryos were fixed overnight in 4% paraformaldehyde. Next, embryos were dehydrated in an increasing methanol series and subsequently placed in a 2:1 mixture of benzyl benzoate and benzyl alcohol. Embryos were mounted on a hollow glass slide and covered with a coverslip.

Plastic Sectioning

Embryos and larvae stained by whole-mount in situ hybridization were transferred from benzyl benzoate/benzyl alcohol to 100% methanol and incubated for 10 min. Specimens were washed twice with 100% ethanol for 10 min and incubated overnight in 100% Technovit 8100 infiltration solution (Kulzer) at 4° C. Next, specimens were transferred to a mold and embedded overnight in Technovit 8100 embedding medium (Kulzer) deprived of air at 4° C. Sections of 7 μm thickness were cut with a microtome (Reichert-Jung 2050), stretched on water and mounted on glass slides. Sections were dried overnight. Counterstaining was done by 0.05% neutral red for 12 sec, followed by extensive washing with water. Sections were preserved with Pertex and mounted under a coverslip.

Image Acquisition

Embryos and larvae stained by whole-mount in situ hybridization were analyzed with Zeiss Axioplan and Leica MZFLIII microscopes and subsequently photographed with digital cameras. Sections were analyzed with a Nikon Eclipse E600 microscope and photographed with a digital camera (Nikon, DXM1200). Images were adjusted with Adobe Photoshop 7.0 software.

TABLE 2
MicroRNA expression patterns in zebrafish embryonic development
determined by whole mount in situ hybridization of embryos using
LNA-substituted miRNA detection probes.

| MicroRNA | Class* | In situ expression pattern in zebrafish |
|---|---|---|
| miR-1 | A | Body, head and fin muscles |
| miR-122a | A | Liver; pancreas |
| miR-124a | A | Differentiated cells of brain; spinal cord and eyes; cranial ganglia |
| miR-128a | A | Brain (specific neurons in fore- mid- and hindbrain); spinal cord; |
| | | cranial nerves/ganglia |
| miR-133a | A | Body, head and fin muscles |
| miR-138 | A | Outflow tract of the heart; brain; cranial nerves/ganglia; |
| | | undefin. bilateral structure in head; neurons in spinal cord |
| miR-144 | A | Blood |
| miR-194 | A | Gut and gall bladder; liver; pronephros |
| miR-206 | A | Body, head and fin muscles |
| miR-219 | A | Brain (mid- and hindbrain); spinal cord |
| miR-338 | A | Lateral line; cranial ganglia |
| miR-9 | A | Proliferating cells of brain, spinal cord and eyes |
| miR-9* | A | Proliferating cells of brain, spinal cord and eyes |
| miR-200a | A | Nose epithelium; lateral line organs; epidermis; gut |
| | | (proctodeum); taste buds |
| miR-132 | A | Brain (specific neurons in fore- and midbrain) |
| miR-142-5p | A | Thymic primordium |
| miR-7 | A | Neurons in forebrain; diencephalon/hypothalamus; pancreatic |
| | | islet |
| miR-143 | A | Gut and gall bladder; swimbladder; heart; nose |
| miR-145 | A | Gut and gall bladder; gills; swimbladder; branchial arches; fins; |
| | | outflow tract of the heart; ear |
| miR-181a | A | Brain (tectum, telencephalon); eyes; thymic primordium; gills |
| miR-181b | A | Brain (tectum, telencephalon); eyes; thymic primordium; gills |
| miR-215 | A | Gut and gall bladder |
| let-7a | A | Brain; spinal cord |
| let-7b | A | Brain; spinal cord |
| miR-125a | A | Brain; spinal cord; cranial ganglia |
| miR-125b | A | Brain; spinal cord; cranial ganglia |
| miR-142-3p | A | Thymic primordium; blood cells |
| miR-200b | A | Nose epithelium; lateral line organs; epidermis; gut |
| | | (proctodeum); taste buds |
| miR-218 | A | Brain (neurons and/or cranial nerves/ganglia in hindbrain); |
| | | spinal cord |
| miR-222 | A | Neurons and/or cranial ganglia in forebrain and midbrain; |
| | | rhombomere in early stages |
| miR-23a | A | Pharyngeal arches; oral cavity; posterior tail; cardiac valves |
| miR-27a | A | Undefined structures in branchial arches; tip of tail in early |
| | | stages |
| miR-34a | A | Brain (cerebellum); neurons in spinal cord |
| miR-375 | A | Pituitary gland; pancreatic islet |
| miR-99a | A | Brain (hindbrain, diencephalon); spinal cord |
| let-7i | A | Brain (tectum, diencephalon) |
| miR-100 | A | Brain (hindbrain, diencephalon); spinal cord |
| miR-103 | A | Brain; spinal cord |
| miR-107 | A | Brain; spinal cord |
| miR-126 | A | Bloodvessels and heart |
| miR-137 | A | Brain (neurons and/or cranial nerves/ganglia in fore-, mid- and |
| | | hindbrain); spinal cord |
| miR-140 | A | Cartilage of pharyngeal arches, head skeleton and fins |
| miR-140* | A | Cartilage of pharyngeal arches, head skeleton and fins |
| miR-141 | A | Nose epithelium; lateral line organs; epidermis; gut |
| | | (proctodeum); taste buds |
| miR-150 | A | Cardiac valves; undefined structures in epithelium of branchial |
| | | arches |
| miR-182 | A | Nose epithelium; haircells of lateral line organs and ear; cranial |
| | | ganglia; rods, cones and bipolar cells of eye; epiphysis |
| miR-183 | A | Nose epithelium; haircells of lateral line organs and ear; cranial |
| | | ganglia; rods, cones and bipolar cells of eye; epiphysis |
| miR-184 | A | Lens; hatching gland in early stages |
| miR-199a | A | Epithelia surrounding cartilage of pharyngeal arches, oral cavity |
| | | and pectoral fins; epidermis of head; tailbud |
| miR-199a* | A | Epithelia surrounding cartilage of pharyngeal arches, oral cavity |
| | | and pectoral fins; epidermis of head; tailbud |
| miR-203 | A | Most outer layer of epidermis |
| miR-204 | A | Neural crest; pigment cells of skin and eye; swimbladder |
| miR-205 | A | Epidermis; epithelia of branchial arches; intersegmental cells; |
| | | not in sensory epithelia |
| miR-221 | A | Brain (Neurons and/or cranial ganglia in forebrain and midbrain; |
| | | rhombomere in early stages) |
| miR-7b | A | Brain (fore-, mid- and hindbrain); spinal cord |
| miR-96 | A | Nose epithelium; haircells of lateral line organs and ear; cranial |
| | | ganglia; rods, cones and bipolar cells of eye; epiphysis |
| miR-217 | B | Brain (tectum, hindbrain); spinal cord; proliferative cells of eyes; |
| | | pancreas |
| miR-126* | B | ND |
| miR-31 | B | Ubiquitous |
| miR-216 | B | Brain (tectum); spinal cord; proliferative cells of eyes; pancreas; |
| | | body muscles |
| miR-30a-5p | B | Pronephros; cells in epidermis; lens in early stages |
| miR-153 | B | Brain (fore- mid- and hindbrain, diencephalon/hypothalamus) |
| miR-15a | C | Ubiquitous (head, spinal cord, gut, outline somites, neuromasts) |
| miR-17-5p | C | Ubiquitous (head, spinal cord, gut, outline somites, neuromasts) |
| miR-18 | C | Ubiquitous (head, spinal cord, gut, outline somites, neuromasts) |
| miR-195 | C | Ubiquitous |
| miR-19b | C | Ubiquitous (head, spinal cord, gut, outline somites, neuromasts) |
| miR-20 | C | Ubiquitous (head, spinal cord, gut, outline somites, neuromasts) |
| miR-26a | C | Ubiquitous (head, spinal cord, gut, outline somites, neuromasts) |
| miR-92 | C | Ubiquitous (head, spinal cord, gut, outline somites, neuromasts) |
| let-7c | C | Brain; spinal cord |
| miR-101 | C | ND |
| miR-16 | C | Brain |
| miR-21 | C | Cardiac valves; otoliths in ear; rhombomere in early stages |
| miR-30b | C | Pronephros; cells in epidermis |
| miR-30c | C | Pronephros; cells in epidermis and epithelia of branchial arches; |
| | | neurons in hindbrain |
| miR-26b | C | Ubiquitous (head, spinal cord, gut, outline somites, neuromasts) |
| let-7g | C | Ubiquitous (head, spinal cord, gut, outline somites, neuromasts) |
| miR-19a | C | Ubiquitous (head, spinal cord, gut, outline somites, neuromasts) |
| miR-210 | C | Ubiquitous (head, spinal cord, gut, outline somites, neuromasts) |
| miR-22 | C | Ubiquitous |
| miR-25 | C | Ubiquitous (head, spinal cord, gut, outline somites, neuromasts) |
| miR-93 | C | Ubiquitous (head, spinal cord, gut, outline somites, neuromasts) |
| miR-189 | D | ND |
| miR-30a-3p | D | ND |
| miR-34b | D | Cells in pronephric duct; nose |
| miR-129* | D | ND |
| miR-135a | D | ND |
| miR-182* | D | ND |
| miR-187 | D | ND |
| miR-220 | D | ND |
| miR-301 | D | ND |
| miR-223 | D | ND |
| let-7f | — | Brain; spinal cord |
| miR-108 | — | Ubiquitous |
| miR-10a | — | Posterior trunk; later restricted to spinal cord |
| miR-10b | — | Posterior trunk; later restricted to spinal cord |
| miR-129 | — | Brain |
| miR-130a | — | ND |
| miR-139 | — | Nose; neuromasts |
| miR-146 | — | Neurons in forebrain; branchial arches and head skeletion |
| miR-148a | — | ND |
| miR-152 | — | Ubiquitous |
| miR-155 | — | ND |
| miR-190 | — | ND |
| miR-193 | — | ND |
| miR-196a | — | Posterior trunk; later restricted to spinal cord |
| miR-213 | — | Nose (epithelium or olfactory neurons), eyes (ganglion cell layer) |
| miR-214 | — | Epithelia surrounding cartilage of pharyngeal arches, oral cavity |
| | | and pectoral fins; epidermis of head; tailbud |
| miR-24 | — | Pharyngeal arches; oral cavity; posterior tail; cardiac valves |
| miR-27b | — | Cells in branchial arches |
| miR-29a | — | ND |
| miR-29b | — | ND |
| miR-29c | — | ND |
| miR-98 | — | Brain |

*Main class in which expression patterns were compared: A, specific expression; B, marginal specific expression or very low absolute expression; C, ubiquitous expression. D, no detectable expression.

Wienholds et al., Science, 2005, 309, 310-311 (published after the effective date of the data above) relates to the findings referred to in Table 2—that reference also includes a number of figures which visually demonstrates the tissue distribution of a number of miRNAs. Wienholds et al. is consequently incorporated by reference herein.

TABLE 3
List of LNA-substituted detection probes
useful as specificity controls in detection
of vertebrate microRNAs.

| | | Self- |
|---|---|---|
| | | comp |
| Probe name | Sequence 5′-3′ | score |
| hsa-miR206/ | ccamCacActmCtcTtamCatTcca | 8 |
| LNA/2MM | | |
| hsa-miR206/ | ccamCacActmCccTtamCatTcca | 8 |
| LNA/1MM | | |
| hsa-miR124a/ | tggmCatTcaAagmCgtGccTtaa | 60 |
| LNA/2MM | | |
| hsa-miR124a/ | tggmCatTcaAcgmCgtGccTtaa | 60 |
| LNA/1MM | | |
| hsa-miR122a/ | acAaamCacmCacmCgtmCacActmCca | 18 |
| LNA/2MM | | |
| hsa-miR122a/ | acAaamCacmCatmCgtmCacActmCca | 18 |
| LNA/1MM | | |

LNA nucleotides are depicted by capital letters,
DNA nucleotides by lowercase letters,
mC denotes LNA methyl-cytosine.

The above demonstrates that it is possible to map an animal's miRNA against various tissues, and it is thus possible to determine the origin of a cell based on a determination of miRNA from said cell.

This has interesting implications. As mentioned above, it is a known clinical problem to determine the exact origin of a number of metastatic cancers and this has several consequences. First of all, it is not possible to locate the primary tumour (which may be much smaller than the metastatic tumour which has been detected), but it is in such cases also difficult if not impossible to determine the optimum treatment because of lack of knowledge of the tissue origin of the primary tumour.

Cancer of unknown primary site is a common clinical entity, accounting for 2% of all cancer diagnoses in the Surviellance, Epidemiology, and End Results (SEER) registries between 1973 and 1987 (C. Muir. Cancer of unknown primary site Cancer 1995. 75: 353-356). In spite of the frequency of this syndrome, relatively little attention has been given to this group of patients, and systematic study of the entity has lagged behind that of other areas in oncology. Widespread pessimism concerning the therapy and prognosis of these patients has been the major reason for the lack of effort in this area. The patient with carcinoma of unknown primary site is commonly stereotyped as an elderly, debilitated individual with metastases at multiple visceral sites. Early attempts at systemic therapy yielded low response rates and had a negligible effect on survival, thereby strengthening arguments for a nihilistic approach to these patients. The heterogeneity of this group has also made the design of therapeutic studies difficult; it is well recognized that cancers with different biologies from many primary sites are represented. In the past 10 years, substantial improvements have been made in the management and treatment of some patients with carcinoma of unknown primary site. The identification of treatable patients within this heterogeneous group has been made possible by the recognition of several clinical syndromes that predict chemotherapy responsiveness, and also by the development of specialized pathologic techniques that can aid in tumor characterization. Therefore, the optimal management of patients with cancer of unknown primary site now requires appropriate clinical and pathologic evaluation to identify treatable subgroups, followed by the administration of specific therapy. Many patients with adenocarcinoma of unknown primary site have widespread metastases and poor performance status at the time of diagnosis. The outlook for most of these patients is poor, with median survival of 4 to 6 months. However, subsets of patients with a much more favorable outlook are contained within this large group, and optimal initial evaluation enables the identification of these treatable subsets. In addition, empiric chemotherapy incorporating newer agents has produced higher response rates and probably improves the survival of patients with good performance status.

Fine-needle aspiration biopsy (FNA) provides adequate amounts of tissue for definitive diagnosis of poorly differentiated tumors, and identification of the primary source in about one fourth of cases (C. V. Reyes, K. S. Thompson, J. D. Jensen, and A. M. Chouelhury. Metastasis of unknown origin: the role of fine needle aspiration cytology Diagn Cytopathol 1998. 18: 319-322).

As one example, most patients with squamous cell carcinoma involving inguinal lymph nodes have a detectable primary site in the genital or anorectal area. In women, careful examination of the vulva, vagina, and cervix is important, with biopsy of any suspicious areas. Men should undergo a careful inspection of the penis. Digital examination and anoscopy should be performed in both sexes to exclude lesions in the anorectal area. Identification of a primary site in these patients is important, since curative therapy is available for carcinomas of the vulva, vagina, cervix, and anus even after they spread to regional lymph nodes. For the occasional patient in whom no primary site is identified, surgical resection with or without radiation therapy to the inguinal area sometimes results in long-term survival (A. Guarischi, T. J. Keane, and T. Elhakim. Metastatic inguinal nodes from an unknown primary neoplasm. A review of 56 cases Cancer 1987. 59: 572-577). Hence, clearly it is advantageous to be able to determine the origin of tumors and improved recognition of treatable subsets within the large heterogeneous population of patients with carcinoma of unknown primary site would represents a definite advance in the management and treatment of these patients. This will also allow treatable subsets to be defined with appropriate clinical and pathologic evaluation; Table X provides a summary of currently known subsets of carcinomas of unknown origin and outlines the recommended evaluation and treatment thereof. Clearly, identifying the primary site in cases of metastatic carcinoma of unknown origin has profound clinical importance in managing cancer patients. Currently, identification of the site of origin of a metastatic carcinoma is time consuming and often requires expensive whole-body imaging or invasive exploratory surgery.

TABLE X

| | Clinical Evaluation (in addition | | | |
|---|---|---|---|---|
| | to history, Physical exam, routine | | Specific Subsets for | |
| Histopathology | laboratory, chest radiography) | Special Pathologic Studies | Therapy | Therapy |
| Adenocarcinoma | CT scan of abdomen | Men: PSA stain | 1) Women, axillary node | Treat as primary breast cancer |
| (well- | Men: serum PSA | Women: ER, PR stain | involvement | |
| differentiated | Women: Mammograms | | | |
| or | | | 2) Women, peritoneal | Treat as stage III prostate cancer |
| moderately | | | carcinomatosis | |
| differentiated) | Additional studies to | | 3) Men, blastic bone | Treat as stage IV prostate cancer |
| | evaluate signs, symptoms | | metastases, or high serum | |
| | | | PSA or tumor PSA | |
| | | | staining | |
| | | | 4) Solitary metastatic | Definitive local therapy |
| | | | lesion | |
| Squamous | Cervical presentation: Direct | — | Cervical adenopathy | Treat as locally advanced |
| carcinoma | laryngoscopy, nasopharyngoscopy, | | | head/neck cancer |
| | bronchoscopy | | Inguinal adenopathy | Inguinal LND ± radiation therapy |
| Poorly | CT abdomen, chest Serum, | Immunoperoxidase staining, | 1) Features of EGCT | Treat as nonseminomatous ECGT |
| differentiated | HCG, AFP | electron microscopy, | | |
| carcinoma | Additional studies to | cytogenetic studies | | |
| | evaluate signs, symptoms | | 2) Other patients | Empiric platinum or |
| | | | | paclitaxel/platinum regimen |
| Neuroendocrine | CT abdomen, chest Additional | Immunoperoxidase staining | 1) Low grade | Treat as advanced carcinoid |
| carcinoma | studies to evaluate signs, | | | tumor |
| | symptoms | | 2) Small cell carcinoma | Empiric platinum/etoposide or |
| | | | | platinum/etoposide/paclitaxel |
| | | | 3) Poorly differentiated | |

CT = computed tomography;
PSA = prostate-specific antigen;
HCG = human chorionic gonadotropin;
AFP = alpha-fetoprotein;
ER = estrogen receptor,
PR = progesterone receptor;
EGCT = extragonadal germcell tumor,
LND = lymph node dissection.

As previously described, microRNAs have emerged as important non-coding RNAs, involved in a wide variety of regulatory functions during cell growth, development and differentiation. Some reports clearly indicate that microRNA expression may be indicative of cell differentiation state, which again is an indication of organ o tissue specification. This finding has been confirmed in the experiments using LNA FISH probes on whole mount preparations in different developmental stages in zebra fish, where a large number of microRNAs display a very distinct tissue or organ-specific distribution. As outlined in the figures herein and in summary in table 2 many microRNAs are expressed only in single organs or tissues. For example, mir-122a is expressed primarily in liver and pancreas, mir-215 is expressed primarily in gut and gall bladder, mir-204 is primarily expressed in the neural crest, in pigment cells of skin and eye and in the swimbladder, mir-142-5p in the thymic primordium etc. This catalogue of mir tissue expression profiles may serve as the basis for a diagnostic tool determining the tissue origin of tumors of unknown origin. If, for example a tumour sample from a given sample expresses a microRNA pattern typical of another tissue type, this may be predictive of the tumour origin. For example, if a lymph cancer type expresses microRNA markers characteristic of liver cells (eg. Mir-122a), this may be indicative that the primary tumour resides within the liver. Hence, the detailed microRNA expression pattern in zebrafish provided may serve as the basis for a diagnostic measurement of clinical tumour samples providing valuable information about tumour origin.

So, since it is possible to map miRNA in cells vs. the tissue origin of these cells, the present invention presents a convenient means for detection of tissue origin of such tumours.

Hence, the present invention in general relates to a method for determining tissue origin of tumours comprising probing cells of the tumour with a collection of probes which is capable of mapping miRNA to a tissue origin.

### Example 13A

Generation of miRNA expression profiles for colon cancer samples by applying the LNA array technology.

Experimential Design:

A glandular metastasis (GM) originated from a primary colon cancer and a corresponding normal jejunum (NJ) biopsy from the same patient was available. Furthermore two control total RNA samples (Human colon (Lot #055P011102051E, Cat #7986) and lymph node (Lot #105P010605002A, Cat #7894) obtained from Ambion, Tex.) were included. All samples were one by one hybridized in competition with a common human total RNA pool and applied to the miRCURY™ LNA array microarray kit (Exiqon, Denmark).

Total RNA from the GM and NJ biopsies were purified at Copenhagen County Hospital in Herlev using standard extraction procedures. The quality of the total RNA was verified by an Agilent 2100 Bioanalyzer profile.

The test samples were labeled with Hy3™ fluorescent label (Exiqon, Denmark) using 2 μg total RNA following the procedure described in the miRCURY™ LNA Array labeling kit protocol. The human tissue (HT) total RNA pool consists of total RNA from 25 different human tissues (all samples were purchased from Ambion, Tex.). For each of the test samples, 2 μg human total RNA pool was labeled with Hy5™ fluorescent label (Exiqon, Denmark) according to the manufacturer's recommendations. A Hy3™-labeled test sample and a Hy5™-labeled human pool sample were mixed and applied to the miRCURY™ LNA array. The hybridization was performed in a Tecan HS400/HS4800 hybridization station according to the miRCURY™ LNA array microarray kit manual. The miRCURY™ LNA array microarray slides were scanned by a ScanArray 4000 XL scanner (Packard Biochip Technologies, USA) and the image analysis was carried out using the ImaGene 6.1.0 software (BioDiscovery, Inc., USA).

Design Layout:

| | | Slide | | |
|---|---|---|---|---|
| | Array | batch | Hy3 ™ | |
| Array name | barcode | no. | channel | Hy5 ™ channel |
| GM | 13452608 | 1010 | GM total RNA | HT-pool total RNA |
| NJ | 13452609 | 1010 | NJ total RNA | HT-pool total RNA |
| Colon | 13452610 | 1010 | Colon total RNA | HT-pool total RNA |
| Lymph node | 13457690 | 1010 | Lymph node | HT-pool total RNA |
| | | | total RNA | |

Results:

The quality of the logarithm-transformed raw intensities from the microarray slides was assessed using different diagnostic plots (histograms, MA-plots and scatter plots). FIG. 12 shows the graphs of the intensities before and after global Lowess normalization. The distribution of the log-transformed raw intensities from the GM sample showed a bimodal distribution, however, after normalization only one peak was observed (FIG. 12A).

The sum raw intensities from the Hy5™ signals imply a slide-to-slide variation. This variation could be due to time-dependent ozone exposure causing fading of the Hy5™ dye before scanning. Therefore, it is problematic to base the data analysis on the ratios of Hy3™/Hy5™ intensities as these would depend on the variable Hy5™ signals, thus under optimal conditions should be close to constant. In order to avoid this problem the Hy3™ intensities were treated as absolute intensities similar to single sample hybridization. The Hy3™ intensities were median-scaled before comparison across microarrays.

Discussion:

In order to find the relationship between the samples one-way hierarchical clustering was applied based on Pearson correlation coefficient and centroid linkage method. Also the sum of squared distances (SSQ) was calculated pair-wise for all miRNA across the microarray (FIG. 13). The distances between the samples were illustrated in a Principal Components Analysis (PCA) plot (FIG. 14).

Conclusion:

Both the hierarchical clustering and the PCA plot show that the GM miRNA expression profile is closest to the Colon expression profile. The distance to the NJ expression profile for the GM profile was farther away. Lastly, the expression profiles between the GM and Lymph node provided the biggest distance. In general the Lymph node expression profile was not close to any of the other samples. These findings underscore the principles discussed in Example 13, namely that it is possible to determine the origin of a tumour based on its miRNA expression profile.

miRNA typing according to the principles of the present example can be applied to RNA from a variety of normal tissues and tumour tissues (of known origin) and over time a database is build up, which consists of miRNA expression profiles from normal and/or tumour tissue and/or specifically metastatic tumors.

When subjecting RNA from a tumour tissue sample, the resulting miRNA profile can be analysed for its degree of identity with each of the profiles of the database—the closest matching profiles are those having the highest likelyhood of representing a tumour having the same origin (but also other characteristics of clinical significance, such as degree of malignancy, prognosis, optimum treatment regimen and predictition of treatment success). The miRNA profile may of course be combined with other tumour origin determination techniques, cf. e.g. Xiao-Jun Ma et al., Arch Pathol Lab Med 130, 465-473, which demonstrates molecular classification of human cancers into 39 tumour classes using a microarray designed to detect RT-PCR amplified mRNA derived from expression of 92 tumor-related genes. The presently presented technology allows for an approach which is equivalent safe for the use of a miRNA detection assay instead of an mRNA detection assay.

### Example 14

Detection of microRNAs by In Situ Hybridization in Paraffin-Embedded Mouse Brain Sections Using 3′ Digoxigenin-Labeled LNA Probe

A. Deparaffinization of the Sections

(i) xylene 3×5 min, (ii) ethanol 100% for 2×5 min, ethanol 70% for 5 min, ethanol 50% for 5 min, ethanol 25% for 5 min and in DEPC-treated water for 1 min.

B. Deproteinization of Sections

(i) 2×5 min in PBS; 5 min in Proteinase K at 10 ug/ml at 37° C. (add Prot. K 20 mg/ml to warm Prot. K buffer 20 min before incubation); 30 sec in 0.2% Glycine in PBS and 2×30 sec in PBS.

C. Fixation

Sections were fixed for 10 min in 4% PFA, and the slides rinsed 2× in PBS

D. Prehybridization

Prehybridization was carried out for 2 hours at the final hybridization temperature (ca 22 degrees below the predicted Tm of the LNA probe) in hybridization buffer (50% Formamide, 5×SSC, 0.1% Tween, 9.2 mM citric acid for adjustment to pH6, 50 ug/ml heparin, 500 ug/ml yeast RNA) in a humidified chamber (50% formamide, 5×SSC). Use DAKO Pen.

E. Hybridization

The 3′ DIG-labeled LNA probe was diluted to 20 nM in hybridization buffer and 200 ul of hybridization mixture was added per slide. The slides were hybridized overnight covered with Nescofilm in a humidified chamber. The slides were rinsed in 2×SCC and then washed at hybridization temperature 3 times 30 min in 50% formamide, 2xSSC, and finally 5×5 min in PBST at room temperature.

F. Immunological Detection

The slides were blocked for 1 hour in blocking buffer (2% sheep serum, 2 mg/ml BSA in PBST) at room temperature, incubated overnight with anti-DIG antibody (1:2000 anti-DIG-AP Fab fragments in blockingbuffer) in a humidified chamber at 4° C., washed 5-7 times 5 min in PBST and 3 times 5 min in AP buffer (see below).

G. Colour Reaction (Room Temperature, in Dark)

The light-sensitive colour reaction (NBT/BCIP) was carried out for 1 h-48 h (400 ul/slide) in a humidified chamber; the slides were washed for 3×5 min in PBST, and mounted in aqeous mounting medium (glycerol) or dehydrate and mount in Entellan.

The results are shown in FIGS. 5 and 6. It surprisingly appears that it is possible to detect target nucleotide sequences in these paraffin embedded sections. Previously it has been noted that it is very difficult to utilise fixated and embedded sections for hybridization assays. This is due to a variety of factor: First of all, RNA is degraded over time, so the use of long hybridization probes to detect RNA becomes increaingly difficult over time. Secondly, the very structure of a fixated and embedded section is such that it appears to be diffucult for hybridization probes to contact their target sequences.

Without being limited to any theory, it is believed that the short hybridization probes of the present invention overcome these disadvantages by being able to diffuse readily in a fixated and embedded section and by being able to hybridize with short fragments of degraded RNA still present in the section.

It should be noted that the present finding also opens for the possibility of detecting DNA in archived fixated and embedded samples. It is then e.g. possible, when using the short but highly specific probes of the present invention, to detect e.g. viral DNA in such aged samples, a possibility which to the best of the inventors' knowledge has not been available prior to the findings in the present invention.

H. Buffers Used in Example 14.

H1. AP Buffer

100 ml Tris (100 mM) 12.1 g/l

20 ml 5M NaCl (100 mM) 5.84 g/l

5 ml 1M MgCl2 (5 mM)

700 ml sterile H2O, pH 9.5 and fill up to 1 liter

H2. Colour Solution (Light Sensitive)

45 ul 75 mg/ml NBT (in 70% dimethylformamide)

35 ul 50 mg/ml BCIP-phosphate (in 100% dimethylformamide)

2.4 mg Levamisole

in 10 ml AP buffer.

### Example 15

Specificity and Sensitivity Assessment of microRNA Detection in Zebrafish, Xenopus laevis and Mouse by Whole Mount In Situ Hybridization of Embryos Using LNA-Substituted miRNA Detection Probes

Experimental Material

Zebrafish, mouse and Xenopus tropicalis were kept under standard conditions. For all in situ hybridizations on zebrafish we used 72 hour old homozygous albino embryos. For Xenopus tropicalis 3 day old embryos were used and for mouse we used 9.5 or 10.5 dpc embryos.

Design and Synthesis of LNA-Modified Oligonucleotide Probes

The LNA-modified DNA oligonucleotide probes are listed in Table 15-I. LNA probes were labeled with digoxigenin-ddUTP using the 3′-end labeling kit (Roche) according to the manufacturers recommendations and purified using sephadex G25 MicroSpin columns (Amersham).

TABLE 15-I
List of short LNA-substituted detection probes
for detection of microRNA expression in zebrafish
by whole mount in situ hybridization of embryos

| | | Calc |
|---|---|---|
| Probe name | Sequence 5′-3′ | Tm |
| hsa-miR124a/LNA | tggmCatTcamCcgmCgtGccTtaa | 80 |
| hsa-miR124a/LNA-2 | gmCatTcamCcgmCgtGccTtaa | 78 |
| hsa-miR124a/LNA-4 | atTcamCcgmCgtGccTtaa | 72 |
| hsa-miR124a/LNA-6 | TcamCcgmCgtGccTtaa | 71 |
| hsa-miR124a/LNA-8 | amCcgmCgtGccTtaa | 70 |
| hsa-miR124a/LNA-10 | cgmCgtGccTtaa | 60 |
| hsa-miR124a/LNA-12 | mCgtGccTtaa | 46 |
| hsa-miR124a/LNA-14 | tGccTtaa | 27 |
| hsa-miR206/LNA | ccamCacActTccTtamCatTcca | 73 |
| hsa-miR206/LNA-2 | amCacActTccTtamCatTcca | 70 |
| hsa-miR206/LNA-4 | acActTccTtamCatTcca | 64 |
| hsa-miR206/LNA-6 | ActTccTtamCatTcca | 58 |
| hsa-miR206/LNA-8 | tTccTtamCatTcca | 55 |
| hsa-miR206/LNA-10 | ccTtamCatTcca | 49 |
| hsa-miR206/LNA-12 | TtamCatTcca | 35 |
| hsa-miR206/LNA-14 | amCatTcca | 32 |
| hsa-miR124a/ | amCcgmCgtAccTtaa | 70 |
| LNA-8/MM | | |
| hsa-miR206/LNA-8/MM | tTccTtaAatTcca | 55 |

LNA nucleotides are depicted by capital letters,
DNA nucleotides by lowercase letters,
mC denotes LNA methyl-cytosine.

Whole Mount In Situ Hybridizations

All washing and incubation steps were performed in 2 ml eppendorf tubes. Embryos were fixed overnight at 4° C. in 4% paraformaldehyde in PBS and subsequently transferred through a graded series (25% MeOH in PBST (PBS containing 0.1% Tween-20), 50% MeOH in PBST, 75% MeOH in PBST) to 100% methanol and stored at −20° C. up to several months. At the first day of the in situ hybridization embryos were rehydrated by successive incubations for 5 min in 75% MeOH in PBST, 50% MeOH in PBST, 25% MeOH in PBST and 100% PBST (4×5 min). Fish, mouse and Xenopus embryos were treated with proteinaseK (10 μg/ml in PBST) for 45 min at 37° C., refixed for 20 min in 4% paraformaldehyde in PBS and washed 3×5 min with PBST. After a short wash in water, endogenous alkaline phosphatase activity was blocked by incubation-of the embryos in 0.1 M tri-ethanolamine and 2.5% acetic anhydride for 10 min, followed by a short wash in water and 5×5 min washing in PBST. The embryos were then transferred to hybridization buffer (50% Formamide, 5×SSC, 0.1% Tween, 9.2 mM citric acid, 50 ug/ml heparin, 500 ug/ml yeast RNA) for 2-3 hour at the hybridization temperature. Hybridization was performed in fresh pre-heated hybridization buffer containing 10 nM of labeled LNA probe. Post-hybridization washes were done at the hybridization temperature by successive incubations for 15 min in HM—(hybridization buffer without heparin and yeast RNA), 75% HM-/25% 2×SSCT (SSC containing 0.1% Tween-20), 50% HM-/50% 2×SSCT, 25% HM-/75% 2×SSCT, 100% 2×SSCT and 2×30 min in 0.2×SSCT. Subsequently, embryos were transferred to PBST through successive incubations for 10 min in 75% 0.2×SSCT/25% PBST, 50% 0.2×SSCT/50% PBST, 25% 0.2×SSCT/75% PBST and 100% PBST. After blocking for 1 hour in blocking buffer (2% sheep serum/2 mg:ml BSA in PBST), the embryos were incubated overnight at 4° C. in blocking buffer containing anti-DIG-AP FAB fragments (Roche, 1/2000). The next day, zebrafish embryos were washed 6×15 min in PBST, mouse and X. tropicalis embryos were washed 6×1 hour in TBST containing 2 mM levamisole and then for 2 days at 4° C. with regular refreshment of the wash buffer. After the post-antibody washes, the embryos were washed 3×5 min in staining buffer (100 mM tris HCl pH9.5, 50 mM MgCl2, 100 mM NaCl, 0.1% tween 20). Staining was done in buffer supplied with 4.5 μl/ml NBT (Roche, 50 mg/ml stock) and 3.5 μl/ml BCIP (Roche, 50 mg/ml stock). The reaction was stopped with 1 mM EDTA in PBST and the embryos were stored at 4° C. The embryos were mounted in Murray's solution (2:1 benzylbenzoate:benzylalcohol) via an increasing methanol series (25% MeOH in PBST, 50% MeOH in PBST, 75% MeOH in PBST, 100% MeOH) prior to imaging.

Image Acquisition

Embryos and larvae stained by whole-mount in situ hybridization were analyzed with Zeiss Axioplan and Leica MZFLIII microscopes and subsequently photographed with digital cameras. Sections were analyzed with a Nikon Eclipse E600 microscope and photographed with a digital camera (Nikon, DXM1200). Images were adjusted with Adobe Photoshop 7.0 software.

Results

We first compared the ability of LNA-modified DNA probes to detect miR-206, miR-124a and miR-122a in 72 h zebrafish embryos with unmodified DNA probes of identical length and sequence. These three miRNAs are strongly expressed in the muscles, central nervous system and liver respectively. Both probe types could be easily labeled with digoxigenin (DIG) using standard 3′ end labeling procedures. Labeling efficiency was checked by dot-blot analysis. Equal labeling was obtained for both LNA-modified and unmodified DNA probes (FIG. 7a). As depicted in FIG. 7b, expected signals were obtained for all three miRNAs when LNA-modified probes were used for hybridization. In contrast, no such expression patterns could be seen with corresponding DNA probes under the same hybridization conditions. Lowering of the hybridization temperature resulted in high background signals for all three DNA probes Similar experiments to detect miRNAs in fish embryos using in vitro synthesized RNA probes, that carried a concatamer against the mature miRNA, were also unsuccessful. These results indicate that LNA-modified probes are well suited for sensitive in situ detection of miRNAs

Determination of the Optimal Hybridization Temperature for LNA-Modified Probes

The introduction of LNA modifications in a DNA oligonucleotide probe increases the Tm value against complementary RNA with 2-10° C. per LNA monomer. Since the Tm values of LNA-modified probes can be calculated using a thermodynamic nearest neighbor model 35 we decided to determine the optimal hybridization temperature for detecting miRNAs in zebrafish using LNA-modified probes, in relation to their Tm values (Table 15-I). The probes for miR-122a (liver specific) and miR-206 (muscle specific) have a calculated Tm value of 78° C. and 73° C. respectively. For miR-122a an optimal signal was obtained at a hybridization temperature of 58° C. and the probe for miR-206 gave the best signal at a temperature of 54° C. (FIG. 8a). A decrease or an increase in the hybridization temperature results in either higher background staining or complete loss of the hybridization signal. Thus, optimal results are obtained with hybridization temperatures of ˜21-22° C. below the predicted Tm value of the LNA probe.

Apart from adjusting the hybridization temperature, standard in situ procedures also make use of higher formamide concentrations to increase the hybridization stringency. We used a formamide concentration of 50% and did not investigate the effects of formamide concentration on LNA-based miRNA in situ detection further, as the hybridization temperatures were in a convenient range.

Determination of the Optimal Hybridization Time for LNA-Modified Probes

The standard zebrafish in situ protocol requires overnight hybridization. This may be necessary for long riboprobes used for mRNA in situ hybridization. We investigated the optimal hybridization time for LNA-based miRNA in situ hybridization. Significant in situ staining was obtained even after ten minutes of hybridization for miR-122a and miR-206 in 72 hour fish embryos (FIG. 8b). After one hour of hybridization the signal strength was comparable to the staining obtained after an overnight hybridization. This indicates that the hybridization times can be easily shortened for in situs using LNA probes, which would reduce the overall miRNA in situ protocol for zebrafish from three to two days.

Determination of the Specificity of LNA-Modified Probes

Many miRNAs belong to miRNA families. Some of the family members differ by one or two bases only, e.g. let-7c and let-7e (two mismatches) or miR-10a and miR-10b (one mismatch) and it might be that these do not have identical expression patterns. Indeed, from recent work it is clear that let-7c and let-7e have different expression patterns-in the limb buds of the early mouse embryo. To examine the specificity of LNA-modified probes we set out to perform in situ hybridizations with single and double mismatched probes for miR-124a, miR-206 and miR-122a (Table 15-I) under the same hybridization conditions as the fully complementary probe (FIG. 9). For miR-122a and miR-206 specific staining was lost upon introduction of a single central mismatch in the LNA probe. For the miR-124a probe two central mismatches were needed for adequate discrimination. These data demonstrate the high specificity of LNA-based miRNA in situ hybridization.

To investigate if the in situ signal is fully coming from mature miRNAs or also from precursors, we designed probes against star and loop sequences of miR-183 and miR-217. miR-183 is specific for the haircells of the lateral line organ and the ear, rods and cones and bipolar cells in the eye and sensory epithelia in the nose, while miR-217 is specific for the exocrine pancreas. We could not detect any pattern with probes against star and loop sequences for these miRNAs, suggesting that LNA-modified probes mainly detect mature miRNAs.

Reduction of the LNA Probe Length

In our initial in situ miRNA detection experiments, we used LNA-modified probes complementary to the complete mature miRNA sequence. Next, we decided to determine the minimal probe length, by which it would still be possible to get specific staining. Therefore, we systematically shortened the probes against miR-124a and miR-206 and performed in situ hybridization on 72 h zebrafish embryos with hybridization temperatures adjusted to 21° C. below the Tm value of the shortened probes. We could specifically detect miR-206 and miR-124a with shortened versions of the LNA probes complementary to a 12-nt region at the 5′-end of the miRNA (FIG. 10). In situ staining was virtually lost when 10-nt or 8-nt probes were used, although the 10-nt miR-124a probe gave a weak hybridization signal in the brain.

We expect that shorter LNA probes would exhibit significantly enhanced mismatch discrimination. As described above, in the case of miR-124a a single mismatch in a 22-mer LNA-modified probe was not sufficient for adequate discrimination. We thus tested single mismatch versions of the 14-mer LNA probes for miR-206 and miR-124a and found that in both cases the hybridization signal was completely lost (FIG. 10).

Detection of miRNAs in Xenopus laevis and Mouse Embryos

Thus far, we have reported the use of LNA probes for the detection of miRNAs only in the zebrafish embryo. To explore the usefulness of the LNA probe technology for detection of miRNAs in other organisms, we performed whole mount in situ hybridization on mouse and Xenopus tropicalis embryos with probes for miR-124a and miR-1, both of which are known to be abundant and tissue specific miRNAs (FIG. 11a and b). miR-124a was specific for tissues of the central nervous system in both organisms. miR-1 was expressed in the body wall muscles and the muscles of the head in Xenopus. In mouse, miR-1 was mainly expressed in the somitic muscles and the heart. These data are in agreement with the expression patterns in zebrafish and with expression studies based on dissected tissues from mouse, which show that miR-124a is brain specific and miR-1 is a muscle specific miRNA. Recently, a LacZ fusion construct of miR-1 also demonstrated that miR-1 is expressed in the heart and the somites of the early mouse embryo.

Next, we decided to determine the whole mount expression patterns in mouse embryos for miR-1, miR-206, miR-17, miR-20, miR-124a, miR-9, miR-126, miR-219, miR-196a, miR-10b and miR-10a, where the patterns were similar to what we previously observed in the zebrafish. In addition, miR-10a and miR-196a were found to be active in the posterior trunk in mouse embryos as visualized by miRNA-responsive sensors and we also found these miRNAs to be expressed in the same regions. For miR-182, miR-96, miR-183 and miR-125b the expression patterns were different compared to zebrafish. miR-182, miR-96 and miR-183 are expressed in the cranial and dorsal root ganglia. In zebrafish the same miRNAs show expression in the haircells of the lateral line neuromasts and the inner ear but also in the cranial ganglia. miR-125b is expressed at the midbrain hindbrain boundary in the early mouse embryo, whereas in zebrafish this miRNA is expressed in the brain and spinal cord.

### Example 16

Detection of Primary Site of Head and Neck Cancer

Head and Neck Cancer Assay

RNA Extraction (Trizol Protocol)

Before use, all samples were kept at −80° C.

Two samples—ca. 100 mg of each—were used for RNA extraction:

PT (primary tumor)

1 C (normal adjacent tissue, one cm from the primary tumor)

Phase Separation

200 μl chloroform/1 ml Trizol (originally used) was added, the tubes were shaken by hand for 15 seconds, and left at room temp for 2-3 minutes.

The samples were centrifuged at no more than 12,000×g for 15 minutes at 2-8 C.

RNA Precipitation

Following centrifugation, three phases were visible within the tube. The aqueous phase (top) was transferred to a fresh tube, ensuring that the solution was not contaminated with the other phases. Contamination is obvious by presence of any flakes or unclear liquid.

500 μl isopropanol/1 mL TRIZOL (originally used) were added to the new tube and incubated at room temperature for 10 minutes.

The samples were centrifuged at 12,000×g for 10 minutes at 2-8° C.

RNA Wash and Resuspension

Following centrifugation, the supernatant was removed.

The RNA pellet was washed with 1 ml of 75% EtOH/1 ml TRIZOL (originally used) and vortexed.

The samples were centrifuged at 7,500×g for 5 minutes at 2-8° C.

The supernatant was removed and the remaining EtOH was allowed to air dry

The pellet was redissolved in 25 μl of RNase free water and stored at −80° C. until use.

QC of the RNA was performed with the Agilent 2100 BioAnalyser using the Agilent RNA6000Nano kit. RNA concentrations were measured in a NanoDrop ND-1000 spectrophotometer. The PT'was only 71 ng/μL, so it was concentrated in a speedvac for 15 min to 342 ng/μL. The 1 C was 230 ng/μL, and was used as is.

RNA Labelling and Hybridization

Essentially, the instructions detailed in the “miRCURY Array labelling kit Instruction Manual”, issued by Exiqon A/S, were followed; in particular, the labelling is performed according to the disclosure in Gabor L. Igloi, Nonradioactive labeling of RNA, Anal Biochem. 1996 Jan. 1; 233(1):124-9.

All kit reagents were thawed on ice for 15 min, vortexed and spun down for 10 min.

In a 0.2 mL Eppendorf tube, the following reagents were added:

2.5× labeling buffer, 8 μL

Fluorescent label, 2 μL

1 μg total-RNA (2.92 μL (PT) and 4.35 μL (1 C))

Labeling enzyme, 2 μL

Nuclease-free water to 20 μL (5.08 μL (PT) and 3.65 μL (1 C))

Each microcentrifuge tube was vortexed and spun for 10 min.

Incubation at 0° C. for 1 hour was followed by 15 min at 65° C. Subsequently, the samples were kept on ice.

For hybridization, the 12-chamber TECAN HS4800Pro hybridization station was used.

25 μL 2× hybridization buffer was added to each sample, vortexed and spun.

Incubation at 95° C. for 3 min was followed by centrifugation for 2 min.

The hybridization chambers were primed with 1× Hyb buffer.

50 μl of the target preparation was injected into the Hyb station and incubated at 60° C. for 16 hours (overnight).

The slides were washed at 60° C. for 1 min with Buffer A twice, at 23° C. for 1 min with Buffer B twice, at 23° C. for 1 min with Buffer C twice, at 23° C. for 30 sec with Buffer C once.

The slides were dried for 5 min.

Scanning was performed in a ScanArray 4000XL (Packard Bioscience).

Experimental Design

The experimental design is depicted in FIG. 15.

Six RNA samples from different tissue origin were labeled with Hy3™ and a common reference (one tube for each RNA sample) was labeled with Hy5™ (the detectable moieties Hy3 and Hy5 are Oyster®-556 and Oyster®-656, resp. from Denovo Biolabels GmbH). Tissue RNA samples were mixed pair wise with common reference and hybridized on the miRCURY™ LNA Array (v.8.0). LNA array v 8.0 contains LNA spiked capture probes for 344 human microRNAs as registered and annotated in miRBase release 8.0 (February 2006) at The Wellcome Trust Sanger Institute, cf.

htto://microrna.sanger.ac.uk/sequences/index.shtml).

The quantified signals were normalized using the global Lowess (LOcally WEighted Scatterplot Smoothing) regression algorithm. The unsupervised hierarchical clustering is performed on log 2(Hy3/Hy5) ratios which passed the filtering criteria on variation across samples; standard deviation>0.50 (95 of 332 miRNAs passed).

Sample list:
Tongue
Throat
Esophagus (Normal adjacent tissue)
Esophagus (Tumor)
Lymph node
Tonsil
Common reference (pool of 31 total RNAs from different tissue origin)

Results

The heat map diagram in FIG. 16 shows the result of a two-way unsupervised hierarchical clustering of genes and samples. Each row represents a miRNA and each column represents a sample. The miRNA clustering tree is shown on the left, and the sample clustering tree appears at the top. The color scale shown at the bottom illustrates the relative expression level of a miRNA across all samples: red color represents an expression level above mean, blue color represents expression lower than the mean.

The six samples cluster in three groups; Tongue and Throat in one group, Esophagus (tumor and normal) in a second group and Lymph node and Tonsil in a third group.

The additional heat map diagram in FIG. 17 shows the result of a two-way supervised hierarchical clustering of genes and samples. A comparison of the two Esophagus samples (tumor and normal adjacent tissue) and the rest of the samples has been made identifying 39 miRNAs (out of 332 miRNAs) which distinguish between the two groups with more than two-fold up—or downregulation. The corresponding PCA plot shown in FIG. 18 shows clustering of three groups, however Esophagus Tumor and normal adjacent tissue are to some extent different.

Also the additional heat map diagram in FIG. 19 shows the result of another two-way supervised hierarchical clustering of genes and samples. A comparison of the two Esophagus samples (tumor and normal adjacent tissue) and the rest of the samples has been made identifying 23 miRNAs (out of 332 miRNAs) which are equally expressed (differs less than 50%) in tumor and normal tissue but distinguish between esophagus and the rest of the samples with more than two-fold up- or downregulation. The PCA plot in FIG. 20 shows the clustering of three groups.

1. A method for specifically identifying, in a mammal, the primary tissue origen of tumor cells in a sample, said method comprising
a) contacting a sample derived from a sample containing tumour cells from with at least one detection probe, which is a member from a collection of detection probes wherein each member of said collection comprises a recognition sequence consisting of nucleobases and affinity enhancing nucleobase analogues, and wherein the recognition sequences exhibit a combination of high melting temperatures and low self-complementarity scores, said melting temperatures being the melting temperature of the duplex between the recognition sequence and its complementary RNA sequence, said collection of detection probes being capable of specifically identifying target RNA sequences in all miRNAs of said mammal and said sample being contacted with said at least one detection probe under conditions that facilitate hybridization between said detection probe and RNA complementary to the recognition sequence of the detection probe, and
b) subsequently detecting hybridization between said at least one detection probe and RNA complementary to the recogntion sequence of the detection probe.

2. A method for specifically identifying, in a mammal, the tissue of origin of a tumour of unknown origin, said method comprising
a) contacting a sample derived from a sample containing tumour cells of said tumour with at least one detection probe, which is a member from a collection of detection probes wherein each member of said collection comprises a recognition sequence consisting of nucleobases and affinity enhancing nucleobase analogues, and wherein the recognition sequences exhibit a combination of high melting temperatures and low self-complementarity scores, said melting temperatures being the melting temperature of the duplex between the recognition sequence and its complementary RNA sequence, said collection of detection probes being capable of specifically identifying target RNA sequences in all miRNAs of said mammal and said sample being contacted with said at least one detection probe under conditions that facilitate hybridization between said detection probe and RNA complementary to the recognition sequence of the detection probe, and
b) subsequently detecting hybridization between said at least one detection probe the RNA complementary complementary to the detection probe.

3. The method according to claim 1 or 2, wherein at least 80% of the detection probes in the collection include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

4. The method according to claim 3, wherein at least 90% of the detection probes in the collection include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

5. The method according to claim 3, wherein at least 95% of the detection probes in the collection include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

6. The method according to claim 3, wherein all of the detection probes in the collection include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

7. The method according to any one of the preceding claims, wherein the melting temperature or the measure of melting temperature is at least 10° C., such as at least 15, at least 20, at least 25, at least 30, at least 35, at least 40, at least 45, and at least 50° C. higher than a melting temperature or measure of melting temperature of the self-complementarity score.

8. The method according to any one of the preceding claims, wherein the collection comprises at least 10 detection probes, 15 detection probes, such as at least 20, at least 25, at least 50, at least 75, at least 100, at least 200, at least 500, at least 1000, and at least 2000 members.

9. The method according to any one of the preceding claims, wherein the miRNA is mature miRNA.

10. The method according to any one of the preceding claims, wherein the mammal is a human being.

11. The method according to any one of the preceding claims, wherein the affinity-enhancing nucleobase analogues are regularly spaced between the nucleobases in at least 80% of the members of said collection, such as in at least 90% or at least 95% of said collection.

12. The method according to any one of the preceding claims, wherein the 3′ and 5′ nucleobases are not substituted by affinity enhancing nucleobase analogues.

13. The method according to any one of the preceding claims, wherein the presence of the affinity enhancing nucleobases in the recognition sequence confers an increase in the binding affinity between a detection probe and its complementary target RNA sequence relative to the binding affinity exhibited by a corresponding probe, which only include nucleobases.

14. The method according to any one of the preceding claims, wherein the affinity enhancing nucleobase analogues are LNA nucleobases.

15. The method according to any one of the preceding claims, wherein the affinity enhancing nucleobase analogues are regularly spaced as every 2^{nd}, every 3^{rd}, every 4^{th} or every 5^{th} nucleobase in the recognition sequence, preferably as every 3^{rd} nucleobase.

16. The method according to any one of the preceding claims, wherein the recognition sequence is at least a 6-mer, such as at least a 7-mer, at least an 8-mer, at least a 9-mer, at least a 10-mer, at least an 11-mer, at least a 12-mer, at least a 13-mer, at least a 14-mer, at least a 15-mer, at least a 16-mer, at least a 17-mer, at least an 18-mer, at least a 19-mer, at least a 20-mer, at least a 21-mer, at least a 22-mer, at least a 23-mer, and at least a 24-mer.

17. The method according to any one of claims 1-15, wherein the recognition sequence is at most a 25-mer, such as at most a 24-mer, at most a 23-mer, at most a 22-mer, at most a 21-mer, at most a 20-mer, at most a 19-mer, at most an 18-mer, at most a 17-mer, at most a 16-mer, at most a 15-mer, at most a 14-mer, at most a 13-mer, at most a 12-mer, at most an 11-mer, at most a 10-mer, at most a 9-mer, at most an 8-mer, at most a 7-mer, and at most a 6-mer.

18. The method according to any one of the preceding claims, wherein at least 80% of the detection probes comprise recognition sequences of the same length, such as at least 90% or at least 95%.

19. The method according to claim 18, wherein all detection probes contain affinity enhancing nucleobase analogues with the same regular spacing in the recognition sequences.

20. The method according to any one of the preceding claims, wherein at least one of the nucleobases in the recognition sequence is substituted with its corresponding selectively binding complementary (SBC) nucleobase.

21. The method according to any one of the preceding claims, wherein the nucleobases in the sequence are selected from ribonucleotides and deoxyribonucleotides.

22. The method according to claim 21, wherein the recognition sequence consists of affinity enhancing nucleobase analogues together with either ribonucleotides or deoxyribonucleotides.

23. The method according to any one of the preceding claims, wherein each detection probe is covalently bonded to a solid support.

24. The method according to claim 23, wherein the solid support is selected from a bead, a microarray, a chip, a strip, a chromatographic matrix, a microtiter plate, and a fiber.

25. The method according to any one of the preceding claims, wherein each detection probe includes a detection moiety and/or a ligand, optionally in the recognition sequence.

26. The method according to any one of the preceding claims, wherein each detection probe includes a photochemically active group, a thermochemically active group, a chelating group, a reporter group, or a ligand that facilitates the direct of indirect detection of the probe or the immobilisation of the probe onto a solid support.

27. The method according to any one of the preceding claims, wherein the detection probe includes a recognition sequence selected from the LNA containing recognition sequences set forth in table U and/or includes a recognition sequence capable of binding specifically to a miRNA set forth in Table T.

28. The method according to claim any one of the preceding claims, wherein at least one miRNA species is detected in the sample comprising RNA from the sample comprising tumour cells, thus providing a miRNA expression profile from the tumour, and subsequently comparing said miRNA expression profile with previously established miRNA expression profiles from normal tissue and/or tumour tissue.

29. The method according to claim any one of the preceding claims, wherein the sample is total RNA from the sample containing tumour cells.

30. The method according to claim 28 or 29, wherein comparison between the miRNA expression profile from the tumour and the previously established miRNA expression profiles provides for an indication of the origin of the tumour, the patient's prognosis, the optimum treatment regimen of the tumour and/or a prediction of the outcome of a given anti-tumour treatment.

31. The method according to any one of the preceding claims, wherein the miRNA has a length of at most 30 residues, such as at most 29, 28, 27, 26, 25, 24, 23, 22, 21, 20, 19, or 18 residues.

32. The method according to any one of claims 1-30, wherein the miRNA has a length of at least 15 residues, such as at least 16, 17, 18, 19, 20, 21, 22, 23, 24, 25, 26, 27, 28, 29 or 30 residues.

33. The method according to any one of the preceding claims, wherein the miRNA is present in a fixated, embedded sample such as a formalin fixated paraffin embedded sample.

34. The method according to any one of the preceding claims, which is used in diagnosis, prognosis, therapy outcome prediction, and therapy.

35. The method according to any one of the preceding claims, wherein the tumour of unknown origin is compared to an expression pattern characteristic of a) tumours derived lymph nodes and tonsils, b) tumours derived from tongue and throat and c) tumours derived from the esophagus.

36. The method according to any one of the preceding claims, wherein the metastatic tumour is a carcinoma.

37. A method of for the treatment of cancer, said method comprising
a. isolating RNA from at least one tissue sample from a patient suffering from cancer,
b. establishing an miRNA expression profile utilising RNA isolated in step a and determining at least one feature of said cancer which conforms with the miRNA expression profile,
c. based on the identification feature determined in step b) diagnosing the physiological status of the cancer disease in said patient, and
d. selecting and applying an appropriate form of therapy for said patient based on the said diagnosis.

38. The method according to claim 37, wherein the at least one feature of said cancer is selected from one or more of the group consisting of: presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.

39. The method of for the treatment of cancer according to claim 38, wherein the at least one feature of said cancer is determination of the origin of said cancer, wherein said cancer is a metestasis and/or a secondary cancer which is remote from the cancer of origin, such as the primary cancer.

40. The method for the treatment of cancer according to claim 38 or 39, wherein the treatment comprises one or more of the therapies selected from the group consisting of: chemotherapy; hormone treatment; invasive surgery; radiotherapy; and adjuvant systemic therapy.


SEQUENCE LISTING (880 sequences)

SEQ ID NO: 1 - DNA, 22 bp, Artificial Sequence
  Synthetic
  misc_feature (3)..(3): c is m5c
  misc_feature (12)..(12): c is m5c
  misc_feature (15)..(15): c is m5c
  misc_feature (18)..(18): c is m5c
  misc_feature (21)..(21): c is m5c
aactatacaatctactacctca

SEQ ID NO: 2 - DNA, 23 bp, Artificial Sequence
  Synthetic
  misc_feature (2)..(2): c is m5c
  misc_feature (20)..(20): c is m5c
tcagttttgcatggatttgcaca

SEQ ID NO: 3 - DNA, 24 bp, Artificial Sequence
  Synthetic
actacctgcactgtaagcactttg

SEQ ID NO: 4 - DNA, 24 bp, Artificial Sequence
  Synthetic
  misc_feature (4)..(4): c is m5c
  misc_feature (13)..(13): c is m5c
atccaatcagttcctgatgcagta

SEQ ID NO: 5 - DNA, 21 bp, Artificial Sequence
  Synthetic
acatggttagatcaagcacaa

SEQ ID NO: 6 - DNA, 24 bp, Artificial Sequence
  Synthetic
  misc_feature (6)..(6): c is m5c
gagacccagtagccagatgtagct

SEQ ID NO: 7 - DNA, 19 bp, Artificial Sequence
  Synthetic
  misc_feature (3)..(3): c is m5c
  misc_feature (9)..(9): c is m5c
  misc_feature (12)..(12): c is m5c
  misc_feature (15)..(15): c is m5c
  misc_feature (18)..(18): c is m5c
agcacaaactactacctca

SEQ ID NO: 8 - DNA, 20 bp, Artificial Sequence
  Synthetic
cagaacttagccactgtgaa

SEQ ID NO: 9 - DNA, 23 bp, Artificial Sequence
  Synthetic
  misc_feature (13)..(13): c is m5c
  misc_feature (19)..(19): c is m5c
gctttgacaatactattgcactg

SEQ ID NO: 10 - DNA, 21 bp, Artificial Sequence
  Synthetic
gctgagtgtaggatgtttaca

SEQ ID NO: 11 - DNA, 22 bp, Artificial Sequence
  Synthetic
cacaagttcggatctacgggtt

SEQ ID NO: 12 - DNA, 23 bp, Artificial Sequence
  Synthetic
  misc_feature (3)..(3): c is m5c
  misc_feature (6)..(6): c is m5c
  misc_feature (18)..(18): c is m5c
  misc_feature (21)..(21): c is m5c
aacaaccagctaagacactgcca

SEQ ID NO: 13 - DNA, 21 bp, Artificial Sequence
  Synthetic
  misc_feature (16)..(16): c is m5c
  misc_feature (19)..(19): c is m5c
aacaaaatcactagtcttcca

SEQ ID NO: 14 - DNA, 22 bp, Artificial Sequence
  Synthetic
  misc_feature (4)..(4): c is m5c
tcacaagttagggtctcaggga

SEQ ID NO: 15 - DNA, 22 bp, Artificial Sequence
  Synthetic
acagctggttgaaggggaccaa

SEQ ID NO: 16 - DNA, 22 bp, Artificial Sequence
  Synthetic
  misc_feature (4)..(4): c is m5c
  misc_feature (13)..(13): c is m5c
cttcagttatcacagtactgta

SEQ ID NO: 17 - DNA, 21 bp, Artificial Sequence
  Synthetic
  misc_feature (7)..(7): c is m5c
  misc_feature (16)..(16): c is m5c
aatgcccctaaaaatccttat

SEQ ID NO: 18 - DNA, 23 bp, Artificial Sequence
  Synthetic
  misc_feature (8)..(8): c is m5c
  misc_feature (14)..(14): c is m5c
tgatagccctgtacaatgctgct

SEQ ID NO: 19 - DNA, 20 bp, Artificial Sequence
  Synthetic
  misc_feature (4)..(4): c is m5c
  misc_feature (13)..(13): c is m5c
tcacttttgtgactatgcaa

SEQ ID NO: 20 - DNA, 22 bp, Artificial Sequence
  Synthetic
  misc_feature (2)..(2): c is m5c
  misc_feature (8)..(8): c is m5c
acaaattcggttctacagggta

SEQ ID NO: 21 - DNA, 22 bp, Artificial Sequence
  Synthetic
  misc_feature (4)..(4): c is m5c
  misc_feature (10)..(10): c is m5c
acacaaattcggttctacaggg

SEQ ID NO: 22 - DNA, 22 bp, Artificial Sequence
  Synthetic
tccacatggagttgctgttaca

SEQ ID NO: 23 - DNA, 23 bp, Artificial Sequence
  Synthetic
  misc_feature (12)..(12): c is m5c
gaacaggtagtctgaacactggg

SEQ ID NO: 24 - DNA, 22 bp, Artificial Sequence
  Synthetic
  misc_feature (4)..(4): c is m5c
aaccaatgtgcagactactgta

SEQ ID NO: 25 - DNA, 23 bp, Artificial Sequence
  Synthetic
ctacctgcactataagcacttta

SEQ ID NO: 26 - DNA, 21 bp, Artificial Sequence
  Synthetic
  misc_feature (9)..(9): c is m5c
  misc_feature (15)..(15): c is m5c
  misc_feature (18)..(18): c is m5c
ctgcctgtctgtgcctgctgt

SEQ ID NO: 27 - DNA, 21 bp, Artificial Sequence
  Synthetic
agaattgcgtttggacaatca

SEQ ID NO: 28 - DNA, 21 bp, Artificial Sequence
  Synthetic
  misc_feature (20)..(20): c is m5c
ggggtatttgacaaactgaca

SEQ ID NO: 29 - DNA, 21 bp, Artificial Sequence
  Synthetic
  misc_feature (8)..(8): c is m5c
ggaaatccctggcaatgtgat

SEQ ID NO: 30 - DNA, 22 bp, Artificial Sequence
  Synthetic
  misc_feature (20)..(20): c is m5c
ctgttcctgctgaactgagcca

SEQ ID NO: 31 - DNA, 22 bp, Artificial Sequence
  Synthetic
  misc_feature (4)..(4): c is m5c
agcctatcctggattacttgaa

SEQ ID NO: 32 - DNA, 21 bp, Artificial Sequence
  Synthetic
  misc_feature (12)..(12): c is m5c
gcattattactcacggtacga

SEQ ID NO: 33 - DNA, 21 bp, Artificial Sequence
  Synthetic
  misc_feature (3)..(3): c is m5c
cgcgtaccaaaagtaataatg

SEQ ID NO: 34 - DNA, 22 bp, Artificial Sequence
  Synthetic
  misc_feature (9)..(9): c is m5c
  misc_feature (15)..(15): c is m5c
aaaagagaccggttcactgtga

SEQ ID NO: 35 - DNA, 21 bp, Artificial Sequence
  Synthetic
  misc_feature (3)..(3): c is m5c
  misc_feature (9)..(9): c is m5c
aacaaaatcacaagtcttcca

SEQ ID NO: 36 - DNA, 22 bp, Artificial Sequence
  Synthetic
  misc_feature (3)..(3): c is m5c
  misc_feature (12)..(12): c is m5c
  misc_feature (15)..(15): c is m5c
  misc_feature (18)..(18): c is m5c
  misc_feature (21)..(21): c is m5c
aaccatacaacctactacctca

SEQ ID NO: 37 - DNA, 22 bp, Artificial Sequence
  Synthetic
  misc_feature (3)..(3): c is m5c
  misc_feature (6)..(6): c is m5c
  misc_feature (12)..(12): c is m5c
  misc_feature (15)..(15): c is m5c
  misc_feature (18)..(18): c is m5c
  misc_feature (21)..(21): c is m5c
aaccacacaacctactacctca

SEQ ID NO: 38 - DNA, 23 bp, Artificial Sequence
  Synthetic
  misc_feature (2)..(2): c is m5c
  misc_feature (8)..(8): c is m5c
  misc_feature (14)..(14): c is m5c
tcatagccctgtacaatgctgct

SEQ ID NO: 39 - DNA, 22 bp, Artificial Sequence
  Synthetic
  misc_feature (7)..(7): c is m5c
  misc_feature (13)..(13): c is m5c
  misc_feature (16)..(16): c is m5c
agcaagcccagaccgcaaaaag

SEQ ID NO: 40 - DNA, 22 bp, Artificial Sequence
  Synthetic
  misc_feature (20)..(20): c is m5c
atgctttttggggtaagggctt

SEQ ID NO: 41 - DNA, 20 bp, Artificial Sequence
  Synthetic
  misc_feature (3)..(3): c is m5c
  misc_feature (18)..(18): c is m5c
gcccttttaacattgcactg

SEQ ID NO: 42 - DNA, 22 bp, Artificial Sequence
  Synthetic
cgaccatggctgtagactgtta

SEQ ID NO: 43 - DNA, 23 bp, Artificial Sequence
  Synthetic
  misc_feature (4)..(4): c is m5c
  misc_feature (19)..(19): c is m5c
tcacataggaataaaaagccata

SEQ ID NO: 44 - DNA, 22 bp, Artificial Sequence
  Synthetic
ctacgcgtattcttaagcaata

SEQ ID NO: 45 - DNA, 20 bp, Artificial Sequence
  Synthetic
  misc_feature (10)..(10): c is m5c
acatcgttaccagacagtta

SEQ ID NO: 46 - DNA, 23 bp, Artificial Sequence
  Synthetic
  misc_feature (2)..(2): c is m5c
  misc_feature (17)..(17): c is m5c
tccataaagtaggaaacactaca

SEQ ID NO: 47 - DNA, 20 bp, Artificial Sequence
  Synthetic
  misc_feature (7)..(7): c is m5c
gtagtgctttctactttatg

SEQ ID NO: 48 - DNA, 24 bp, Artificial Sequence
  Synthetic
  misc_feature (3)..(3): c is m5c
aacccaccgacagcaatgaatgtt

SEQ ID NO: 49 - DNA, 23 bp, Artificial Sequence
  Synthetic
cagtgaattctaccagtgccata

SEQ ID NO: 50 - DNA, 22 bp, Artificial Sequence
  Synthetic
  misc_feature (3)..(3): c is m5c
  misc_feature (12)..(12): c is m5c
  misc_feature (21)..(21): c is m5c
acctaatatatcaaacatatca

SEQ ID NO: 51 - DNA, 21 bp, Artificial Sequence
  Synthetic
ctgggactttgtaggccagtt

SEQ ID NO: 52 - DNA, 23 bp, Artificial Sequence
  Synthetic
  misc_feature (2)..(2): c is m5c
  misc_feature (20)..(20): c is m5c
tcagttttgcatagatttgcaca

SEQ ID NO: 53 - DNA, 23 bp, Artificial Sequence
  Synthetic
caggcataggatgacaaagggaa

SEQ ID NO: 54 - DNA, 22 bp, Artificial Sequence
  Synthetic
cagactccggtggaatgaagga

SEQ ID NO: 55 - DNA, 21 bp, Artificial Sequence
  Synthetic
  misc_feature (3)..(3): c is m5c
  misc_feature (9)..(9): c is m5c
cacagttgccagctgagatta

SEQ ID NO: 56 - DNA, 23 bp, Artificial Sequence
  Synthetic
  misc_feature (5)..(5): c is m5c
  misc_feature (14)..(14): c is m5c
gaaacccagcagacaatgtagct

SEQ ID NO: 57 - DNA, 22 bp, Artificial Sequence
  Synthetic
  misc_feature (7)..(7): c is m5c
tcagaccgagacaagtgcaatg

SEQ ID NO: 58 - DNA, 22 bp, Artificial Sequence
  Synthetic
  misc_feature (4)..(4): c is m5c
taaccgatttcaaatggtgcta

SEQ ID NO: 59 - DNA, 22 bp, Artificial Sequence
  Synthetic
  misc_feature (2)..(2): c is m5c
  misc_feature (11)..(11): c is m5c
  misc_feature (20)..(20): c is m5c
acactgatttcaaatggtgcta

SEQ ID NO: 60 - DNA, 23 bp, Artificial Sequence
  Synthetic
  misc_feature (2)..(2): c is m5c
gctgagagtgtaggatgtttaca

SEQ ID NO: 61 - DNA, 21 bp, Artificial Sequence
  Synthetic
  misc_feature (18)..(18): c is m5c
ctaccatagggtaaaaccact

SEQ ID NO: 62 - DNA, 21 bp, Artificial Sequence
  Synthetic
  misc_feature (6)..(6): c is m5c
actttcggttatctagcttta

SEQ ID NO: 63 - DNA, 22 bp, Artificial Sequence
  Synthetic
  misc_feature (2)..(2): c is m5c
acaggccgggacaagtgcaata

SEQ ID NO: 64 - DNA, 23 bp, Artificial Sequence
  Synthetic
  misc_feature (20)..(20): c is m5c
agcaaaaatgtgctagtgccaaa

SEQ ID NO: 65 - DNA, 22 bp, Artificial Sequence
  Synthetic
cacaagatcggatctacgggtt

SEQ ID NO: 66 - DNA, 24 bp, Artificial Sequence
  Synthetic
aagggattcctgggaaaactggac

SEQ ID NO: 67 - DNA, 22 bp, Artificial Sequence
  Synthetic
  misc_feature (3)..(3): c is m5c
cccctatcacgattagcattaa

SEQ ID NO: 68 - DNA, 22 bp, Artificial Sequence
  Synthetic
  misc_feature (3)..(3): c is m5c
aaccgatttcaaatggtgctag

SEQ ID NO: 69 - DNA, 23 bp, Artificial Sequence
  Synthetic
  misc_feature (4)..(4): c is m5c
  misc_feature (16)..(16): c is m5c
gtccgtggttctaccctgtggta

SEQ ID NO: 70 - DNA, 22 bp, Artificial Sequence
  Synthetic
  misc_feature (4)..(4): c is m5c
  misc_feature (16)..(16): c is m5c
ccacacacttccttacattcca

SEQ ID NO: 71 - DNA, 22 bp, Artificial Sequence
  Synthetic
  misc_feature (4)..(4): c is m5c
  misc_feature (10)..(10): c is m5c
  misc_feature (13)..(13): c is m5c
tggcattcaccgcgtgccttaa

SEQ ID NO: 72 - DNA, 23 bp, Artificial Sequence
  Synthetic
  misc_feature (6)..(6): c is m5c
  misc_feature (9)..(9): c is m5c
  misc_feature (15)..(15): c is m5c
  misc_feature (21)..(21): c is m5c
acaaacaccattgtcacactcca

SEQ ID NO: 73 - DNA, 21 bp, Artificial Sequence
  Synthetic
  misc_feature (3)..(3): c is m5c
  misc_feature (15)..(15): c is m5c
tacatacttctttacattcca

SEQ ID NO: 74 - DNA, 23 bp, Artificial Sequence
  Synthetic
  misc_feature (6)..(6): c is m5c
actcaccgacagcgttgaatgtt

SEQ ID NO: 75 - DNA, 23 bp, Artificial Sequence
  Synthetic
  misc_feature (14)..(14): c is m5c
  misc_feature (17)..(17): c is m5c
cacaaattcggatctacagggta

SEQ ID NO: 76 - DNA, 21 bp, Artificial Sequence
  Synthetic
  misc_feature (19)..(19): c is m5c
ccaacaacatgaaactaccta

SEQ ID NO: 77 - DNA, 22 bp, Artificial Sequence
  Synthetic
  misc_feature (3)..(3): c is m5c
  misc_feature (12)..(12): c is m5c
  misc_feature (15)..(15): c is m5c
  misc_feature (18)..(18): c is m5c
  misc_feature (21)..(21): c is m5c
aactatacaacctactacctca

SEQ ID NO: 78 - DNA, 23 bp, Artificial Sequence
  Synthetic
  misc_feature (6)..(6): c is m5c
  misc_feature (9)..(9): c is m5c
  misc_feature (18)..(18): c is m5c
tcatacagctagataaccaaaga

SEQ ID NO: 79 - DNA, 20 bp, Artificial Sequence
  Synthetic
  misc_feature (4)..(4): c is m5c
  misc_feature (10)..(10): c is m5c
  misc_feature (16)..(16): c is m5c
agccgctgtcacacgcacag

SEQ ID NO: 80 - DNA, 21 bp, Artificial Sequence
  Synthetic
  misc_feature (6)..(6): c is m5c
  misc_feature (9)..(9): c is m5c
  misc_feature (12)..(12): c is m5c
tagtacatcatctatactgta

SEQ ID NO: 81 - DNA, 22 bp, Artificial Sequence
  Synthetic
  misc_feature (12)..(12): c is m5c
  misc_feature (18)..(18): c is m5c
caacaaaatcactgatgctgga

SEQ ID NO: 82 - DNA, 20 bp, Artificial Sequence
  Synthetic
  misc_feature (16)..(16): c is m5c
ggctgcaacacaagacacga

SEQ ID NO: 83 - DNA, 23 bp, Artificial Sequence
  Synthetic
  misc_feature (14)..(14): c is m5c
catcattaccaggcagtattaga

SEQ ID NO: 84 - DNA, 21 bp, Artificial Sequence
  Synthetic
  misc_feature (2)..(2): c is m5c
  misc_feature (8)..(8): c is m5c
  misc_feature (20)..(20): c is m5c
cccttatcagttctccgtcca

SEQ ID NO: 85 - DNA, 21 bp, Artificial Sequence
  Synthetic
  misc_feature (12)..(12): c is m5c
  misc_feature (15)..(15): c is m5c
gcggaacttagccactgtgaa

SEQ ID NO: 86 - DNA, 19 bp, Artificial Sequence
  Synthetic
  misc_feature (10)..(10): c is m5c
ctgtcaattcataggtcat

SEQ ID NO: 87 - DNA, 20 bp, Artificial Sequence
  Synthetic
  misc_feature (18)..(18): c is m5c
agtggtcctaaacatttcac

SEQ ID NO: 88 - DNA, 20 bp, Artificial Sequence
  Synthetic
ccaatatttacgtgctgcta

SEQ ID NO: 89 - DNA, 19 bp, Artificial Sequence
  Synthetic
  misc_feature (14)..(14): c is m5c
aagttctgtcatgcactga

SEQ ID NO: 90 - DNA, 22 bp, Artificial Sequence
  Synthetic
ctacctgcacaaacagcacttt

SEQ ID NO: 91 - DNA, 22 bp, Artificial Sequence
  Synthetic
  misc_feature (7)..(7): c is m5c
  misc_feature (10)..(10): c is m5c
  misc_feature (13)..(13): c is m5c
acagttcttcagctggcagctt

SEQ ID NO: 92 - DNA, 22 bp, Artificial Sequence
  Synthetic
  misc_feature (5)..(5): c is m5c
ggtacagtcaacggtcgatggt

SEQ ID NO: 93 - DNA, 21 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
cagctatgccaacatcttgcc

SEQ ID NO: 94 - DNA, 23 bp, Artificial Sequence
  Synthetic
  misc_feature (2)..(2): c is m5c
  misc_feature (14)..(14): c is m5c
actgttatcagctcagtaggcac

SEQ ID NO: 95 - DNA, 22 bp, Artificial Sequence
  Synthetic
  misc_feature (4)..(4): c is m5c
  misc_feature (7)..(7): c is m5c
  misc_feature (16)..(16): c is m5c
  misc_feature (19)..(19): c is m5c
tatctgcactaaatgcacctta

SEQ ID NO: 96 - DNA, 22 bp, Artificial Sequence
  Synthetic
  misc_feature (8)..(8): c is m5c
  misc_feature (17)..(17): c is m5c
  misc_feature (20)..(20): c is m5c
cacaaaccattctgtgctgcta

SEQ ID NO: 97 - DNA, 23 bp, Artificial Sequence
  Synthetic
  misc_feature (5)..(5): c is m5c
  misc_feature (8)..(8): c is m5c
  misc_feature (17)..(17): c is m5c
  misc_feature (20)..(20): c is m5c
caatcagctaacaacactgccta

SEQ ID NO: 98 - DNA, 22 bp, Artificial Sequence
  Synthetic
  misc_feature (19)..(19): c is m5c
acaaagttctgtaatgcactga

SEQ ID NO: 99 - DNA, 22 bp, Artificial Sequence
  Synthetic
  misc_feature (3)..(3): c is m5c
  misc_feature (15)..(15): c is m5c
cacaggttaagggtctcaggga

SEQ ID NO: 100 - DNA, 18 bp, Artificial Sequence
  Synthetic
  misc_feature (6)..(6): c is m5c
  misc_feature (12)..(12): c is m5c
agacacatgcactgtaga

SEQ ID NO: 101 - DNA, 22 bp, Artificial Sequence
  Synthetic
cactggtacaaggattgggaga

SEQ ID NO: 102 - DNA, 20 bp, Artificial Sequence
  Synthetic
  misc_feature (7)..(7): c is m5c
  misc_feature (19)..(19): c is m5c
ggctgtcaattcataggtca

SEQ ID NO: 103 - DNA, 22 bp, Artificial Sequence
  Synthetic
  misc_feature (19)..(19): c is m5c
aacaacacaacttactacctca

SEQ ID NO: 104 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
actgtacaaacaactacctca

SEQ ID NO: 105 - DNA, 23 bp, Artificial Sequence
  Synthetic
  misc_feature (10)..(10): c is m5c
  misc_feature (22)..(22): c is m5c
gcttccagtcggggatgtttaca

SEQ ID NO: 106 - DNA, 22 bp, Artificial Sequence
  Synthetic
  misc_feature (4)..(4): c is m5c
aacctatcctggattacttgaa

SEQ ID NO: 107 - DNA, 21 bp, Artificial Sequence
  Synthetic
  misc_feature (11)..(11): c is m5c
caacaccagtctgataagcta

SEQ ID NO: 108 - DNA, 21 bp, Artificial Sequence
  Synthetic
  misc_feature (4)..(4): c is m5c
  misc_feature (13)..(13): c is m5c
acccttggaattcagttctca

SEQ ID NO: 109 - DNA, 22 bp, Artificial Sequence
  Synthetic
  misc_feature (13)..(13): c is m5c
  misc_feature (19)..(19): c is m5c
tgtgagttctaccattgccaaa

SEQ ID NO: 110 - DNA, 21 bp, Artificial Sequence
  Synthetic
tagttggcaagtctagaacca

SEQ ID NO: 111 - DNA, 21 bp, Artificial Sequence
  Synthetic
  misc_feature (8)..(8): c is m5c
aagtgtccgatacggttgtgg

SEQ ID NO: 112 - DNA, 23 bp, Artificial Sequence
  Synthetic
  misc_feature (3)..(3): c is m5c
  misc_feature (12)..(12): c is m5c
gcccatcaaagctggctgtgata

SEQ ID NO: 113 - DNA, 22 bp, Artificial Sequence
  Synthetic
  misc_feature (13)..(13): c is m5c
aaaaagaacagccactgtgata

SEQ ID NO: 114 - DNA, 23 bp, Artificial Sequence
  Synthetic
  misc_feature (2)..(2): c is m5c
  misc_feature (5)..(5): c is m5c
  misc_feature (11)..(11): c is m5c
acaacaaaatcactagtcttcca

SEQ ID NO: 115 - DNA, 21 bp, Artificial Sequence
  Synthetic
taggagagagaaaaagactga

SEQ ID NO: 116 - DNA, 23 bp, Artificial Sequence
  Synthetic
  misc_feature (9)..(9): c is m5c
  misc_feature (18)..(18): c is m5c
tgtcgtaccagatagtgcattta

SEQ ID NO: 117 - DNA, 22 bp, Artificial Sequence
  Synthetic
  misc_feature (15)..(15): c is m5c
aaacggacgaaagtcccaccga

SEQ ID NO: 118 - DNA, 22 bp, Artificial Sequence
  Synthetic
ttaatgagtgtggatctagtca

SEQ ID NO: 119 - DNA, 22 bp, Artificial Sequence
  Synthetic
  misc_feature (17)..(17): c is m5c
taggacaaactttacccagtgc

SEQ ID NO: 120 - DNA, 22 bp, Artificial Sequence
  Synthetic
aaaggccgggaagtgtgcaata

SEQ ID NO: 121 - DNA, 22 bp, Artificial Sequence
  Synthetic
  misc_feature (10)..(10): c is m5c
  misc_feature (16)..(16): c is m5c
tgagataaacaaagcccagtga

SEQ ID NO: 122 - DNA, 23 bp, Artificial Sequence
  Synthetic
aatcagctttcaaaatgatctca

SEQ ID NO: 123 - DNA, 21 bp, Artificial Sequence
  Synthetic
  misc_feature (14)..(14): c is m5c
  misc_feature (16)..(16): c is m5c
  misc_feature (20)..(20): c is m5c
gatattggcgcggctcaatca

SEQ ID NO: 124 - DNA, 21 bp, Artificial Sequence
  Synthetic
  misc_feature (11)..(11): c is m5c
  misc_feature (14)..(14): c is m5c
  misc_feature (20)..(20): c is m5c
gatattggcgcggctcaatca

SEQ ID NO: 125 - DNA, 21 bp, Artificial Sequence
  Synthetic
  misc_feature (6)..(6): c is m5c
  misc_feature (8)..(8): c is m5c
  misc_feature (10)..(10): c is m5c
  misc_feature (18)..(18): c is m5c
tagagctcccttcaatccaaa

SEQ ID NO: 126 - DNA, 21 bp, Artificial Sequence
  Synthetic
  misc_feature (8)..(8): c is m5c
  misc_feature (17)..(17): c is m5c
tagagctcccttcaatccaaa

SEQ ID NO: 127 - DNA, 21 bp, Artificial Sequence
  Synthetic
  misc_feature (2)..(2): c is m5c
ccccgatgtagtcactttcaa

SEQ ID NO: 128 - DNA, 21 bp, Artificial Sequence
  Synthetic
  misc_feature (14)..(14): c is m5c
  misc_feature (17)..(17): c is m5c
  misc_feature (20)..(20): c is m5c
tagatcatgctggcagcttca

SEQ ID NO: 129 - DNA, 20 bp, Artificial Sequence
  Synthetic
  misc_feature (6)..(6): c is m5c
  misc_feature (9)..(9): c is m5c
  misc_feature (18)..(18): c is m5c
gggagctcccttcagtccaa

SEQ ID NO: 130 - DNA, 19 bp, Artificial Sequence
  Synthetic
gttcaagaaagctgtggaa

SEQ ID NO: 131 - DNA, 20 bp, Artificial Sequence
  Synthetic
  misc_feature (4)..(4): c is m5c
  misc_feature (10)..(10): c is m5c
  misc_feature (19)..(19): c is m5c
gtgctcactctcttctgtca

SEQ ID NO: 132 - DNA, 21 bp, Artificial Sequence
  Synthetic
  misc_feature (4)..(4): c is m5c
  misc_feature (7)..(7): c is m5c
  misc_feature (10)..(10): c is m5c
  misc_feature (13)..(13): c is m5c
atgcagcatcatcaagattct

SEQ ID NO: 133 - DNA, 22 bp, Artificial Sequence
  Synthetic
  misc_feature (4)..(4): c is m5c
  misc_feature (10)..(10): c is m5c
  misc_feature (16)..(16): c is m5c
ccacacactctcttacattcca

SEQ ID NO: 134 - DNA, 22 bp, Artificial Sequence
  Synthetic
  misc_feature (4)..(4): c is m5c
  misc_feature (10)..(10): c is m5c
  misc_feature (16)..(16): c is m5c
ccacacactcccttacattcca

SEQ ID NO: 135 - DNA, 22 bp, Artificial Sequence
  Synthetic
  misc_feature (4)..(4): c is m5c
  misc_feature (13)..(13): c is m5c
tggcattcaaagcgtgccttaa

SEQ ID NO: 136 - DNA, 22 bp, Artificial Sequence
  Synthetic
  misc_feature (4)..(4): c is m5c
  misc_feature (13)..(13): c is m5c
tggcattcaacgcgtgccttaa

SEQ ID NO: 137 - DNA, 23 bp, Artificial Sequence
  Synthetic
  misc_feature (6)..(6): c is m5c
  misc_feature (9)..(9): c is m5c
  misc_feature (12)..(12): c is m5c
  misc_feature (15)..(15): c is m5c
  misc_feature (21)..(21): c is m5c
acaaacaccaccgtcacactcca

SEQ ID NO: 138 - DNA, 23 bp, Artificial Sequence
  Synthetic
  misc_feature (6)..(6): c is m5c
  misc_feature (9)..(9): c is m5c
  misc_feature (12)..(12): c is m5c
  misc_feature (15)..(15): c is m5c
  misc_feature (21)..(21): c is m5c
acaaacaccatcgtcacactcca

SEQ ID NO: 139 - DNA, 22 bp, Artificial Sequence
  Synthetic
  misc_feature (5)..(5): c is m5c
  misc_feature (8)..(8): c is m5c
tgagctacagtgcttcatctca

SEQ ID NO: 140 - DNA, 23 bp, Artificial Sequence
  Synthetic
  misc_feature (5)..(5): c is m5c
  misc_feature (11)..(11): c is m5c
  misc_feature (14)..(14): c is m5c
ttcgccctctcaacccagctttt

SEQ ID NO: 141 - DNA, 22 bp, Artificial Sequence
  Synthetic
  misc_feature (8)..(8): c is m5c
  misc_feature (17)..(17): c is m5c
  misc_feature (20)..(20): c is m5c
cacaaaccattatgtgctgcta

SEQ ID NO: 142 - DNA, 22 bp, Artificial Sequence
  Synthetic
  misc_feature (3)..(3): c is m5c
cgccaatatttacgtgctgcta

SEQ ID NO: 143 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
actgtacaaactactacctca

SEQ ID NO: 144 - DNA, 22 bp, Artificial Sequence
  Synthetic
  misc_feature (3)..(3): c is m5c
  misc_feature (15)..(15): c is m5c
  misc_feature (18)..(18): c is m5c
  misc_feature (21)..(21): c is m5c
aactatacaacatactacctca

SEQ ID NO: 145 - DNA, 22 bp, Artificial Sequence
  Synthetic
  misc_feature (3)..(3): c is m5c
  misc_feature (15)..(15): c is m5c
  misc_feature (18)..(18): c is m5c
  misc_feature (21)..(21): c is m5c
aactatacaatatactacctca

SEQ ID NO: 146 - DNA, 22 bp, Artificial Sequence
  Synthetic
  misc_feature (5)..(5): c is m5c
  misc_feature (8)..(8): c is m5c
  misc_feature (11)..(11): c is m5c
tgagctacagcgcttcatctca

SEQ ID NO: 147 - DNA, 24 bp, Artificial Sequence
  Synthetic
aagggattcctcggaaaactggac

SEQ ID NO: 148 - DNA, 23 bp, Artificial Sequence
  Synthetic
  misc_feature (5)..(5): c is m5c
  misc_feature (14)..(14): c is m5c
ttcgccctctaaacccagctttt

SEQ ID NO: 149 - DNA, 22 bp, Artificial Sequence
  Synthetic
  misc_feature (4)..(4): c is m5c
agcctatcctcgattacttgaa

SEQ ID NO: 150 - DNA, 22 bp, Artificial Sequence
  Synthetic
cacaagatcgcatctacgggtt

SEQ ID NO: 151 - DNA, 22 bp, Artificial Sequence
  Synthetic
  misc_feature (8)..(8): c is m5c
  misc_feature (11)..(11): c is m5c
  misc_feature (17)..(17): c is m5c
  misc_feature (20)..(20): c is m5c
cacaaaccatcatgtgctgcta

SEQ ID NO: 152 - DNA, 22 bp, Artificial Sequence
  Synthetic
  misc_feature (3)..(3): c is m5c
cgccaatattttcgtgctgcta

SEQ ID NO: 153 - DNA, 22 bp, Artificial Sequence
  Synthetic
  misc_feature (11)..(11): c is m5c
  misc_feature (20)..(20): c is m5c
ctgttcctgccgaactgagcca

SEQ ID NO: 154 - DNA, 21 bp, Artificial Sequence
  Synthetic
  misc_feature (2)..(2): c is m5c
  misc_feature (14)..(14): c is m5c
  misc_feature (17)..(17): c is m5c
  misc_feature (20)..(20): c is m5c
actgtacaaaatactacctca

SEQ ID NO: 155 - DNA, 22 bp, Artificial Sequence
  Synthetic
tgaggtagtaggttgtatagtt

SEQ ID NO: 156 - DNA, 21 bp, Artificial Sequence
  Synthetic
tggaatgtaaagaagtatgta

SEQ ID NO: 157 - DNA, 22 bp, Artificial Sequence
  Synthetic
  misc_feature (4)..(4): c is m5c
  misc_feature (7)..(7): c is m5c
tagcagcacgtaaatattggcg

SEQ ID NO: 158 - DNA, 22 bp, Artificial Sequence
  Synthetic
  misc_feature (4)..(4): c is m5c
  misc_feature (7)..(7): c is m5c
  misc_feature (19)..(19): c is m5c
aagctgccagttgaagaactgt

SEQ ID NO: 159 - DNA, 22 bp, Artificial Sequence
  Synthetic
ttcaagtaattcaggataggtt

SEQ ID NO: 160 - DNA, 23 bp, Artificial Sequence
  Synthetic
  misc_feature (7)..(7): c is m5c
  misc_feature (10)..(10): c is m5c
  misc_feature (16)..(16): c is m5c
tgtaaacatcctacactctcagc

SEQ ID NO: 161 - DNA, 22 bp, Artificial Sequence
  Synthetic
tggagtgtgacaatggtgtttg

SEQ ID NO: 162 - DNA, 21 bp, Artificial Sequence
  Synthetic
cattattacttttggtacgcg

SEQ ID NO: 163 - DNA, 21 bp, Artificial Sequence
  Synthetic
  misc_feature (7)..(7): c is m5c
tcgtaccgtgagtaataatgc

SEQ ID NO: 164 - DNA, 22 bp, Artificial Sequence
  Synthetic
  misc_feature (8)..(8): c is m5c
ttggtccccttcaaccagctgt

SEQ ID NO: 165 - DNA, 22 bp, Artificial Sequence
  Synthetic
  misc_feature (11)..(11): c is m5c
tgagatgaagcactgtagctca

SEQ ID NO: 166 - DNA, 22 bp, Artificial Sequence
  Synthetic
tacagtatagatgatgtactag

SEQ ID NO: 167 - DNA, 22 bp, Artificial Sequence
  Synthetic
  misc_feature (7)..(7): c is m5c
  misc_feature (10)..(10): c is m5c
  misc_feature (13)..(13): c is m5c
acagttcttcaactggcagctt

SEQ ID NO: 168 - DNA, 22 bp, Artificial Sequence
  Synthetic
  misc_feature (4)..(4): c is m5c
aacctatcctgaattacttgaa

SEQ ID NO: 169 - DNA, 22 bp, Artificial Sequence
  Synthetic
  misc_feature (5)..(5): c is m5c
  misc_feature (8)..(8): c is m5c
  misc_feature (14)..(14): c is m5c
  misc_feature (20)..(20): c is m5c
caaacaccattgtcacactcca

SEQ ID NO: 170 - DNA, 22 bp, Artificial Sequence
  Synthetic
  misc_feature (7)..(7): c is m5c
  misc_feature (10)..(10): c is m5c
  misc_feature (13)..(13): c is m5c
ctagtacatcatctatactgta

SEQ ID NO: 171 - DNA, 22 bp, Artificial Sequence
  Synthetic
tgaggtagtaagttgtatagtt

SEQ ID NO: 172 - DNA, 21 bp, Artificial Sequence
  Synthetic
tggaatgtaaggaagtatgta

SEQ ID NO: 173 - DNA, 22 bp, Artificial Sequence
  Synthetic
tagcagcacggaaatattggcg

SEQ ID NO: 174 - DNA, 22 bp, Artificial Sequence
  Synthetic
aagctgccaggtgaagaactgt

SEQ ID NO: 175 - DNA, 22 bp, Artificial Sequence
  Synthetic
ttcaagtaatgcaggataggtt

SEQ ID NO: 176 - DNA, 23 bp, Artificial Sequence
  Synthetic
  misc_feature (7)..(7): c is m5c
  misc_feature (10)..(10): c is m5c
  misc_feature (16)..(16): c is m5c
tgtaaacatcatacactctcagc

SEQ ID NO: 177 - DNA, 22 bp, Artificial Sequence
  Synthetic
tggagtgtgaaaatggtgtttg

SEQ ID NO: 178 - DNA, 21 bp, Artificial Sequence
  Synthetic
cattattactgttggtacgcg

SEQ ID NO: 179 - DNA, 21 bp, Artificial Sequence
  Synthetic
  misc_feature (2)..(2): c is m5c
tcgtaccgtgggtaataatgc

SEQ ID NO: 180 - DNA, 22 bp, Artificial Sequence
  Synthetic
  misc_feature (7)..(7): c is m5c
  misc_feature (16)..(16): c is m5c
  misc_feature (19)..(19): c is m5c
ttggtcccctgcaaccagctgt

SEQ ID NO: 181 - DNA, 22 bp, Artificial Sequence
  Synthetic
tgagatgaagaactgtagctca

SEQ ID NO: 182 - DNA, 22 bp, Artificial Sequence
  Synthetic
tacagtataggtgatgtactag

SEQ ID NO: 183 - DNA, 22 bp, Artificial Sequence
  Synthetic
  misc_feature (8)..(8): c is m5c
  misc_feature (11)..(11): c is m5c
aactatacaacttactacctca

SEQ ID NO: 184 - DNA, 21 bp, Artificial Sequence
  Synthetic
  misc_feature (7)..(7): c is m5c
  misc_feature (10)..(10): c is m5c
  misc_feature (19)..(19): c is m5c
tacatacttccttacattcca

SEQ ID NO: 185 - DNA, 22 bp, Artificial Sequence
  Synthetic
  misc_feature (3)..(3): c is m5c
  misc_feature (12)..(12): c is m5c
cgccaatatttccgtgctgcta

SEQ ID NO: 186 - DNA, 22 bp, Artificial Sequence
  Synthetic
  misc_feature (2)..(2): c is m5c
  misc_feature (17)..(17): c is m5c
  misc_feature (20)..(20): c is m5c
acagttcttcacctggcagctt

SEQ ID NO: 187 - DNA, 22 bp, Artificial Sequence
  Synthetic
  misc_feature (3)..(3): c is m5c
  misc_feature (9)..(9): c is m5c
  misc_feature (12)..(12): c is m5c
aacctatcctgcattacttgaa

SEQ ID NO: 188 - DNA, 23 bp, Artificial Sequence
  Synthetic
  misc_feature (2)..(2): c is m5c
gctgagagtgtatgatgtttaca

SEQ ID NO: 189 - DNA, 22 bp, Artificial Sequence
  Synthetic
  misc_feature (5)..(5): c is m5c
  misc_feature (8)..(8): c is m5c
  misc_feature (14)..(14): c is m5c
  misc_feature (20)..(20): c is m5c
caaacaccattttcacactcca

SEQ ID NO: 190 - DNA, 21 bp, Artificial Sequence
  Synthetic
  misc_feature (3)..(3): c is m5c
cgcgtaccaacagtaataatg

SEQ ID NO: 191 - DNA, 21 bp, Artificial Sequence
  Synthetic
  misc_feature (2)..(2): c is m5c
  misc_feature (11)..(11): c is m5c
  misc_feature (14)..(14): c is m5c
gcattattacccacggtacga

SEQ ID NO: 192 - DNA, 22 bp, Artificial Sequence
  Synthetic
  misc_feature (19)..(19): c is m5c
acagctggttgcaggggaccaa

SEQ ID NO: 193 - DNA, 22 bp, Artificial Sequence
  Synthetic
  misc_feature (21)..(21): c is m5c
tgagctacagttcttcatctca

SEQ ID NO: 194 - DNA, 22 bp, Artificial Sequence
  Synthetic
  misc_feature (12)..(12): c is m5c
  misc_feature (18)..(18): c is m5c
ctagtacatcacctatactgta

SEQ ID NO: 195 - DNA, 21 bp, Artificial Sequence
  Synthetic
  misc_feature (9)..(9): c is m5c
tgtgctcactctcttctgtcg

SEQ ID NO: 196 - DNA, 21 bp, Artificial Sequence
  Synthetic
  misc_feature (4)..(4): c is m5c
  misc_feature (19)..(19): c is m5c
gtgctctctttcttctgtcaa

SEQ ID NO: 197 - DNA, 21 bp, Artificial Sequence
  Synthetic
  misc_feature (4)..(4): c is m5c
  misc_feature (19)..(19): c is m5c
gtgctctctatcttctgtcaa

SEQ ID NO: 198 - DNA, 20 bp, Artificial Sequence
  Synthetic
  misc_feature (8)..(8): c is m5c
gtgctctctatcttctgtca

SEQ ID NO: 199 - DNA, 20 bp, Artificial Sequence
  Synthetic
  misc_feature (3)..(3): c is m5c
  misc_feature (9)..(9): c is m5c
  misc_feature (12)..(12): c is m5c
tgctttgtctacatttggga

SEQ ID NO: 200 - DNA, 21 bp, Artificial Sequence
  Synthetic
  misc_feature (10)..(10): c is m5c
  misc_feature (13)..(13): c is m5c
tagagctcccttcaatccaaa

SEQ ID NO: 201 - DNA, 21 bp, Artificial Sequence
  Synthetic
  misc_feature (10)..(10): c is m5c
  misc_feature (13)..(13): c is m5c
aagagctcccttcaatccaaa

SEQ ID NO: 202 - DNA, 21 bp, Artificial Sequence
  Synthetic
  misc_feature (10)..(10): c is m5c
  misc_feature (13)..(13): c is m5c
aggagctcccttcaatccaaa

SEQ ID NO: 203 - DNA, 21 bp, Artificial Sequence
  Synthetic
  misc_feature (4)..(4): c is m5c
  misc_feature (16)..(16): c is m5c
tggcatacagggagccaggca

SEQ ID NO: 204 - DNA, 21 bp, Artificial Sequence
  Synthetic
  misc_feature (4)..(4): c is m5c
  misc_feature (13)..(13): c is m5c
  misc_feature (19)..(19): c is m5c
ccccgatgtagtcactttcaa

SEQ ID NO: 205 - DNA, 21 bp, Artificial Sequence
  Synthetic
  misc_feature (19)..(19): c is m5c
ctggatgcagaggtttatcga

SEQ ID NO: 206 - DNA, 24 bp, Artificial Sequence
  Synthetic
  misc_feature (11)..(11): c is m5c
  misc_feature (17)..(17): c is m5c
atcgaagttccaagtcctcttcaa

SEQ ID NO: 207 - DNA, 21 bp, Artificial Sequence
  Synthetic
  misc_feature (10)..(10): c is m5c
  misc_feature (19)..(19): c is m5c
tgcacgtgccctgcttctcca

SEQ ID NO: 208 - DNA, 21 bp, Artificial Sequence
  Synthetic
  misc_feature (10)..(10): c is m5c
  misc_feature (19)..(19): c is m5c
cgcacgtgccctgcttctcca

SEQ ID NO: 209 - DNA, 21 bp, Artificial Sequence
  Synthetic
  misc_feature (13)..(13): c is m5c
  misc_feature (19)..(19): c is m5c
gggggatgaagcctggtccga

SEQ ID NO: 210 - DNA, 21 bp, Artificial Sequence
  Synthetic
  misc_feature (13)..(13): c is m5c
  misc_feature (19)..(19): c is m5c
gggggatgaagcctggtccga

SEQ ID NO: 211 - DNA, 21 bp, Artificial Sequence
  Synthetic
ggggaatgaagcctggtccga

SEQ ID NO: 212 - skipped

SEQ ID NO: 213 - DNA, 21 bp, Artificial Sequence
  Synthetic
  misc_feature (14)..(14): c is m5c
  misc_feature (17)..(17): c is m5c
aagatcatgctggcagcttaa

SEQ ID NO: 214 - DNA, 22 bp, Artificial Sequence
  Synthetic
  misc_feature (15)..(15): c is m5c
  misc_feature (18)..(18): c is m5c
  misc_feature (21)..(21): c is m5c
ccagatcatgctggcagcttca

SEQ ID NO: 215 - DNA, 21 bp, Artificial Sequence
  Synthetic
  misc_feature (4)..(4): c is m5c
  misc_feature (19)..(19): c is m5c
ttcccgacctgcaccaagcga

SEQ ID NO: 216 - DNA, 21 bp, Artificial Sequence
  Synthetic
tcggcaagtcatccttggctg

SEQ ID NO: 217 - DNA, 21 bp, Artificial Sequence
  Synthetic
ccggcaagtcatccttggctg

SEQ ID NO: 218 - DNA, 21 bp, Artificial Sequence
  Synthetic
  misc_feature (20)..(20): c is m5c
cggcaagtcatccttggctca

SEQ ID NO: 219 - DNA, 21 bp, Artificial Sequence
  Synthetic
  misc_feature (17)..(17): c is m5c
agccaaggtcaacttgccgga

SEQ ID NO: 220 - DNA, 21 bp, Artificial Sequence
  Synthetic
caggcaagtcatccttggcta

SEQ ID NO: 221 - DNA, 21 bp, Artificial Sequence
  Synthetic
  misc_feature (11)..(11): c is m5c
  misc_feature (14)..(14): c is m5c
  misc_feature (20)..(20): c is m5c
gatattgacacggctcaatca

SEQ ID NO: 222 - DNA, 21 bp, Artificial Sequence
  Synthetic
  misc_feature (14)..(14): c is m5c
  misc_feature (17)..(17): c is m5c
cgtgatattggcacggctcaa

SEQ ID NO: 223 - skipped

SEQ ID NO: 224 - DNA, 20 bp, Artificial Sequence
  Synthetic
  misc_feature (7)..(7): c is m5c
gtgaatcttaatggtgctgc

SEQ ID NO: 225 - DNA, 21 bp, Artificial Sequence
  Synthetic
  misc_feature (4)..(4): c is m5c
  misc_feature (7)..(7): c is m5c
  misc_feature (10)..(10): c is m5c
  misc_feature (13)..(13): c is m5c
ctgcagcatcatcaagattct

SEQ ID NO: 226 - DNA, 21 bp, Artificial Sequence
  Synthetic
  misc_feature (4)..(4): c is m5c
  misc_feature (7)..(7): c is m5c
  misc_feature (10)..(10): c is m5c
  misc_feature (13)..(13): c is m5c
ctgcagcatcatcaagattct

SEQ ID NO: 227 - DNA, 21 bp, Artificial Sequence
  Synthetic
  misc_feature (20)..(20): c is m5c
atgcagcatcatcaagattcc

SEQ ID NO: 228 - DNA, 22 bp, Artificial Sequence
  Synthetic
  misc_feature (10)..(10): c is m5c
  misc_feature (19)..(19): c is m5c
gtgatttctctctgcaagcgaa

SEQ ID NO: 229 - DNA, 20 bp, Artificial Sequence
  Synthetic
  misc_feature (10)..(10): c is m5c
  misc_feature (13)..(13): c is m5c
gggagctcccttcagtccaa

SEQ ID NO: 230 - DNA, 20 bp, Artificial Sequence
  Synthetic
  misc_feature (10)..(10): c is m5c
  misc_feature (13)..(13): c is m5c
aggagctcccttcagtccaa

SEQ ID NO: 231 - DNA, 21 bp, Artificial Sequence
  Synthetic
  misc_feature (14)..(14): c is m5c
gatcaatgcgatccctttgga

SEQ ID NO: 232 - DNA, 20 bp, Artificial Sequence
  Synthetic
  misc_feature (17)..(17): c is m5c
ggaggtggacagaatgccaa

SEQ ID NO: 233 - DNA, 21 bp, Artificial Sequence
  Synthetic
  misc_feature (8)..(8): c is m5c
  misc_feature (14)..(14): c is m5c
gagttcccccaaacacttcag

SEQ ID NO: 234 - DNA, 21 bp, Artificial Sequence
  Synthetic
  misc_feature (7)..(7): c is m5c
  misc_feature (10)..(10): c is m5c
  misc_feature (16)..(16): c is m5c
  misc_feature (19)..(19): c is m5c
gagtccccccaaacacttcag

SEQ ID NO: 235 - DNA, 21 bp, Artificial Sequence
  Synthetic
  misc_feature (8)..(8): c is m5c
  misc_feature (14)..(14): c is m5c
gagttcccccaaacacttcag

SEQ ID NO: 236 - DNA, 21 bp, Artificial Sequence
  Synthetic
  misc_feature (8)..(8): c is m5c
  misc_feature (14)..(14): c is m5c
gagttcccccaaacacttcag

SEQ ID NO: 237 - DNA, 21 bp, Artificial Sequence
  Synthetic
cagttcaagaaagctgtggaa

SEQ ID NO: 238 - DNA, 21 bp, Artificial Sequence
  Synthetic
aagttcaagaaagctgtggaa

SEQ ID NO: 239 - DNA, 21 bp, Artificial Sequence
  Synthetic
  misc_feature (9)..(9): c is m5c
  misc_feature (12)..(12): c is m5c
catcaacgctgcactcaatga

SEQ ID NO: 240 - DNA, 21 bp, Artificial Sequence
  Synthetic
  misc_feature (12)..(12): c is m5c
catcaacgatgcactcaatga

SEQ ID NO: 241 - DNA, 21 bp, Artificial Sequence
  Synthetic
  misc_feature (10)..(10): c is m5c
aaggggtgacctgagaacaca

SEQ ID NO: 242 - DNA, 21 bp, Artificial Sequence
  Synthetic
  misc_feature (10)..(10): c is m5c
caggggtgacctgagaacaca

SEQ ID NO: 243 - DNA, 21 bp, Artificial Sequence
  Synthetic
  misc_feature (11)..(11): c is m5c
  misc_feature (14)..(14): c is m5c
  misc_feature (20)..(20): c is m5c
cagggcaaatctcctttggca

SEQ ID NO: 244 - DNA, 21 bp, Artificial Sequence
  Synthetic
  misc_feature (6)..(6): c is m5c
  misc_feature (9)..(9): c is m5c
cagggcaactctcctttggca

SEQ ID NO: 245 - DNA, 21 bp, Artificial Sequence
  Synthetic
  misc_feature (13)..(13): c is m5c
cggggcaaatctcctttggca

SEQ ID NO: 246 - DNA, 21 bp, Artificial Sequence
  Synthetic
  misc_feature (13)..(13): c is m5c
cgaggcaaatctcctttggca

SEQ ID NO: 247 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (20)..(20): c is m5c
ccgggcaaatctcctttggca

SEQ ID NO: 248 - DNA, 21 bp, Artificial Sequence
  Synthetic
  misc_feature (5)..(5): c is m5c
  misc_feature (14)..(14): c is m5c
gtgacttataatactctcata

SEQ ID NO: 249 - DNA, 21 bp, Artificial Sequence
  Synthetic
  misc_feature (4)..(4): c is m5c
  misc_feature (13)..(13): c is m5c
tgtcggtcgacaccagtttcg

SEQ ID NO: 250 - DNA, 22 bp, Artificial Sequence
  Synthetic
  misc_feature (17)..(17): c is m5c
cagaggtttaataggcctcgaa

SEQ ID NO: 251 - DNA, 21 bp, Artificial Sequence
  Synthetic
  misc_feature (18)..(18): c is m5c
cgagtttgtgcgtgaatctaa

SEQ ID NO: 252 - DNA, 24 bp, Artificial Sequence
  Synthetic
  misc_feature (2)..(2): c is m5c
  misc_feature (5)..(5): c is m5c
  misc_feature (8)..(8): c is m5c
  misc_feature (11)..(11): c is m5c
  misc_feature (14)..(14): c is m5c
gctgccgcaaccgccagcgttaat

SEQ ID NO: 253 - DNA, 24 bp, Artificial Sequence
  Synthetic
  misc_feature (15)..(15): c is m5c
agttatgggttagacccaactcat

SEQ ID NO: 254 - DNA, 21 bp, Artificial Sequence
  Synthetic
  misc_feature (9)..(9): c is m5c
  misc_feature (15)..(15): c is m5c
ctggattacaatagcattcta

SEQ ID NO: 255 - DNA, 21 bp, Artificial Sequence
  Synthetic
  misc_feature (2)..(2): c is m5c
accaaaagtatatgatttaaa

SEQ ID NO: 256 - DNA, 21 bp, Artificial Sequence
  Synthetic
  misc_feature (2)..(2): c is m5c
  misc_feature (14)..(14): c is m5c
gccagggaagaggcagtgcat

SEQ ID NO: 257 - DNA, 21 bp, Artificial Sequence
  Synthetic
  misc_feature (4)..(4): c is m5c
gtgcagaacaagagaaactat

SEQ ID NO: 258 - DNA, 21 bp, Artificial Sequence
  Synthetic
tgacgatgatgatgaagatga

SEQ ID NO: 259 - DNA, 21 bp, Artificial Sequence
  Synthetic
atgttctgtttctgctctgtt

SEQ ID NO: 260 - DNA, 21 bp, Artificial Sequence
  Synthetic
  misc_feature (5)..(5): c is m5c
  misc_feature (20)..(20): c is m5c
tgaacagtgtacgtacgaacc

SEQ ID NO: 261 - DNA, 21 bp, Artificial Sequence
  Synthetic
tcgaacaaattcactaccttc

SEQ ID NO: 262 - DNA, 21 bp, Artificial Sequence
  Synthetic
  misc_feature (4)..(4): c is m5c
ggtcagttcatcatcacatta

SEQ ID NO: 263 - DNA, 21 bp, Artificial Sequence
  Synthetic
  misc_feature (4)..(4): c is m5c
  misc_feature (7)..(7): c is m5c
  misc_feature (10)..(10): c is m5c
  misc_feature (13)..(13): c is m5c
caacatcctcagcattcataa

SEQ ID NO: 264 - DNA, 21 bp, Artificial Sequence
  Synthetic
  misc_feature (8)..(8): c is m5c
tgcatttccgtgattagttta

SEQ ID NO: 265 - DNA, 21 bp, Artificial Sequence
  Synthetic
  misc_feature (9)..(9): c is m5c
cgtaaggacaaatttccaaaa

SEQ ID NO: 266 - DNA, 21 bp, Artificial Sequence
  Synthetic
  misc_feature (4)..(4): c is m5c
  misc_feature (16)..(16): c is m5c
tcacacttgaggtctcaggga

SEQ ID NO: 267 - DNA, 21 bp, Artificial Sequence
  Synthetic
  misc_feature (13)..(13): c is m5c
cggaatgcgtctcatacaaaa

SEQ ID NO: 268 - DNA, 21 bp, Artificial Sequence
  Synthetic
  misc_feature (4)..(4): c is m5c
  misc_feature (10)..(10): c is m5c
  misc_feature (13)..(13): c is m5c
tggcattcaccgcgtgcctta

SEQ ID NO: 269 - DNA, 23 bp, Artificial Sequence
  Synthetic
ccgtgaattcatgcagtgccatt

SEQ ID NO: 270 - DNA, 23 bp, Artificial Sequence
  Synthetic
  misc_feature (12)..(12): c is m5c
tttcctggtcgcacaactaatac

SEQ ID NO: 271 - DNA, 24 bp, Artificial Sequence
  Synthetic
  misc_feature (7)..(7): c is m5c
tcctgcctgttgttcacgagctta

SEQ ID NO: 272 - DNA, 23 bp, Artificial Sequence
  Synthetic
  misc_feature (18)..(18): c is m5c
tcaccgcagttaagatgcattta

SEQ ID NO: 273 - DNA, 23 bp, Artificial Sequence
  Synthetic
  misc_feature (4)..(4): c is m5c
  misc_feature (19)..(19): c is m5c
tcccgcacatgcgcattgctcaa

SEQ ID NO: 274 - DNA, 21 bp, Artificial Sequence
  Synthetic
aagggtattctcgagcaataa

SEQ ID NO: 275 - DNA, 23 bp, Artificial Sequence
  Synthetic
  misc_feature (3)..(3): c is m5c
  misc_feature (6)..(6): c is m5c
  misc_feature (12)..(12): c is m5c
agcgtcattacctgacagtatta

SEQ ID NO: 276 - DNA, 23 bp, Artificial Sequence
  Synthetic
  misc_feature (2)..(2): c is m5c
  misc_feature (8)..(8): c is m5c
  misc_feature (20)..(20): c is m5c
ccagtacctaattgtagtacaaa

SEQ ID NO: 277 - DNA, 21 bp, Artificial Sequence
  Synthetic
  misc_feature (6)..(6): c is m5c
cagtacttttgtgcagtacaa

SEQ ID NO: 278 - DNA, 22 bp, Artificial Sequence
  Synthetic
agcgaaaatttggaggccagta

SEQ ID NO: 279 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
tcatttctcacacctacctca

SEQ ID NO: 280 - DNA, 24 bp, Artificial Sequence
  Synthetic
  misc_feature (19)..(19): c is m5c
cataccactttgtacaaccaaaga

SEQ ID NO: 281 - DNA, 22 bp, Artificial Sequence
  Synthetic
  misc_feature (18)..(18): c is m5c
gagctacttggaggggaccaat

SEQ ID NO: 282 - DNA, 22 bp, Artificial Sequence
  Synthetic
  misc_feature (5)..(5): c is m5c
  misc_feature (11)..(11): c is m5c
agctcctacccaatacatgtaa

SEQ ID NO: 283 - DNA, 23 bp, Artificial Sequence
  Synthetic
  misc_feature (5)..(5): c is m5c
  misc_feature (11)..(11): c is m5c
tgagcgttatccgagcacgtgta

SEQ ID NO: 284 - DNA, 22 bp, Artificial Sequence
  Synthetic
  misc_feature (2)..(2): c is m5c
  misc_feature (5)..(5): c is m5c
  misc_feature (17)..(17): c is m5c
gcaacactcaaaaatcctgtga

SEQ ID NO: 285 - DNA, 22 bp, Artificial Sequence
  Synthetic
  misc_feature (2)..(2): c is m5c
  misc_feature (17)..(17): c is m5c
ccgtgccaacagttgactgtga

SEQ ID NO: 286 - DNA, 24 bp, Artificial Sequence
  Synthetic
aataagagcggcaccactacttaa

SEQ ID NO: 287 - DNA, 23 bp, Artificial Sequence
  Synthetic
gttacctgcggcactactactta

SEQ ID NO: 288 - DNA, 21 bp, Artificial Sequence
  Synthetic
agttagtgttagtgaggtgtg

SEQ ID NO: 289 - DNA, 23 bp, Artificial Sequence
  Synthetic
  misc_feature (5)..(5): c is m5c
  misc_feature (11)..(11): c is m5c
tatacagttgcaaaagatttgca

SEQ ID NO: 290 - DNA, 21 bp, Artificial Sequence
  Synthetic
  misc_feature (4)..(4): c is m5c
aaccagattaggatgagattt

SEQ ID NO: 291 - DNA, 25 bp, Artificial Sequence
  Synthetic
  misc_feature (2)..(2): c is m5c
  misc_feature (8)..(8): c is m5c
  misc_feature (11)..(11): c is m5c
  misc_feature (14)..(14): c is m5c
  misc_feature (20)..(20): c is m5c
accaaaactgcttctaattcttgcc

SEQ ID NO: 292 - DNA, 22 bp, Artificial Sequence
  Synthetic
  misc_feature (5)..(5): c is m5c
  misc_feature (8)..(8): c is m5c
  misc_feature (17)..(17): c is m5c
  misc_feature (20)..(20): c is m5c
caaccagctaaccacactgcct

SEQ ID NO: 293 - DNA, 22 bp, Artificial Sequence
  Synthetic
  misc_feature (5)..(5): c is m5c
  misc_feature (17)..(17): c is m5c
cttgcaagttttcacccggtga

SEQ ID NO: 294 - DNA, 21 bp, Artificial Sequence
  Synthetic
  misc_feature (6)..(6): c is m5c
  misc_feature (9)..(9): c is m5c
  misc_feature (18)..(18): c is m5c
gataccaacacatgatacttg

SEQ ID NO: 295 - DNA, 21 bp, Artificial Sequence
  Synthetic
  misc_feature (16)..(16): c is m5c
aggagcagcaacaaacaaggt

SEQ ID NO: 296 - DNA, 21 bp, Artificial Sequence
  Synthetic
catagctcaggctaaaacaaa

SEQ ID NO: 297 - DNA, 21 bp, Artificial Sequence
  Synthetic
  misc_feature (12)..(12): c is m5c
ggatttgttcgcgttgctcat

SEQ ID NO: 298 - DNA, 21 bp, Artificial Sequence
  Synthetic
  misc_feature (16)..(16): c is m5c
tccgtcaatgactggcatttt

SEQ ID NO: 299 - DNA, 22 bp, Artificial Sequence
  Synthetic
  misc_feature (4)..(4): c is m5c
  misc_feature (7)..(7): c is m5c
  misc_feature (16)..(16): c is m5c
ccacgactaaggataccaattg

SEQ ID NO: 300 - DNA, 22 bp, Artificial Sequence
  Synthetic
  misc_feature (5)..(5): c is m5c
  misc_feature (17)..(17): c is m5c
attgcgaattttcacccggtga

SEQ ID NO: 301 - DNA, 21 bp, Artificial Sequence
  Synthetic
ttgtgaacgggattacggtca

SEQ ID NO: 302 - DNA, 22 bp, Artificial Sequence
  Synthetic
  misc_feature (5)..(5): c is m5c
  misc_feature (17)..(17): c is m5c
ataccaggttgtctcccggtga

SEQ ID NO: 303 - DNA, 22 bp, Artificial Sequence
  Synthetic
  misc_feature (5)..(5): c is m5c
  misc_feature (17)..(17): c is m5c
ctaaccgtttttcacccggtga

SEQ ID NO: 304 - DNA, 22 bp, Artificial Sequence
  Synthetic
  misc_feature (15)..(15): c is m5c
ctagctgattgacacccggtga

SEQ ID NO: 305 - DNA, 21 bp, Artificial Sequence
  Synthetic
  misc_feature (16)..(16): c is m5c
tgggagtttttcacccggtga

SEQ ID NO: 306 - DNA, 20 bp, Artificial Sequence
  Synthetic
  misc_feature (14)..(14): c is m5c
ctgtagatgttaacccggtg

SEQ ID NO: 307 - DNA, 23 bp, Artificial Sequence
  Synthetic
gcgacagcaagtaaactgtgata

SEQ ID NO: 308 - DNA, 21 bp, Artificial Sequence
  Synthetic
  misc_feature (13)..(13): c is m5c
agctgaatgtgtctctagtca

SEQ ID NO: 309 - DNA, 22 bp, Artificial Sequence
  Synthetic
  misc_feature (15)..(15): c is m5c
  misc_feature (21)..(21): c is m5c
tgaagagagcgactccatgaca

SEQ ID NO: 310 - DNA, 22 bp, Artificial Sequence
  Synthetic
tgaagagagcgcctccatgaca

SEQ ID NO: 311 - DNA, 23 bp, Artificial Sequence
  Synthetic
  misc_feature (4)..(4): c is m5c
  misc_feature (7)..(7): c is m5c
  misc_feature (10)..(10): c is m5c
  misc_feature (16)..(16): c is m5c
  misc_feature (19)..(19): c is m5c
  misc_feature (22)..(22): c is m5c
tcgcatctactgagcctacctca

SEQ ID NO: 312 - DNA, 22 bp, Artificial Sequence
  Synthetic
tctgcagcttctcgtggtgctt

SEQ ID NO: 313 - DNA, 24 bp, Artificial Sequence
  Synthetic
  misc_feature (3)..(3): c is m5c
  misc_feature (18)..(18): c is m5c
aacccaagaatatcagacatatca

SEQ ID NO: 314 - DNA, 23 bp, Artificial Sequence
  Synthetic
aacatggcaaggagctacgggta

SEQ ID NO: 315 - DNA, 24 bp, Artificial Sequence
  Synthetic
  misc_feature (3)..(3): c is m5c
agcacggaaacatatgtacgggtg

SEQ ID NO: 316 - DNA, 23 bp, Artificial Sequence
  Synthetic
ctcggcagaaaaatatacgggta

SEQ ID NO: 317 - DNA, 24 bp, Artificial Sequence
  Synthetic
  misc_feature (4)..(4): c is m5c
acacacagctcgatctacagggta

SEQ ID NO: 318 - DNA, 22 bp, Artificial Sequence
  Synthetic
  misc_feature (5)..(5): c is m5c
attgccgtactgaacgatctca

SEQ ID NO: 319 - DNA, 23 bp, Artificial Sequence
  Synthetic
tggactagaaaatgtgcataata

SEQ ID NO: 320 - DNA, 23 bp, Artificial Sequence
  Synthetic
  misc_feature (17)..(17): c is m5c
gagcagagtcaaggttctagtca

SEQ ID NO: 321 - DNA, 22 bp, Artificial Sequence
  Synthetic
  misc_feature (16)..(16): c is m5c
ctgtaagctagattacatatca

SEQ ID NO: 322 - DNA, 23 bp, Artificial Sequence
  Synthetic
  misc_feature (7)..(7): c is m5c
tccgtacacgcttcagtgtcatg

SEQ ID NO: 323 - DNA, 24 bp, Artificial Sequence
  Synthetic
  misc_feature (7)..(7): c is m5c
tctactctttctaggaggttgtga

SEQ ID NO: 324 - DNA, 23 bp, Artificial Sequence
  Synthetic
  misc_feature (15)..(15): c is m5c
ctgggaacaccaatcacgtatta

SEQ ID NO: 325 - DNA, 19 bp, Artificial Sequence
  Synthetic
  misc_feature (4)..(4): c is m5c
  misc_feature (7)..(7): c is m5c
tcactacccatgtctttca

SEQ ID NO: 326 - DNA, 19 bp, Artificial Sequence
  Synthetic
  misc_feature (2)..(2): c is m5c
  misc_feature (8)..(8): c is m5c
  misc_feature (11)..(11): c is m5c
  misc_feature (14)..(14): c is m5c
  misc_feature (17)..(17): c is m5c
gctatgccaacatctgcct

SEQ ID NO: 327 - DNA, 23 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
actgaactgccaacatcttgcca

SEQ ID NO: 328 - DNA, 22 bp, Artificial Sequence
  Synthetic
  misc_feature (7)..(7): c is m5c
  misc_feature (10)..(10): c is m5c
  misc_feature (16)..(16): c is m5c
tctagactgccatttcttgcca

SEQ ID NO: 329 - DNA, 22 bp, Artificial Sequence
  Synthetic
  misc_feature (17)..(17): c is m5c
tgaaggcggttggtagctttaa

SEQ ID NO: 330 - DNA, 22 bp, Artificial Sequence
  Synthetic
tggacagctatggcctgatgaa

SEQ ID NO: 331 - DNA, 22 bp, Artificial Sequence
  Synthetic
  misc_feature (17)..(17): c is m5c
agctttggtaacctagctttat

SEQ ID NO: 332 - DNA, 23 bp, Artificial Sequence
  Synthetic
  misc_feature (9)..(9): c is m5c
  misc_feature (12)..(12): c is m5c
tcggctttcaactaatgatctca

SEQ ID NO: 333 - DNA, 22 bp, Artificial Sequence
  Synthetic
  misc_feature (6)..(6): c is m5c
  misc_feature (12)..(12): c is m5c
  misc_feature (21)..(21): c is m5c
actagctttcacgatgatctca

SEQ ID NO: 334 - DNA, 22 bp, Artificial Sequence
  Synthetic
  misc_feature (2)..(2): c is m5c
actggctttcacgatgatctca

SEQ ID NO: 335 - DNA, 22 bp, Artificial Sequence
  Synthetic
  misc_feature (4)..(4): c is m5c
acactgaatttatatggtgcta

SEQ ID NO: 336 - DNA, 22 bp, Artificial Sequence
  Synthetic
  misc_feature (19)..(19): c is m5c
gacagcattgcaaactacctca

SEQ ID NO: 337 - DNA, 24 bp, Artificial Sequence
  Synthetic
  misc_feature (2)..(2): c is m5c
gcacgccttttcaaatactttgta

SEQ ID NO: 338 - DNA, 23 bp, Artificial Sequence
  Synthetic
  misc_feature (14)..(14): c is m5c
  misc_feature (20)..(20): c is m5c
gactgtggcaaagcattcactta

SEQ ID NO: 339 - DNA, 20 bp, Artificial Sequence
  Synthetic
  misc_feature (2)..(2): c is m5c
  misc_feature (5)..(5): c is m5c
  misc_feature (11)..(11): c is m5c
acacctgaaactttgctcac

SEQ ID NO: 340 - DNA, 22 bp, Artificial Sequence
  Synthetic
  misc_feature (5)..(5): c is m5c
ggggcattcaaacaacatatca

SEQ ID NO: 341 - DNA, 21 bp, Artificial Sequence
  Synthetic
  misc_feature (13)..(13): c is m5c
cgaaatgcgtctcatacaaaa

SEQ ID NO: 342 - DNA, 23 bp, Artificial Sequence
  Synthetic
  misc_feature (12)..(12): c is m5c
gcacatcaaagctggctgtgata

SEQ ID NO: 343 - DNA, 22 bp, Artificial Sequence
  Synthetic
  misc_feature (4)..(4): c is m5c
  misc_feature (10)..(10): c is m5c
gttcagaatcatgtcgaaagct

SEQ ID NO: 344 - DNA, 27 bp, Artificial Sequence
  Synthetic
acgatggaaaagataaccagtgtcatt

SEQ ID NO: 345 - DNA, 23 bp, Artificial Sequence
  Synthetic
  misc_feature (12)..(12): c is m5c
tctcctggtcgcacaactaatac

SEQ ID NO: 346 - DNA, 24 bp, Artificial Sequence
  Synthetic
  misc_feature (7)..(7): c is m5c
ttctgcctgttgatcacgagctta

SEQ ID NO: 347 - DNA, 22 bp, Artificial Sequence
  Synthetic
  misc_feature (6)..(6): c is m5c
  misc_feature (18)..(18): c is m5c
tcaggccggggagagtgcaata

SEQ ID NO: 348 - DNA, 24 bp, Artificial Sequence
  Synthetic
  misc_feature (17)..(17): c is m5c
aagctgttcgagaattctcaggga

SEQ ID NO: 349 - DNA, 23 bp, Artificial Sequence
  Synthetic
tctgaatggcatcggagtacaaa

SEQ ID NO: 350 - DNA, 23 bp, Artificial Sequence
  Synthetic
  misc_feature (7)..(7): c is m5c
ccagtacctatgtgtagtacaaa

SEQ ID NO: 351 - DNA, 21 bp, Artificial Sequence
  Synthetic
cagtacttttgtgtagtacaa

SEQ ID NO: 352 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
tcatttctcgcacctacctca

SEQ ID NO: 353 - DNA, 21 bp, Artificial Sequence
  Synthetic
  misc_feature (2)..(2): c is m5c
  misc_feature (14)..(14): c is m5c
  misc_feature (17)..(17): c is m5c
tcgaagcaaaggcctacgcaa

SEQ ID NO: 354 - DNA, 23 bp, Artificial Sequence
  Synthetic
  misc_feature (7)..(7): c is m5c
  misc_feature (10)..(10): c is m5c
  misc_feature (13)..(13): c is m5c
  misc_feature (22)..(22): c is m5c
gatatcccgccgcgatcgtaccg

SEQ ID NO: 355 - DNA, 22 bp, Artificial Sequence
  Synthetic
  misc_feature (5)..(5): c is m5c
  misc_feature (11)..(11): c is m5c
agctcctacccgaaacatgtaa

SEQ ID NO: 356 - DNA, 23 bp, Artificial Sequence
  Synthetic
  misc_feature (17)..(17): c is m5c
aagaagagaataggctctagtca

SEQ ID NO: 357 - DNA, 23 bp, Artificial Sequence
  Synthetic
  misc_feature (5)..(5): c is m5c
  misc_feature (11)..(11): c is m5c
tgagcgttatccgtgcacgtgta

SEQ ID NO: 358 - DNA, 22 bp, Artificial Sequence
  Synthetic
  misc_feature (7)..(7): c is m5c
  misc_feature (16)..(16): c is m5c
gcaacgctcaaaagtcctgtga

SEQ ID NO: 359 - DNA, 22 bp, Artificial Sequence
  Synthetic
  misc_feature (2)..(2): c is m5c
  misc_feature (17)..(17): c is m5c
ccatgccaacagttgactgtga

SEQ ID NO: 360 - DNA, 21 bp, Artificial Sequence
  Synthetic
ggtcagtgttagtgaggtgtg

SEQ ID NO: 361 - DNA, 23 bp, Artificial Sequence
  Synthetic
  misc_feature (2)..(2): c is m5c
  misc_feature (5)..(5): c is m5c
  misc_feature (11)..(11): c is m5c
cctacagtcgcgaaagatttgca

SEQ ID NO: 362 - DNA, 21 bp, Artificial Sequence
  Synthetic
  misc_feature (7)..(7): c is m5c
  misc_feature (10)..(10): c is m5c
  misc_feature (19)..(19): c is m5c
tacagtcttctatgcattcca

SEQ ID NO: 363 - DNA, 22 bp, Artificial Sequence
  Synthetic
tcactgggtactcctgatactc

SEQ ID NO: 364 - DNA, 21 bp, Artificial Sequence
  Synthetic
  misc_feature (10)..(10): c is m5c
  misc_feature (13)..(13): c is m5c
aaaaggattcctctcaaaacc

SEQ ID NO: 365 - DNA, 21 bp, Artificial Sequence
  Synthetic
  misc_feature (4)..(4): c is m5c
taccagattaggatgagattt

SEQ ID NO: 366 - DNA, 20 bp, Artificial Sequence
  Synthetic
  misc_feature (4)..(4): c is m5c
ctacaagagttcgacatcac

SEQ ID NO: 367 - DNA, 19 bp, Artificial Sequence
  Synthetic
cgtgaaaactaaaaagcta

SEQ ID NO: 368 - DNA, 20 bp, Artificial Sequence
  Synthetic
atcagaaaacatcgagaaac

SEQ ID NO: 369 - DNA, 20 bp, Artificial Sequence
  Synthetic
  misc_feature (13)..(13): c is m5c
  misc_feature (16)..(16): c is m5c
  misc_feature (19)..(19): c is m5c
cataacaacaaccacccgcc

SEQ ID NO: 370 - DNA, 20 bp, Artificial Sequence
  Synthetic
  misc_feature (4)..(4): c is m5c
  misc_feature (7)..(7): c is m5c
  misc_feature (13)..(13): c is m5c
  misc_feature (16)..(16): c is m5c
  misc_feature (19)..(19): c is m5c
ataccacccttcctccctca

SEQ ID NO: 371 - DNA, 20 bp, Artificial Sequence
  Synthetic
  misc_feature (7)..(7): c is m5c
  misc_feature (19)..(19): c is m5c
gctttgccaaagtcttgcct

SEQ ID NO: 372 - DNA, 20 bp, Artificial Sequence
  Synthetic
  misc_feature (10)..(10): c is m5c
tgcagcagacacttcacggg

SEQ ID NO: 373 - DNA, 24 bp, Artificial Sequence
  Synthetic
  misc_feature (2)..(2): c is m5c
  misc_feature (23)..(23): c is m5c
accaaactgcttctaattcttgcc

SEQ ID NO: 374 - DNA, 20 bp, Artificial Sequence
  Synthetic
  misc_feature (8)..(8): c is m5c
agttttgccagagtcttgcc

SEQ ID NO: 375 - DNA, 20 bp, Artificial Sequence
  Synthetic
ctccactgctacatcatgcc

SEQ ID NO: 376 - DNA, 20 bp, Artificial Sequence
  Synthetic
  misc_feature (9)..(9): c is m5c
  misc_feature (15)..(15): c is m5c
  misc_feature (18)..(18): c is m5c
aatgctttcccacccggcga

SEQ ID NO: 377 - DNA, 18 bp, Artificial Sequence
  Synthetic
  misc_feature (5)..(5): c is m5c
  misc_feature (8)..(8): c is m5c
  misc_feature (14)..(14): c is m5c
  misc_feature (17)..(17): c is m5c
caaacacccatgcctaca

SEQ ID NO: 378 - DNA, 20 bp, Artificial Sequence
  Synthetic
  misc_feature (5)..(5): c is m5c
  misc_feature (8)..(8): c is m5c
cagccgacacagtacgggca

SEQ ID NO: 379 - DNA, 22 bp, Artificial Sequence
  Synthetic
  misc_feature (2)..(2): c is m5c
  misc_feature (5)..(5): c is m5c
  misc_feature (17)..(17): c is m5c
actgctagtttccacccggtga

SEQ ID NO: 380 - DNA, 21 bp, Artificial Sequence
  Synthetic
  misc_feature (6)..(6): c is m5c
  misc_feature (9)..(9): c is m5c
aataccaacacatggcaattg

SEQ ID NO: 381 - DNA, 21 bp, Artificial Sequence
  Synthetic
  misc_feature (12)..(12): c is m5c
tgatttgttcgcgttgctcaa

SEQ ID NO: 382 - DNA, 21 bp, Artificial Sequence
  Synthetic
  misc_feature (2)..(2): c is m5c
tcctgcaacgactggcattta

SEQ ID NO: 383 - DNA, 21 bp, Artificial Sequence
  Synthetic
  misc_feature (15)..(15): c is m5c
ccttgacagggataccaattg

SEQ ID NO: 384 - DNA, 21 bp, Artificial Sequence
  Synthetic
  misc_feature (2)..(2): c is m5c
  misc_feature (5)..(5): c is m5c
tcgtcagagaaagaccagtga

SEQ ID NO: 385 - DNA, 22 bp, Artificial Sequence
  Synthetic
  misc_feature (5)..(5): c is m5c
  misc_feature (17)..(17): c is m5c
catgcgaattttcacccggtga

SEQ ID NO: 386 - DNA, 22 bp, Artificial Sequence
  Synthetic
  misc_feature (2)..(2): c is m5c
  misc_feature (5)..(5): c is m5c
  misc_feature (17)..(17): c is m5c
actgcaagtgttcacccggtga

SEQ ID NO: 387 - DNA, 22 bp, Artificial Sequence
  Synthetic
  misc_feature (2)..(2): c is m5c
  misc_feature (5)..(5): c is m5c
  misc_feature (17)..(17): c is m5c
actccagtttttctcccggtga

SEQ ID NO: 388 - DNA, 22 bp, Artificial Sequence
  Synthetic
  misc_feature (5)..(5): c is m5c
  misc_feature (17)..(17): c is m5c
caagctgatttacacccggtga

SEQ ID NO: 389 - DNA, 22 bp, Artificial Sequence
  Synthetic
  misc_feature (9)..(9): c is m5c
  misc_feature (15)..(15): c is m5c
tcatcacacgtgatcgatgata

SEQ ID NO: 390 - DNA, 22 bp, Artificial Sequence
  Synthetic
  misc_feature (5)..(5): c is m5c
  misc_feature (17)..(17): c is m5c
ttagctgatgtacacccggtga

SEQ ID NO: 391 - DNA, 22 bp, Artificial Sequence
  Synthetic
  misc_feature (17)..(17): c is m5c
taggtgatttttcacccggtga

SEQ ID NO: 392 - DNA, 24 bp, Artificial Sequence
  Synthetic
  misc_feature (3)..(3): c is m5c
  misc_feature (18)..(18): c is m5c
aacccaagaataccagacatatca

SEQ ID NO: 393 - DNA, 23 bp, Artificial Sequence
  Synthetic
aacatggataggagctacgggta

SEQ ID NO: 394 - DNA, 24 bp, Artificial Sequence
  Synthetic
  misc_feature (3)..(3): c is m5c
agcacggaaacaaatgtacgggtg

SEQ ID NO: 395 - DNA, 24 bp, Artificial Sequence
  Synthetic
ctcggattatgaagattacgggta

SEQ ID NO: 396 - DNA, 23 bp, Artificial Sequence
  Synthetic
ctcagcagaaacttatacgggta

SEQ ID NO: 397 - DNA, 22 bp, Artificial Sequence
  Synthetic
ctcagcggaaacattacgggta

SEQ ID NO: 398 - DNA, 23 bp, Artificial Sequence
  Synthetic
  misc_feature (7)..(7): c is m5c
  misc_feature (19)..(19): c is m5c
  misc_feature (22)..(22): c is m5c
tacaacccaaaatggatccgcca

SEQ ID NO: 399 - DNA, 23 bp, Artificial Sequence
  Synthetic
  misc_feature (8)..(8): c is m5c
catcatcctgataaacgattcga

SEQ ID NO: 400 - DNA, 23 bp, Artificial Sequence
  Synthetic
tgaactagaaaatgtgcataata

SEQ ID NO: 401 - DNA, 23 bp, Artificial Sequence
  Synthetic
  misc_feature (22)..(22): c is m5c
gagatgagtaacggttctagtca

SEQ ID NO: 402 - DNA, 22 bp, Artificial Sequence
  Synthetic
  misc_feature (16)..(16): c is m5c
ctgtaagctagattacatatca

SEQ ID NO: 403 - DNA, 24 bp, Artificial Sequence
  Synthetic
  misc_feature (12)..(12): c is m5c
  misc_feature (15)..(15): c is m5c
  misc_feature (21)..(21): c is m5c
tttccaactcgcttcagtgtcata

SEQ ID NO: 404 - DNA, 23 bp, Artificial Sequence
  Synthetic
ttcggtaacgcttcagtgtcata

SEQ ID NO: 405 - DNA, 23 bp, Artificial Sequence
  Synthetic
ttcggttacgcttcagtgtcata

SEQ ID NO: 406 - DNA, 23 bp, Artificial Sequence
  Synthetic
  misc_feature (2)..(2): c is m5c
  misc_feature (8)..(8): c is m5c
  misc_feature (14)..(14): c is m5c
  misc_feature (20)..(20): c is m5c
tcacatccctaatcagtgtcatg

SEQ ID NO: 407 - DNA, 21 bp, Artificial Sequence
  Synthetic
  misc_feature (2)..(2): c is m5c
  misc_feature (5)..(5): c is m5c
tctacacttttgagtcttcga

SEQ ID NO: 408 - DNA, 21 bp, Artificial Sequence
  Synthetic
tctacactttttaattttcga

SEQ ID NO: 409 - DNA, 23 bp, Artificial Sequence
  Synthetic
  misc_feature (10)..(10): c is m5c
atggaaacaccaacgacgtatta

SEQ ID NO: 410 - DNA, 20 bp, Artificial Sequence
  Synthetic
  misc_feature (2)..(2): c is m5c
  misc_feature (8)..(8): c is m5c
  misc_feature (11)..(11): c is m5c
  misc_feature (14)..(14): c is m5c
gctatgccaacatcttgcct

SEQ ID NO: 411 - DNA, 23 bp, Artificial Sequence
  Synthetic
  misc_feature (7)..(7): c is m5c
  misc_feature (10)..(10): c is m5c
  misc_feature (22)..(22): c is m5c
actgaactgcctacatcttgcca

SEQ ID NO: 412 - DNA, 22 bp, Artificial Sequence
  Synthetic
  misc_feature (21)..(21): c is m5c
tgtagactgccatttcttgcca

SEQ ID NO: 413 - DNA, 22 bp, Artificial Sequence
  Synthetic
  misc_feature (6)..(6): c is m5c
tgaagccggttggtagctttaa

SEQ ID NO: 414 - DNA, 22 bp, Artificial Sequence
  Synthetic
  misc_feature (7)..(7): c is m5c
  misc_feature (10)..(10): c is m5c
  misc_feature (13)..(13): c is m5c
  misc_feature (16)..(16): c is m5c
  misc_feature (19)..(19): c is m5c
tcaaggcttcatcaacaacgaa

SEQ ID NO: 415 - DNA, 21 bp, Artificial Sequence
  Synthetic
  misc_feature (4)..(4): c is m5c
  misc_feature (16)..(16): c is m5c
  misc_feature (19)..(19): c is m5c
gcacaaacaaccaggcctcca

SEQ ID NO: 416 - DNA, 22 bp, Artificial Sequence
  Synthetic
  misc_feature (17)..(17): c is m5c
agctttggtaacctagctttat

SEQ ID NO: 417 - DNA, 22 bp, Artificial Sequence
  Synthetic
  misc_feature (6)..(6): c is m5c
  misc_feature (12)..(12): c is m5c
  misc_feature (21)..(21): c is m5c
actagctttcacgatgatctca

SEQ ID NO: 418 - DNA, 22 bp, Artificial Sequence
  Synthetic
  misc_feature (4)..(4): c is m5c
ttactgaatttatatggtgcta

SEQ ID NO: 419 - DNA, 22 bp, Artificial Sequence
  Synthetic
  misc_feature (3)..(3): c is m5c
  misc_feature (15)..(15): c is m5c
  misc_feature (18)..(18): c is m5c
  misc_feature (21)..(21): c is m5c
tacaatattacatactacctca

SEQ ID NO: 420 - DNA, 24 bp, Artificial Sequence
  Synthetic
  misc_feature (2)..(2): c is m5c
gcacgacttttcaaatactttgta

SEQ ID NO: 421 - DNA, 23 bp, Artificial Sequence
  Synthetic
  misc_feature (14)..(14): c is m5c
  misc_feature (20)..(20): c is m5c
gactgtggcaaagcattcactta

SEQ ID NO: 422 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
actatacaacctactacctca

SEQ ID NO: 423 - DNA, 22 bp, Artificial Sequence
  Synthetic
  misc_feature (4)..(4): c is m5c
  misc_feature (16)..(16): c is m5c
ctccatacttctttacattcca

SEQ ID NO: 424 - DNA, 21 bp, Artificial Sequence
  Synthetic
  misc_feature (13)..(13): c is m5c
  misc_feature (16)..(16): c is m5c
acaaattcggatctacagggt

SEQ ID NO: 425 - DNA, 22 bp, Artificial Sequence
  Synthetic
  misc_feature (17)..(17): c is m5c
cacaagttcggatttacgggtt

SEQ ID NO: 426 - DNA, 21 bp, Artificial Sequence
  Synthetic
  misc_feature (10)..(10): c is m5c
gcaagaactcagactgtgatg

SEQ ID NO: 427 - DNA, 23 bp, Artificial Sequence
  Synthetic
  misc_feature (22)..(22): c is m5c
accagtacctgatgtaatactca

SEQ ID NO: 428 - DNA, 23 bp, Artificial Sequence
  Synthetic
  misc_feature (6)..(6): c is m5c
  misc_feature (12)..(12): c is m5c
  misc_feature (15)..(15): c is m5c
cttggcattcaccgcgtgcctta

SEQ ID NO: 429 - DNA, 22 bp, Artificial Sequence
  Synthetic
  misc_feature (15)..(15): c is m5c
actcatcaaaatggctgtgata

SEQ ID NO: 430 - DNA, 22 bp, Artificial Sequence
  Synthetic
  misc_feature (15)..(15): c is m5c
actcgtcaaaatggctgtgata

SEQ ID NO: 431 - DNA, 22 bp, Artificial Sequence
  Synthetic
  misc_feature (3)..(3): c is m5c
  misc_feature (9)..(9): c is m5c
  misc_feature (21)..(21): c is m5c
gcccttatcagttctccgtcca

SEQ ID NO: 432 - DNA, 21 bp, Artificial Sequence
  Synthetic
cggggcgagagaatgataagg

SEQ ID NO: 433 - DNA, 21 bp, Artificial Sequence
  Synthetic
  misc_feature (5)..(5): c is m5c
  misc_feature (11)..(11): c is m5c
  misc_feature (17)..(17): c is m5c
tagccgctgtcacacgcacaa

SEQ ID NO: 434 - DNA, 23 bp, Artificial Sequence
  Synthetic
caagaattgcgtttggacaatca

SEQ ID NO: 435 - DNA, 24 bp, Artificial Sequence
  Synthetic
gtgaattcttccagtgccattaac

SEQ ID NO: 436 - DNA, 21 bp, Artificial Sequence
  Synthetic
  misc_feature (8)..(8): c is m5c
  misc_feature (11)..(11): c is m5c
  misc_feature (17)..(17): c is m5c
gtgaattctcccagtgccaag

SEQ ID NO: 437 - DNA, 26 bp, Artificial Sequence
  Synthetic
  misc_feature (5)..(5): c is m5c
  misc_feature (20)..(20): c is m5c
attacccgttagtgtcggtcacaaaa

SEQ ID NO: 438 - DNA, 23 bp, Artificial Sequence
  Synthetic
  misc_feature (5)..(5): c is m5c
  misc_feature (20)..(20): c is m5c
cgcgcgctacttcaggtacctga

SEQ ID NO: 439 - DNA, 22 bp, Artificial Sequence
  Synthetic
agagcacggtatgaagttccta

SEQ ID NO: 440 - DNA, 23 bp, Artificial Sequence
  Synthetic
  misc_feature (16)..(16): c is m5c
  misc_feature (19)..(19): c is m5c
cgtaggaactctatacctcgctg

SEQ ID NO: 441 - DNA, 22 bp, Artificial Sequence
  Synthetic
agagcacggtattaagttccta

SEQ ID NO: 442 - DNA, 27 bp, Artificial Sequence
  Synthetic
  misc_feature (26)..(26): c is m5c
tatcatttcatatgcaacgtaaataca

SEQ ID NO: 443 - DNA, 23 bp, Artificial Sequence
  Synthetic
acaaagagagcaattccatgaca

SEQ ID NO: 444 - DNA, 22 bp, Artificial Sequence
  Synthetic
  misc_feature (13)..(13): c is m5c
  misc_feature (16)..(16): c is m5c
actgtcgacggacagctctctt

SEQ ID NO: 445 - DNA, 22 bp, Artificial Sequence
  Synthetic
  misc_feature (16)..(16): c is m5c
actgtcgacggatagctctctt

SEQ ID NO: 446 - DNA, 28 bp, Artificial Sequence
  Synthetic
  misc_feature (2)..(2): c is m5c
  misc_feature (11)..(11): c is m5c
acagacaaagcctagtagaggctagatt

SEQ ID NO: 447 - DNA, 21 bp, Artificial Sequence
  Synthetic
  misc_feature (8)..(8): c is m5c
agaattaccagctgatattta

SEQ ID NO: 448 - DNA, 29 bp, Artificial Sequence
  Synthetic
  misc_feature (21)..(21): c is m5c
caattgctggaatcaagttgctgacttca

SEQ ID NO: 449 - DNA, 22 bp, Artificial Sequence
  Synthetic
  misc_feature (4)..(4): c is m5c
gcactgatttcgaatggtgcta

SEQ ID NO: 450 - DNA, 23 bp, Artificial Sequence
  Synthetic
  misc_feature (13)..(13): c is m5c
  misc_feature (22)..(22): c is m5c
agcacgagtgttcggtctagtca

SEQ ID NO: 451 - DNA, 21 bp, Artificial Sequence
  Synthetic
  misc_feature (4)..(4): c is m5c
gtgcaaacgattttcaacaca

SEQ ID NO: 452 - DNA, 23 bp, Artificial Sequence
  Synthetic
catgaaatgaaatcgacatgaaa

SEQ ID NO: 453 - DNA, 26 bp, Artificial Sequence
  Synthetic
  misc_feature (4)..(4): c is m5c
  misc_feature (10)..(10): c is m5c
  misc_feature (13)..(13): c is m5c
agtcgcaggctccacttaaatattta

SEQ ID NO: 454 - DNA, 23 bp, Artificial Sequence
  Synthetic
  misc_feature (12)..(12): c is m5c
gctcatcaaagctggctgtgata

SEQ ID NO: 455 - DNA, 23 bp, Artificial Sequence
  Synthetic
  misc_feature (12)..(12): c is m5c
gctcctcaaagctggctgtgata

SEQ ID NO: 456 - DNA, 22 bp, Artificial Sequence
  Synthetic
  misc_feature (10)..(10): c is m5c
  misc_feature (16)..(16): c is m5c
tgagacacactttgcccagtga

SEQ ID NO: 457 - DNA, 23 bp, Artificial Sequence
  Synthetic
  misc_feature (10)..(10): c is m5c
  misc_feature (19)..(19): c is m5c
accagtttcctgtgaaacctaaa

SEQ ID NO: 458 - DNA, 23 bp, Artificial Sequence
  Synthetic
ctcacatttacaaattgagatta

SEQ ID NO: 459 - DNA, 23 bp, Artificial Sequence
  Synthetic
cagagcacctgatgaagtacaat

SEQ ID NO: 460 - DNA, 22 bp, Artificial Sequence
  Synthetic
  misc_feature (11)..(11): c is m5c
ttgagagtcactaagtacctga

SEQ ID NO: 461 - DNA, 22 bp, Artificial Sequence
  Synthetic
  misc_feature (2)..(2): c is m5c
  misc_feature (8)..(8): c is m5c
  misc_feature (20)..(20): c is m5c
gcacaggcacagagtgaccccc

SEQ ID NO: 462 - DNA, 20 bp, Artificial Sequence
  Synthetic
  misc_feature (3)..(3): c is m5c
ctcactcaaggaggttgtga

SEQ ID NO: 463 - DNA, 22 bp, Artificial Sequence
  Synthetic
  misc_feature (5)..(5): c is m5c
  misc_feature (14)..(14): c is m5c
ctcacagtataatcctgtgatt

SEQ ID NO: 464 - DNA, 22 bp, Artificial Sequence
  Synthetic
  misc_feature (17)..(17): c is m5c
taggacaaactttacccagtgc

SEQ ID NO: 465 - DNA, 22 bp, Artificial Sequence
  Synthetic
  misc_feature (2)..(2): c is m5c
tcaggccggtgaatgtgcaata

SEQ ID NO: 466 - DNA, 22 bp, Artificial Sequence
  Synthetic
  misc_feature (2)..(2): c is m5c
tcaggccgtctcaagtgcaata

SEQ ID NO: 467 - DNA, 22 bp, Artificial Sequence
  Synthetic
tcgggctgtgaaaagtgcaata

SEQ ID NO: 468 - DNA, 21 bp, Artificial Sequence
  Synthetic
  misc_feature (2)..(2): c is m5c
  misc_feature (14)..(14): c is m5c
ccgaacttattggctcgaata

SEQ ID NO: 469 - DNA, 22 bp, Artificial Sequence
  Synthetic
  misc_feature (2)..(2): c is m5c
  misc_feature (11)..(11): c is m5c
  misc_feature (14)..(14): c is m5c
gctttctgagcaacaatcaaaa

SEQ ID NO: 470 - DNA, 22 bp, Artificial Sequence
  Synthetic
  misc_feature (4)..(4): c is m5c
cgccagtaagcggaaaaagaca

SEQ ID NO: 471 - DNA, 24 bp, Artificial Sequence
  Synthetic
  misc_feature (2)..(2): c is m5c
  misc_feature (23)..(23): c is m5c
actggataccaccagctgtgttca

SEQ ID NO: 472 - DNA, 23 bp, Artificial Sequence
  Synthetic
  misc_feature (10)..(10): c is m5c
  misc_feature (22)..(22): c is m5c
tcagctatgccgacatcttgcca

SEQ ID NO: 473 - DNA, 22 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
cagctattccgacatcttgcca

SEQ ID NO: 474 - DNA, 21 bp, Artificial Sequence
  Synthetic
  misc_feature (17)..(17): c is m5c
  misc_feature (20)..(20): c is m5c
caatgcgactacaatgcacct

SEQ ID NO: 475 - DNA, 22 bp, Artificial Sequence
  Synthetic
  misc_feature (5)..(5): c is m5c
  misc_feature (8)..(8): c is m5c
  misc_feature (17)..(17): c is m5c
  misc_feature (20)..(20): c is m5c
caaccagctaaccacactgcca

SEQ ID NO: 476 - DNA, 21 bp, Artificial Sequence
  Synthetic
  misc_feature (12)..(12): c is m5c
tcaatggttgtctagctttat

SEQ ID NO: 477 - DNA, 23 bp, Artificial Sequence
  Synthetic
  misc_feature (19)..(19): c is m5c
catatcacaacgatcgttccttt

SEQ ID NO: 478 - DNA, 23 bp, Artificial Sequence
  Synthetic
  misc_feature (2)..(2): c is m5c
  misc_feature (5)..(5): c is m5c
  misc_feature (11)..(11): c is m5c
acaacaaaatcactagtcttcca

SEQ ID NO: 479 - DNA, 22 bp, Artificial Sequence
  Synthetic
  misc_feature (4)..(4): c is m5c
atgctttggtaatctagcttta

SEQ ID NO: 480 - DNA, 23 bp, Artificial Sequence
  Synthetic
  misc_feature (3)..(3): c is m5c
  misc_feature (6)..(6): c is m5c
  misc_feature (12)..(12): c is m5c
gacatctttacctgacagtatta

SEQ ID NO: 481 - DNA, 21 bp, Artificial Sequence
  Synthetic
  misc_feature (3)..(3): c is m5c
  misc_feature (6)..(6): c is m5c
cacacctgaaattttgctcaa

SEQ ID NO: 482 - DNA, 22 bp, Artificial Sequence
  Synthetic
ataggccgggacaagtgcaatg

SEQ ID NO: 483 - DNA, 22 bp, Artificial Sequence
  Synthetic
  misc_feature (2)..(2): c is m5c
gcaggccgggactagtgcaatt

SEQ ID NO: 484 - DNA, 23 bp, Artificial Sequence
  Synthetic
catacagctaaaatcaccaaaga

SEQ ID NO: 485 - DNA, 22 bp, Artificial Sequence
  Synthetic
  misc_feature (16)..(16): c is m5c
tctacagctagaataccaaaga

SEQ ID NO: 486 - DNA, 24 bp, Artificial Sequence
  Synthetic
  misc_feature (22)..(22): c is m5c
gttacgtatactgaaggtataccg

SEQ ID NO: 487 - DNA, 22 bp, Artificial Sequence
  Synthetic
  misc_feature (2)..(2): c is m5c
  misc_feature (20)..(20): c is m5c
tcaggatacattcagtatacgt

SEQ ID NO: 488 - DNA, 22 bp, Artificial Sequence
  Synthetic
cacaagttcggaattacgggtt

SEQ ID NO: 489 - DNA, 22 bp, Artificial Sequence
  Synthetic
  misc_feature (8)..(8): c is m5c
  misc_feature (17)..(17): c is m5c
  misc_feature (20)..(20): c is m5c
aaacggacgaaagtccctccga

SEQ ID NO: 490 - DNA, 22 bp, Artificial Sequence
  Synthetic
  misc_feature (17)..(17): c is m5c
taagacaaacttcacccagtgc

SEQ ID NO: 491 - DNA, 24 bp, Artificial Sequence
  Synthetic
  misc_feature (23)..(23): c is m5c
attggataccaccagctgtgttca

SEQ ID NO: 492 - DNA, 24 bp, Artificial Sequence
  Synthetic
  misc_feature (11)..(11): c is m5c
  misc_feature (23)..(23): c is m5c
atcagctatgccgacatcttgcca

SEQ ID NO: 493 - DNA, 24 bp, Artificial Sequence
  Synthetic
  misc_feature (11)..(11): c is m5c
  misc_feature (23)..(23): c is m5c
btcagctattccgacatcttgcca

SEQ ID NO: 494 - DNA, 21 bp, Artificial Sequence
  Synthetic
  misc_feature (12)..(12): c is m5c
tcaatggttgtctagctttat

SEQ ID NO: 495 - DNA, 22 bp, Artificial Sequence
  Synthetic
  misc_feature (16)..(16): c is m5c
tctacagctagaataccaaaga

SEQ ID NO: 496 - DNA, 22 bp, Artificial Sequence
  Synthetic
cccaccgacagcaatgaatgtt

SEQ ID NO: 497 - DNA, 21 bp, Artificial Sequence
  Synthetic
  misc_feature (7)..(7): c is m5c
  misc_feature (19)..(19): c is m5c
ggctgtcaattcataggtcat

SEQ ID NO: 498 - DNA, 22 bp, Artificial Sequence
  Synthetic
  misc_feature (20)..(20): c is m5c
caagtggtcctaaacatttcac

SEQ ID NO: 499 - DNA, 22 bp, Artificial Sequence
  Synthetic
  misc_feature (4)..(4): c is m5c
aggcataggatgacaaagggaa

SEQ ID NO: 500 - DNA, 22 bp, Artificial Sequence
  Synthetic
  misc_feature (6)..(6): c is m5c
  misc_feature (12)..(12): c is m5c
  misc_feature (18)..(18): c is m5c
ttagccgctgtcacacgcacag

SEQ ID NO: 501 - DNA, 22 bp, Artificial Sequence
  Synthetic
  misc_feature (5)..(5): c is m5c
ggtacaatcaacggtcaatggt

SEQ ID NO: 502 - DNA, 22 bp, Artificial Sequence
  Synthetic
  misc_feature (4)..(4): c is m5c
  misc_feature (10)..(10): c is m5c
caacaaaatcactagtcttcca

SEQ ID NO: 503 - DNA, 21 bp, Artificial Sequence
  Synthetic
  misc_feature (5)..(5): c is m5c
  misc_feature (8)..(8): c is m5c
  misc_feature (14)..(14): c is m5c
agcacgtcacttccactaaga

SEQ ID NO: 504 - DNA, 22 bp, Artificial Sequence
  Synthetic
gcaagggcgaatgcagaaaata

SEQ ID NO: 505 - DNA, 22 bp, Artificial Sequence
  Synthetic
  misc_feature (16)..(16): c is m5c
aactccggggctgatcaggtta

SEQ ID NO: 506 - DNA, 23 bp, Artificial Sequence
  Synthetic
ttcaatttctgccgcaaaagata

SEQ ID NO: 507 - DNA, 22 bp, Artificial Sequence
  Synthetic
gctatctgctgcaacagaattt

SEQ ID NO: 508 - DNA, 23 bp, Artificial Sequence
  Synthetic
  misc_feature (6)..(6): c is m5c
  misc_feature (12)..(12): c is m5c
  misc_feature (18)..(18): c is m5c
gtgtgcttacacacttcccgtta

SEQ ID NO: 509 - DNA, 21 bp, Artificial Sequence
  Synthetic
  misc_feature (7)..(7): c is m5c
  misc_feature (10)..(10): c is m5c
actatgcaacccactacctct

SEQ ID NO: 510 - DNA, 21 bp, Artificial Sequence
  Synthetic
  misc_feature (2)..(2): c is m5c
  misc_feature (5)..(5): c is m5c
  misc_feature (11)..(11): c is m5c
  misc_feature (14)..(14): c is m5c
  misc_feature (17)..(17): c is m5c
  misc_feature (20)..(20): c is m5c
acagcacaaactactacctca

SEQ ID NO: 511 - DNA, 22 bp, Artificial Sequence
  Synthetic
  misc_feature (8)..(8): c is m5c
aactattcaatctactacctca

SEQ ID NO: 512 - DNA, 22 bp, Artificial Sequence
  Synthetic
  misc_feature (4)..(4): c is m5c
  misc_feature (7)..(7): c is m5c
  misc_feature (16)..(16): c is m5c
tacctgcactgtaagcactttt

SEQ ID NO: 513 - DNA, 22 bp, Artificial Sequence
  Synthetic
  misc_feature (4)..(4): c is m5c
  misc_feature (10)..(10): c is m5c
  misc_feature (13)..(13): c is m5c
tggcattcactgcgtgccttaa

SEQ ID NO: 514 - DNA, 22 bp, Artificial Sequence
  Synthetic
  misc_feature (4)..(4): c is m5c
atgcccttttaatattgcactg

SEQ ID NO: 515 - DNA, 22 bp, Artificial Sequence
  Synthetic
  misc_feature (6)..(6): c is m5c
  misc_feature (18)..(18): c is m5c
acgccctttcattattgcactg

SEQ ID NO: 516 - DNA, 21 bp, Artificial Sequence
  Synthetic
  misc_feature (18)..(18): c is m5c
tagctggttgaaggggaccaa

SEQ ID NO: 517 - DNA, 22 bp, Artificial Sequence
  Synthetic
gcagctggttgaaggggaccaa

SEQ ID NO: 518 - DNA, 22 bp, Artificial Sequence
  Synthetic
ctacgcgtattcttaagcaata

SEQ ID NO: 519 - DNA, 17 bp, Artificial Sequence
  Synthetic
  misc_feature (7)..(7): c is m5c
  misc_feature (10)..(10): c is m5c
  misc_feature (13)..(13): c is m5c
  misc_feature (16)..(16): c is m5c
gattcacaacaccagct

SEQ ID NO: 520 - DNA, 22 bp, Artificial Sequence
  Synthetic
  misc_feature (5)..(5): c is m5c
  misc_feature (14)..(14): c is m5c
aacccatggaattcagttctca

SEQ ID NO: 521 - DNA, 22 bp, Artificial Sequence
  Synthetic
  misc_feature (19)..(19): c is m5c
acaaagttctgtagtgcactga

SEQ ID NO: 522 - DNA, 22 bp, Artificial Sequence
  Synthetic
  misc_feature (7)..(7): c is m5c
tgcaaaccatgatgtgctgcta

SEQ ID NO: 523 - DNA, 22 bp, Artificial Sequence
  Synthetic
  misc_feature (4)..(4): c is m5c
  misc_feature (13)..(13): c is m5c
caccaatatttacgtgctgcta

SEQ ID NO: 524 - DNA, 20 bp, Artificial Sequence
  Synthetic
  misc_feature (9)..(9): c is m5c
  misc_feature (12)..(12): c is m5c
acaagtgccttcactgcagt

SEQ ID NO: 525 - DNA, 22 bp, Artificial Sequence
  Synthetic
  misc_feature (3)..(3): c is m5c
  misc_feature (9)..(9): c is m5c
  misc_feature (21)..(21): c is m5c
acccttatcagttctccgtcca

SEQ ID NO: 526 - DNA, 22 bp, Artificial Sequence
  Synthetic
  misc_feature (4)..(4): c is m5c
  misc_feature (7)..(7): c is m5c
  misc_feature (16)..(16): c is m5c
  misc_feature (19)..(19): c is m5c
tatctgcactagatgcacctta

SEQ ID NO: 527 - DNA, 22 bp, Artificial Sequence
  Synthetic
  misc_feature (4)..(4): c is m5c
  misc_feature (7)..(7): c is m5c
  misc_feature (16)..(16): c is m5c
  misc_feature (19)..(19): c is m5c
taactgcactagatgcacctta

SEQ ID NO: 528 - DNA, 22 bp, Artificial Sequence
  Synthetic
  misc_feature (3)..(3): c is m5c
  misc_feature (12)..(12): c is m5c
  misc_feature (21)..(21): c is m5c
acctaatatatcaaacatatca

SEQ ID NO: 529 - DNA, 21 bp, Artificial Sequence
  Synthetic
  misc_feature (7)..(7): c is m5c
  misc_feature (10)..(10): c is m5c
  misc_feature (19)..(19): c is m5c
tacatacttcttaacattcca

SEQ ID NO: 530 - DNA, 23 bp, Artificial Sequence
  Synthetic
  misc_feature (16)..(16): c is m5c
atcatcattaccaggcagtatta

SEQ ID NO: 531 - DNA, 22 bp, Artificial Sequence
  Synthetic
  misc_feature (8)..(8): c is m5c
cagattccggtggaatgaaggg

SEQ ID NO: 532 - DNA, 21 bp, Artificial Sequence
  Synthetic
  misc_feature (7)..(7): c is m5c
  misc_feature (19)..(19): c is m5c
gtctgtcaattcataggtcat

SEQ ID NO: 533 - DNA, 24 bp, Artificial Sequence
  Synthetic
  misc_feature (5)..(5): c is m5c
  misc_feature (14)..(14): c is m5c
gagacccagtagccagatgtagtt

SEQ ID NO: 534 - DNA, 21 bp, Artificial Sequence
  Synthetic
  misc_feature (7)..(7): c is m5c
  misc_feature (13)..(13): c is m5c
ggtaatccctggcaatgtgat

SEQ ID NO: 535 - DNA, 21 bp, Artificial Sequence
  Synthetic
  misc_feature (3)..(3): c is m5c
gcctatcctggattacttgaa

SEQ ID NO: 536 - DNA, 21 bp, Artificial Sequence
  Synthetic
  misc_feature (12)..(12): c is m5c
  misc_feature (15)..(15): c is m5c
gcagaacttagccactgtgaa

SEQ ID NO: 537 - DNA, 21 bp, Artificial Sequence
  Synthetic
  misc_feature (3)..(3): c is m5c
aaccgatttcaaatggtgcta

SEQ ID NO: 538 - DNA, 23 bp, Artificial Sequence
  Synthetic
  misc_feature (3)..(3): c is m5c
  misc_feature (12)..(12): c is m5c
  misc_feature (21)..(21): c is m5c
aacactgatttcaaatggtgcta

SEQ ID NO: 539 - DNA, 20 bp, Artificial Sequence
  Synthetic
  misc_feature (2)..(2): c is m5c
accgatttcaaatggtgcta

SEQ ID NO: 540 - DNA, 25 bp, Artificial Sequence
  Synthetic
  misc_feature (21)..(21): c is m5c
atgctttgacaatattattgcactg

SEQ ID NO: 541 - DNA, 22 bp, Artificial Sequence
  Synthetic
  misc_feature (18)..(18): c is m5c
tcactaaaacatggaagcactt

SEQ ID NO: 542 - DNA, 22 bp, Artificial Sequence
  Synthetic
  misc_feature (13)..(13): c is m5c
  misc_feature (16)..(16): c is m5c
gctgcaaacatccgactgaaag

SEQ ID NO: 543 - DNA, 22 bp, Artificial Sequence
  Synthetic
  misc_feature (5)..(5): c is m5c
cttccagtcgaggatgtttaca

SEQ ID NO: 544 - DNA, 22 bp, Artificial Sequence
  Synthetic
agctgagtgtaggatgtttaca

SEQ ID NO: 545 - DNA, 22 bp, Artificial Sequence
  Synthetic
  misc_feature (4)..(4): c is m5c
cttccagtcggggatgtttaca

SEQ ID NO: 546 - DNA, 20 bp, Artificial Sequence
  Synthetic
  misc_feature (3)..(3): c is m5c
tccagtcaaggatgtttaca

SEQ ID NO: 547 - DNA, 22 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
cagctatgccaacatcttgcct

SEQ ID NO: 548 - DNA, 21 bp, Artificial Sequence
  Synthetic
gcaacttagtaatgtgcaata

SEQ ID NO: 549 - DNA, 19 bp, Artificial Sequence
  Synthetic
  misc_feature (17)..(17): c is m5c
caatgcaactacaatgcac

SEQ ID NO: 550 - DNA, 23 bp, Artificial Sequence
  Synthetic
  misc_feature (12)..(12): c is m5c
  misc_feature (15)..(15): c is m5c
  misc_feature (21)..(21): c is m5c
caatcagctaactacactgcctg

SEQ ID NO: 551 - DNA, 23 bp, Artificial Sequence
  Synthetic
  misc_feature (6)..(6): c is m5c
  misc_feature (9)..(9): c is m5c
  misc_feature (18)..(18): c is m5c
  misc_feature (21)..(21): c is m5c
gcaatcagctaactacactgcct

SEQ ID NO: 552 - DNA, 22 bp, Artificial Sequence
  Synthetic
  misc_feature (10)..(10): c is m5c
aacaaaaatcactagtcttcca

SEQ ID NO: 553 - DNA, 21 bp, Artificial Sequence
  Synthetic
caggccgggacaagtgcaata

SEQ ID NO: 554 - DNA, 21 bp, Artificial Sequence
  Synthetic
  misc_feature (7)..(7): c is m5c
  misc_feature (10)..(10): c is m5c
  misc_feature (13)..(13): c is m5c
actatacaacctcctacctca

SEQ ID NO: 555 - DNA, 20 bp, Artificial Sequence
  Synthetic
  misc_feature (9)..(9): c is m5c
acaggagtctgagcatttga

SEQ ID NO: 556 - DNA, 24 bp, Artificial Sequence
  Synthetic
gctacctgcactgtaagcactttt

SEQ ID NO: 557 - DNA, 21 bp, Artificial Sequence
  Synthetic
atctgcactgtcagcacttta

SEQ ID NO: 558 - DNA, 23 bp, Artificial Sequence
  Synthetic
cacaggttaaagggtctcaggga

SEQ ID NO: 559 - DNA, 22 bp, Artificial Sequence
  Synthetic
  misc_feature (4)..(4): c is m5c
  misc_feature (10)..(10): c is m5c
  misc_feature (19)..(19): c is m5c
agccaagctcagacggatccga

SEQ ID NO: 560 - DNA, 22 bp, Artificial Sequence
  Synthetic
  misc_feature (9)..(9): c is m5c
  misc_feature (15)..(15): c is m5c
gaaagagaccggttcactgtga

SEQ ID NO: 561 - DNA, 21 bp, Artificial Sequence
  Synthetic
  misc_feature (6)..(6): c is m5c
  misc_feature (12)..(12): c is m5c
  misc_feature (15)..(15): c is m5c
gcaagcccagaccgcaaaaag

SEQ ID NO: 562 - DNA, 22 bp, Artificial Sequence
  Synthetic
  misc_feature (5)..(5): c is m5c
  misc_feature (20)..(20): c is m5c
atgcccttttaacattgcactg

SEQ ID NO: 563 - DNA, 22 bp, Artificial Sequence
  Synthetic
  misc_feature (5)..(5): c is m5c
  misc_feature (20)..(20): c is m5c
atgccctttcatcattgcactg

SEQ ID NO: 564 - DNA, 21 bp, Artificial Sequence
  Synthetic
  misc_feature (3)..(3): c is m5c
  misc_feature (18)..(18): c is m5c
ccctctggtcaaccagtcaca

SEQ ID NO: 565 - DNA, 22 bp, Artificial Sequence
  Synthetic
  misc_feature (19)..(19): c is m5c
cacataggaatgaaaagccata

SEQ ID NO: 566 - DNA, 23 bp, Artificial Sequence
  Synthetic
tccatcatcaaaacaaatggagt

SEQ ID NO: 567 - DNA, 18 bp, Artificial Sequence
  Synthetic
agacacgtgcactgtaga

SEQ ID NO: 568 - DNA, 22 bp, Artificial Sequence
  Synthetic
  misc_feature (2)..(2): c is m5c
  misc_feature (5)..(5): c is m5c
  misc_feature (11)..(11): c is m5c
ccatctttaccagacagtgtta

SEQ ID NO: 569 - DNA, 20 bp, Artificial Sequence
  Synthetic
  misc_feature (18)..(18): c is m5c
gcagaagcatttccacacac

SEQ ID NO: 570 - DNA, 22 bp, Artificial Sequence
  Synthetic
  misc_feature (19)..(19): c is m5c
acaaagttctgtgatgcactga

SEQ ID NO: 571 - DNA, 22 bp, Artificial Sequence
  Synthetic
  misc_feature (13)..(13): c is m5c
  misc_feature (19)..(19): c is m5c
ggagtgaagacacggagccaga

SEQ ID NO: 572 - DNA, 22 bp, Artificial Sequence
  Synthetic
cactggtacaagggttgggaga

SEQ ID NO: 573 - DNA, 22 bp, Artificial Sequence
  Synthetic
  misc_feature (18)..(18): c is m5c
cctcaaggagcttcagtctagt

SEQ ID NO: 574 - DNA, 22 bp, Artificial Sequence
  Synthetic
  misc_feature (2)..(2): c is m5c
  misc_feature (17)..(17): c is m5c
cccaagttctgtcatgcactga

SEQ ID NO: 575 - DNA, 22 bp, Artificial Sequence
  Synthetic
  misc_feature (20)..(20): c is m5c
cgaaggcaacacggataaccta

SEQ ID NO: 576 - DNA, 22 bp, Artificial Sequence
  Synthetic
  misc_feature (8)..(8): c is m5c
  misc_feature (11)..(11): c is m5c
aataggtcaaccgtgtatgatt

SEQ ID NO: 577 - DNA, 22 bp, Artificial Sequence
  Synthetic
  misc_feature (7)..(7): c is m5c
tgtaaaccatgatgtgctgcta

SEQ ID NO: 578 - DNA, 20 bp, Artificial Sequence
  Synthetic
  misc_feature (9)..(9): c is m5c
  misc_feature (12)..(12): c is m5c
acaagtgccttcactgcagt

SEQ ID NO: 579 - DNA, 22 bp, Artificial Sequence
  Synthetic
  misc_feature (2)..(2): c is m5c
actcaccgacaggttgaatgtt

SEQ ID NO: 580 - DNA, 18 bp, Artificial Sequence
  Synthetic
  misc_feature (8)..(8): c is m5c
  misc_feature (14)..(14): c is m5c
  misc_feature (17)..(17): c is m5c
gaactgcctttctctcca

SEQ ID NO: 581 - DNA, 23 bp, Artificial Sequence
  Synthetic
  misc_feature (6)..(6): c is m5c
aagcccaaaaggagaattctttg

SEQ ID NO: 582 - DNA, 21 bp, Artificial Sequence
  Synthetic
  misc_feature (17)..(17): c is m5c
cggctgcaacacaagacacga

SEQ ID NO: 583 - DNA, 22 bp, Artificial Sequence
  Synthetic
  misc_feature (2)..(2): c is m5c
  misc_feature (14)..(14): c is m5c
accctccaccatgcaagggatg

SEQ ID NO: 584 - DNA, 23 bp, Artificial Sequence
  Synthetic
actgatatcagctcagtaggcac

SEQ ID NO: 585 - DNA, 22 bp, Artificial Sequence
  Synthetic
agctgcttttgggattccgttg

SEQ ID NO: 586 - DNA, 21 bp, Artificial Sequence
  Synthetic
ggctgtcaattcataggtcag

SEQ ID NO: 587 - DNA, 21 bp, Artificial Sequence
  Synthetic
  misc_feature (19)..(19): c is m5c
ccaacaacatgaaactaccta

SEQ ID NO: 588 - DNA, 21 bp, Artificial Sequence
  Synthetic
  misc_feature (2)..(2): c is m5c
  misc_feature (5)..(5): c is m5c
  misc_feature (8)..(8): c is m5c
ccaacaacaggaaactaccta

SEQ ID NO: 589 - DNA, 22 bp, Artificial Sequence
  Synthetic
gctgggtggagaaggtggtgaa

SEQ ID NO: 590 - DNA, 19 bp, Artificial Sequence
  Synthetic
  misc_feature (2)..(2): c is m5c
  misc_feature (8)..(8): c is m5c
  misc_feature (11)..(11): c is m5c
cctatctcccctctggacc

SEQ ID NO: 591 - DNA, 23 bp, Artificial Sequence
  Synthetic
  misc_feature (17)..(17): c is m5c
gaacagatagtctaaacactggg

SEQ ID NO: 592 - DNA, 23 bp, Artificial Sequence
  Synthetic
  misc_feature (16)..(16): c is m5c
gtcatcattaccaggcagtatta

SEQ ID NO: 593 - DNA, 22 bp, Artificial Sequence
  Synthetic
  misc_feature (12)..(12): c is m5c
  misc_feature (15)..(15): c is m5c
ccatcattacccggcagtatta

SEQ ID NO: 594 - DNA, 22 bp, Artificial Sequence
  Synthetic
  misc_feature (20)..(20): c is m5c
ctagtggtcctaaacatttcac

SEQ ID NO: 595 - DNA, 22 bp, Artificial Sequence
  Synthetic
  misc_feature (6)..(6): c is m5c
  misc_feature (15)..(15): c is m5c
  misc_feature (18)..(18): c is m5c
acaagctttttgctcgtcttat

SEQ ID NO: 596 - DNA, 22 bp, Artificial Sequence
  Synthetic
  misc_feature (2)..(2): c is m5c
  misc_feature (5)..(5): c is m5c
  misc_feature (8)..(8): c is m5c
  misc_feature (20)..(20): c is m5c
tcaacatcagtctgataagcta

SEQ ID NO: 597 - DNA, 22 bp, Artificial Sequence
  Synthetic
  misc_feature (6)..(6): c is m5c
  misc_feature (12)..(12): c is m5c
  misc_feature (18)..(18): c is m5c
tcagccgctgtcacacgcacag

SEQ ID NO: 598 - DNA, 22 bp, Artificial Sequence
  Synthetic
  misc_feature (4)..(4): c is m5c
aggcgaaggatgacaaagggaa

SEQ ID NO: 599 - DNA, 21 bp, Artificial Sequence
  Synthetic
ggccgtgactggagactgtta

SEQ ID NO: 600 - DNA, 21 bp, Artificial Sequence
  Synthetic
aaagtgtcagatacggtgtgg

SEQ ID NO: 601 - DNA, 23 bp, Artificial Sequence
  Synthetic
  misc_feature (5)..(5): c is m5c
  misc_feature (11)..(11): c is m5c
  misc_feature (20)..(20): c is m5c
taaacggaaccactagtgacttg

SEQ ID NO: 602 - DNA, 21 bp, Artificial Sequence
  Synthetic
  misc_feature (12)..(12): c is m5c
  misc_feature (15)..(15): c is m5c
gcggaacttagccactgtgaa

SEQ ID NO: 603 - DNA, 22 bp, Artificial Sequence
  Synthetic
  misc_feature (3)..(3): c is m5c
ctcaatagactgtgagctcctt

SEQ ID NO: 604 - DNA, 21 bp, Artificial Sequence
  Synthetic
  misc_feature (18)..(18): c is m5c
acaggattgagggggggccct

SEQ ID NO: 605 - DNA, 22 bp, Artificial Sequence
  Synthetic
  misc_feature (20)..(20): c is m5c
atgtatgtgggacggtaaacca

SEQ ID NO: 606 - DNA, 21 bp, Artificial Sequence
  Synthetic
  misc_feature (3)..(3): c is m5c
aaccgatttcagatggtgcta

SEQ ID NO: 607 - DNA, 23 bp, Artificial Sequence
  Synthetic
  misc_feature (3)..(3): c is m5c
  misc_feature (12)..(12): c is m5c
  misc_feature (21)..(21): c is m5c
aacactgatttcaaatggtgcta

SEQ ID NO: 608 - DNA, 20 bp, Artificial Sequence
  Synthetic
  misc_feature (2)..(2): c is m5c
accgatttcaaatggtgcta

SEQ ID NO: 609 - DNA, 23 bp, Artificial Sequence
  Synthetic
  misc_feature (18)..(18): c is m5c
tcaccaaaacatggaagcactta

SEQ ID NO: 610 - DNA, 22 bp, Artificial Sequence
  Synthetic
aaagcaagtacatccacgttta

SEQ ID NO: 611 - DNA, 23 bp, Artificial Sequence
  Synthetic
  misc_feature (18)..(18): c is m5c
ctactaaaacatggaagcactta

SEQ ID NO: 612 - DNA, 23 bp, Artificial Sequence
  Synthetic
  misc_feature (9)..(9): c is m5c
  misc_feature (12)..(12): c is m5c
agaaagcacttccatgttaaagt

SEQ ID NO: 613 - DNA, 23 bp, Artificial Sequence
  Synthetic
  misc_feature (18)..(18): c is m5c
ccactgaaacatggaagcactta

SEQ ID NO: 614 - DNA, 22 bp, Artificial Sequence
  Synthetic
  misc_feature (4)..(4): c is m5c
  misc_feature (10)..(10): c is m5c
  misc_feature (13)..(13): c is m5c
cagcaggtacccccatgttaaa

SEQ ID NO: 615 - DNA, 23 bp, Artificial Sequence
  Synthetic
  misc_feature (6)..(6): c is m5c
  misc_feature (18)..(18): c is m5c
acactcaaacatggaagcactta

SEQ ID NO: 616 - DNA, 22 bp, Artificial Sequence
  Synthetic
  misc_feature (2)..(2): c is m5c
gctgtaaacatccgactgaaag

SEQ ID NO: 617 - DNA, 21 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
cagctatgccagcatcttgcc

SEQ ID NO: 618 - DNA, 22 bp, Artificial Sequence
  Synthetic
agaggtcgaccgtgtaatgtgc

SEQ ID NO: 619 - DNA, 22 bp, Artificial Sequence
  Synthetic
ccagcagcacctggggcagtgg

SEQ ID NO: 620 - DNA, 23 bp, Artificial Sequence
  Synthetic
  misc_feature (12)..(12): c is m5c
acaccaatgccctaggggatgcg

SEQ ID NO: 621 - DNA, 23 bp, Artificial Sequence
  Synthetic
  misc_feature (2)..(2): c is m5c
  misc_feature (8)..(8): c is m5c
acacttactggacacctactagg

SEQ ID NO: 622 - DNA, 20 bp, Artificial Sequence
  Synthetic
  misc_feature (13)..(13): c is m5c
ctggaggaagggcccagagg

SEQ ID NO: 623 - DNA, 22 bp, Artificial Sequence
  Synthetic
acggaagggcagagagggccag

SEQ ID NO: 624 - DNA, 23 bp, Artificial Sequence
  Synthetic
  misc_feature (2)..(2): c is m5c
  misc_feature (11)..(11): c is m5c
tctctgcaggccgtgtgctttgc

SEQ ID NO: 625 - DNA, 21 bp, Artificial Sequence
  Synthetic
  misc_feature (14)..(14): c is m5c
ttctaggataggcccaggggc

SEQ ID NO: 626 - DNA, 23 bp, Artificial Sequence
  Synthetic
  misc_feature (2)..(2): c is m5c
  misc_feature (17)..(17): c is m5c
acatttttcgttattgctcttga

SEQ ID NO: 627 - DNA, 23 bp, Artificial Sequence
  Synthetic
  misc_feature (19)..(19): c is m5c
aaaggcatcatataggagctgga

SEQ ID NO: 628 - DNA, 23 bp, Artificial Sequence
  Synthetic
  misc_feature (13)..(13): c is m5c
  misc_feature (19)..(19): c is m5c
tcaacaaaatcactgatgctgga

SEQ ID NO: 629 - DNA, 21 bp, Artificial Sequence
  Synthetic
tgagctcctggaggacaggga

SEQ ID NO: 630 - DNA, 23 bp, Artificial Sequence
  Synthetic
ggctataaagtaactgagacgga

SEQ ID NO: 631 - DNA, 24 bp, Artificial Sequence
  Synthetic
gacgggtgcgatttctgtgtgaga

SEQ ID NO: 632 - DNA, 21 bp, Artificial Sequence
  Synthetic
  misc_feature (2)..(2): c is m5c
  misc_feature (17)..(17): c is m5c
  misc_feature (20)..(20): c is m5c
gccctggactaggagtcagca

SEQ ID NO: 633 - DNA, 23 bp, Artificial Sequence
  Synthetic
  misc_feature (5)..(5): c is m5c
  misc_feature (8)..(8): c is m5c
  misc_feature (17)..(17): c is m5c
  misc_feature (20)..(20): c is m5c
caatcagctaatgacactgccta

SEQ ID NO: 634 - DNA, 22 bp, Artificial Sequence
  Synthetic
  misc_feature (5)..(5): c is m5c
gtacccctggagattctgataa

SEQ ID NO: 635 - DNA, 22 bp, Artificial Sequence
  Synthetic
  misc_feature (2)..(2): c is m5c
  misc_feature (5)..(5): c is m5c
tcaccattgctaaagtgcaatt

SEQ ID NO: 636 - DNA, 22 bp, Artificial Sequence
  Synthetic
  misc_feature (4)..(4): c is m5c
aaacgtggaatttcctctatgt

SEQ ID NO: 637 - DNA, 21 bp, Artificial Sequence
  Synthetic
aaagatcaaccatgtattatt

SEQ ID NO: 638 - DNA, 21 bp, Artificial Sequence
  Synthetic
  misc_feature (9)..(9): c is m5c
  misc_feature (12)..(12): c is m5c
ccaggttccaccccagcaggc

SEQ ID NO: 639 - DNA, 21 bp, Artificial Sequence
  Synthetic
  misc_feature (6)..(6): c is m5c
acactcaaaagatggcggcac

SEQ ID NO: 640 - DNA, 23 bp, Artificial Sequence
  Synthetic
  misc_feature (4)..(4): c is m5c
  misc_feature (13)..(13): c is m5c
acgctcaaatgtcgcagcacttt

SEQ ID NO: 641 - DNA, 23 bp, Artificial Sequence
  Synthetic
  misc_feature (4)..(4): c is m5c
  misc_feature (7)..(7): c is m5c
  misc_feature (13)..(13): c is m5c
acaccccaaaatcgaagcacttc

SEQ ID NO: 642 - DNA, 22 bp, Artificial Sequence
  Synthetic
  misc_feature (7)..(7): c is m5c
  misc_feature (10)..(10): c is m5c
  misc_feature (13)..(13): c is m5c
ggaaagcgcccccattttgagt

SEQ ID NO: 643 - DNA, 22 bp, Artificial Sequence
  Synthetic
cacttatcaggttgtattataa

SEQ ID NO: 644 - DNA, 22 bp, Artificial Sequence
  Synthetic
  misc_feature (6)..(6): c is m5c
  misc_feature (15)..(15): c is m5c
tcacgcgagccgaacgaacaaa

SEQ ID NO: 645 - DNA, 21 bp, Artificial Sequence
  Synthetic
  misc_feature (13)..(13): c is m5c
acgtggattttcctctatgat

SEQ ID NO: 646 - DNA, 22 bp, Artificial Sequence
  Synthetic
  misc_feature (12)..(12): c is m5c
acaaaagttgcctttgtgtgat

SEQ ID NO: 647 - DNA, 22 bp, Artificial Sequence
  Synthetic
  misc_feature (2)..(2): c is m5c
  misc_feature (17)..(17): c is m5c
acacaggacctggagtcaggag

SEQ ID NO: 648 - DNA, 19 bp, Artificial Sequence
  Synthetic
  misc_feature (7)..(7): c is m5c
tacgttccatagtctacca

SEQ ID NO: 649 - DNA, 22 bp, Artificial Sequence
  Synthetic
  misc_feature (19)..(19): c is m5c
aagatgtggaccatattacata

SEQ ID NO: 650 - DNA, 22 bp, Artificial Sequence
  Synthetic
  misc_feature (2)..(2): c is m5c
  misc_feature (17)..(17): c is m5c
  misc_feature (20)..(20): c is m5c
gcgcatgttctatggtcaacca

SEQ ID NO: 651 - DNA, 22 bp, Artificial Sequence
  Synthetic
  misc_feature (2)..(2): c is m5c
  misc_feature (14)..(14): c is m5c
acagagagcttgcccttgtata

SEQ ID NO: 652 - DNA, 22 bp, Artificial Sequence
  Synthetic
  misc_feature (7)..(7): c is m5c
  misc_feature (10)..(10): c is m5c
  misc_feature (16)..(16): c is m5c
  misc_feature (19)..(19): c is m5c
cgaatccaccacgaacaacttc

SEQ ID NO: 653 - DNA, 22 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
agccacaatcaccttctgatct

SEQ ID NO: 654 - DNA, 20 bp, Artificial Sequence
  Synthetic
tatgaacaatttctaggaat

SEQ ID NO: 655 - DNA, 22 bp, Artificial Sequence
  Synthetic
  misc_feature (4)..(4): c is m5c
  misc_feature (7)..(7): c is m5c
  misc_feature (13)..(13): c is m5c
  misc_feature (19)..(19): c is m5c
ggccttctgaccctaagtccag

SEQ ID NO: 656 - DNA, 22 bp, Artificial Sequence
  Synthetic
  misc_feature (3)..(3): c is m5c
ggccttctgactccaagtccag

SEQ ID NO: 657 - DNA, 22 bp, Artificial Sequence
  Synthetic
  misc_feature (10)..(10): c is m5c
  misc_feature (16)..(16): c is m5c
ctgaggggcctcagaccgagct

SEQ ID NO: 658 - DNA, 22 bp, Artificial Sequence
  Synthetic
ttcaaaacatgaattgctgctg

SEQ ID NO: 659 - DNA, 21 bp, Artificial Sequence
  Synthetic
  misc_feature (3)..(3): c is m5c
  misc_feature (9)..(9): c is m5c
  misc_feature (12)..(12): c is m5c
  misc_feature (18)..(18): c is m5c
ggcggacacgacattcccgat

SEQ ID NO: 660 - DNA, 22 bp, Artificial Sequence
  Synthetic
ctacctgcacgaacagcacttt

SEQ ID NO: 661 - DNA, 22 bp, Artificial Sequence
  Synthetic
  misc_feature (16)..(16): c is m5c
tgctcaataaatacccgttgaa

SEQ ID NO: 662 - DNA, 22 bp, Artificial Sequence
  Synthetic
  misc_feature (19)..(19): c is m5c
gcaaaaatgtgctagtgccaaa

SEQ ID NO: 663 - DNA, 22 bp, Artificial Sequence
  Synthetic
  misc_feature (8)..(8): c is m5c
  misc_feature (11)..(11): c is m5c
aacaatacaacttactacctca

SEQ ID NO: 664 - DNA, 22 bp, Artificial Sequence
  Synthetic
cgcaaggtcggttctacgggtg

SEQ ID NO: 665 - DNA, 22 bp, Artificial Sequence
  Synthetic
agaaaggcagcaggtcgtatag

SEQ ID NO: 666 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
actatacaatctactacctca

SEQ ID NO: 667 - DNA, 22 bp, Artificial Sequence
  Synthetic
  misc_feature (4)..(4): c is m5c
  misc_feature (7)..(7): c is m5c
  misc_feature (13)..(13): c is m5c
cttcagctatcacagtactgta

SEQ ID NO: 668 - DNA, 22 bp, Artificial Sequence
  Synthetic
  misc_feature (4)..(4): c is m5c
  misc_feature (7)..(7): c is m5c
  misc_feature (16)..(16): c is m5c
tacctgcactgttagcactttg

SEQ ID NO: 669 - DNA, 20 bp, Artificial Sequence
  Synthetic
  misc_feature (3)..(3): c is m5c
  misc_feature (9)..(9): c is m5c
  misc_feature (12)..(12): c is m5c
ggcattcaccgcgtgcctta

SEQ ID NO: 670 - DNA, 21 bp, Artificial Sequence
  Synthetic
  misc_feature (3)..(3): c is m5c
  misc_feature (9)..(9): c is m5c
  misc_feature (18)..(18): c is m5c
gccaagctcagacggatccga

SEQ ID NO: 671 - DNA, 22 bp, Artificial Sequence
  Synthetic
  misc_feature (4)..(4): c is m5c
  misc_feature (19)..(19): c is m5c
cccctctggtcaaccagtcaca

SEQ ID NO: 672 - DNA, 23 bp, Artificial Sequence
  Synthetic
ctacgcgtattcttaagcaataa

SEQ ID NO: 673 - DNA, 22 bp, Artificial Sequence
  Synthetic
  misc_feature (4)..(4): c is m5c
ctaccatagggtaaaaccactg

SEQ ID NO: 674 - DNA, 22 bp, Artificial Sequence
  Synthetic
  misc_feature (3)..(3): c is m5c
  misc_feature (15)..(15): c is m5c
tccgtggttctaccctgtggta

SEQ ID NO: 675 - DNA, 22 bp, Artificial Sequence
  Synthetic
  misc_feature (16)..(16): c is m5c
ccataaagtaggaaacactaca

SEQ ID NO: 676 - DNA, 21 bp, Artificial Sequence
  Synthetic
  misc_feature (2)..(2): c is m5c
  misc_feature (11)..(11): c is m5c
  misc_feature (14)..(14): c is m5c
cctcaaggagcctcagtctag

SEQ ID NO: 677 - DNA, 22 bp, Artificial Sequence
  Synthetic
  misc_feature (4)..(4): c is m5c
gatcacttttgtgactatgcaa

SEQ ID NO: 678 - DNA, 22 bp, Artificial Sequence
  Synthetic
  misc_feature (3)..(3): c is m5c
cccctatcacaattagcattaa

SEQ ID NO: 679 - DNA, 21 bp, Artificial Sequence
  Synthetic
  misc_feature (10)..(10): c is m5c
  misc_feature (13)..(13): c is m5c
tacaagtgccctcactgcagt

SEQ ID NO: 680 - DNA, 22 bp, Artificial Sequence
  Synthetic
  misc_feature (18)..(18): c is m5c
ccggctgcaacacaagacacga

SEQ ID NO: 681 - DNA, 18 bp, Artificial Sequence
  Synthetic
  misc_feature (9)..(9): c is m5c
tgtcaattcataggtcag

SEQ ID NO: 682 - DNA, 21 bp, Artificial Sequence
  Synthetic
  misc_feature (2)..(2): c is m5c
  misc_feature (11)..(11): c is m5c
gccaatatttctgtgctgcta

SEQ ID NO: 683 - DNA, 21 bp, Artificial Sequence
  Synthetic
  misc_feature (15)..(15): c is m5c
agaacaatgccttactgagta

SEQ ID NO: 684 - DNA, 22 bp, Artificial Sequence
  Synthetic
  misc_feature (2)..(2): c is m5c
  misc_feature (5)..(5): c is m5c
  misc_feature (11)..(11): c is m5c
tcttcccatgcgctatacctct

SEQ ID NO: 685 - DNA, 21 bp, Artificial Sequence
  Synthetic
  misc_feature (20)..(20): c is m5c
ctagtggtcctaaacatttca

SEQ ID NO: 686 - DNA, 23 bp, Artificial Sequence
  Synthetic
gagggaggagagccaggagaagc

SEQ ID NO: 687 - DNA, 22 bp, Artificial Sequence
  Synthetic
  misc_feature (4)..(4): c is m5c
aggcaaaggatgacaaagggaa

SEQ ID NO: 688 - DNA, 21 bp, Artificial Sequence
  Synthetic
  misc_feature (3)..(3): c is m5c
  misc_feature (12)..(12): c is m5c
gtctgtcaaatcataggtcat

SEQ ID NO: 689 - DNA, 21 bp, Artificial Sequence
  Synthetic
  misc_feature (3)..(3): c is m5c
  misc_feature (9)..(9): c is m5c
cacagttgccagctgagatta

SEQ ID NO: 690 - DNA, 24 bp, Artificial Sequence
  Synthetic
  misc_feature (3)..(3): c is m5c
atccagtcagttcctgatgcagta

SEQ ID NO: 691 - DNA, 22 bp, Artificial Sequence
  Synthetic
  misc_feature (4)..(4): c is m5c
  misc_feature (13)..(13): c is m5c
aaacccagcagacaatgtagct

SEQ ID NO: 692 - DNA, 23 bp, Artificial Sequence
  Synthetic
  misc_feature (5)..(5): c is m5c
  misc_feature (11)..(11): c is m5c
  misc_feature (20)..(20): c is m5c
taaacggaaccactagtgactta

SEQ ID NO: 693 - DNA, 23 bp, Artificial Sequence
  Synthetic
  misc_feature (10)..(10): c is m5c
  misc_feature (13)..(13): c is m5c
aaaaagtgcccccatagtttgag

SEQ ID NO: 694 - DNA, 23 bp, Artificial Sequence
  Synthetic
  misc_feature (5)..(5): c is m5c
  misc_feature (20)..(20): c is m5c
ggcacacaaagtggaagcacttt

SEQ ID NO: 695 - DNA, 22 bp, Artificial Sequence
  Synthetic
agagagggcctccactttgatg

SEQ ID NO: 696 - DNA, 23 bp, Artificial Sequence
  Synthetic
  misc_feature (6)..(6): c is m5c
  misc_feature (12)..(12): c is m5c
  misc_feature (21)..(21): c is m5c
acactcaaaacctggcggcactt

SEQ ID NO: 697 - DNA, 22 bp, Artificial Sequence
  Synthetic
  misc_feature (10)..(10): c is m5c
  misc_feature (13)..(13): c is m5c
caaaagagcccccagtttgagt

SEQ ID NO: 698 - DNA, 22 bp, Artificial Sequence
  Synthetic
  misc_feature (2)..(2): c is m5c
  misc_feature (11)..(11): c is m5c
acactacaaactctgcggcact

SEQ ID NO: 699 - DNA, 22 bp, Artificial Sequence
  Synthetic
  misc_feature (6)..(6): c is m5c
acacacaaaagggaagcacttt

SEQ ID NO: 700 - DNA, 23 bp, Artificial Sequence
  Synthetic
  misc_feature (4)..(4): c is m5c
agactcaaaagtagtagcacttt

SEQ ID NO: 701 - DNA, 21 bp, Artificial Sequence
  Synthetic
  misc_feature (5)..(5): c is m5c
  misc_feature (11)..(11): c is m5c
catgcacatgcacacatacat

SEQ ID NO: 702 - DNA, 22 bp, Artificial Sequence
  Synthetic
  misc_feature (8)..(8): c is m5c
  misc_feature (11)..(11): c is m5c
ggaagaacagccctcctctgcc

SEQ ID NO: 703 - DNA, 22 bp, Artificial Sequence
  Synthetic
  misc_feature (14)..(14): c is m5c
gaagagagcttgcccttgcata

SEQ ID NO: 704 - DNA, 23 bp, Artificial Sequence
  Synthetic
  misc_feature (13)..(13): c is m5c
  misc_feature (19)..(19): c is m5c
gctttgacaatactattgcactg

SEQ ID NO: 705 - DNA, 21 bp, Artificial Sequence
  Synthetic
ctgtaaacatccgactgaaag

SEQ ID NO: 706 - DNA, 22 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
cagctatgccagcatcttgcct

SEQ ID NO: 707 - DNA, 20 bp, Artificial Sequence
  Synthetic
tgttgcagcgcttcatgttt

SEQ ID NO: 708 - DNA, 22 bp, Artificial Sequence
  Synthetic
tccaaaacatgaattgctgctg

SEQ ID NO: 709 - DNA, 22 bp, Artificial Sequence
  Synthetic
  misc_feature (11)..(11): c is m5c
caccaatgccctaggggatgcg

SEQ ID NO: 710 - DNA, 23 bp, Artificial Sequence
  Synthetic
  misc_feature (4)..(4): c is m5c
  misc_feature (13)..(13): c is m5c
  misc_feature (16)..(16): c is m5c
  misc_feature (19)..(19): c is m5c
acacttactgagcacctactagg

SEQ ID NO: 711 - DNA, 21 bp, Artificial Sequence
  Synthetic
  misc_feature (16)..(16): c is m5c
actggaggaagggcccagagg

SEQ ID NO: 712 - DNA, 22 bp, Artificial Sequence
  Synthetic
aaaaaggttagctgggtgtgtt

SEQ ID NO: 713 - DNA, 23 bp, Artificial Sequence
  Synthetic
  misc_feature (2)..(2): c is m5c
  misc_feature (11)..(11): c is m5c
tctctgcaggccctgtgctttgc

SEQ ID NO: 714 - DNA, 23 bp, Artificial Sequence
  Synthetic
  misc_feature (19)..(19): c is m5c
aaaggcatcatataggagctgaa

SEQ ID NO: 715 - DNA, 21 bp, Artificial Sequence
  Synthetic
  misc_feature (2)..(2): c is m5c
  misc_feature (11)..(11): c is m5c
  misc_feature (14)..(14): c is m5c
actgaccgaccgaccgatcga

SEQ ID NO: 716 - DNA, 23 bp, Artificial Sequence
  Synthetic
  misc_feature (2)..(2): c is m5c
acagtcaggctttggctagatca

SEQ ID NO: 717 - DNA, 21 bp, Artificial Sequence
  Synthetic
  misc_feature (9)..(9): c is m5c
gcactggactaggggtcagca

SEQ ID NO: 718 - DNA, 23 bp, Artificial Sequence
  Synthetic
  misc_feature (10)..(10): c is m5c
  misc_feature (22)..(22): c is m5c
agaggcaggcactcgggcagaca

SEQ ID NO: 719 - DNA, 23 bp, Artificial Sequence
  Synthetic
caatcagctaattacactgccta

SEQ ID NO: 720 - DNA, 23 bp, Artificial Sequence
  Synthetic
tgaaagtgtatgggctttgtgaa

SEQ ID NO: 721 - DNA, 24 bp, Artificial Sequence
  Synthetic
  misc_feature (7)..(7): c is m5c
  misc_feature (16)..(16): c is m5c
  misc_feature (19)..(19): c is m5c
caggctcaaagggctcctcaggga

SEQ ID NO: 722 - DNA, 23 bp, Artificial Sequence
  Synthetic
  misc_feature (11)..(11): c is m5c
  misc_feature (14)..(14): c is m5c
aaccaggttccaccccagcaggc

SEQ ID NO: 723 - DNA, 21 bp, Artificial Sequence
  Synthetic
  misc_feature (13)..(13): c is m5c
acgtggattttcctctacgat

SEQ ID NO: 724 - DNA, 22 bp, Artificial Sequence
  Synthetic
  misc_feature (14)..(14): c is m5c
aaagtggatgttcctctatgat

SEQ ID NO: 725 - DNA, 21 bp, Artificial Sequence
  Synthetic
  misc_feature (10)..(10): c is m5c
  misc_feature (16)..(16): c is m5c
  misc_feature (19)..(19): c is m5c
cctacgttccatagtctacca

SEQ ID NO: 726 - DNA, 22 bp, Artificial Sequence
  Synthetic
  misc_feature (16)..(16): c is m5c
  misc_feature (19)..(19): c is m5c
aagatgtggaccatactacata

SEQ ID NO: 727 - DNA, 22 bp, Artificial Sequence
  Synthetic
  misc_feature (4)..(4): c is m5c
  misc_feature (10)..(10): c is m5c
  misc_feature (13)..(13): c is m5c
  misc_feature (16)..(16): c is m5c
agccacagtcaccttctgatct

SEQ ID NO: 728 - DNA, 20 bp, Artificial Sequence
  Synthetic
tgtgaacaatttctaggaat

SEQ ID NO: 729 - DNA, 23 bp, Artificial Sequence
  Synthetic
  misc_feature (11)..(11): c is m5c
aaggggttcaccgagcaacattc

SEQ ID NO: 730 - DNA, 22 bp, Artificial Sequence
  Synthetic
  misc_feature (7)..(7): c is m5c
aacaggccatctgtgttatatt

SEQ ID NO: 731 - DNA, 23 bp, Artificial Sequence
  Synthetic
actgagggttagtggaccgtgtt

SEQ ID NO: 732 - DNA, 23 bp, Artificial Sequence
  Synthetic
  misc_feature (13)..(13): c is m5c
acggctagtggaccaggtgaagt

SEQ ID NO: 733 - DNA, 22 bp, Artificial Sequence
  Synthetic
  misc_feature (5)..(5): c is m5c
  misc_feature (8)..(8): c is m5c
  misc_feature (17)..(17): c is m5c
catacagctagataaccaaaga

SEQ ID NO: 734 - DNA, 23 bp, Artificial Sequence
  Synthetic
ctacctgcacgaacagcactttg

SEQ ID NO: 735 - DNA, 20 bp, Artificial Sequence
  Synthetic
acaagatcggatctacgggt

SEQ ID NO: 736 - DNA, 21 bp, Artificial Sequence
  Synthetic
  misc_feature (9)..(9): c is m5c
tgtgctctctctcttctgtca

SEQ ID NO: 737 - DNA, 21 bp, Artificial Sequence
  Synthetic
  misc_feature (9)..(9): c is m5c
tatgctcactctcttctgtcg

SEQ ID NO: 738 - DNA, 21 bp, Artificial Sequence
  Synthetic
  misc_feature (10)..(10): c is m5c
  misc_feature (13)..(13): c is m5c
cagagctcccttcaatccaaa

SEQ ID NO: 739 - DNA, 21 bp, Artificial Sequence
  Synthetic
  misc_feature (10)..(10): c is m5c
  misc_feature (13)..(13): c is m5c
tggagctcccttcaatccaat

SEQ ID NO: 740 - DNA, 21 bp, Artificial Sequence
  Synthetic
  misc_feature (10)..(10): c is m5c
  misc_feature (13)..(13): c is m5c
cggagctcccttcaatccaat

SEQ ID NO: 741 - DNA, 21 bp, Artificial Sequence
  Synthetic
  misc_feature (10)..(10): c is m5c
  misc_feature (13)..(13): c is m5c
aggagctcccttcaatccaat

SEQ ID NO: 742 - DNA, 21 bp, Artificial Sequence
  Synthetic
  misc_feature (10)..(10): c is m5c
  misc_feature (13)..(13): c is m5c
tagagctcccttcaatccaag

SEQ ID NO: 743 - DNA, 21 bp, Artificial Sequence
  Synthetic
  misc_feature (4)..(4): c is m5c
  misc_feature (16)..(16): c is m5c
cggcatacagggagccaggca

SEQ ID NO: 744 - DNA, 21 bp, Artificial Sequence
  Synthetic
  misc_feature (15)..(15): c is m5c
tggcattcagggagccaggca

SEQ ID NO: 745 - DNA, 21 bp, Artificial Sequence
  Synthetic
  misc_feature (19)..(19): c is m5c
ctggatgcagaggcttatcga

SEQ ID NO: 746 - DNA, 21 bp, Artificial Sequence
  Synthetic
  misc_feature (5)..(5): c is m5c
  misc_feature (11)..(11): c is m5c
  misc_feature (14)..(14): c is m5c
  misc_feature (17)..(17): c is m5c
  misc_feature (20)..(20): c is m5c
tgcacgtaccctgcttctcca

SEQ ID NO: 747 - DNA, 21 bp, Artificial Sequence
  Synthetic
  misc_feature (10)..(10): c is m5c
  misc_feature (19)..(19): c is m5c
agcacgtgccctgcttctcca

SEQ ID NO: 748 - DNA, 21 bp, Artificial Sequence
  Synthetic
  misc_feature (10)..(10): c is m5c
  misc_feature (19)..(19): c is m5c
ctcacgtgccctgcttctcca

SEQ ID NO: 749 - DNA, 21 bp, Artificial Sequence
  Synthetic
ggggaatgaagcctggtccga

SEQ ID NO: 750 - DNA, 21 bp, Artificial Sequence
  Synthetic
gaggaatgaagcctggtccga

SEQ ID NO: 751 - DNA, 21 bp, Artificial Sequence
  Synthetic
gaggaatgaagcctgatccga

SEQ ID NO: 752 - DNA, 21 bp, Artificial Sequence
  Synthetic
agggattgaagcctggtccga

SEQ ID NO: 753 - DNA, 21 bp, Artificial Sequence
  Synthetic
  misc_feature (14)..(14): c is m5c
  misc_feature (17)..(17): c is m5c
  misc_feature (20)..(20): c is m5c
cagatcatgctggcagcttca

SEQ ID NO: 754 - DNA, 21 bp, Artificial Sequence
  Synthetic
  misc_feature (3)..(3): c is m5c
  misc_feature (9)..(9): c is m5c
  misc_feature (12)..(12): c is m5c
  misc_feature (15)..(15): c is m5c
gtcccgatctgcaccaagcga

SEQ ID NO: 755 - DNA, 21 bp, Artificial Sequence
  Synthetic
  misc_feature (4)..(4): c is m5c
  misc_feature (19)..(19): c is m5c
ttcccgagctgcaccaagcct

SEQ ID NO: 756 - DNA, 21 bp, Artificial Sequence
  Synthetic
ccggcaattcatccttggcta

SEQ ID NO: 757 - DNA, 21 bp, Artificial Sequence
  Synthetic
ccggcaagtcatccttggcta

SEQ ID NO: 758 - DNA, 21 bp, Artificial Sequence
  Synthetic
taggcaagtcatccttggcta

SEQ ID NO: 759 - DNA, 21 bp, Artificial Sequence
  Synthetic
taggcaagtcattcttggcta

SEQ ID NO: 760 - DNA, 22 bp, Artificial Sequence
  Synthetic
ccggcaagtttgtccttggcta

SEQ ID NO: 761 - DNA, 21 bp, Artificial Sequence
  Synthetic
catgggcagtctccttggcta

SEQ ID NO: 762 - DNA, 21 bp, Artificial Sequence
  Synthetic
  misc_feature (9)..(9): c is m5c
gatattggcacggctcaatca

SEQ ID NO: 763 - DNA, 21 bp, Artificial Sequence
  Synthetic
  misc_feature (9)..(9): c is m5c
  misc_feature (18)..(18): c is m5c
gatattggctcggctcacctc

SEQ ID NO: 764 - DNA, 21 bp, Artificial Sequence
  Synthetic
agtgatattggttcggctcac

SEQ ID NO: 765 - DNA, 21 bp, Artificial Sequence
  Synthetic
  misc_feature (20)..(20): c is m5c
gtgcagcatcatcaagattca

SEQ ID NO: 766 - DNA, 20 bp, Artificial Sequence
  Synthetic
  misc_feature (10)..(10): c is m5c
  misc_feature (13)..(13): c is m5c
gggagcacccttcagtccaa

SEQ ID NO: 767 - DNA, 22 bp, Artificial Sequence
  Synthetic
  misc_feature (10)..(10): c is m5c
  misc_feature (16)..(16): c is m5c
agatcaatgcgatccctttgga

SEQ ID NO: 768 - DNA, 21 bp, Artificial Sequence
  Synthetic
  misc_feature (7)..(7): c is m5c
  misc_feature (10)..(10): c is m5c
  misc_feature (16)..(16): c is m5c
  misc_feature (19)..(19): c is m5c
gagttcccccaaatacttcac

SEQ ID NO: 769 - DNA, 21 bp, Artificial Sequence
  Synthetic
  misc_feature (8)..(8): c is m5c
  misc_feature (14)..(14): c is m5c
gagttcccccaaacacttcac

SEQ ID NO: 770 - DNA, 21 bp, Artificial Sequence
  Synthetic
  misc_feature (8)..(8): c is m5c
  misc_feature (14)..(14): c is m5c
gagttcccccaagcacttcac

SEQ ID NO: 771 - DNA, 21 bp, Artificial Sequence
  Synthetic
  misc_feature (7)..(7): c is m5c
  misc_feature (10)..(10): c is m5c
  misc_feature (16)..(16): c is m5c
  misc_feature (19)..(19): c is m5c
gatttcccccaaacgcttcac

SEQ ID NO: 772 - DNA, 21 bp, Artificial Sequence
  Synthetic
  misc_feature (8)..(8): c is m5c
  misc_feature (14)..(14): c is m5c
gagtttccccaaacacttcac

SEQ ID NO: 773 - DNA, 21 bp, Artificial Sequence
  Synthetic
  misc_feature (14)..(14): c is m5c
gagttcctccaaacacttcac

SEQ ID NO: 774 - DNA, 21 bp, Artificial Sequence
  Synthetic
  misc_feature (9)..(9): c is m5c
  misc_feature (12)..(12): c is m5c
catcaacgctgcactcaatga

SEQ ID NO: 775 - DNA, 21 bp, Artificial Sequence
  Synthetic
  misc_feature (18)..(18): c is m5c
caggggcgacctgagaacaca

SEQ ID NO: 776 - DNA, 21 bp, Artificial Sequence
  Synthetic
  misc_feature (11)..(11): c is m5c
  misc_feature (14)..(14): c is m5c
  misc_feature (20)..(20): c is m5c
cagggcaattctcctttggca

SEQ ID NO: 777 - DNA, 21 bp, Artificial Sequence
  Synthetic
  misc_feature (11)..(11): c is m5c
  misc_feature (14)..(14): c is m5c
  misc_feature (20)..(20): c is m5c
ctgggcaaatctcctttggca

SEQ ID NO: 778 - DNA, 21 bp, Artificial Sequence
  Synthetic
  misc_feature (6)..(6): c is m5c
  misc_feature (9)..(9): c is m5c
cagggcagctctcctttggca

SEQ ID NO: 779 - DNA, 21 bp, Artificial Sequence
  Synthetic
  misc_feature (6)..(6): c is m5c
  misc_feature (9)..(9): c is m5c
tagggcaactctcctttggca

SEQ ID NO: 780 - DNA, 21 bp, Artificial Sequence
  Synthetic
  misc_feature (13)..(13): c is m5c
cggggcaaatttcctttggca

SEQ ID NO: 781 - DNA, 21 bp, Artificial Sequence
  Synthetic
  misc_feature (2)..(2): c is m5c
  misc_feature (14)..(14): c is m5c
gccagggaagaggcagtgcag

SEQ ID NO: 782 - DNA, 21 bp, Artificial Sequence
  Synthetic
  misc_feature (4)..(4): c is m5c
gtgcagaacaagtgaaactag

SEQ ID NO: 783 - DNA, 21 bp, Artificial Sequence
  Synthetic
ggacgatgatgatgaggatga

SEQ ID NO: 784 - DNA, 21 bp, Artificial Sequence
  Synthetic
  misc_feature (4)..(4): c is m5c
ctgctctgcttctgttctgtt

SEQ ID NO: 785 - DNA, 21 bp, Artificial Sequence
  Synthetic
  misc_feature (12)..(12): c is m5c
tgaacagtgtacggacgaaca

SEQ ID NO: 786 - DNA, 21 bp, Artificial Sequence
  Synthetic
  misc_feature (6)..(6): c is m5c
  misc_feature (12)..(12): c is m5c
tggaacaaattcactacattc

SEQ ID NO: 787 - DNA, 21 bp, Artificial Sequence
  Synthetic
  misc_feature (9)..(9): c is m5c
  misc_feature (12)..(12): c is m5c
  misc_feature (15)..(15): c is m5c
cgtcatttcatcatcacatta

SEQ ID NO: 788 - DNA, 21 bp, Artificial Sequence
  Synthetic
atcatttccgtgattaattta

SEQ ID NO: 789 - DNA, 21 bp, Artificial Sequence
  Synthetic
  misc_feature (13)..(13): c is m5c
  misc_feature (16)..(16): c is m5c
cgtaaggacaaacttccaaaa

SEQ ID NO: 790 - DNA, 22 bp, Artificial Sequence
  Synthetic
agaaaggcagcaggtcgtatag

SEQ ID NO: 791 - DNA, 22 bp, Artificial Sequence
  Synthetic
acagctggttgaaggggaccaa

SEQ ID NO: 792 - DNA, 21 bp, Artificial Sequence
  Synthetic
  misc_feature (3)..(3): c is m5c
  misc_feature (18)..(18): c is m5c
ccctctggtcaaccagtcaca

SEQ ID NO: 793 - DNA, 24 bp, Artificial Sequence
  Synthetic
  misc_feature (4)..(4): c is m5c
  misc_feature (16)..(16): c is m5c
tgtccgtggttctaccctgtggta

SEQ ID NO: 794 - DNA, 22 bp, Artificial Sequence
  Synthetic
  misc_feature (2)..(2): c is m5c
  misc_feature (11)..(11): c is m5c
  misc_feature (14)..(14): c is m5c
cctcaaggagcctcagtctagt

SEQ ID NO: 795 - DNA, 22 bp, Artificial Sequence
  Synthetic
tactagactgtgagctcctcga

SEQ ID NO: 796 - DNA, 21 bp, Artificial Sequence
  Synthetic
ggctgtcaattcataggtcag

SEQ ID NO: 797 - DNA, 21 bp, Artificial Sequence
  Synthetic
tgtaagtgctcgtaatgcagt

SEQ ID NO: 798 - DNA, 22 bp, Artificial Sequence
  Synthetic
  misc_feature (20)..(20): c is m5c
ctagtggtcctaaacatttcac

SEQ ID NO: 799 - DNA, 23 bp, Artificial Sequence
  Synthetic
  misc_feature (5)..(5): c is m5c
  misc_feature (17)..(17): c is m5c
catgcatacatgcacacatacat

SEQ ID NO: 800 - DNA, 22 bp, Artificial Sequence
  Synthetic
  misc_feature (20)..(20): c is m5c
atgtatgtgggacggtaaacca

SEQ ID NO: 801 - DNA, 22 bp, Artificial Sequence
  Synthetic
  misc_feature (14)..(14): c is m5c
gaagagagcttgcccttgcata

SEQ ID NO: 802 - DNA, 25 bp, Artificial Sequence
  Synthetic
  misc_feature (15)..(15): c is m5c
  misc_feature (21)..(21): c is m5c
atgctttgacaatactattgcactg

SEQ ID NO: 803 - DNA, 19 bp, Artificial Sequence
  Synthetic
  misc_feature (4)..(4): c is m5c
  misc_feature (10)..(10): c is m5c
  misc_feature (13)..(13): c is m5c
accctcatgcccctcaagg

SEQ ID NO: 804 - DNA, 20 bp, Artificial Sequence
  Synthetic
  misc_feature (13)..(13): c is m5c
aaaagtaactagcacaccac

SEQ ID NO: 805 - DNA, 21 bp, Artificial Sequence
  Synthetic
agactagatatggaagggtga

SEQ ID NO: 806 - DNA, 20 bp, Artificial Sequence
  Synthetic
  misc_feature (7)..(7): c is m5c
tctgggcacacggagggaga

SEQ ID NO: 807 - DNA, 23 bp, Artificial Sequence
  Synthetic
  misc_feature (2)..(2): c is m5c
acggtcaggctttggctagatca

SEQ ID NO: 808 - DNA, 23 bp, Artificial Sequence
  Synthetic
  misc_feature (14)..(14): c is m5c
agaggcaggcactcaggcagaca

SEQ ID NO: 809 - DNA, 19 bp, Artificial Sequence
  Synthetic
  misc_feature (10)..(10): c is m5c
tgggcgacccagagggaca

SEQ ID NO: 810 - DNA, 22 bp, Artificial Sequence
  Synthetic
agaggttaagacagcagggctg

SEQ ID NO: 811 - DNA, 23 bp, Artificial Sequence
  Synthetic
  misc_feature (3)..(3): c is m5c
  misc_feature (6)..(6): c is m5c
  misc_feature (18)..(18): c is m5c
  misc_feature (21)..(21): c is m5c
aacaaccagctaagacactgcca

SEQ ID NO: 812 - DNA, 23 bp, Artificial Sequence
  Synthetic
caatcagctaattacactgccta

SEQ ID NO: 813 - DNA, 24 bp, Artificial Sequence
  Synthetic
  misc_feature (7)..(7): c is m5c
  misc_feature (10)..(10): c is m5c
  misc_feature (19)..(19): c is m5c
  misc_feature (22)..(22): c is m5c
cgcaatcagctaactacactgcct

SEQ ID NO: 814 - DNA, 24 bp, Artificial Sequence
  Synthetic
gtgaaagtgtatgggctttgtgaa

SEQ ID NO: 815 - DNA, 21 bp, Artificial Sequence
  Synthetic
  misc_feature (3)..(3): c is m5c
  misc_feature (12)..(12): c is m5c
  misc_feature (15)..(15): c is m5c
  misc_feature (18)..(18): c is m5c
tactatgcaacctactactct

SEQ ID NO: 816 - DNA, 21 bp, Artificial Sequence
  Synthetic
caacaaacatttaatgaggcc

SEQ ID NO: 817 - DNA, 22 bp, Artificial Sequence
  Synthetic
  misc_feature (10)..(10): c is m5c
tatggcagactgtgatttgttg

SEQ ID NO: 818 - DNA, 20 bp, Artificial Sequence
  Synthetic
  misc_feature (7)..(7): c is m5c
tggatgcagaggtttatcga

SEQ ID NO: 819 - DNA, 20 bp, Artificial Sequence
  Synthetic
gggaatgaagcctggtccga

SEQ ID NO: 820 - DNA, 20 bp, Artificial Sequence
  Synthetic
  misc_feature (10)..(10): c is m5c
  misc_feature (13)..(13): c is m5c
  misc_feature (19)..(19): c is m5c
atattggcgcggctcaatca

SEQ ID NO: 821 - DNA, 20 bp, Artificial Sequence
  Synthetic
  misc_feature (13)..(13): c is m5c
  misc_feature (16)..(16): c is m5c
gtgatattggcacggctcaa

SEQ ID NO: 822 - DNA, 20 bp, Artificial Sequence
  Synthetic
  misc_feature (3)..(3): c is m5c
  misc_feature (6)..(6): c is m5c
  misc_feature (9)..(9): c is m5c
  misc_feature (12)..(12): c is m5c
tgcagcatcatcaagattct

SEQ ID NO: 823 - DNA, 21 bp, Artificial Sequence
  Synthetic
  misc_feature (3)..(3): c is m5c
cgcgtaccaaaagtaataatg

SEQ ID NO: 824 - DNA, 21 bp, Artificial Sequence
  Synthetic
  misc_feature (6)..(6): c is m5c
  misc_feature (9)..(9): c is m5c
  misc_feature (12)..(12): c is m5c
tagtacatcatctatactgta

SEQ ID NO: 825 - DNA, 21 bp, Artificial Sequence
  Synthetic
  misc_feature (4)..(4): c is m5c
  misc_feature (7)..(7): c is m5c
gagctacagtgcttcatctca

SEQ ID NO: 826 - DNA, 22 bp, Artificial Sequence
  Synthetic
  misc_feature (11)..(11): c is m5c
taacgcgagccgaacgaacaaa

SEQ ID NO: 827 - DNA, 23 bp, Artificial Sequence
  Synthetic
  misc_feature (2)..(2): c is m5c
  misc_feature (14)..(14): c is m5c
actgttatcagctcagtaggcac

SEQ ID NO: 828 - DNA, 18 bp, Artificial Sequence
  Synthetic
  misc_feature (6)..(6): c is m5c
  misc_feature (12)..(12): c is m5c
agacacatgcactgtaga

SEQ ID NO: 829 - DNA, 21 bp, Artificial Sequence
  Synthetic
  misc_feature (2)..(2): c is m5c
  misc_feature (11)..(11): c is m5c
  misc_feature (14)..(14): c is m5c
  misc_feature (17)..(17): c is m5c
  misc_feature (20)..(20): c is m5c
actgtacaaacaactacctca

SEQ ID NO: 830 - DNA, 21 bp, Artificial Sequence
  Synthetic
  misc_feature (10)..(10): c is m5c
gcatcgttaccagacagtgtt

SEQ ID NO: 831 - DNA, 21 bp, Artificial Sequence
  Synthetic
  misc_feature (2)..(2): c is m5c
  misc_feature (11)..(11): c is m5c
gccaatatttctgtgctgcta

SEQ ID NO: 832 - DNA, 22 bp, Artificial Sequence
  Synthetic
  misc_feature (13)..(13): c is m5c
  misc_feature (16)..(16): c is m5c
acagcaaacatccaactgaaag

SEQ ID NO: 833 - DNA, 23 bp, Artificial Sequence
  Synthetic
  misc_feature (6)..(6): c is m5c
  misc_feature (9)..(9): c is m5c
  misc_feature (12)..(12): c is m5c
  misc_feature (15)..(15): c is m5c
  misc_feature (21)..(21): c is m5c
acaaacaccaccgtcacactcca

SEQ ID NO: 834 - DNA, 23 bp, Artificial Sequence
  Synthetic
  misc_feature (6)..(6): c is m5c
  misc_feature (9)..(9): c is m5c
  misc_feature (12)..(12): c is m5c
  misc_feature (15)..(15): c is m5c
  misc_feature (21)..(21): c is m5c
acaaacaccatcgtcacactcca

SEQ ID NO: 835 - DNA, 20 bp, Artificial Sequence
  Synthetic
  misc_feature (2)..(2): c is m5c
  misc_feature (8)..(8): c is m5c
  misc_feature (11)..(11): c is m5c
gcattcaccgcgtgccttaa

SEQ ID NO: 836 - DNA, 18 bp, Artificial Sequence
  Synthetic
  misc_feature (6)..(6): c is m5c
  misc_feature (9)..(9): c is m5c
attcaccgcgtgccttaa

SEQ ID NO: 837 - DNA, 16 bp, Artificial Sequence
  Synthetic
  misc_feature (4)..(4): c is m5c
  misc_feature (7)..(7): c is m5c
tcaccgcgtgccttaa

SEQ ID NO: 838 - DNA, 12 bp, Artificial Sequence
  Synthetic
  misc_feature (3)..(3): c is m5c
cgcgtgccttaa

SEQ ID NO: 839 - DNA, 10 bp, Artificial Sequence
  Synthetic
  misc_feature (1)..(1): c is m5c
cgtgccttaa

SEQ ID NO: 840 - DNA, 8 bp, Artificial Sequence
  Synthetic
tgccttaa

SEQ ID NO: 841 - DNA, 20 bp, Artificial Sequence
  Synthetic
  misc_feature (2)..(2): c is m5c
  misc_feature (14)..(14): c is m5c
acacacttccttacattcca

SEQ ID NO: 842 - DNA, 18 bp, Artificial Sequence
  Synthetic
  misc_feature (12)..(12): c is m5c
acacttccttacattcca

SEQ ID NO: 843 - DNA, 16 bp, Artificial Sequence
  Synthetic
  misc_feature (10)..(10): c is m5c
acttccttacattcca

SEQ ID NO: 844 - DNA, 14 bp, Artificial Sequence
  Synthetic
  misc_feature (8)..(8): c is m5c
ttccttacattcca

SEQ ID NO: 845 - DNA, 12 bp, Artificial Sequence
  Synthetic
  misc_feature (6)..(6): c is m5c
ccttacattcca

SEQ ID NO: 846 - DNA, 10 bp, Artificial Sequence
  Synthetic
  misc_feature (4)..(4): c is m5c
ttacattcca

SEQ ID NO: 847 - DNA, 8 bp, Artificial Sequence
  Synthetic
  misc_feature (2)..(2): c is m5c
acattcca

SEQ ID NO: 848 - DNA, 14 bp, Artificial Sequence
  Synthetic
  misc_feature (2)..(2): c is m5c
  misc_feature (5)..(5): c is m5c
accgcgtaccttaa

SEQ ID NO: 849 - DNA, 14 bp, Artificial Sequence
  Synthetic
ttccttaaattcca

SEQ ID NO: 850 - DNA, 19 bp, Artificial Sequence
  Synthetic
  misc_feature (5)..(5): c is m5c
  misc_feature (8)..(8): c is m5c
  misc_feature (17)..(17): c is m5c
catacttctttacattcca

SEQ ID NO: 851 - DNA, 18 bp, Artificial Sequence
  Synthetic
  misc_feature (7)..(7): c is m5c
tcaaagctggctgtgata

SEQ ID NO: 852 - DNA, 18 bp, Artificial Sequence
  Synthetic
  misc_feature (6)..(6): c is m5c
tcacacttgaggtctcag

SEQ ID NO: 853 - DNA, 13 bp, Artificial Sequence
  Synthetic
  misc_feature (2)..(2): c is m5c
  misc_feature (5)..(5): c is m5c
accgcgtgcctta

SEQ ID NO: 854 - DNA, 20 bp, Artificial Sequence
  Synthetic
tgctttgtctacatttgggg

SEQ ID NO: 855 - DNA, 21 bp, Artificial Sequence
  Synthetic
ctgggcaagtctcctttggca

SEQ ID NO: 856 - DNA, 21 bp, Artificial Sequence
  Synthetic
  misc_feature (7)..(7): c is m5c
actatgcaacctactacctct

SEQ ID NO: 857 - DNA, 22 bp, Artificial Sequence
  Synthetic
  misc_feature (5)..(5): c is m5c
ggtacaatcaacggtcgatggt

SEQ ID NO: 858 - DNA, 21 bp, Artificial Sequence
  Synthetic
  misc_feature (4)..(4): c is m5c
  misc_feature (7)..(7): c is m5c
  misc_feature (10)..(10): c is m5c
  misc_feature (13)..(13): c is m5c
caacatcgtcagcattcatca

SEQ ID NO: 859 - DNA, 17 bp, Artificial Sequence
  Synthetic
gggactttgtaggccag

SEQ ID NO: 860 - DNA, 18 bp, Artificial Sequence
  Syntehtic
  misc_feature (12)..(12): c is m5c
gaatcttgtcccgcaggt

SEQ ID NO: 861 - DNA, 18 bp, Artificial Sequence
  Synthetic
ggactttgagggccagtt

SEQ ID NO: 862 - DNA, 17 bp, Artificial Sequence
  Synthetic
  misc_feature (5)..(5): c is m5c
  misc_feature (8)..(8): c is m5c
agtgcttccctccagag

SEQ ID NO: 863 - DNA, 19 bp, Artificial Sequence
  Synthetic
  misc_feature (12)..(12): c is m5c
  misc_feature (15)..(15): c is m5c
agtgcttcttacctccaga

SEQ ID NO: 864 - DNA, 17 bp, Artificial Sequence
  Synthetic
  misc_feature (9)..(9): c is m5c
aactgttcccgctgcta

SEQ ID NO: 865 - DNA, 20 bp, Artificial Sequence
  Synthetic
  misc_feature (6)..(6): c is m5c
  misc_feature (12)..(12): c is m5c
  misc_feature (18)..(18): c is m5c
ttttcccatgccctatacct

SEQ ID NO: 866 - DNA, 18 bp, Artificial Sequence
  Synthetic
  misc_feature (6)..(6): c is m5c
  misc_feature (9)..(9): c is m5c
  misc_feature (12)..(12): c is m5c
aatgacacctccctgtga

SEQ ID NO: 867 - DNA, 16 bp, Artificial Sequence
  Synthetic
  misc_feature (11)..(11): c is m5c
gaaaaacgccccctgg

SEQ ID NO: 868 - DNA, 16 bp, Artificial Sequence
  Synthetic
  misc_feature (11)..(11): c is m5c
gaaaaacgccccctgg

SEQ ID NO: 869 - DNA, 18 bp, Artificial Sequence
  Synthetic
  misc_feature (11)..(11): c is m5c
aagtgcttccctcaagag

SEQ ID NO: 870 - DNA, 18 bp, Artificial Sequence
  Synthetic
  misc_feature (5)..(5): c is m5c
agtgctttcttttggaga

SEQ ID NO: 871 - DNA, 19 bp, Artificial Sequence
  Synthetic
  misc_feature (2)..(2): c is m5c
tctttgcagatgagactga

SEQ ID NO: 872 - DNA, 20 bp, Artificial Sequence
  Synthetic
  misc_feature (11)..(11): c is m5c
cttcagttatcacagtactg

SEQ ID NO: 873 - DNA, 16 bp, Artificial Sequence
  Synthetic
  misc_feature (2)..(2): c is m5c
tcctgggaaaactgga

SEQ ID NO: 874 - DNA, 18 bp, Artificial Sequence
  Syntetic
  misc_feature (3)..(3): c is m5c
  misc_feature (12)..(12): c is m5c
caccattgtcacactcca

SEQ ID NO: 875 - DNA, 19 bp, Artificial Sequence
  Synthetic
  misc_feature (10)..(10): c is m5c
gaaagagaccggttcactg

SEQ ID NO: 876 - DNA, 16 bp, Artificial Sequence
  Synthetic
  misc_feature (16)..(16): c is m5c
agtgaagacacggagc

SEQ ID NO: 877 - DNA, 21 bp, Artificial Sequence
  Synthetic
  misc_feature (2)..(2): c is m5c
  misc_feature (5)..(5): c is m5c
  misc_feature (8)..(8): c is m5c
ccatcatcaaaacaaatggag

SEQ ID NO: 878 - DNA, 23 bp, Artificial Sequence
  Synthetic
  misc_feature (10)..(10): c is m5c
  misc_feature (17)..(17): c is m5c
tcagctattccgacatcttgcca

SEQ ID NO: 879 - DNA, 23 bp, Artificial Sequence
  Synthetic
  misc_feature (12)..(12): c is m5c
gaacaggtagtctaaacactggg

SEQ ID NO: 880 - DNA, 14 bp, Artificial Sequence
  Synthetic
  misc_feature (2)..(2): c is m5c
  misc_feature (5)..(5): c is m5c
accgcgtgccttaa